SEQUENCE LISTING (22 sequences)

SEQ ID NO: 1 - protein, 11 aa, Mus sp.
KASRDVAIAVA

SEQ ID NO: 2 - protein, 7 aa, Mus sp.
WASTRHT

SEQ ID NO: 3 - protein, 9 aa, Mus sp.
HQYSSYPFT

SEQ ID NO: 4 - protein, 10 aa, Mus sp.
GNTFTDQTIH

SEQ ID NO: 5 - protein, 17 aa, Mus sp.
YIYPRDDSPKYNENFKG

SEQ ID NO: 6 - protein, 11 aa, Mus sp.
PDRSGYAWFIY

SEQ ID NO: 7 - protein, 10 aa, synthetic construct
  REGION 1..10: note = Description of Artificial Sequence: Humanized sequence
GYTFTDQTIH

SEQ ID NO: 8 - protein, 10 aa, synthetic construct
  REGION 1..10: note = Description of Artificial Sequence: Humanized sequence
GFTFTDQTIH

SEQ ID NO: 9 - protein, 10 aa, synthetic construct
  REGION 1..10: note = Description of Artificial Sequence: Humanized sequence
GGTFTDQTIH

SEQ ID NO: 10 - protein, 107 aa, synthetic construct
  REGION 1..107: note = Description of Artificial Sequence: Humanized sequence
DIQMTQSPSSLSASVGDRVTITCKASRDVAIAVAWYQQKPGKVPKLLLFWASTRHTGVPDRFSGSGSGTDFTLTISSLQPEDLADYYCHQYSSYPFTFGQGTKLEIK

SEQ ID NO: 11 - protein, 107 aa, synthetic construct
  REGION 1..107: note = Description of Artificial Sequence: Humanized sequence
DIQMTQSPSSLSASVGDRVTITCKASRDVAIAVAWYQQKPGKVPKLLIYWASTRHTGVPSRFSGSGSRTDFTLTISSLQPEDVADYFCHQYSSYPFTFGSGTKLEIK

SEQ ID NO: 12 - protein, 107 aa, synthetic construct
  REGION 1..107: note = Description of Artificial Sequence: Humanized sequence
DIQMTQSPSSLSASVGDRVTITCKASRDVAIAVAWYQQKPGKVPKLLLYWASTRHTGVPSRFSGSGSRTDFTLTISSLQPEDVATYYCHQYSSYPFTFGSGTKLEIK

SEQ ID NO: 13 - protein, 107 aa, synthetic construct
  REGION 1..107: note = Description of Artificial Sequence: Humanized sequence
DIQMTQSPSSLSASVGDRVTITCKASRDVAIAVAWYQQKPGKVPKLLLFWASTRHTGVPDRFSGSGSRTDFTLTISSLQPEDLADYYCHQYSSYPFTFGSGTKLEIK

SEQ ID NO: 14 - protein, 120 aa, synthetic construct
  REGION 1..120: note = Description of Artificial Sequence: Humanized sequence
QVQLVQSGAEVKKPGSSVKVSCKASGYTFTDQTIHWMRQAPGQGLEWIGYIYPRDDSPKYNENFKGKVTITADKSTSTAYMELSSLRSEDTAVYYCAIPDRSGYAWFIYWGQGTLVTVSS

SEQ ID NO: 15 - protein, 120 aa, synthetic construct
  REGION 1..120: note = Description of Artificial Sequence: Humanized sequence
QVQLVQSGAEVKKPGSSVKVSCKASGFTFTDQTIHWVRQAPGQGLEWMGYIYPRDDSPKYNENFKGKVTLTADKSTSTAYMELSSLRSEDTAVYYCAIPDRSGYAWFIYWGQGTLVTVSS

SEQ ID NO: 16 - protein, 120 aa, synthetic construct
  REGION 1..120: note = Description of Artificial Sequence: Humanized sequence
QVQLVQSGAEVKKPGSSVKTSCKASGGTFTDQTIHWVRQRPGQGLEWMGYIYPRDDSPKYNENFKGRVTITADKSTSTAYMELSSLRSEDTAVYYCAIPDRSGYAWFIYWGQGTLVTVSS

SEQ ID NO: 17 - protein, 120 aa, synthetic construct
  REGION 1..120: note = Description of Artificial Sequence: Humanized sequence
QVQLVQSGAEVKKPGSSVKVSCKASGGTFTDQTIHWVRQAPGQGLEWMGYIYPRDDSPKYNENFKGRVTLTADKSTSTAYMELSSLRSEDTAVYFCARPDRSGYAWFIYWGQGTLVTVSS

SEQ ID NO: 18 - protein, 214 aa, synthetic construct
  REGION 1..214: note = Description of Artificial Sequence: Humanized sequence
DIQMTQSPSSLSASVGDRVTITCKASRDVAIAVAWYQQKPGKVPKLLIYWASTRHTGVPSRFSGSGSRTDFTLTISSLQPEDVADYFCHQYSSYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 449 aa, synthetic construct
  REGION 1..449: note = Description of Artificial Sequence: Humanized sequence
QVQLVQSGAEVKKPGSSVKVSCKASGYTFTDQTIHWMRQAPGQGLEWIGYIYPRDDSPKYNENFKGKVTITADKSTSTAYMELSSLRSEDTAVYYCAIPDRSGYAWFIYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 20 - protein, 449 aa, synthetic construct
  REGION 1..449: note = Description of Artificial Sequence: Humanized sequence
QVQLVQSGAEVKKPGSSVKVSCKASGFTFTDQTIHWVRQAPGQGLEWMGYIYPRDDSPKYNENFKGKVTLTADKSTSTAYMELSSLRSEDTAVYYCAIPDRSGYAWFIYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 21 - protein, 214 aa, synthetic construct
  REGION 1..214: note = Description of Artificial Sequence: Humanized sequence
DIQMTQSPSSLSASVGDRVTITCKASRDVAIAVAWYQQKPGKVPKLLLFWASTRHTGVPDRFSGSGSGTDFTLTISSLQPEDLADYYCHQYSSYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 189 aa, Homo sapiens
MLGSRAVMLLLLLPWTAQGRAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSP

(19) United States
(12) Patent Application Publication
(10) Pub. No.: US 2026/0308246 A1
(43) Pub. Date: Oct. 8, 2026

(54) METHODS OF TREATING INFLAMMATORY DISEASES

(71) Applicant: BOEHRINGER INGELHEIM INTERNATIONAL GMBH, Ingelheim am Rhein (DE)

(72) Inventors: Patrick BAUM, Biberach an der Riss (DE); Mary Ruth FLACK, Ridgefield, CT (US); Annette Bettina GALLER, Armsheim (DE); Bojan LALOVIC, Cedar Knolls, NJ (US); Kaori MAINO, Tokyo (JP); Steven John PADULA, Southbury, CT (US); Paul SCHOLL, Redding, CT (US); Koji SHA, Tokyo (JP); Sudha VISVANATHAN, Hartsdale, NY (US); Patrick GARIDEL, Biberach an der Riss (DE); Torsten SCHULTZ-FADEMRECHT, Aepfingen (DE)

(73) Assignee: BOEHRINGER INGELHEIM INTERNATIONAL GMBH, Ingelheim am Rhein (DE)

(21) Appl. No.: 19/716,936

(22) Filed: Jun. 23, 2026

Related U.S. Application Data
(63) Continuation-in-part of application No. 18/941,883, filed on Nov. 8, 2024.
Application No. 18/941,883 is a continuation of application No. 16/724,225, filed on Dec. 21, 2019, now abandoned.
Application No. 16/724,225 is a continuation of application No. 15/486,448, filed on Apr. 13, 2017, now abandoned.
(60) Provisional application No. 62/323,075, filed on Apr. 15, 2016.
(60) Provisional application No. 62/336,815, filed on May 16, 2016.
(60) Provisional application No. 62/396,855, filed on Sep. 20, 2016.
(60) Provisional application No. 62/400,116, filed on Sep. 27, 2016.
(60) Provisional application No. 62/463,959, filed on Feb. 27, 2017.

(51) Int. Cl. C07K 16/24; A61K 39/00; A61K 39/395; A61P 17/06; C12Q 1/6883

(52) U.S. Cl. CPC C07K 16/244; A61K 39/395; A61P 17/06; C12Q 1/6883; A61K 2039/54; C07K 2317/21; C07K 2317/24; C07K 2317/76; C07K 2317/94; C12Q 2600/118; C12Q 2600/158

(57) ABSTRACT

This invention generally relates to methods for the treatment of IL-23 related diseases, in particular inflammatory diseases, such as psoriasis or psoriatic arthritis, utilizing anti-IL-23A antibodies. Also disclosed herein are formulations comprising anti-IL-23p19 antibodies, such as risankizumab, which bind the p19 subunit of human IL-23. More specifically, pharmaceutical formulations comprising a high concentration of the anti-IL-23p19 antibody risankizumab, as well as related products and uses for the treatment of various diseases and disorders, are disclosed.

### CROSS-REFERENCE TO RELATED APPLICATIONS

This application is a Continuation In Part of application Ser. No. 18/941,883, filed Nov. 8, 2024, which is a Continuation of application Ser. No. 16/724,225, filed Dec. 21, 2019, which is a Continuation of application Ser. No. 15/486,448, filed Apr. 13, 2017, which claims priority from U.S. Provisional Application Nos. 62/323,075, filed Apr. 15, 2016, 62/336,815, filed May 16, 2016, 62/396,855, filed Sep. 20, 2016, 62/400,116, filed Sep. 27, 2016, and 62/463,959, filed Feb. 27, 2017, each of which is incorporated herein by reference in its entirety.

### SEQUENCE LISTING

The instant application contains a Sequence Listing which has been submitted in XML format via Patent Center and is hereby incorporated by reference in its entirety. Said XML copy, created on May 1, 2026, is named 114578-0168.xml and is 28,535 bytes.

### TECHNICAL FIELD OF THE INVENTION

This invention generally relates to method of treating inflammatory diseases, for example psoriasis, psoriatic arthritis or axial (spinal) spondyloarthritis (ax-SpA), including ankylosing spondylitis (AS, also called radiographic ax-SpA) and non-radiographic ax-SpA, utilizing anti-IL-23A antibodies. This invention also generally relates to formulations comprising anti-IL-23p19 antibodies, such as risankizumab, which bind the p19 subunit of human IL-23. More specifically, pharmaceutical formulations comprising a high concentration of the anti-IL-23p19 antibody risankizumab, as well as related products and uses for the treatment of various diseases and disorders, are disclosed. Disclosed herein are stable liquid pharmaceutical formulations, comprising 150 mg/ml of the antibody risankizumab.

### BACKGROUND OF THE INVENTION

Human IL-23 is composed of a common subunit (p40) with IL-12 and a unique p19 subunit. Despite this shared p40 subunit, the roles for IL-23 and IL-12 are quite different. IL-12 is important for Th1 responses via promotion of Th1 cell differentiation, proliferation and activation. In contrast, IL-23 supports the development and maintenance of a set of CD4⁺ T helper cells termed Th17 cells due to their ability to produce IL-17 and related cytokines. IL-23 is involved in chronic autoimmune inflammation and the modulation of IL-23 activity provides effective therapies against autoimmune diseases.

One of the autoimmune diseases where IL-23 plays a central role is psoriasis, a chronic, immune-mediated inflammatory disease characterized by the hyper proliferation of keratinocytes and skin-infiltrating T-lymphocytes that overexpress pro-inflammatory mediators. The disease is a chronic, painful immune-mediated inflammatory skin disease and has a lifelong remitting and relapsing course with varying factors that trigger exacerbations in susceptible individuals, thus making treatment challenging. The uncontrolled inflammation of psoriasis may contribute to commonly associated comorbidities, including cardiovascular (CV) disease (including hypertension and increased risk for myocardial infarction, stroke, and CV death), obesity, type 2 diabetes, arthritis, and chronic renal disease. Psoriasis is also associated with serious psychiatric comorbidities, including depression, anxiety, and suicidality, as well as substance abuse.

Psoriasis is a chronic, immune-mediated, inflammatory skin disease, with a global incidence of approximately 2%, associated with significant morbidity and can have a substantial psychosocial impact on quality of life and well-being of patients. Plaque psoriasis is the most common form and affects approximately 80-90% of patients, manifesting as raised plaques on the skin; the disease usually begins in late adolescence and early adulthood and may persist through adult life. The extent of the affected body surface area (BSA) and the degree of skin manifestations, including erythema, induration, and scaling, defines the severity of psoriasis with approximately 20-30% of patients having moderate-to-severe disease. Approximately one third of patients with psoriasis have associated joint inflammation (psoriatic arthritis (PsA)) resulting in pain and disability. Axial (spinal) spondyloarthritis (ax-SpA), including ankylosing Spondylitis (AS, also called radiographic axial spondyloarthritis (ax-SpA)) and non-radiographic axial spondyloarthritis (ax-SpA), are an inflammatory disease involving primarily the axial skeleton and sacroiliac joints.

Psoriasis, psoriatic arthritis and axial (spinal) spondyloarthritis (ax-SpA), which includes ankylosing spondylitis and non-radiographic ax-SpA, are multifactorial autoimmune diseases whose exact aetiology is unknown. Multiple genome-wide association studies have linked variants in the genes for the IL-23 receptor to psoriasis susceptibility. Human IL-23 is primarily produced by antigen presenting cells and induces differentiation of T helper 17 (Th17) cells. This results in the production of IL-17 and IL-22, which may mediate the development of the epidermal hyperplasia and tissue inflammation observed in psoriasis.

There is a need for treatment options for inflammatory diseases, in particular psoriasis, psoriatic arthritis, axial (spinal) spondyloarthritis (ax-SpA), including ankylosing spondylitis and non-radiographic ax-SpA, that lead to favorable outcomes for patients, for example in terms of efficacy, safety and/or tolerability of the treatment.

A highly efficient and specific inhibitor of IL-23 is the antibody risankizumab. Risankizumab is a humanized immunoglobulin G1 (IgG1) monoclonal antibody that is directed against the p19 subunit of IL-23. Binding of risankizumab to IL-23 p19 inhibits the action of IL-23 to induce and sustain T helper (Th) 17 type cells, innate lymphoid cells, γδT cells, and natural killer (NK) cells responsible for tissue inflammation, destruction and aberrant tissue repair. Risankizumab is especially effective in the treatment of autoimmune and inflammatory diseases, in particular psoriasis. Clinical studies revealed excellent safety and efficacy of risankizumab in the treatment of plaque psoriasis. The recommended dose approved for treatment of psoriasis is 150 mg which is administered subcutaneously as two 75 mg injections, on week 0, 4 and thereafter every 12 weeks.

The requirement for injection of larger medicament volumes presents a challenge, especially in patients with chronic conditions who have markedly lower rates of drug adherence and persistence than patients with acute conditions. Administration by subcutaneous route is preferred for therapeutic indications where home (self) medication is desirable, for example, for chronic diseases such as psoriasis. The subcutaneous administration route is, however, limited by the injection volume as attributable to tissue backpressure and injection pain. This also depends on the injected formulation. Most drugs that are administered by subcutaneous injection, such as risankizumab, are commonly used in unit dosages with volumes not exceeding 1 ml. Therefore, for higher volumes, such as greater than 2 ml, multiple injections are typically used, but this approach may increase the attrition rate or reduce patient adherence.

Therefore, to allow the administration of a high dose of antibody such as risankizumab with a single injection, there is a need for pharmaceutical formulations with increased antibody concentration. However, increasing the antibody concentration in antibody formulations can cause problems with stability, e.g. aggregation resulting in the formation of high molecular weight species (HMWS) and increased viscosity. Therefore, it is a great challenge to provide a stable high concentration liquid antibody formulation that is suitable for parenteral administration, such as subcutaneous injection.

### SUMMARY OF THE INVENTION

The present invention addresses the above needs and provides methods for treating inflammatory diseases, in particular methods comprising administering an anti-IL-23A antibody to a patient in certain amounts and/or at certain intervals. In one aspect, a method of the present invention is for the treatment of psoriasis or psoriatic arthritis. In one aspect, a method of the present invention is for the treatment of axial (spinal) spondyloarthritis (ax-SpA), including ankylosing spondylitis (AS) and non-radiographic ax-SpA. Accordingly, in one aspect, a method of the present invention is for the treatment of ankylosing spondylitis (AS) and in one aspect, a method of the present invention is for the treatment of non-radiographic ax-SpA.

The methods of the present invention provide the advantage of enabling patients to experience clinical improvement while receiving fewer administrations of the anti-IL-23A antibody.

In one embodiment, the present invention provides a method for treating an inflammatory disease, in one aspect for treating psoriasis, psoriatic arthritis or axial (spinal) spondyloarthritis (ax-SpA), including ankylosing spondylitis and non-radiographic ax-SpA, comprising administering to a patient an anti-IL-23A antibody, said method comprising:
a) administering an initial dose of the anti-IL-23A antibody to the patient;
b) administering a first maintenance dose of the anti-IL-23A antibody to the patient 4 to 24 weeks, for example 4 to 16 weeks, for example 4 to 12 weeks, after said initial dose is administered; and
c) administering at least one additional maintenance dose to the patient 4 to 24 weeks, for example 4 to 16 weeks, for example 4 to 12 weeks, after said first maintenance dose is administered.

In one embodiment, the present invention provides a method for treating an inflammatory disease, in one aspect for treating psoriasis, psoriatic arthritis or axial (spinal) spondyloarthritis (ax-SpA), including ankylosing spondylitis and non-radiographic ax-SpA, comprising administering to a patient an anti-IL-23A antibody, said method comprising:
a) administering an initial dose of the anti-IL-23A antibody to the patient;
b) administering a first maintenance dose of the anti-IL-23A antibody to the patient 8 to 24 weeks, for example 8 to 16 weeks, for example 8 to 12 weeks, for example 6 to 24 weeks, for example 6 to 16 weeks, for example 6 to 12 weeks, after said initial dose is administered; and
c) administering at least one additional maintenance dose to the patient 8 to 24 weeks, for example 8 to 16 weeks, for example 8 to 12 weeks, for example 6 to 24 weeks, for example 6 to 16 weeks, for example 6 to 12 weeks, after said first maintenance dose is administered.

In one embodiment, the first maintenance dose is administered to the patient 4, 6, 8, 12, 16, 20 or 24 weeks after the initial dose is administered.

In one embodiment, the at least one additional maintenance dose is administered to the patient 4, 6, 8, 12, 16, 20 or 24 weeks after the first maintenance dose is administered.

In one embodiment, the first maintenance dose is administered to the patient 4, 6, 8, 12, 16, 20 or 24 weeks after the initial dose is administered and the at least one additional maintenance dose is administered to the patient 4, 6, 8, 12, 16, 20 or 24 weeks after the first maintenance dose is administered.

In additional embodiments:
the first maintenance dose is administered to the patient 4 weeks after the initial dose is administered and the at least one additional maintenance dose is administered to the patient 4 weeks after the first maintenance dose is administered; or
the first maintenance dose is administered to the patient 6 weeks after the initial dose is administered and the at least one additional maintenance dose is administered to the patient 6 weeks after the first maintenance dose is administered; or
the first maintenance dose is administered to the patient 8 weeks after the initial dose is administered and the at least one additional maintenance dose is administered to the patient 8 weeks after the first maintenance dose is administered; or
the first maintenance dose is administered to the patient 12 weeks after the initial dose is administered and the at least one additional maintenance dose is administered to the patient 12 weeks after the first maintenance dose is administered; or
the first maintenance dose is administered to the patient 16 weeks after the initial dose is administered and the at least one additional maintenance dose is administered to the patient 16 weeks after the first maintenance dose is administered; or
the first maintenance dose is administered to the patient 20 weeks after the initial dose is administered and the at least one additional maintenance dose is administered to the patient 20 weeks after the first maintenance dose is administered; or
the first maintenance dose is administered to the patient 24 weeks after the initial dose is administered and the at least one additional maintenance dose is administered to the patient 24 weeks after the first maintenance dose is administered.

In one embodiment, in any one of the methods above, the initial dose comprises 15 to 300 mg, for example 15 to 250 mg, for example 90 to 180 mg, of the anti-IL-23A antibody.

In one embodiment, in any one of the methods above, the first maintenance dose and the at least one additional maintenance dose comprise 15 to 300 mg, for example 15 to 250 mg, for example 90 to 180 mg, of the anti-IL-23A antibody.

In one embodiment, in any one of the methods above, the initial dose comprises 70 to 90 mg, 80 to 100 mg, 90 to 110 mg, 100 to 120 mg, 110 to 130 mg, 120 to 140 mg, 130 to 150 mg, 140 to 160 mg, 150 to 170 mg, 160 to 180 mg, 170 to 190 mg, 180 to 200 mg, 190 to 210 mg, 200 to 220 mg, 210 to 230 mg, 220 to 240 mg, 230 to 250 mg, 240 to 260 mg, 250 to 270 mg, 260 to 280 mg, 270 to 290 mg or 280 to 300 mg of the anti-IL-23A antibody.

In one embodiment, in any one of the methods above, the first maintenance dose and the at least one additional maintenance dose comprise 70 to 90 mg, 80 to 100 mg, 90 to 110 mg, 100 to 120 mg, 110 to 130 mg, 120 to 140 mg, 130 to 150 mg, 140 to 160 mg, 150 to 170 mg, 160 to 180 mg, 170 to 190 mg, 180 to 200 mg, 190 to 210 mg, 200 to 220 mg, 210 to 230 mg, 220 to 240 mg, 230 to 250 mg, 240 to 260 mg, 250 to 270 mg, 260 to 280 mg, 270 to 290 mg or 280 to 300 mg of the anti-IL-23A antibody.

In one embodiment, in any one of the methods above, the initial dose comprises 18, 20, 30, 40, 50, 60, 70, 80, 90, 100, 110, 120, 130, 140, 150, 160, 170, 180, 190, 200, 210, 220, 230, 240, 250, 260, 270, 280, 290 or 300 mg of the anti-IL-23A antibody. In one embodiment, in any one of the methods above, the initial dose comprises 75 mg of the anti-IL-23A antibody.

In one embodiment, in any one of the methods above, the first maintenance dose and the at least one additional maintenance dose comprise 18, 20, 30, 40, 50, 60, 70, 80, 90, 100, 110, 120, 130, 140, 150, 160, 170, 180, 190, 200, 210, 220, 230, 240, 250, 260, 270, 280, 290 or 300 mg of the anti-IL-23A antibody. In one embodiment, in any one of the methods above, the first maintenance dose and the at least one additional maintenance dose comprise 75 mg of the anti-IL-23A antibody.

In one embodiment, in any one of the methods above, the initial dose, the first maintenance dose and the at least one additional maintenance dose comprise 18, 20, 30, 40, 50, 60, 70, 80, 90, 100, 110, 120, 130, 140, 150, 160, 170, 180, 190, 200, 210, 220, 230, 240, 250, 260, 270, 280, 290 or 300 mg of the anti-IL-23A antibody. In one embodiment, in any one of the methods above, the initial dose, the first maintenance dose and the at least one additional maintenance dose comprise 75 mg of the anti-IL-23A antibody.

In one embodiment, the present invention provides a method for treating an inflammatory disease, in one aspect for treating psoriasis, psoriatic arthritis or axial (spinal) spondyloarthritis (ax-SpA), including ankylosing spondylitis and non-radiographic ax-SpA, comprising administering to a patient an anti-IL-23A antibody, said method comprising:
a) administering an initial dose of the anti-IL-23A antibody to the patient;
b) administering a first maintenance dose of the anti-IL-23A antibody to the patient 12 weeks after said initial dose is administered; and
c) administering at least one additional maintenance dose to the patient 12 weeks after said first maintenance dose is administered.

In one embodiment, in said method, the initial dose and the maintenance dose comprise 150 mg of said anti-IL-23A antibody. In one embodiment, in said method, the initial dose and the maintenance dose comprise 75 mg of said anti-IL-23A antibody.

In one embodiment, the present invention provides a method for treating an inflammatory disease, in one aspect for treating psoriasis, psoriatic arthritis or axial (spinal) spondyloarthritis (ax-SpA), including ankylosing spondylitis and non-radiographic ax-SpA, comprising administering to a patient an anti-IL-23A antibody, said method comprising:
a) administering an initial dose of the anti-IL-23A antibody to the patient;
b) administering a first maintenance dose of the anti-IL-23A antibody to the patient 8 weeks after said initial dose is administered; and
c) administering at least one additional maintenance dose to the patient 8 weeks after said first maintenance dose is administered.

In one embodiment, in said method, the initial dose and the maintenance dose comprise 90 mg of said anti-IL-23A antibody. In one embodiment, in said method, the initial dose and the maintenance dose comprise 150 mg of said anti-IL-23A antibody.

In one embodiment, the present invention provides a method for treating an inflammatory disease, in one aspect for treating psoriasis, psoriatic arthritis or axial (spinal) spondyloarthritis (ax-SpA), including ankylosing spondylitis and non-radiographic ax-SpA, comprising administering to a patient an anti-IL-23A antibody, said method comprising:
a) administering an initial dose of the anti-IL-23A antibody to the patient;
b) administering a first maintenance dose of the anti-IL-23A antibody to the patient 16 weeks after said initial dose is administered; and
c) administering at least one additional maintenance dose to the patient 16 weeks after said first maintenance dose is administered.

In one embodiment, in said method, the initial dose and the maintenance dose comprise 150 mg of said anti-IL-23A antibody.

In one embodiment, the present invention provides a method for treating an inflammatory disease, in one aspect for treating psoriasis, psoriatic arthritis or axial (spinal) spondyloarthritis (ax-SpA), including ankylosing spondylitis and non-radiographic ax-SpA, comprising administering to a patient an anti-IL-23A antibody, said method comprising:
a) administering an initial dose of the anti-IL-23A antibody to the patient;
b) administering a first maintenance dose of the anti-IL-23A antibody to the patient 6 weeks after said initial dose is administered; and
c) administering at least one additional maintenance dose to the patient 6 weeks after said first maintenance dose is administered.

In one embodiment, in said method, the initial dose and the maintenance dose comprise 150 mg of said anti-IL-23A antibody.

In one embodiment, the present invention provides a method for treating an inflammatory disease, in one aspect for treating psoriasis, psoriatic arthritis or axial (spinal) spondyloarthritis (ax-SpA), including ankylosing spondylitis and non-radiographic ax-SpA, comprising administering to a patient an anti-IL-23A antibody, said method comprising:
a) administering an initial dose of the anti-IL-23A antibody to the patient;
b) administering a first maintenance dose of the anti-IL-23A antibody to the patient 4 weeks after said initial dose is administered; and
c) administering at least one additional maintenance dose to the patient 4 weeks after said first maintenance dose is administered.

In one embodiment, in said method, the initial dose and the maintenance dose comprise 150 mg of said anti-IL-23A antibody.

In one embodiment, the present invention further provides a method for treating an inflammatory disease, in one aspect for treating psoriasis, psoriatic arthritis or axial (spinal) spondyloarthritis (ax-SpA), including ankylosing spondylitis and non-radiographic ax-SpA, comprising administering to a patient an anti-IL-23A antibody, said method comprising:
a) administering an initial dose of said anti-IL-23A antibody to the patient;
b) administering a loading dose of said anti-IL-23A antibody to the patient 1 to 6 weeks after said initial dose is administered;
c) administering a first maintenance dose of said anti-IL-23A antibody to the patient 8 to 24 weeks, for example 8 to 16 weeks, for example 8 to 12 weeks, for example 6 to 24 weeks, for example 6 to 16 weeks, for example 6 to 12 weeks, after said loading dose is administered; and
d) administering at least one additional maintenance dose to the patient 8 to 24 weeks, for example 8 to 16 weeks, for example 8 to 12 weeks, for example 6 to 24 weeks, for example 6 to 16 weeks, for example 6 to 12 weeks, after said first maintenance dose is administered.

In one embodiment, the loading dose is administered to the patient 1, 2, 3, 4, 5 or 6 weeks the said initial dose is administered.

In one embodiment, the first maintenance dose is administered to the patient 6, 8, 12, 16, 20 or 24 weeks after the loading dose is administered.

In one embodiment, the at least one additional maintenance dose is administered to the patient 6, 8, 12, 16, 20 or 24 weeks after the first maintenance dose is administered.

In one embodiment, the first maintenance dose is administered to the patient 6, 8, 12, 16, 20 or 24 weeks after the initial dose is administered and the at least one additional maintenance dose is administered to the patient 6, 8, 12, 16, 20 or 24 weeks after the first maintenance dose is administered.

In one embodiment, the first maintenance dose is administered to the patient 6, 8, 12, 16, 20 or 24 weeks after the loading dose is administered and the at least one additional maintenance dose is administered to the patient 6, 8, 12, 16, 20 or 24 weeks after the first maintenance dose is administered.

In additional embodiments:
the first maintenance dose is administered to the patient 6 weeks after the initial dose is administered and the at least one additional maintenance dose is administered to the patient 6 weeks after the first maintenance dose is administered; or
the first maintenance dose is administered to the patient 8 weeks after the initial dose is administered and the at least one additional maintenance dose is administered to the patient 8 weeks after the first maintenance dose is administered; or
the first maintenance dose is administered to the patient 12 weeks after the initial dose is administered and the at least one additional maintenance dose is administered to the patient 12 weeks after the first maintenance dose is administered; or
the first maintenance dose is administered to the patient 16 weeks after the initial dose is administered and the at least one additional maintenance dose is administered to the patient 16 weeks after the first maintenance dose is administered; or
the first maintenance dose is administered to the patient 20 weeks after the initial dose is administered and the at least one additional maintenance dose is administered to the patient 20 weeks after the first maintenance dose is administered; or
the first maintenance dose is administered to the patient 24 weeks after the initial dose is administered and the at least one additional maintenance dose is administered to the patient 24 weeks after the first maintenance dose is administered.

In additional embodiments:
the first maintenance dose is administered to the patient 6 weeks after the loading dose is administered and the at least one additional maintenance dose is administered to the patient 6 weeks after the first maintenance dose is administered; or
the first maintenance dose is administered to the patient 8 weeks after the loading dose is administered and the at least one additional maintenance dose is administered to the patient 8 weeks after the first maintenance dose is administered; or
the first maintenance dose is administered to the patient 12 weeks after the loading dose is administered and the at least one additional maintenance dose is administered to the patient 12 weeks after the first maintenance dose is administered; or
the first maintenance dose is administered to the patient 16 weeks after the loading dose is administered and the at least one additional maintenance dose is administered to the patient 16 weeks after the first maintenance dose is administered; or
the first maintenance dose is administered to the patient 20 weeks after the loading dose is administered and the at least one additional maintenance dose is administered to the patient 20 weeks after the first maintenance dose is administered; or
the first maintenance dose is administered to the patient 24 weeks after the loading dose is administered and the at least one additional maintenance dose is administered to the patient 24 weeks after the first maintenance dose is administered.

In one embodiment, in any one of the methods above, the initial dose comprises 15 to 300 mg, for example 15 to 250 mg, for example 90 to 180 mg, of the anti-IL-23A antibody.

In one embodiment, in any one of the methods above, the loading dose comprises 15 to 300 mg, for example 15 to 250 mg, for example 90 to 180 mg, of the anti-IL-23A antibody.

In one embodiment, in any one of the methods above, the first maintenance dose and the at least one additional maintenance dose comprise 15 to 300 mg, for example 15 to 250 mg, for example 90 to 180 mg, of the anti-IL-23A antibody.

In one embodiment, in any one of the methods above, the initial dose comprises 70 to 90 mg, 80 to 100 mg, 90 to 110 mg, 100 to 120 mg, 110 to 130 mg, 120 to 140 mg, 130 to 150 mg, 140 to 160 mg, 150 to 170 mg, 160 to 180 mg, 170 to 190 mg, 180 to 200 mg, 190 to 210 mg, 200 to 220 mg, 210 to 230 mg, 220 to 240 mg, 230 to 250 mg, 240 to 260 mg, 250 to 270 mg, 260 to 280 mg, 270 to 290 mg or 280 to 300 mg of the anti-IL-23A antibody.

In one embodiment, in any one of the methods above, the loading dose comprises 70 to 90 mg, 80 to 100 mg, 90 to 110 mg, 100 to 120 mg, 110 to 130 mg, 120 to 140 mg, 130 to 150 mg, 140 to 160 mg, 150 to 170 mg, 160 to 180 mg, 170 to 190 mg, 180 to 200 mg, 190 to 210 mg, 200 to 220 mg, 210 to 230 mg, 220 to 240 mg, 230 to 250 mg, 240 to 260 mg, 250 to 270 mg, 260 to 280 mg, 270 to 290 mg or 280 to 300 mg of the anti-IL-23A antibody.

In one embodiment, in any one of the methods above, the first maintenance dose and the at least one additional maintenance dose comprise 70 to 90 mg, 80 to 100 mg, 90 to 110 mg, 100 to 120 mg, 110 to 130 mg, 120 to 140 mg, 130 to 150 mg, 140 to 160 mg, 150 to 170 mg, 160 to 180 mg, 170 to 190 mg, 180 to 200 mg, 190 to 210 mg, 200 to 220 mg, 210 to 230 mg, 220 to 240 mg, 230 to 250 mg, 240 to 260 mg, 250 to 270 mg, 260 to 280 mg, 270 to 290 mg or 280 to 300 mg of the anti-IL-23A antibody.

In one embodiment, in any one of the methods above, the initial dose comprises 18, 20, 30, 40, 50, 60, 70, 80, 90, 100, 110, 120, 130, 140, 150, 160, 170, 180, 190, 200, 210, 220, 230, 240, 250, 260, 270, 280, 290 or 300 mg of the anti-IL-23A antibody. In one embodiment, in any one of the methods above, the initial dose comprises 75 mg of the anti-IL-23A antibody.

In one embodiment, in any one of the methods above, the first maintenance dose and the at least one additional maintenance dose comprise 18, 20, 30, 40, 50, 60, 70, 80, 90, 100, 110, 120, 130, 140, 150, 160, 170, 180, 190, 200, 210, 220, 230, 240, 250, 260, 270, 280, 290 or 300 mg of the anti-IL-23A antibody. In one embodiment, in any one of the methods above, the first maintenance dose and the at least one additional maintenance dose comprise 75 mg of the anti-IL-23A antibody.

In one embodiment, in any one of the methods above, the initial dose and the loading dose comprise 18, 20, 30, 40, 50, 60, 70, 80, 90, 100, 110, 120, 130, 140, 150, 160, 170, 180, 190, 200, 210, 220, 230, 240, 250, 260, 270, 280, 290 or 300 mg of the anti-IL-23A antibody. In one embodiment, in any one of the methods above, the initial dose and the loading dose comprise 75 mg of the anti-IL-23A antibody.

In one embodiment, in any one of the methods above, the initial dose, the loading dose, the first maintenance dose and the at least one additional maintenance dose comprise 18, 20, 30, 40, 50, 60, 70, 80, 90, 100, 110, 120, 130, 140, 150, 160, 170, 180, 190, 200, 210, 220, 230, 240, 250, 260, 270, 280, 290 or 300 mg of the anti-IL-23A antibody. In one embodiment, in any one of the methods above, the initial dose, the loading dose, the first maintenance dose and the at least one additional maintenance dose comprise 75 mg of the anti-IL-23A antibody.

In one embodiment, the present invention further provides a method for treating an inflammatory disease, in one aspect for treating psoriasis, psoriatic arthritis or axial (spinal) spondyloarthritis (ax-SpA), including ankylosing spondylitis and non-radiographic ax-SpA, comprising administering to a patient an anti-IL-23A antibody, said method comprising:
a) administering an initial dose of said anti-IL-23A antibody to the patient;
b) administering a loading dose of said anti-IL-23A antibody to the patient 4 weeks after said initial dose is administered;
c) administering a first maintenance dose of said anti-IL-23A antibody to the patient 12 weeks after said loading dose is administered; and
d) administering at least one additional maintenance dose to the patient 12 weeks after said first maintenance dose is administered.

In one embodiment, in said method, the initial dose, the loading dose and the maintenance dose comprise 150 mg of said anti-IL-23A antibody. In one embodiment, in said method, the initial dose and the loading dose comprise 300 mg of said anti-IL-23A antibody and the maintenance dose comprises 150 mg of said anti-IL-23A antibody.

In one embodiment, in said method, the initial dose and the loading dose comprise 180 mg of said anti-IL-23A antibody and the maintenance dose comprises 90 mg of said anti-IL-23A antibody. In one embodiment, in said method, the initial dose, the loading dose and the maintenance dose comprise 75 mg of said anti-IL-23A antibody.

In one embodiment, the present invention further provides a method for treating an inflammatory disease, in one aspect for treating psoriasis, psoriatic arthritis or axial (spinal) spondyloarthritis (ax-SpA), including ankylosing spondylitis and non-radiographic ax-SpA, comprising administering to a patient an anti-IL-23A antibody, said method comprising:
a) administering an initial dose of said anti-IL-23A antibody to the patient;
b) administering a loading dose of said anti-IL-23A antibody to the patient 4 weeks after said initial dose is administered;
c) administering a first maintenance dose of said anti-IL-23A antibody to the patient 8 weeks after said loading dose is administered; and
d) administering at least one additional maintenance dose to the patient 8 weeks after said first maintenance dose is administered.

In one embodiment, in said method, the initial dose, the loading dose and the maintenance dose comprise 90 mg of said anti-IL-23A antibody. In one embodiment, in said method, the initial dose, the loading dose and the maintenance dose comprise 150 mg of said anti-IL-23A antibody.

In one embodiment, the present invention further provides a method for treating an inflammatory disease, in one aspect for treating psoriasis, psoriatic arthritis or axial (spinal) spondyloarthritis (ax-SpA), including ankylosing spondylitis and non-radiographic ax-SpA, comprising administering to a patient an anti-IL-23A antibody, said method comprising:
a) administering an initial dose of said anti-IL-23A antibody to the patient;
b) administering a loading dose of said anti-IL-23A antibody to the patient 4 weeks after said initial dose is administered;
c) administering a first maintenance dose of said anti-IL-23A antibody to the patient 16 weeks after said loading dose is administered; and
d) administering at least one additional maintenance dose to the patient 16 weeks after said first maintenance dose is administered.

In one embodiment, in said method, the initial dose, the loading dose and the maintenance dose comprise 150 mg of said anti-IL-23A antibody.

In one embodiment, the present invention further provides a method for treating an inflammatory disease, in one aspect for treating psoriasis, psoriatic arthritis or axial (spinal) spondyloarthritis (ax-SpA), including ankylosing spondylitis and non-radiographic ax-SpA, comprising administering to a patient an anti-IL-23A antibody, said method comprising:
a) administering an initial dose of said anti-IL-23A antibody to the patient;
b) administering a loading dose of said anti-IL-23A antibody to the patient 4 weeks after said initial dose is administered;
c) administering a first maintenance dose of said anti-IL-23A antibody to the patient 6 weeks after said loading dose is administered; and
d) administering at least one additional maintenance dose to the patient 6 weeks after said first maintenance dose is administered.

In one embodiment, in said method, the initial dose, the loading dose and the maintenance dose comprise 150 mg of said anti-IL-23A antibody.

In one embodiment, the present invention further provides a method for treating an inflammatory disease comprising administering to a patient 15 to 300 mg, for example 15 to 250 mg, for example 90 to 180 mg, of an anti-IL-23A antibody. In one embodiment, the inflammatory disease is psoriasis, psoriatic arthritis or axial (spinal) spondyloarthritis (ax-SpA), including ankylosing spondylitis and non-radiographic ax-SpA.

In one embodiment, the method comprises administering to a patient 70 to 90 mg, 80 to 100 mg, 90 to 110 mg, 100 to 120 mg, 110 to 130 mg, 120 to 140 mg, 130 to 150 mg, 140 to 160 mg, 150 to 170 mg, 160 to 180 mg, 170 to 190 mg, 180 to 200 mg, 190 to 210 mg, 200 to 220 mg, 210 to 230 mg, 220 to 240 mg, 230 to 250 mg, 240 to 260 mg, 250 to 270 mg, 260 to 280 mg, 270 to 290 mg or 280 to 300 mg of an anti-IL-23A antibody.

In one embodiment, the method comprises administering to a patient 18, 20, 30, 40, 50, 60, 70, 80, 90, 100, 110, 120, 130, 140, 150, 160, 170, 180, 190, 200, 210, 220, 230, 240, 250, 260, 270, 280, 290 or 300 mg of an anti-IL-23A antibody. In one embodiment, the method comprises administering to a patient 75 mg of an anti-IL-23A antibody.

In one embodiment, the anti-IL-23A antibody is administered as an initial dose, a loading dose or a maintenance dose.

In one embodiment, the present invention further provides a method for treating an inflammatory disease, in one aspect for treating psoriasis or psoriatic arthritis, comprising administering to a patient an anti-IL-23A antibody, said method comprising:
a) administering an initial dose of said anti-IL-23A antibody to the patient; and
b) administering a second dose of said anti-IL-23A antibody to the patient when the patient no longer maintains a certain PASI score, for example PASI 90, PASI 75, PASI 100 or PASI 50.

In one aspect, the initial dose and the second dose are doses as described herein.

In one embodiment, the present invention further provides a method for treating an inflammatory disease, in one aspect for treating psoriasis or psoriatic arthritis, comprising administering to a patient an anti-IL-23A antibody, said method comprising:
a) administering an initial dose of said anti-IL-23A antibody to the patient;
b) administering a loading dose of said anti-IL-23A antibody to the patient 1 to 6 weeks, for example 4 weeks, after said initial dose is administered; and
c) administering a third dose of said anti-IL-23A antibody to the patient when the patient no longer maintains a certain PASI score, for example PASI 90, PASI 75, PASI 100 or PASI 50.

In one aspect, the initial dose, the loading dose and the third dose are doses as described herein.

In one embodiment, in any one of the methods above, the anti-IL-23A antibody is Antibody A, Antibody B, Antibody C or Antibody D.

In one embodiment, in any one of the methods above, the method is for treating plaque psoriasis, for example chronic plaque psoriasis.

In one embodiment, in any one of the methods above, the method is for treating moderate to severe chronic plaque psoriasis, for example in a patient who is a candidate for systemic therapy or phototherapy.

In one embodiment, in any one of the methods above, the method is for treating moderate to severe chronic plaque psoriasis, for example in a patient who failed to respond to, or who has a contraindication to, or is intolerant to other systemic therapy including ciclosporin, methotrexate, psoralen or ultraviolet-A light (PUVA).

In one embodiment, in any one of the methods above, the method is for treating pustular psoriasis.

In one embodiment, in any one of the methods above, the method is for treating erythodermic psoriasis (also known as psoriatic erythroderma).

In one embodiment, in any one of the methods above, the method is for treating generalized pustular psoriasis (GPP). In one embodiment, the present invention provides a method for treating generalized pustular psoriasis (GPP) comprising administering to a patient an anti-IL-23A antibody, for example an anti-IL-23A antibody as described herein.

In one embodiment, in any one of the methods above, the method is for treating psoriatic arthritis, for example active psoriatic arthritis.

In one embodiment, the anti-IL-23A antibody is used alone or in combination with one or more non-biologic DMARDs (Disease-Modifying Antirheumatic Drug), for example for treating psoriatic arthritis, for example active psoriatic arthritis, for example to reduce signs and symptoms. In one embodiment, the anti-IL-23A antibody is used or indicated to inhibit the progression of structural damage, and/or improve physical function.

In one embodiment, the anti-IL-23A antibody is used alone or in combination with methotrexate (MTX), for example for the treatment of psoriatic arthritis, for example active psoriatic arthritis, for example when the response to previous non-biological DMARD therapy has been inadequate. In one aspect, the anti-IL-23A antibody is used to reduce the rate of progression of peripheral joint damage as measured by X-ray and/or to improve physical function.

In one embodiment, in any one of the methods above, the anti-IL-23A antibody is administered by subcutaneous administration.

In one aspect, in a method of the present invention, 90-180 mg of the anti-IL-23 antibody is administered to the patient every 6-12 weeks, for example every 8-12 weeks, with or without a loading dose.

In one aspect, in a method of the present invention, a first maintenance dose is administered to the patient up to 32 weeks after the administration of the initial dose. In one aspect, in a method of the present invention, the first maintenance dose is administered to the patient 28 weeks or 32 weeks after the administration of the initial dose.

In one aspect, in a method of the present invention, a first maintenance dose is administered to the patient up to 32 weeks after the administration of the loading dose. In one aspect, in a method of the present invention, the first maintenance dose is administered to the patient 28 weeks or 32 weeks after the administration of the loading dose.

In one aspect, in a method of the present invention, at least one additional maintenance dose is administered to the patient up to 32 weeks after the administration of the first maintenance dose. In one aspect, in a method of the present invention, at least one additional maintenance dose is administered to the patient 28 weeks or 32 weeks after the administration of
the first maintenance dose.

In one embodiment, the present invention further provides a method for treating psoriasis, said method comprising administering Antibody A to a patient previously treated with a first agent. In one aspect, the PASI score or the sPGA score of said patient improves after the administration of Antibody A. In one aspect, the patient reaches PASI 90 after the administration of Antibody A. In one aspect, the patient reaches PASI 100 after the administration of Antibody A. In one aspect, the patient reaches a sPGA score of clear (score of 0) or almost clear (score of 1) after the administration of Antibody A. In one aspect, the patient did not reach PASI 90 before the administration of Antibody A. In one aspect, the patient did not reach PASI 75 before the administration of Antibody A. In one aspect, the patient did not reach PASI 50 before the administration of Antibody A. In one aspect, the patient did not reach a sPGA score of clear (score of 0) or almost clear (score of 1) before the administration of Antibody A. In one aspect, the first agent is a biological drug, for example a monoclonal antibody. In one aspect, the first agent is ustekinumab or adalimumab.

In one embodiment, the present invention further provides a method for treating psoriasis, said method comprising identifying a patient treated with a first agent, wherein the therapeutic effect of said first agent in said patient is insufficient to treat psoriasis; and administering Antibody A to said patient. In one aspect, the therapeutic effect of Antibody A in said patient is sufficient to treat psoriasis. In one aspect, the patient does not reach PASI 90 before the administration of Antibody A. In one aspect, the patient does not reach PASI 75 before the administration of Antibody A. In one aspect, the patient does not reach PASI 50 before the administration of Antibody A. In one aspect, the patient does not reach a sPGA score of clear (score of 0) or almost clear (score of 1) before the administration of Antibody A. In one aspect, the patient reaches PASI 90 after the administration of Antibody A. In one aspect, the patient reaches PASI 100 after the administration of Antibody A. In one aspect, the patient reaches a sPGA score of clear (score of 0) or almost clear (score of 1) after the administration of Antibody A. In one aspect, the first agent is a biological drug, for example a monoclonal antibody. In one aspect, the first agent is ustekinumab or adalimumab.

In one embodiment, the present invention further provides a method for treating psoriasis comprising switching a patient who does not fully respond to a first agent from treatment with said first agent to treatment with Antibody A. In one aspect, a patient does not fully respond when a PASI score of 90 is not reached. In one aspect, a patient does not fully respond when a PASI score of 75 is not reached. In one aspect, a patient does not fully respond when a PASI score of 50 is not reached. In one aspect, a patient does not fully respond when a sPGA score of clear (score of 0) or almost clear (score of 1) is not reached. In one aspect, the first agent is a biological drug, for example a monoclonal antibody. In one aspect, the first agent is ustekinumab or adalimumab.

In one embodiment, in any one of the methods above, the patient is an adult patient.

In one aspect, the present invention provides an anti-IL-23A antibody for use in the treatment of a disease as described herein.

In one aspect, the present invention provides an anti-IL-23A antibody for use in the treatment of a disease, for example an inflammatory disease, for example psoriasis, psoriatic arthritis or axial (spinal) spondyloarthritis (ax-SpA), including Ankylosing Spondylitis and non-radiographic ax-SpA, by administration in certain amounts and/or at certain intervals as described herein.

In one aspect, the present invention provides for the use of an anti-IL-23A antibody for the preparation of a medicament for the treatment of a disease as described herein.

In one aspect, the present invention provides for the use of an anti-IL-23A antibody for the preparation of a medicament for the treatment of a disease, for example an inflammatory disease, for example psoriasis, psoriatic arthritis or axial (spinal) spondyloarthritis (ax-SpA), including Ankylosing Spondylitis and non-radiographic ax-SpA, by administration in certain amounts and/or at certain intervals as described herein.

In one embodiment, in any one of the methods or uses above, the anti-IL-23A antibody is disclosed below.

In one embodiment, the anti-IL-23A antibody comprises a light chain variable region comprising the amino acid sequence of SEQ ID NO:1 (CDR1-L); the amino acid sequence of SEQ ID NO:2 (CDR2-L); and the amino acid sequence of SEQ ID NO:3 (CDR3-L); and a heavy chain variable region comprising the amino acid sequence of SEQ ID NO: 4, 7, 8 or 9 (CDR1-H); the amino acid sequence of SEQ ID NO:5 (CDR2-H); and the amino acid sequence of SEQ ID NO:6 (CDR3-H).

In one embodiment, the anti-IL-23A antibody comprises a light chain variable region comprising the amino acid sequence of SEQ ID NO:1 (CDR1-L); the amino acid sequence of SEQ ID NO:2 (CDR2-L); and the amino acid sequence of SEQ ID NO:3 (CDR3-L); and a heavy chain variable region comprising the amino acid sequence of SEQ ID NO:4 (CDR1-H); the amino acid sequence of SEQ ID NO:5 (CDR2-H); and the amino acid sequence of SEQ ID NO:6 (CDR3-H).

In one embodiment, the anti-IL-23A antibody comprises a light chain variable region comprising the amino acid sequence of SEQ ID NO:1 (CDR1-L); the amino acid sequence of SEQ ID NO:2 (CDR2-L); and the amino acid sequence of SEQ ID NO:3 (CDR3-L); and a heavy chain variable region comprising the amino acid sequence of SEQ ID NO:7 (CDR1-H); the amino acid sequence of SEQ ID NO:5 (CDR2-H); and the amino acid sequence of SEQ ID NO:6 (CDR3-H).

In one embodiment, the anti-IL-23A antibody comprises a light chain variable region comprising the amino acid sequence of SEQ ID NO:1 (CDR1-L); the amino acid sequence of SEQ ID NO:2 (CDR2-L); and the amino acid sequence of SEQ ID NO:3 (CDR3-L); and a heavy chain variable region comprising the amino acid sequence of SEQ ID NO:8 (CDR1-H); the amino acid sequence of SEQ ID NO:5 (CDR2-H); and the amino acid sequence of SEQ ID NO:6 (CDR3-H).

In one embodiment, the anti-IL-23A antibody comprises a light chain variable region comprising the amino acid sequence of SEQ ID NO:1 (CDR1-L); the amino acid sequence of SEQ ID NO:2 (CDR2-L); and the amino acid sequence of SEQ ID NO:3 (CDR3-L); and a heavy chain variable region comprising the amino acid sequence of SEQ ID NO:9 (CDR1-H); the amino acid sequence of SEQ ID NO:5 (CDR2-H); and the amino acid sequence of SEQ ID NO:6 (CDR3-H).

In one embodiment, the anti-IL-23A antibody comprises a light chain variable region comprising the amino acid sequence of any one of SEQ ID NO:10, 11, 12 or 13; and a heavy chain variable region comprising the amino acid sequence any one of SEQ ID NO:14, 15, 16 or 17.

In one embodiment, the anti-IL-23A antibody comprises a light chain variable region comprising the amino acid sequence of SEQ ID NO:11 and a heavy chain variable region comprising the amino acid sequence SEQ ID NO: 14.

In one embodiment, the anti-IL-23A antibody comprises a light chain variable region comprising the amino acid sequence of SEQ ID NO:11 and a heavy chain variable region comprising the amino acid sequence SEQ ID NO: 15.

In one embodiment, the anti-IL-23A antibody comprises a light chain variable region comprising the amino acid sequence of SEQ ID NO: 10 and a heavy chain variable region comprising the amino acid sequence SEQ ID NO: 14.

In one embodiment, the anti-IL-23A antibody comprises a light chain variable region comprising the amino acid sequence of SEQ ID NO: 10 and a heavy chain variable region comprising the amino acid sequence SEQ ID NO: 15.

In one embodiment, the anti-IL-23A antibody comprises the amino acid sequence SEQ ID NO:14 or 15 linked to a human IgG1, IgG2, IgG3, IgG4, IgM, IgA or IgE heavy chain constant region. In one embodiment, the anti-IL-23A antibody comprises the amino acid sequence of SEQ ID NO: 14 or 15 linked to a human IgG1 heavy chain constant region. In one embodiment, the anti-IL-23A antibody comprises the amino acid sequence of SEQ ID NO:10 or 11 linked to a human kappa or lambda light chain constant region.

In one embodiment, In one embodiment, the anti-IL-23A antibody comprises the amino acid sequence of SEQ ID NO:14 or 15 linked to a human IgG1 heavy chain constant region; and the amino acid sequence of SEQ ID NO: 10 or 11 linked to a human kappa light chain constant region.

In one embodiment, the anti-IL-23A antibody is a humanized monoclonal antibody comprising a light chain variable region comprising the amino acid sequence selected from the group consisting of any one of SEQ ID NO:10, 11, 12 and 13 and a heavy chain variable region comprising the amino acid sequence selected from the group consisting of any one of SEQ ID NO:14, 15, 16 and 17.

In one embodiment, the anti-IL-23A antibody is a humanized monoclonal antibody comprising a light chain variable region comprising the amino acid sequence of SEQ ID NO:11 and a heavy chain variable region comprising the amino acid sequence SEQ ID NO:14.

In one embodiment, the anti-IL-23A antibody is a humanized monoclonal antibody comprising a light chain variable region comprising the amino acid sequence of SEQ ID NO:11 and a heavy chain variable region comprising the amino acid sequence SEQ ID NO:15.

In one embodiment, the anti-IL-23A antibody is a humanized monoclonal antibody comprising a light chain variable region comprising the amino acid sequence of SEQ ID NO:10 and a heavy chain variable region comprising the amino acid sequence SEQ ID NO:14.

In one embodiment, the anti-IL-23A antibody is a humanized monoclonal antibody comprising a light chain variable region comprising the amino acid sequence of SEQ ID NO:10 and a heavy chain variable region comprising the amino acid sequence SEQ ID NO:15.

In one embodiment, the anti-IL-23A antibody comprises a light chain comprising the amino acid sequence of SEQ ID NO:18 or 21 and a heavy chain comprising the amino acid sequence of SEQ ID NO:19 or 20.

In one embodiment, the anti-IL-23A antibody comprises a light chain comprising the amino acid sequence of SEQ ID NO:18 and a heavy chain comprising the amino acid sequence of SEQ ID NO:19.

In one embodiment, the anti-IL-23A antibody comprises a light chain comprising the amino acid sequence of SEQ ID NO:18 and a heavy chain comprising the amino acid sequence of SEQ ID NO:20.

In one embodiment, the anti-IL-23A antibody comprises a light chain comprising the amino acid sequence of SEQ ID NO:21 and a heavy chain comprising the amino acid sequence of SEQ ID NO:19.

In one embodiment, the anti-IL-23A antibody comprises a light chain comprising the amino acid sequence of SEQ ID NO: 21 and a heavy chain comprising the amino acid sequence of SEQ ID NO: 20.

In one embodiment, the anti-IL-23A antibody is Antibody A, Antibody B, Antibody C or Antibody D.

In one embodiment, the anti-IL-23A antibody is as disclosed in WO2007/005955, WO2007/024846, WO2007/027714, WO2007/076524, WO2008/103432 or WO2012/061448.

In one embodiment, the present invention provides a method for detecting the presence or absence of a beneficial response in a patient after administration of an IL-23A antagonist, comprising: a) obtaining a biological sample from the patient; b) measuring in said sample the level of expression of one or more genes; c) comparing the level in b) to the level of expression of the one or more genes in a control; and d) determining whether or not the difference in levels between the sample and the control reflects a beneficial response in the patient, wherein the one or more genes is one or more genes described herein. In one aspect, the patient suffers from psoriasis. In one aspect, the control is from one or more subjects that do not suffer from psoriasis. In one aspect, the control is from one or more subjects treated with another agent, for example an anti-IL-12/IL-23 antibody, for example ustekinumab. In one aspect, the IL-23A antagonist is an anti-IL-23A antibody or an antigen binding fragment thereof, for example Antibody A, Antibody B, Antibody C or Antibody D.

In one embodiment, the anti-IL-23A antibody modulates the genes as described in Example 5. In one embodiment, the anti-IL-23A antibody modulates the pathways as described in Example 5. In one embodiment, the anti-IL-23A antibody modulates one of the genes as described in Example 5. In one embodiment, the anti-IL-23A antibody modulates one of the pathways as described in Example 5.

The present disclosure also provides a liquid antibody formulation that comprises 150 mg/ml of the antibody as defined herein. The antibody is risankizumab or an antibody comprising the same heavy and light chain sequences as risankizumab. No formulations of said antibody having such a high antibody concentration were described or available in the art and by providing such high concentration antibody formulation, the present disclosure makes an important contribution to the art. Despite the high antibody concentration, the formulations according to the present disclosure are stable and are suitable for therapeutic use. As is demonstrated in the examples, the formulations according to the present disclosure comprising 150 mg/ml of the antibody risankizumab provide advantageous stability characteristics and are well suitable for subcutaneous administration. They can provide long-term stability. Advantageously, a 150 mg dose of the antibody can be administered with a single 1 ml injection.

According to a first aspect of the present disclosure, a liquid pharmaceutical formulation is provided comprising 150 mg/ml of an anti-IL-23p19 antibody, wherein the antibody comprises a light chain amino acid sequence according to SEQ ID NO: 18 and a heavy chain amino acid sequence according to SEQ ID NO: 19.

According to a first sub-aspect of this first aspect, the liquid pharmaceutical formulation comprises
a) 150 mg/ml of an anti-IL-23p19 antibody, wherein the antibody comprises a light chain amino acid sequence according to SEQ ID NO: 18 and a heavy chain amino acid sequence according to SEQ ID NO: 19;
b) a polyol; and
c) a surfactant.
   This formulation may additionally comprise d) a buffer. Furthermore, the present disclosure provides buffer-free formulations comprising 150 mg/ml of the antibody. As is disclosed herein, the liquid pharmaceutical formulations according to the first sub-aspect are stable.

According to a second sub-aspect of this first aspect, a stable liquid pharmaceutical formulation is provided comprising
a) 150 mg/ml of an anti-IL-23p19 antibody, wherein the antibody comprises a light chain amino acid sequence according to SEQ ID NO: 18 and a heavy chain amino acid sequence according to SEQ ID NO: 19;
b) a tonicity modifier; and
c) a surfactant,
   wherein the formulation has a pH of 5.5-5.9 and the formulation is isotonic. This formulation may additionally comprise d) a buffer.

The formulations according to the first and second sub-aspect of the 150 mg/ml antibody formulation according to the first aspect may also be provided in lyophilized form.

In related aspects, a sealed container is provided which contains a formulation according to the present disclosure.

In related aspects, the present disclosure pertains to the formulations according to the present disclosure or the container containing a formulation according to the present disclosure for therapeutic treatment of a human subject. The disease to be treated may be selected from psoriasis and inflammatory bowel disease. In further embodiments, the disease to be treated may be selected from psoriatic arthritis and Crohn's disease.

Other objects, features, advantages and aspects of the present application will become apparent to those skilled in the art from the following description and appended claims. It should be understood, however, that the following description, appended claims, and specific examples, while indicating preferred embodiments of the application, are given by way of illustration only.

### BRIEF DESCRIPTION OF THE FIGURES

FIG. 1: Rate of patients with >50% reductions from baseline pain-VAS (%).

FIG. 2: Achievement of PASI 50 over time (observed).

FIG. 3: Achievement of PASI 75 over time (observed).

FIG. 4: Achievement of PASI 90 over time (observed).

FIG. 5: Achievement of PASI 100 over time (observed).

FIG. 6 shows the amino acid sequence of the light chain of the antibody (SEQ ID NO: 18).

FIG. 7 shows the amino acid sequence of the heavy chain of the antibody (SEQ ID NO: 19).

FIG. 8 shows an overview of the experimental schedule used to assess freeze-thaw stability of various formulations.

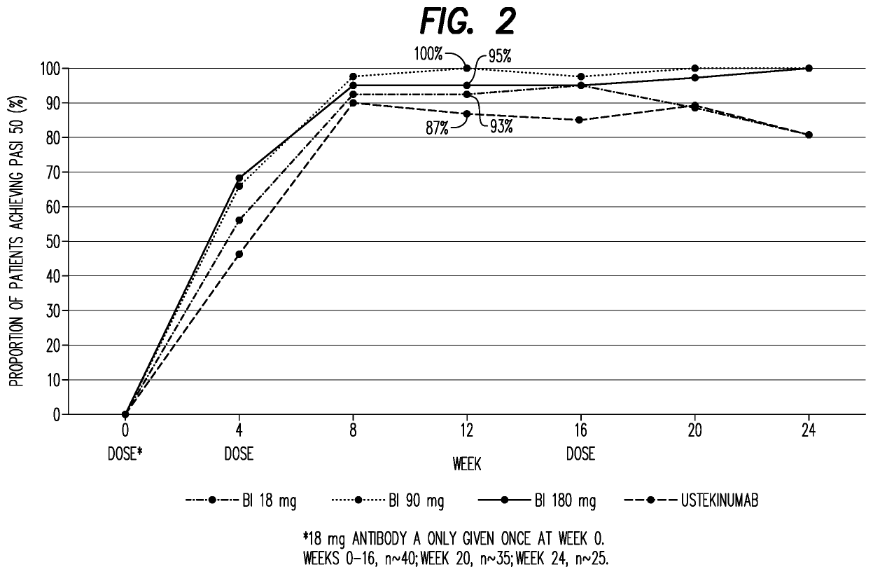

Antibody A is also referred to as “BI” in FIGS. 2 to 5.

### DETAILED DESCRIPTION

### Anti-IL-23p19 Antibodies and Formulations Thereof

The p19 subunit of IL-23 (also referred to herein as “IL-23A”, “IL-23p19” and “p19 subunit”) is a 189 amino acid polypeptide containing a 21 aa leader sequence (Oppmann et al. Immunity 13:715 (2000), SEQ ID NO: 22). The biological activity of the molecule is only detected when it is partnered with the IL-12p40 subunit to form IL-23. IL-23 is predominantly expressed by activated dendritic cells (DCs) and phagocytic cells. The receptor for IL-23 was found to be composed of the IL-12R□1 subunit of IL-12 receptor partnered with a unique subunit called IL-23R (Parham et al. J. Immunol. 168:5699 (2002)). Expression of the receptor is detected primarily on memory T cells and NK cells. Thus, expression of this cytokine:receptor pair appears to be restricted to specific populations of immune cells. While it was first thought that IL-12 and IL-23 would share many functions, the data has shown the picture to be different. Whereas IL-12 has a predominant role in the production of Th1 cells, IL-23 was found to be critically involved in the production and maintenance of a recently recognized Th cell subset termed Th17 (Kikly et al. Curr. Opin. Immunol. 18:670 (2006), Kastelein et al. Ann. Rev. Immunol. 25:221 (2007)). These cells produce IL-17A, IL-17F, IL-22 and other pro-inflammatory cytokines such as IL-6 and TNF-α. As described below, animal model studies on the role of these Th17 cells show their importance as a driving force in chronic inflammation and autoimmunity.

SEQ ID NO: 22:
MLGSRAVMLL LLLPWTAQGR AVPGGSSPAW TQCQQLSQKL
CTLAWSAHPL VGHMDLREEG DEETTNDVPH IQCGDGCDPQ
GLRDNSQFCL QRIHQGLIFY EKLLGSDIFT GEPSLLPDSP
VGQLHASLLG LSQLLQPEGH HWETQQIPSL SPSQPWQRLL
LRFKILRSLQ AFVAVAARVF AHGAATLSP

According to a first aspect, a liquid pharmaceutical formulation is provided comprising 150 mg/ml of an anti-IL-23p19 antibody, wherein the antibody comprises a light chain amino acid sequence according to SEQ ID NO: 18 and a heavy chain amino acid sequence according to SEQ ID NO: 19.

According to a first sub-aspect of this first aspect, a liquid pharmaceutical formulation is provided comprising
a) 150 mg/ml of an anti-IL-23p19 antibody, wherein the antibody comprises a light chain amino acid sequence according to SEQ ID NO: 18 and a heavy chain amino acid sequence according to SEQ ID NO: 19;
b) a polyol; and
c) a surfactant.

The formulation according to this first sub-aspect may additionally comprise d) a buffer. Furthermore, the present disclosure provides buffer-free formulations comprising 150 mg/ml of the antibody. As is disclosed herein, the liquid pharmaceutical formulations according to the first sub-aspect are stable.

According to a second sub-aspect of this first aspect, a stable liquid pharmaceutical formulation is provided comprising
a) 150 mg/ml of an anti-IL-23p19 antibody, wherein the antibody comprises a light chain amino acid sequence according to SEQ ID NO: 18 and a heavy chain amino acid sequence according to SEQ ID NO: 19;
b) a tonicity modifier; and
c) a surfactant,
   wherein the formulation has a pH of 5.5-5.9 and the formulation is isotonic.

The stable formulation according to this second sub-aspect may additionally comprise
d) a buffer.

The formulation according to the present disclosure comprises a high antibody concentration of 150 mg/ml. Despite this high antibody concentration, the liquid pharmaceutical formulations of the present disclosure are stable and advantageously can provide long-term stability. The formulations according to the present disclosure moreover address core administration challenges for a high concentration antibody formulation that is suitable for injection, by providing inter alia a suitable viscosity and good syringeability, whereby the formulation according to the present disclosure is particularly suitable for injection, such as subcutaneous injection. The advantageous properties of these formulations are demonstrated in the examples. The formulation according to the first aspect solves the challenges facing formulations for injection by providing a stable and robust formulation comprising 150 mg/ml of the antibody, thereby enabling the subcutaneous administration of a 150 mg dose of the antibody using a target volume of only 1 ml.

As disclosed herein, the formulations according to the first aspect can be provided as buffer-free or buffer-containing formulations. According to one core embodiment, the liquid pharmaceutical formulation according to the first aspect comprises d) a buffer. In another embodiment, the liquid pharmaceutical formulation does not contain a buffer as additive.

Subsequently, the components of the 150 mg/ml antibody formulation according to the first aspect are described in further detail. In particular, suitable embodiments and characteristics of components a), b), c) and optionally d) that are comprised in the formulations according to the first sub-aspect and the second sub-aspect are disclosed.

### a) The Antibody

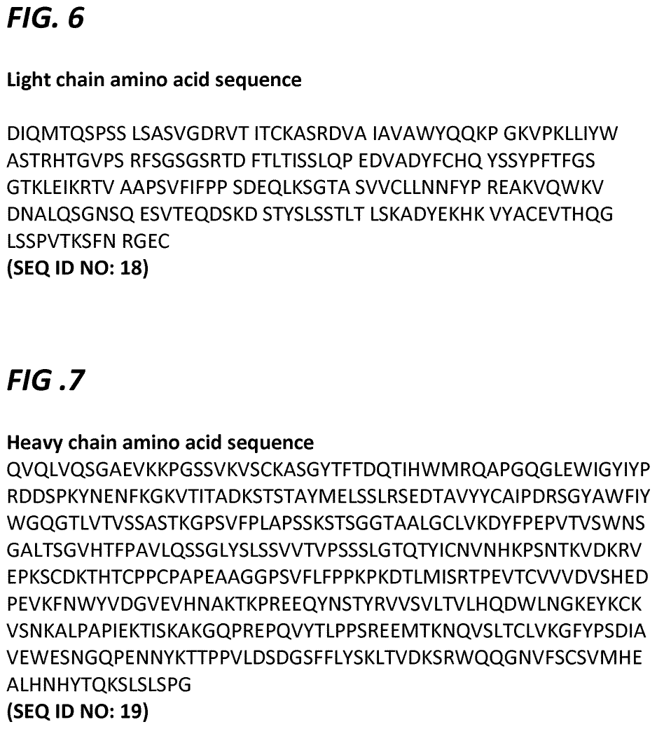

The antibody comprised in the formulation comprises a light chain amino acid sequence according to SEQ ID NO: 18 and a heavy chain amino acid sequence according to SEQ ID NO: 19. SEQ ID NO: 18 and 19 are shown in FIG. 6 and FIG. 7. The light and heavy chains of the antibody risankizumab correspond to the light and heavy chain sequences as shown in SEQ ID NO: 18 and 19. According to one embodiment, the antibody has the same light and heavy chains as the antibody risankizumab (see INN risankizumab, WHO Drug Information, Vol. 29, No. 2, 2015) and such antibody is referred to herein as risankizumab. Advantageously, the present disclosure provides stable, high concentration liquid pharmaceutical formulations for the antibody risankizumab, which is approved for the treatment of psoriasis. The whole disclosure provided herein, is specifically directed and applies to the antibody risankizumab that is comprised in the disclosed formulations. Risankizumab may be recombinantly produced in various host cells and suitable cells for recombinant antibody production are known in the art.

In one embodiment, the antibody is recombinantly produced in a mammalian cell. Suitable mammalian cells are known in the art and comprise rodent as well as human cell lines. In one embodiment, the antibody has been recombinantly produced in a hamster cell. In one embodiment, the antibody has been recombinantly produced in a CHO cell.

### Component b)

The formulation according to the first sub-aspect of the 150 mg/ml formulation according to the first aspect comprises a polyol as component b). Suitable polyols that can be used as excipient in a pharmaceutical formulation are known in the art and are described herein.

The formulation according to the second sub-aspect of the 150 mg/ml formulation according to the first aspect comprises a tonicity modifier as component b). A tonicity modifier is an agent that is suitable to adjust the tonicity of the formulation. Tonicity modifiers useful for adjusting the tonicity of a pharmaceutical formulation are known in the art, and include compounds such as salts and furthermore polyols, such as sugars and sugar alcohols. Therefore, the tonicity modifier used as component b) in the stable 150 mg/ml formulation according to the second sub-aspect may be a polyol as it is used as component b) in the 150 mg/ml formulation according to the first sub-aspect. According to one embodiment, the tonicity modifier that is comprised in the stable liquid formulation according to the second sub-aspect is thus a polyol, optionally a sugar and/or a sugar alcohol.

The term “polyol” as used herein refers to a substance with multiple hydroxyl groups, and includes sugars (reducing and nonreducing sugars) and sugar alcohols. The polyol may comprise at least three, at least four or at least five hydroxyl groups. In certain embodiments, polyols have a molecular weight that is ≤600 Da (e.g., in the range from 120 to 400 Da). A “reducing sugar” is one that contains a free aldehyde or ketone group and can reduce metal ions or react covalently with lysine and other amino groups in proteins. A “nonreducing sugar” is one that lacks a free aldehyde or ketone group and is not oxidised by mild oxidising agents such as Fehling's or Benedict's solutions. Examples for reducing and nonreducing sugars suitable for use in pharmaceutical formulations are known to the skilled person. Nonreducing sugars include e.g. sucrose and trehalose. The use of trehalose is particularly useful as is disclosed herein. Examples of sugar alcohols suitable for use in pharmaceutical formulations are known to the skilled person and include e.g. mannitol and sorbitol. The polyol may be used as tonicity agent in the formulation.

A polyol can act and may be used as tonicity modifier in order to adjust the tonicity. Certain polyols, e.g. sugars, may also act as stabilizer, thereby supporting the stability of the provided formulation.

As disclosed herein, the polyol may be selected from a sugar and a sugar alcohol. Furthermore, combinations of two or more different polyols may be used as component b), as is also demonstrated in the examples. As is shown in the examples, sugars and sugar alcohols, as well as combinations thereof, can be advantageously used in the 150 mg/ml formulation according to the present disclosure. According to one embodiment, the polyol is selected from trehalose, sucrose, sorbitol, mannitol and combinations thereof. According to one embodiment, the formulation only comprises a polyol that is selected from sugars and/or sugar alcohols as component b). According to one embodiment, the formulation only comprises a single polyol as component b).

In specific embodiments, the polyol is a sugar. The polyol may be selected from trehalose and sucrose. As is shown in the examples, the formulation may comprise trehalose as polyol and the use of trehalose is advantageous. Trehalose can be used either alone or in combination with a further polyol, e.g. a further sugar or a sugar alcohol. According to specific embodiments, the formulation only comprises a single sugar, such as trehalose, as single polyol. Using a single polyol as excipient, e.g. to adjust the tonicity, can be advantageous.

According to one embodiment, the polyol is a sugar alcohol. The sugar alcohol may be selected from sorbitol and mannitol. In embodiments, the formulation comprises mannitol as polyol. In further embodiments, the formulation comprises sorbitol. As disclosed herein, mannitol and sorbitol may either be used as single polyol, or may be used in combination either with each other or in combination with a different polyol, such as a sugar or other sugar alcohol.

Sorbitol can be used to provide a stable formulation according to the present disclosure. In certain embodiments, a sorbitol-free formulation is provided. Sorbitol-free formulations are advantageous for patients with hereditary fructose intolerance. In a specific embodiment, the liquid pharmaceutical formulation therefore does not comprise sorbitol. In certain embodiments, the formulation does not comprise a sugar alcohol.

As is demonstrated in the examples, mannitol and/or trehalose can be used as polyol in the formulation of the present disclosure, in order to adjust the desired osmolality. However, the amount of mannitol within the 150 mg/ml formulation is limited by the mannitol solubility and the amount of stock solution, which can be added during the formulation step. Therefore, in embodiments, mannitol is used in combination with a sugar, such as the highly soluble trehalose. It was found that for the antibody formulation disclosed herein, trehalose is advantageous because it is soluble enough to achieve an isotonic formulation with one excipient. Therefore, in certain embodiments, trehalose is used as polyol and trehalose may be the only polyol in the formulation that is used to adjust the isotonicity.

The polyol can be used to adjust the osmolality. In embodiments, the formulation has an osmolality in a range of 200 mOsm/kg to 400 mOsm/kg, such as in a range of 225 mOsm/kg to 375 mOsm/kg. In embodiments, the osmolality is within a range of 250 mOsm/kg to 350 mOsm/kg, such as 275 mOsm/kg to 330 mOsm/kg or 290 mOsm/kg to 320 mOsm/kg. The formulation may be isotonic, wherein “isotonic” means that the formulation of interest has essentially the same osmotic pressure as human blood. Osmolality can be measured, for example, using a vapor pressure or ice-freezing type osmometer.

The concentration of the polyol in the formulation may be at least 80 mM or at least 95 mM. In embodiments, the concentration of the polyol in the formulation is at least 115 mM, at least 125 mM, at least 135 mM, at least 140 mM, at least 150 mM or at least 160 mM. In embodiments, the concentration of the polyol in the formulation is ≤500 mM, ≤450 mM or ≤400 mM. As disclosed herein, also two or more polyols may be used as excipient b). As disclosed herein, in one core embodiment the polyol is a sugar that is used in such concentration.

In one embodiment, the sugar is trehalose. The same applies with respect to the tonicity modifier that is used as component b) in the formulation according to the second sub-aspect. As disclosed herein, the tonicity modifier may be a polyol.

The concentration of the polyol in the formulation according to the first aspect, in particular the first and second sub-aspect thereof, may be in the range of 95 mM to 400 mM, such as 95 mM to 300 mM or 95 mM to 250 mM. Exemplary concentration ranges for a polyol in the formulation include, but are not limited to, 125 mM to 250 mM and 125 mM to 225 mM. The concentration of the polyol in the formulation is in one embodiment in the range of 125 mM to 225 mM. In one embodiment, the concentration of the polyol is in the range of 145 mM to 225 mM. As disclosed herein, in one core embodiment the polyol is a sugar that is used in such a concentration as described herein. In one embodiment, the sugar is trehalose.

According to one embodiment, the polyol is a sugar and wherein the concentration of the sugar is in the range of 125 mM to 250 mM, 150 mM to 250 mM, 150 mM to 200 mM or in the range of 160 mM to 200 mM. In a further embodiment, the concentration of the sugar is in the range of 170 mM to 200 mM. The concentration may be 185 mM. In one embodiment, said sugar is trehalose. Hence, also disclosed herein is a liquid pharmaceutical formulation comprising 150 mg/ml antibody and 185 mM trehalose as polyol. Trehalose may be added e.g. in the form of trehalose dihydrate.

### c) Surfactant

The liquid formulation according to the first aspect further comprises a surfactant. As is demonstrated by the examples, incorporating a surfactant in the 150 mg/ml formulation is advantageous. A surfactant is comprised as component c) in the formulation according to the first and second sub-aspect of the 150 mg/ml formulation according to the first aspect.

According to one embodiment, the surfactant is a non-ionic surfactant. Non-ionic surfactants suitable for pharmaceutical formulations are known in the art and are also described herein. The at least one surfactant may be a polysorbate (e.g. polysorbate 20) or a poloxamer (e.g. poloxamer 188). Combinations of surfactants may also be used. In one core embodiment, the surfactant is a polysorbate. The non-ionic surfactant may be selected from polysorbate 20 and/or polysorbate 80. Combinations may also be used. In one embodiment, the surfactant is polysorbate 20. In one embodiment, the formulation according to the present disclosure comprises a single surfactant, such as a single non-ionic surfactant, e.g. a single polysorbate.

In one embodiment, the concentration of the surfactant in the formulation is at least 0.05 mg/ml. The concentration may be at least 0.075 mg/ml. As is demonstrated in the examples, even low amounts of a surfactant provide a benefit. In embodiments, the surfactant concentration in the formulation is at least 0.1 mg/ml, at least 0.125 mg/ml, at least 0.15 mg/ml, at least 0.175 mg/ml or at least 0.185 mg/ml. In embodiments, the concentration of the surfactant in the formulation is ≤1 mg/ml, optionally ≤0.75 mg/ml or ≤0.5 mg/ml. In embodiments, the surfactant concentration in the formulation is ≤0.4 mg/ml, ≤0.3 mg/ml or ≤0.25 mg/ml. As disclosed herein, the surfactant may be a non-ionic surfactant. As disclosed herein, in core embodiments the surfactant is a polysorbate, optionally selected from polysorbate 20 and/or polysorbate 80. In embodiments, the surfactant is polysorbate 20. Polysorbate 20 can be advantageously used in such concentrations as disclosed herein as is demonstrated by the examples.

The concentration of the surfactant in the formulation may be in a range of 0.05 mg/ml to 0.75 mg/ml. Exemplary concentration ranges for a surfactant in the formulation include, but are not limited to, 0.05 mg/ml to 0.5 mg/ml, 0.075 mg/ml to 0.4 mg/ml or 0.075 mg/ml to 0.3 mg/ml. In embodiments, the concentration of the surfactant in the formulation is in the range of 0.05 mg/ml to 0.5 mg/ml, 0.075 mg/ml to 0.3 mg/ml or 0.1 mg/ml to 0.3 mg/ml. The concentration of the surfactant in the formulation may be 0.2 mg/ml. As is disclosed herein, the surfactant may be a non-ionic surfactant. In core embodiments the surfactant is a polysorbate, optionally selected from polysorbate 20 and/or polysorbate 80. In embodiments, the surfactant is polysorbate 20 which can be advantageously used in such concentration ranges as is demonstrated by the examples.

In specific embodiments, the formulation of the present disclosure comprises 0.2 mg/ml polysorbate 20 as surfactant. This formulation may comprise a sugar as component b), wherein the concentration of the sugar is in the range of 95 mM to 250 mM, 125 mM to 250 mM or 145 mM to 225 mM. The comprised sugar may be trehalose.

pH

The pH of the liquid pharmaceutical formulation, which in core embodiments is an aqueous formulation, may be in the range of pH 5.0 to 7.5, such as pH 5.0 to 7.0.

The pH of the liquid pharmaceutical formulation may be ≤6.8, such as ≤6.7, ≤6.6, ≤6.5, ≤6.4, ≤6.3 or ≤6.2. In embodiments, the pH of the liquid pharmaceutical formulation is ≤6.1, such as ≤6.0 or ≤5.9. In embodiments, the pH of the liquid pharmaceutical formulation is ≥5.2, such as ≥5.3, ≥5.4 or ≥5.5. Exemplary ranges for the pH of the liquid pharmaceutical formulation which has a pH ≥5.2 include but are not limited to 5.2 to 6.8, such as 5.2 to 6.7, 5.2 to 6.6, 5.2 to 6.5, 5.2 to 6.4, 5.2 to 6.3 and 5.2 to 6.2. Exemplary ranges for the pH of the liquid pharmaceutical formulation which has a pH ≥5.3 include but are not limited to 5.3 to 6.8, such as 5.3 to 6.7, 5.3 to 6.6, 5.3 to 6.5, 5.3 to 6.4, 5.3 to 6.3 and 5.3 to 6.2. Exemplary ranges for the pH of a liquid pharmaceutical formulation which has a pH ≥5.4 include but are not limited to 5.4 to 6.8, such as 5.4 to 6.7, 5.4 to 6.6, 5.4 to 6.5, 5.4 to 6.4, 5.4 to 6.3 and 5.4 to 6.2. Exemplary ranges for the pH of the liquid pharmaceutical formulation which has a pH ≥5.5 include but are not limited to 5.5 to 6.8, such as 5.5 to 6.7, 5.5 to 6.6, 5.5 to 6.5, 5.5 to 6.4, 5.5 to 6.3 and 5.5 to 6.2. Exemplary ranges for the pH of the liquid pharmaceutical formulation which has a pH ≥5.6 include but are not limited to 5.6 to 6.8, such as 5.6 to 6.7, 5.6 to 6.6, 5.6 to 6.5, 5.6 to 6.4, 5.6 to 6.3 and 5.6 to 6.2. In further embodiments the pH of the formulation is in a range of 5.6 to 6.0 or 5.6 to 5.9.

According to one embodiment, the pH of the liquid pharmaceutical formulation is in the range of 5.2 to 6.5. According to one embodiment, the pH of the liquid pharmaceutical formulation is in the range of 5.2 to 6.2. Lower pH values showed less aggregation during stability and physical stress studies as can be seen from the examples.

According to one embodiment, the pH of the liquid pharmaceutical formulation is in the range of 5.5 to 6.5. In one embodiment, the pH of the liquid pharmaceutical formulation is in a range of 5.5 to 6.2.

According to one embodiment, the pH is 5.5 to 5.9. In one embodiment, the pH is 5.6 to 5.8. 150 mg/ml risankizumab formulations having such pH were tested in the examples and showed favorable characteristics.

In a further embodiment, the pH of the liquid pharmaceutical formulation is 5.7.

In a further embodiment, the pH of the liquid pharmaceutical formulation is 6.2.

As disclosed herein, the pH of the stable liquid pharmaceutical formulation according to the second sub-aspect is 5.5 to 5.9. It may be in the range of 5.5 to 5.8. In embodiments, the pH of the stable 150 mg/ml formulation according to the second sub-aspect is 5.7.

### d) Buffer

The 150 mg/ml antibody formulation according to the first aspect can be provided as buffer-free or as buffer-containing formulation. According to one core embodiment disclosed herein, the pharmaceutical formulation comprises d) a buffer. Formulations comprising a buffer showed in experiments less increase in glide forces (max and average) compared to buffer-free formulations. Accordingly, a buffer may be used as component d) in the formulation according to the first and second sub-aspect of the 150 mg/ml risankizumab formulation according to the first aspect.

A buffer can be used to maintain the solution pH of the liquid pharmaceutical formulation. Suitable buffers for pharmaceutical formulations are known in the art and are described herein. The buffer may be an organic buffer. According to one embodiment, the buffer has a pKa within 1.5 or 1 pH unit of the final pH of the liquid pharmaceutical formulation at 25° C. In certain embodiments, the buffer has a pKa in the range of pH 4.2 to 7.2 or 4.5 to 7 at 25° C. The buffer may comprise a combination of buffers. In one embodiment, a single buffer is used in the formulation as component d).

The formulation may comprise a carboxylic acid buffer as buffer d).

According to one embodiment, the buffer is selected from an acetate buffer and a succinate buffer. As is demonstrated by the examples, formulations comprising such buffers provide advantageous stability features for the high concentration formulation of the antibody that is herewith provided. In further embodiments, the buffer is a histidine buffer.

In one embodiment, the buffer is an acetate buffer. An acetate buffer may comprise sodium acetate and acetic acid. Other acetate salts may also be used in the acetate buffer.

Further buffers that may be used include but are not limited to citrate, glutamate, glycine, lactate, maleate, phosphate or tartrate buffer.

The presence of a buffer salt may support the stability of the comprised antibody which is according to the present disclosure risankizumab.

According to one embodiment, the buffer d) comprised in the formulation is not a succinate buffer. In certain embodiments, the formulation is free of a succinate buffer. In certain embodiments, a single buffer is used which is an acetate buffer, e.g. provided by an acetate salt (e.g. sodium acetate) and acetic acid.

When used, the buffer will be included in a sufficient amount to maintain the selected pH of the formulation at storage conditions for the product shelf life.

The liquid pharmaceutical formulation disclosed herein may comprise at least 1 mM, at least 2 mM buffer, at least 3 mM buffer. The buffer concentration may be at least 4 mM, at least 4.5 mM or at least 5 mM. In embodiments, the buffer concentration is 100 mM or less, such as 75 mM or less or 50 mM or less. In embodiments, the buffer concentration in the formulation is 80 mM or less, such as 75 mM or less, 70 mM or less, 60 mM or less or 50 mM or less. In further embodiments, the buffer concentration is 45 mM or less, such as 40 mM or less, 35 mM or less, 30 mM or less or 25 mM or less. In further embodiments, the buffer concentration is 20 mM or less or 15 mM or less. Exemplary concentration ranges for the comprised buffer include but are not limited to 3 mM to 100 mM, such as 4 mM to 75 mM, 4 mM to 60 mM and 4 mM to 50 mM. Further exemplary buffer concentration ranges include but are not limited to 4 mM to 45 mM, such as 5 mM to 40 mM, 5 mM to 35 mM and 5 mM to 30 mM. Still further exemplary buffer concentration ranges include but are not limited to 5 mM to 25 mM, such as 5 mM to 20 mM and 5 mM to 15 mM. In one specific embodiment, the buffer concentration is in the range of 7 mM to 12 mM. Suitable buffers are disclosed herein. In one embodiment, the formulation comprises an acetate buffer in such concentration as described.

In embodiments, the buffer concentration is 20 mM or less or 15 mM or less. In further embodiments the buffer concentration is in the range of 4 mM to 50 mM. The buffer concentration of the formulation may be in the range of 5 mM to 25 mM or 5 mM to 20 mM. The buffer concentration may also be in the range of 5 mM to 15 mM or 7 mM to 12 mM. In embodiments, the buffer concentration is 10 mM.

In certain embodiments, the formulation comprises a single buffer. In specific embodiments the single buffer is an acetate buffer.

Specific Embodiments for Buffer-Containing Formulations Comprising 150 mg/Ml Antibody

According to one embodiment, the liquid pharmaceutical formulation comprises
a) 150 mg/ml of the antibody;
b) a sugar;
c) a non-ionic surfactant; and
d) a buffer;
   optionally wherein the pH of the formulation is in a range of pH 5.2 to pH 6.5, e.g. in the range of 5.2 to 6.2 or 5.5 to 6.2.

Suitable concentrations and embodiments for the excipients b) to d) were described above. In one embodiment, the concentration of the sugar is in the range of 145 mM to 225 mM and/or the concentration of the non-ionic surfactant is in the range of 0.05 mg/ml to 0.5 mg/ml or 0.075 mg/ml to 0.3 mg/ml. The sugar may be trehalose and the non-ionic surfactant may be a polysorbate, such as polysorbate 20. The pH may be 5.7. In a further embodiment the pH is 6.2.

According to one embodiment, the liquid pharmaceutical formulation comprises
a) 150 mg/ml of the antibody;
b) trehalose;
c) a polysorbate; and
d) a buffer;
   optionally wherein the pH of the formulation is in a range of pH 5.2 to pH 6.5, e.g. in the range of 5.2 to 6.2 or 5.5 to 6.2.

Suitable concentrations and embodiments for the excipients b) to d) were described above. In one embodiment, the concentration of trehalose is in the range of 145 mM to 225 mM and/or the concentration of the polysorbate is in the range of 0.05 mg/ml to 0.5 mg/ml or 0.075 mg/ml to 0.3 mg/ml. The pH may be 5.7. In a further embodiment the pH is 6.2.

The buffer comprised in these liquid pharmaceutical formulations may be acetate or succinate, optionally wherein the buffer concentration is in the range of 5 mM to 25 mM. The polysorbate may be polysorbate 20.

According to one embodiment, the liquid pharmaceutical formulation comprises
a) 150 mg/ml of the antibody;
b) 170 mM to 200 mM trehalose;
c) 0.1 mg/ml to 0.3 mg/ml polysorbate, optionally polysorbate 20; and
d) a buffer, optionally wherein the buffer is an acetate buffer.

The pH of this formulation may be in a range of pH 5.2 to pH 6.5, e.g. in the range of 5.2 to 6.2 or 5.5 to 6.2.

According to one embodiment, the liquid pharmaceutical formulation comprises
a) 150 mg/ml of the antibody;
b) 185 mM trehalose;
c) 0.2 mg/ml polysorbate 20; and
d) 10 mM acetate buffer;
   wherein the pH is 5.7.

This liquid formulation may be an aqueous formulation and in one embodiment, does not comprise any further additives.

Specific Embodiments for Buffer-Free Formulations Comprising 150 mg/Ml Antibody

As disclosed herein, also buffer-free liquid pharmaceutical formulations, in particular aqueous formulations, are provided. According to one embodiment, the liquid pharmaceutical formulation comprises
a) 150 mg/ml of the antibody;
b) a polyol, optionally wherein the polyol is a sugar or sugar alcohol; and
c) a non-ionic surfactant, optionally a polysorbate;
d) no buffer.

As noted above, the present disclosure also provides buffer-free formulations and no buffer is added as excipient. At 150 mg/ml, the antibody having the light and heavy chain sequences as shown in SEQ ID NO: 18 and 19 has a high buffering capacity. Storage-stable buffer-free formulations can be provided based on the disclosure provided herein as is also shown in the examples.

In embodiments, the pH of the buffer-free formulation is in a range of pH 5.2 to pH 6.5. The pH may be in the range of 5.2 to 6.2 or 5.5 to 6.2. In one embodiment, the pH is 5.7. In a further embodiment, the pH is 6.2.

In embodiments, the buffer-free formulation comprises 80 mM to 250 mM polyol. Suitable polyols such as sugars and sugar alcohols were disclosed in detail above and it is referred to this disclosure. In one embodiment, the sugar is trehalose.

According to one embodiment, the concentration of the non-ionic surfactant in the buffer-free formulation is in the range of 0.05 mg/ml to 0.5 mg/ml, 0.075 mg/ml to 0.4 mg/ml or 0.1 mg/ml to 0.3 mg/ml. According to one embodiment, the non-ionic surfactant is a polysorbate. It may be selected from polysorbate 20 and polysorbate 80 and is one embodiment polysorbate 20.

### Further Optional Components

In one embodiment, the liquid pharmaceutical formulation according to the present disclosure comprises an amino acid as further additive. Suitable embodiments for amino acids that can be added as excipient to a pharmaceutical formulation are known in the art and are also disclosed in the examples.

In one embodiment, the formulation comprises an amino acid which has a charged side chain, optionally a positive-charged side chain. An example of such amino acid is L-arginine.

According to one embodiment, the formulation comprises an amino acid, wherein the amino acid is present in the formulation as a salt, optionally a hydrochloride (HCl) salt.

According to one embodiment, the formulation comprises methionine. According to one embodiment, the formulation comprises amino acid L-proline.

According to one embodiment, the 150 mg/ml formulation according to the present disclosure does not comprise arginine. It was found that arginine containing formulations showed slightly elevated particle count during freeze/thaw stress studies as well as higher turbidity vales, even though there was no increase in turbidity over time. The viscosity was found to be higher. The amount of aggregates was slightly lower compared to other formulations comprising 150 mg/ml antibody but no arginine.

According to one embodiment, the formulation according to the present disclosure does not comprise an amino acid with a positive-charged side chain as excipient. According to one embodiment, the formulation according to the present disclosure does not comprise an amino acid with a charged side chain as excipient. According to one embodiment, the formulation according to the present disclosure does not comprise methionine as excipient. According to one embodiment, the formulation according to the present disclosure does not comprise an amino acid as additive.

Other excipients known in the art can be used in the formulation, as long as they do not negatively affect the stability.

However, in certain embodiments, no additional excipients are comprised in the formulation of the present disclosure. It is a particular advantage that a storage stable formulation of the antibody risankizumab can be provided with a formulation that consists essentially of or consists of a) the antibody (150 mg/ml); component b); c) a surfactant and optionally d) a buffer. As disclosed herein, it is advantageous that the formulation may comprise only a single polyol, a single surfactant and if present, a single buffer. Thereby a non-complex but nevertheless storage stable formulation is provided for the 150 mg/ml formulation of the antibody risankizumab.

### Stability Characteristics

As disclosed herein, advantageously, liquid pharmaceutical formulations comprising 150 mg/ml of the antibody are provided that are stable. Providing such stable, high-concentration formulation of the antibody risankizumab is particularly advantageous for therapeutic uses.

In the embodiments, a stable antibody formulation is a formulation wherein the antibody essentially retains its physical stability and/or biological activity upon storage. Various analytical techniques for measuring protein stability are available in the art and are disclosed herein. Stability can be measured at a selected temperature for a selected time period.

The stability characteristics of different liquid pharmaceutical formulations comprising 150 mg/ml of the antibody according to the present disclosure were tested in the examples and showed advantageous stability characteristics.

In embodiments, the stable liquid pharmaceutical formulation of the present disclosure shows no significant changes at a refrigerated temperature (2-8° C.) for at least 3 months, such as 6 months, or 1 year, or even up to 2 years or longer. A stable liquid formulation includes one that exhibits desired features at temperatures including 25° C. and 40° C. for periods including 1 month, 3 months, 6 months, 12 months, and/or 24 months.

The antibody in particular retains its physical stability in the pharmaceutical formulation, if it shows no significant increase of aggregation, precipitation and/or denaturation upon visual examination of color and/or clarity, or as measured by UV light scattering, size exclusion chromatography (SEC) and/or dynamic light scattering. The changes of protein conformation can be evaluated by fluorescence spectroscopy, which determines the protein tertiary structure, and by FTIR spectroscopy, which determines the protein secondary structure.

The antibody in particular retains its biological activity in the pharmaceutical formulation, if the biological activity of the antibody at a given time is within a predetermined range of the biological activity exhibited at the time the pharmaceutical formulation was prepared. The biological activity of the antibody can be determined, for example, by an antigen binding assay.

Aggregates can differ in origin, size, and type. Aggregates that can affect a biologic product's efficacy or safety are of particular concern, e.g. aggregates that can enhance immune responses and cause adverse clinical effects. High molecular weight aggregates, also called High Molecular Weight Species (HMWS) can be of particular concern. Aggregation can also potentially affect the subcutaneous bioavailability and pharmacokinetics of a therapeutic protein. It is advantageous that the present disclosure provides formulations, wherein the amount of high molecular weight species is low, also over extended storage times. The present disclosure in particular provides stabilized (or stable) aqueous pharmaceutical formulations as demonstrated by the reduced amounts of aggregates and/or reduced aggregate formation rates following storage. As described herein, the stability of such formulations is shown by the reduced amounts of HMWS and/or reduced HMWS formation rates following storage for varied time periods and at varied temperatures. In general, higher stability formulations are associated with lower amounts of HMWS, lower HMWS formation rates, and/or higher antibody main peaks at higher storage temperatures, relative to lower temperatures. As used herein, the term “high molecular weight species” or “HMWS” refers to higher order aggregates of the antibody of the formulations, as well as lower order aggregates of the antibody of the formulations. Lower order aggregates, include, for example, dimer species. The aggregate amounts and rates of formation may be measured or monitored by various techniques, including those disclosed in the examples.

As used herein, the term “low molecular weight species” or “LMWS” in particular refers to fragments of the antibody that are smaller than the monomer, including but not limited to free light chains, free heavy chains, molecules comprising one light chain and one heavy chain, antibody molecules missing one or both light chains, and antibody fragments obtained by cleavage of polypeptide chain(s) such as proteolytic fragments or other enzymatically or chemically degraded antibody molecules.

In certain embodiments, the antibody in the formulation disclosed herein is essentially maintained in monomeric form during storage. In particular embodiments, the formulation may fulfill one or more of the following stability characteristics:
In certain embodiments, following storage at 5° C. for 36 months, at least 94% of the antibody is present as a monomer as measured by UP-SEC, and/or the relative monomer content of the antibody does not decrease by more than 3% or more than 2.5%. In certain embodiments, following storage at 5° C. for 36 months, at least 95% or at least 96% of the antibody is present as a monomer as measured by UP-SEC, and/or the relative monomer content of the antibody does not decrease by more than 2% or more than 1.5%. In certain embodiments, following storage at 5° C. for 24 months, at least 94% of the antibody is present as a monomer as measured by UP-SEC, and/or the relative monomer content of the antibody does not decrease by more than 3% or more than 2.5%. In certain embodiments, following storage at 5° C. for 24 months, at least 95% or at least 96% of the antibody is present as a monomer as measured by UP-SEC, and/or the relative monomer content of the antibody does not decrease by more than 2% or more than 1.5% or more than 1%. In certain embodiments, following storage at 5° C. for 9 months, at least 96% or at least 96.5% of the antibody is present as a monomer as measured by UP-SEC, and/or the relative monomer content of the antibody does not decrease by more than 1.5% or more than 1%. In certain embodiments, following storage at 5° C. for 3 months, at least 96% or at least 97% of the antibody is present as a monomer as measured by UP-SEC, and/or the relative monomer content of the antibody does not decrease by more than 1% or more than 0.7% or more than 0.5%. In certain embodiments, following storage at 25° C. for 12 months, at least 90% or at least 92% of the antibody is present as a monomer as measured by UP-SEC, and/or the relative monomer content of the antibody does not decrease by more than 7% or more than 6% or more than 5%. In certain embodiments, following storage at 25° C. for 3 months, at least 95% of the antibody is present as a monomer as measured by UP-SEC, and/or the relative monomer content of the antibody does not decrease by more than 3% or more than 2%. In certain embodiments, following storage at 25° C. for 1 month, at least 96% of the antibody is present as a monomer as measured by UP-SEC, and/or the relative monomer content of the antibody does not decrease by more than 2% or more than 1%. In certain embodiments, following storage at 40° C. for 3 months, at least 87% or at least 88% of the antibody is present as a monomer as measured by UP-SEC, and/or the relative monomer content of the antibody does not decrease by more than 10% or more than 9% or more than 8%. In certain embodiments, following storage at 40° C. for 1 month, at least 93% or at least 94% of the antibody is present as a monomer as measured by UP-SEC, and/or the relative monomer content of the antibody does not decrease by more than 5% or more than 4%. In certain embodiments, following shaking at 25° C. for 21 days, at least 95% or at least 96% of the antibody is present as a monomer as measured by UP-SEC, and/or the relative monomer content of the antibody does not decrease by more than 2% or more than 1%. The decrease of the relative monomer content is calculated for the indicated storage time and temperature and in particular determined by comparing the relative monomer content at the beginning and at the end of the indicated storage. In particular embodiments, the measurements are performed as is described in the examples.

In certain embodiments, the antibody in the formulation disclosed herein does not form significant amounts of HMWS during storage. In particular, the formulation fulfills one or more of the following stability characteristics:
In certain embodiments, following storage at 5° C. for 36 months, less than 4% or less than 3% of the antibody is present as HMWS as measured by UP-SEC, and/or the relative HMWS content of the antibody does not increase by more than 2% or more than 1.5%. In certain embodiments, following storage at 5° C. for 24 months, less than 4% or less than 3% of the antibody is present as HMWS as measured by UP-SEC, and/or the relative HMWS content of the antibody does not increase by more than 2% or more than 1.5% or more than 1%. In certain embodiments, following storage at 5° C. for 9 months, less than 4% or less than 3% or less than 2.5% of the antibody is present as HMWS as measured by UP-SEC, and/or the relative HMWS content of the antibody does not increase by more than 1% or more than 0.8% or more than 0.6%. In certain embodiments, following storage at 5° C. for 3 months, less than 4% or less than 3% or less than 2.5% of the antibody is present as HMWS as measured by UP-SEC, and/or the relative HMWS content of the antibody does not increase by more than 1% or more than 0.8% or more than 0.6%. In certain embodiments, following storage at 25° C. for 12 months, less than 5% or less than 4% of the antibody is present as HMWS as measured by UP-SEC, and/or the relative HMWS content of the antibody does not increase by more than 3% or more than 2.5% or more than 2%. In certain embodiments, following storage at 25° C. for 3 months, less than 4% or less than 3.5% or less than 3.2% of the antibody is present as HMWS as measured by UP-SEC, and/or the relative HMWS content of the antibody does not increase by more than 2% or more than 1.5%. In certain embodiments, following storage at 25° C. for 1 month, less than 4% or less than 3.5% or less than 3% of the antibody is present as HMWS as measured by UP-SEC, and/or the relative HMWS content of the antibody does not increase by more than 1.5% or more than 1%. In certain embodiments, following storage at 40° C. for 3 months, less than 6.5% or less than 6% or less than 5.5% of the antibody is present as HMWS as measured by UP-SEC, and/or the relative HMWS content of the antibody does not increase by more than 5% or more than 4%. In certain embodiments, following storage at 40° C. for 1 month, less than 5% or less than 4.5% or less than 4% of the antibody is present as HMWS as measured by UP-SEC, and/or the relative HMWS content of the antibody does not increase by more than 2.5% or more than 2%. In certain embodiments, following shaking at 25° C. for 21 days, less than 3% or less than 2% of the antibody is present as HMWS as measured by UP-SEC, and/or the relative HMWS content of the antibody does not increase by more than 2% or more than 1.5% or more than 1%. The increase of the relative HMWS content is calculated for the indicated storage time and temperature and in particular determined by comparing the relative HMWS content at the beginning and at the end of the indicated storage. In particular embodiments, the measurements are performed as is described in the examples.

In further embodiments, the antibody in the formulation disclosed herein does not form significant amounts of LMWS during storage. In particular embodiments, the formulation may fulfill one or more of the following stability characteristics:
In certain embodiments, following storage at 5° C. for 36 months, less than 2% or less than 1.5% of the antibody is present as LMWS as measured by UP-SEC, and/or the relative LMWS content of the antibody does not increase by more than 1.5% or more than 1.5% or more than 0.5%. In certain embodiments, following storage at 5° C. for 24 months, less than 2% or less than 1.5% of the antibody is present as LMWS as measured by UP-SEC, and/or the relative LMWS content of the antibody does not increase by more than 1.5% or more than 1.5% or more than 0.5%. In certain embodiments, following storage at 5° C. for 9 months, less than 2% or less than 1.5% of the antibody is present as LMWS as measured by UP-SEC, and/or the relative LMWS content of the antibody does not increase by more than 1.5% or more than 1.5% or more than 0.5%. In certain embodiments, following storage at 5° C. for 3 months, less than 2% or less than 1.5% or less than 1% of the antibody is present as LMWS as measured by UP-SEC, and/or the relative LMWS content of the antibody does not increase by more than 1% or more than 0.5% or more than 0.25%. In certain embodiments, following storage at 25° C. for 12 months, less than 6% or less than 5% or less than 4.5% of the antibody is present as LMWS as measured by UP-SEC, and/or the relative LMWS content of the antibody does not increase by more than 5% or more than 4% or more than 3%. In certain embodiments, following storage at 25° C. for 3 months, less than 3% or less than 2% or less than 1.8% of the antibody is present as LMWS as measured by UP-SEC, and/or the relative LMWS content of the antibody does not increase by more than 2% or more than 1.5% or more than 1%. In certain embodiments, following storage at 25° C. for 1 month, less than 2% or less than 1.5% or less than 1.2% of the antibody is present as LMWS as measured by UP-SEC, and/or the relative LMWS content of the antibody does not increase by more than 1% or more than 0.6% or more than 0.4%. In certain embodiments, following storage at 40° C. for 3 months, less than 8% or less than 7% or less than 6% of the antibody is present as LMWS as measured by UP-SEC, and/or the relative LMWS content of the antibody does not increase by more than 8% or more than 7% or more than 6%. In certain embodiments, following storage at 40° C. for 1 month, less than 4% or less than 3.5% or less than 3% of the antibody is present as LMWS as measured by UP-SEC, and/or the relative LMWS content of the antibody does not increase by more than 3% or more than 2.5% or more than 2.2%. The increase of the relative LMWS content is calculated for the indicated storage time and temperature and in particular determined by comparing the relative LMWS content at the beginning and at the end of the indicated storage. In particular embodiments, the measurements are performed as is described in the examples.

In certain embodiments, the relative amount of the antibody being in monomeric form, HMWS and/or LMWS is determined using UP-SEC, in particular as described in the examples. For example, an ultra-performance liquid chromatography (UPLC) system such as an Acquity UPLC system of Waters (Milford, MA, USA) comprising a size exclusion chromatography (SEC) column is used. Proteins eluting from the SEC column may be detected by UV absorption at 280 nm and determining of the relative amounts may be done by calculating the area under the curve (AUC) for each elution peak. Peaks may be assigned to the different species by their elution time corresponding to the molecular size of the species. For measuring the relative monomer content, relative HMWS content and/or relative LMWS content of the antibody in the formulation, in particular the monomeric antibody, HMWS and LMWS are separated from each other, if present in the formulation. In particular, the relative content or amount is indicated as a percentage value and the sum of monomeric antibody, HMWS and LMWS is 100%.

In certain embodiments, the turbidity or opalescence of the formulation disclosed herein does not significantly increase during storage. In particular embodiments, the formulation may fulfill one or more of the following stability characteristics:
In certain embodiments, following storage at 5° C. for at least 36 months, the formulation has an opalescence of 12 FNU (Formazin Nephelometry Units) or less or 10 FNU or less, and/or the opalescence does not increase by more than 5 FNU or more than 3 FNU. In certain embodiments, following storage at 5° C. for at least 3, 6, 9, 12, 18 or 24 months, the formulation has an opalescence of 12 FNU (Formazin Nephelometry Units) or less or 10 FNU or less, and/or the opalescence does not increase by more than 5 FNU or more than 3 FNU. In certain embodiments, following storage at 25° C. for at least 1, 3, 6, 9 or 12 months, the formulation has an opalescence of 12 FNU or less or 10 FNU or less, and/or the opalescence does not increase by more than 7 FNU or more than 5 FNU. In certain embodiments, following storage at 40° C. for at least 1 or 3 months, the formulation has an opalescence of 12 FNU or less or 10 FNU or less, and/or the opalescence does not increase by more than 5 FNU or more than 3 FNU. In certain embodiments, following shaking at 25° C. for 21 days, the formulation has an opalescence of 12 FNU or less or 10 FNU or less, and/or the opalescence of the formulation does not increase by more than 3 FNU or more than 2 FNU. The increase of the opalescence is calculated for the indicated storage time and temperature and in particular determined by comparing the opalescence at the beginning and at the end of the indicated storage. In particular embodiments, the measurements are performed as is described in the examples.

In certain embodiments, the opalescence or turbidity is measured according to pharmacopeia or according to industrial standard ISO 7027. In certain embodiments, the opalescence or turbidity of the formulation is determined using a nephelometer such as a HACH Lange opalescence meter of Hach-Lange GmbH (Germany), in particular as described in the examples. Opalescence may be measured at different wavelengths, including at 400-600 nm. In embodiments, the FNA values indicated above are measured at 400-600 nm. Higher FNU values indicate a higher opalescence and turbidity.

In certain embodiments, the antibody in the formulation disclosed herein does not form significant additional amounts of acidic or basic variants during storage. In particular embodiments, the formulation may fulfill one or more of the following stability characteristics:
In certain embodiments, following storage at 5° C. for 36 months, at least 55%, at least 60% or at least 65% of the antibody is present as main peak variants, and/or the relative content of main peak variants of the antibody does not decrease by more than 8% or more than 7% or more than 5%, as determined by ion exchange chromatography (IEC). In certain embodiments, following storage at 5° C. for 24 months, at least 55%, at least 60% or at least 65% of the antibody is present as main peak variants, and/or the relative content of main peak variants of the antibody does not decrease by more than 8% or more than 7% or more than 5%, as determined by ion exchange chromatography (IEC). In certain embodiments, following storage at 5° C. for 6 months, at least 60% or at least 65% of the antibody is present as main peak variants, and/or the relative content of main peak variants of the antibody does not decrease by more than 5% or more than 4%, as determined by ion exchange chromatography (IEC). In certain embodiments, following storage at 5° C. for 3 months, at least 60% or at least 65% of the antibody is present as main peak variants, and/or the relative content of main peak variants of the antibody does not decrease by more than 4% or more than 3% or more than 2%, as determined by ion exchange chromatography (IEC). In certain embodiments, following storage at 25° C. for 12 months, at least 35% or at least 40% or at least 45% of the antibody is present as main peak variants, and/or the relative content of main peak variants of the antibody does not decrease by more than 35% or more than 30% or more than 25%, as determined by ion exchange chromatography (IEC). In certain embodiments, following storage at 25° C. for 3 months, at least 55% or at least 60% of the antibody is present as main peak variants, and/or the relative content of main peak variants of the antibody does not decrease by more than 15% or more than 10%, as determined by ion exchange chromatography (IEC). In certain embodiments, following storage at 25° C. for 1 month, at least 60% or at least 65% of the is present as main peak variants, and/or the relative content of main peak variants of the antibody does not decrease by more than 10% or more than 5%, as determined by ion exchange chromatography (IEC). In certain embodiments, following storage at 5° C. for 36 months, less than 30% or less than 28% of the antibody is present as acidic peak group variants, and/or the relative content of acidic peak group variants of the antibody does not increase by more than 4% or more than 3% or more than 2%, as determined by ion exchange chromatography (IEC). In certain embodiments, following storage at 5° C. for 24 months, less than 30% or less than 28% of the antibody is present as acidic peak group variants, and/or the relative content of acidic peak group variants of the antibody does not increase by more than 4% or more than 3% or more than 2%, as determined by ion exchange chromatography (IEC). In certain embodiments, following storage at 5° C. for 6 months, less than 30% or less than 28% of the antibody is present as acidic peak group variants, and/or the relative content of acidic peak group variants of the antibody does not increase by more than 4% or more than 3% or more than 2%, as determined by ion exchange chromatography (IEC). In certain embodiments, following storage at 5° C. for 3 months, less than 30% or less than 28% of the antibody is present as acidic peak group variants, and/or the relative content of acidic peak group variants of the antibody does not increase by more than 3% or more than 2% or more than 1%, as determined by ion exchange chromatography (IEC). In certain embodiments, following storage at 25° C. for 12 months, less than 50%, less than 45% or less than 40% of the antibody is present as acidic peak group variants, and/or the relative content of acidic peak group variants of the antibody does not increase by more than 30% or more than 25% or more than 20%, as determined by ion exchange chromatography (IEC). In certain embodiments, following storage at 25° C. for 3 months, less than 40% or less than 35% or less than 30% of the antibody is present as acidic peak group variants, and/or the relative content of acidic peak group variants of the antibody does not increase by more than 10% or more than 8% or more than 6%, as determined by ion exchange chromatography (IEC). In certain embodiments, following storage at 25° C. for 1 month, less than 35% or less than 30% or less than 28% of the antibody is present as acidic peak group variants, and/or the relative content of acidic peak group variants of the antibody does not increase by more than 4% or more than 3%, as determined by ion exchange chromatography (IEC). In certain embodiments, following storage at 5° C. for 36 months, less than 20%, less than 17%, less than 15% or less than 13% of the antibody is present as basic peak group variants, and/or the relative content of basic peak group variants of the antibody does not increase by more than 10% or more than 8% or more than 6%, as determined by ion exchange chromatography (IEC). In certain embodiments, following storage at 5° C. for 24 months, less than 20%, less than 17%, less than 15% or less than 13% of the antibody is present as basic peak group variants, and/or the relative content of basic peak group variants of the antibody does not increase by more than 10% or more than 8% or more than 6%, as determined by ion exchange chromatography (IEC). In certain embodiments, following storage at 5° C. for 6 months, less than 15% or less than 10% of the antibody is present as basic peak group variants, and/or the relative content of basic peak group variants of the antibody does not increase by more than 4% or more than 3% or more than 2%, as determined by ion exchange chromatography (IEC). In certain embodiments, following storage at 5° C. for 3 months, less than 15% or less than 10% of the antibody is present as basic peak group variants, and/or the relative content of basic peak group variants of the antibody does not increase by more than 3% or more than 2%, as determined by ion exchange chromatography (IEC). In certain embodiments, following storage at 25° C. for 12 months, less than 30% or less than 25% or less than 22% of the antibody is present as basic peak group variants, and/or the relative content of basic peak group variants of the antibody does not increase by more than 25% or more than 20% or more than 15%, as determined by ion exchange chromatography (IEC). In certain embodiments, following storage at 25° C. for 3 months, less than 20% or less than 15% or less than 12% of the antibody is present as basic peak group variants, and/or the relative content of basic peak group variants of the antibody does not increase by more than 9% or more than 7% or more than 5%, as determined by ion exchange chromatography (IEC). In certain embodiments, following storage at 25° C. for 1 month, less than 15% or less than 10% or less than 9% of the antibody is present as basic peak group variants, and/or the relative content of basic peak group variants of the antibody does not increase by more than 3% or more than 2%, as determined by ion exchange chromatography (IEC). The decrease of the relative content of the main peak variants and the increase of the relative content of the acidic and basic peak group variants are calculated for the indicated storage time and temperature and in particular determined by comparing the relative content of the respective peak variants at the beginning and at the end of the indicated storage. In particular embodiments, the measurements are performed as is described in the examples.

In certain embodiments, the relative amount of the antibody being main peak variants, acidic peak variants and/or basic peak variants is determined using ion exchange chromatography (IEC), in particular as described in the examples. In particular, weak cation exchange chromatography (WCX) is used. For example, a high-performance liquid chromatography (HPLC) system such as an Alliance HPLC system of Waters (Milford, MA, USA) comprising a WCX column is used. Proteins eluting from the WCX column may be detected by UV absorption at 280 nm and determining of the relative amounts may be done by calculating the area under the curve (AUC) for each elution peak or each group of elution peaks. Peaks may be assigned to the different species by their elution conditions corresponding to the surface charge of the antibody species. The main peak is the largest peak in an IEC chromatogram of the non-degraded antibody. For the stability analysis, the measurement can be performed after preparation of the formulation (TO) and then after the indicated storage time under the indicated storage condition. The acidic peak group (APG) includes all peaks prior to the main peak. These peaks include antibody variants which are more acidic than the native antibody variants of the main peak, and/or which have more negative charges on their surface under the chromatography conditions. The basic peak group includes all peaks after the main peak. These peaks include antibody variants which are more acidic than the native antibody variants of the main peak, and/or which have more positive charges on their surface under the chromatography conditions. For measuring the relative amount of main peak variants, acidic peak group variants and/or basic peak group variants of the antibody in the formulation, in particular the main peak is separated from the acidic peak group and the basic peak group, if present in the formulation. In particular, the relative content or amount is indicated as a percentage value and the sum of main peak variants, acidic peak group variants and basic peak group variants is 100%.

In certain embodiments, the antibody in the formulation disclosed herein essentially maintains its specific binding activity to human IL-23 during storage. In particular embodiments, the formulation fulfills one or more of the following stability characteristics:
In certain embodiments, following storage at 5° C. for 36 months, at least 95% or at least 97% of the specific binding activity to IL-23 is measured compared to a reference antibody, wherein the reference antibody has not been stored. In certain embodiments, following storage at 5° C. for 4, 6, 9, 12, 18 or 24 months, at least 95% or at least 97% of the specific binding activity to IL-23 is measured compared to a reference antibody, wherein the reference antibody has not been stored.
In certain embodiments, following storage at 25° C. for 2, 3, 4, 6, 9, 12, or 18 months, at least 93% or at least 96% of the specific binding activity to IL-23 is measured compared to a reference antibody, wherein the reference antibody has not been stored. In certain embodiments, following storage at 40° C. for 3, 4 or 6 months, at least 90% or at least 95% of the specific binding activity to IL-23 is measured compared to a reference antibody, wherein the reference antibody has not been stored. In particular embodiments, the measurements are performed as is described in the examples.

In certain embodiments, the specific binding activity to human IL-23 of the antibody in the formulation is determined using surface plasmon resonance measurements, for example using a Biacore instrument such as Biacore T200 of GE Healthcare Life Science (United Kingdom), in particular as described in the examples.

### Further Characteristics of the Liquid Pharmaceutical Formulation According to the First Aspect

In advantageous embodiments, the liquid pharmaceutical formulation of the present disclosure is an aqueous formulation. All liquid formulations disclosed herein are in one embodiment an aqueous formulation. The following description applies to the 150 mg/ml formulation according to the first aspect and therefore, also applies to the formulations according to the first and second sub-aspect as disclosed herein, unless the specific context indicates otherwise.

According to one embodiment the dynamic viscosity of the liquid pharmaceutical formulation according to the first aspect measured at 20° C. is ≤30 mPas (mPa·s), such as 25 mPas or ≤20 mPas. In embodiments, the dynamic viscosity of the formulation measured at 20° C. is ≤18 mPas, such as ≤16 mPas, ≤15 mPas, ≤14 mPas, ≤13 mPas or ≤12 mPas. In specific embodiments, the dynamic viscosity is such that the formulation is suitable for subcutaneous administration, as is also shown in the examples. The dynamic viscosity may be determined as described in the examples.

According to one embodiment, the formulation of the present disclosure has a conductivity in a range of 0.8 to 5 mS/cm. In embodiments, the conductivity range is 1 to 2 mS/cm or 1.2 to 1.8 mS/cm. In embodiments, the formulation is characterized in that the change of conductivity over a storage time of at least 12 months at 25° C. is ≤1 mS/cm, such as ≤0.75 mS/cm, ≤0.5 mS/cm or ≤0.3 mS/cm.

The liquid formulation according to the present disclosure is a pharmaceutical formulation. A pharmaceutical formulation in particular refers to compositions which are in such form as to permit the active ingredient (here the antibody comprising a light chain as shown in SEQ ID NO: 18 and a heavy chain as shown in SEQ ID NO: 19) to be effective, and which contains no additional components which are toxic to the subjects to which the formulation would be administered.

The formulations according to the first aspect disclosed herein are advantageously suitable for parenteral delivery. Parenteral administration includes e.g. subcutaneous, intramuscular, intradermal, intramedullary injections, as well as intrathecal, direct intraventricular, intravenous, intraperitoneal and intravitreal. Drugs can be administered in a variety of conventional ways, such as intraperitoneal, parenteral, intraarterial or intravenous injection. In one embodiment, the disclosed formulation is an injectable formulation. The formulation disclosed herein is in embodiments suitable for subcutaneous, intravenous, or intramuscular administration. Advantageously, the disclosed formulation is suitable for subcutaneous injection. The 150 mg/ml formulation disclosed herein is particularly advantageous, because overall characteristics are achieved which makes the formulation particularly suitable for subcutaneous administration. The high concentration allows to administer a small volume of the formulation while still achieving a high antibody dose (here e.g. 1 ml for 150 mg dose). Furthermore, the formulations according to the present disclosure show a good syringeability. They moreover have advantageous viscosity and osmolality characteristics and achieve good glide forces (max and average), also upon storage as is disclosed in the examples. In embodiments, the liquid pharmaceutical formulation according to the present disclosure is isotonic with the intended site of administration. For example, if the formulation is intended for administration parenterally, it can be isotonic with blood (which is about 300 mOsm/kg osmolality). Suitable osmolality ranges are described elsewhere.

The liquid antibody formulation can be made by taking the drug substance which is in liquid form (e.g., in an aqueous pharmaceutical formulation) and buffer exchanging and preparing it into the desired buffer as the last step of the purification process. The drug substance in the final buffer may be concentrated to a desired concentration or a more concentrated form of the antibody is diluted to achieve the 150 mg/ml concentration. Concentration of the formulation can be carried out by any suitable method. In one aspect, the concentration process can include ultrafiltration.

The liquid pharmaceutical formulation according to the first aspect is in one core embodiment not a formulation that has been prepared by reconstituting a lyophilized formulation. In this core embodiment, there is no lyophilization step during the preparation of the liquid pharmaceutical formulation. Excipients such as component b) and surfactant c) may be added to the drug substance which may be diluted using the appropriate buffer to final protein concentration of 150 mg/ml. A pharmaceutical formulation to be used for in vivo administration typically is sterile. In certain embodiments, this may be accomplished by filtration through sterile filtration membranes. The final formulated drug substance may thus be filtered (e.g. using 0.22 μm filters) and may be filled into a final container (e.g. glass vials or syringe). The prepared liquid formulation is in this embodiment for direct administration to the patient so that there is no lyophilization or reconstitution step. Such liquid pharmaceutical formulations are disclosed herein and were also made and analysed in the examples.

### Lyophilized and Reconstituted Pharmaceutical Formulations

According to one embodiment, the liquid pharmaceutical formulation according to the first aspect is prepared from a lyophilized pharmaceutical formulation by reconstitution. In embodiments, the liquid pharmaceutical composition described herein is thus a reconstituted formulation. This applies to the liquid formulations according to the first and second sub-aspect of the 150 mg/ml antibody formulation according to the first aspect.

The terms “lyophilization,” or “lyophilized” in particular refer to a process by which the material to be dried is first frozen and then the ice or frozen solvent is removed by sublimation in a vacuum environment. Such technologies are well-known in the art and therefore, are not described in detail herein. An excipient may be included in pre-lyophilized formulations to enhance stability of the lyophilized product upon storage. The lyophilized formulation may comprise a cryoprotectant, which generally includes agents which provide stability to the protein against freezing-induced stresses. They may also offer protection during primary and secondary drying, and long-term product storage. Examples include sugars such as sucrose and trehalose and surfactants such as polysorbates. The lyophilized formulation may also include a lyoprotectant, which includes agents that provide stability to the protein during the drying or dehydration process (primary and secondary drying cycles). This helps to maintain the protein conformation, minimize protein degradation during the lyophilization cycle and improve the long-term product stability. Examples include polyols, such as sugars, e.g. sucrose and trehalose. The liquid pharmaceutical formulations disclosed according to the first aspect comprise excipients that qualify as cryo- and/or lyoprotectant. accordingly, lyophilized formulations may be prepared from such formulations. In an embodiment, the antibody risankizumab is formulated as a lyophilized powder for reconstituting and utilizing for intravenous administration.

A “reconstituted” formulation is one that has been prepared by dissolving a lyophilized pharmaceutical antibody formulation in a diluent such that the antibody is dispersed in the reconstituted formulation. The reconstituted formulation is suitable for administration, and may optionally be suitable for subcutaneous administration.

The lyophilized pharmaceutical formulation is prepared in anticipation of reconstitution at the desired concentration, here 150 mg/ml of the antibody.

According to one embodiment, a lyophilized formulation of an anti-IL-23p19 antibody is provided, wherein the antibody comprises a light chain amino acid sequence according to SEQ ID NO: 18 and a heavy chain amino acid sequence according to SEQ ID NO: 19. According to one embodiment, the lyophilized formulation of the antibody risankizumab is defined in terms of the solution used to make the lyophilized formulation, e.g. the pre-lyophilization solution. This lyophilized formulation is made by lyophilizing the liquid 150 mg/ml antibody formulation according to the first aspect, such as the liquid pharmaceutical formulation according to the first aspect as defined in any one of the below embodiments 1 to 86. As disclosed herein, the liquid formulation is in one embodiment an aqueous formulation. Such aqueous formulation may be used to prepare the lyophilized pharmaceutical formulation.

In yet other embodiments, the lyophilized formulation of the antibody risankizumab is defined in terms of the reconstituted solution generated from the lyophilized formulation. According to one embodiment, a lyophilized formulation of an anti-IL-23p19 antibody is thus provided, wherein the antibody comprises a light chain amino acid sequence according to SEQ ID NO: 18 and a heavy chain amino acid sequence according to SEQ ID NO: 19, said lyophilized formulation providing upon reconstitution the liquid 150 mg/ml antibody formulation according to the first aspect, in particular the first and second sub-aspect thereof. According to embodiments, the lyophilized formulation provides upon reconstitution the liquid pharmaceutical formulation as defined in any one of the below embodiments 1 to 86 or 104 to 119. This risankizumab formulation may be an aqueous formulation.

Also provided is a lyophilized formulation comprising
a) an anti-IL-23p19 antibody, wherein the antibody comprises a light chain amino acid sequence according to SEQ ID NO: 18 and a heavy chain amino acid sequence according to SEQ ID NO: 19, in an amount that upon reconstitution provides an antibody concentration of 150 mg/ml;
b) a polyol;
c) a surfactant; and
d) optionally a buffer.

In one embodiment, the lyophilized pharmaceutical formulation comprises 150 mg of the antibody. The antibody is risankizumab.

Also provided is a lyophilized formulation comprising
a) an anti-IL-23p19 antibody, wherein the antibody comprises a light chain amino acid sequence according to SEQ ID NO: 18 and a heavy chain amino acid sequence according to SEQ ID NO: 19, in an amount that upon reconstitution provides an antibody concentration of 150 mg/ml;
b) a tonicity modifier;
c) a surfactant; and
d) optionally a buffer.

In one embodiment, the lyophilized pharmaceutical formulation comprises 150 mg of the antibody. The antibody is risankizumab.

The comprised components such as suitable polyols for pharmaceutical formulations were already disclosed above in conjunction with the liquid pharmaceutical formulation and it is referred to the above disclosure which also applies here. Suitable polyols include sugars and sugar alcohols, which may also be used in combination. The polyol may have one or more of the characteristics as defined in any one of the below embodiments 6 to 13 of the liquid pharmaceutical formulation according to the first aspect. In one embodiment, the polyol is a sugar, optionally selected from trehalose and sucrose. In one embodiment, the sugar is trehalose.

Suitable surfactants were already disclosed above in conjunction with the liquid pharmaceutical formulation and it is referred to the above disclosure which also applies here. The surfactant may have one or more of the characteristics as defined in any one of the below embodiments 22 to 25 of the liquid pharmaceutical formulation according to the first aspect. In one embodiment, the surfactant is a polysorbate, optionally selected from polysorbate 20 and 80. In one embodiment, the polysorbate is polysorbate 20.

The lyophilized formulation comprises in one embodiment a buffer. Buffers suitable to prepare lyophilized formulations are known in the art and suitable buffers were also disclosed above in conjunction with the liquid pharmaceutical formulation according to the first aspect and it is referred to the above disclosure.

According to one embodiment, the lyophilized formulation is characterized in that the formulation has upon reconstitution a pH as disclosed herein for the liquid pharmaceutical formulation according to the first aspect. Suitable pH values were disclosed above and it is referred to the respective disclosure which also applies here. Upon reconstitution, the pH may be as defined in any one of the below embodiments 31 to 36 of the liquid 150 mg/ml pharmaceutical formulation. Furthermore, upon reconstitution, the pH may be as defined for the formulation according to second sub-aspect. The pH upon reconstitution may be 5.5 to 5.9, e.g. 5.6 to 5.8.

The lyophilized risankizumab formulations of the present disclosure are reconstituted prior to administration. In some instances, it may be desirable to lyophilize the risankizumab formulation in the container in which reconstitution of the antibody is to be carried out in order to avoid a transfer step.

### Containers and Uses

According to a further aspect of the present disclosure, a sealed container is provided which contains the liquid pharmaceutical formulation or the lyophilized pharmaceutical formulation according to the first aspect of the present disclosure. The container may be a vial or pre-filled syringe. In embodiments, the container contains 2 ml or less of the liquid pharmaceutical formulation, optionally 1.5 ml or less or 1 ml or less. Such container may comprise the advantageous stable liquid pharmaceutical formulation according to the first or second sub-aspect of the 150 mg/ml antibody formulation according to the first aspect.

In core embodiments, the container such as the syringe comprises a single dose of 150 mg antibody. As disclosed herein, the antibody is risankizumab.

In one embodiment, the liquid pharmaceutical formulation according to the first aspect of the present disclosure is comprised in a syringe which is equipped with a needle. In particular embodiments, the needle is suitable for subcutaneous administration. The needle may be a 27 Gauge spinal thin-wall needle or other needle suitable for subcutaneous use.

According to one embodiment the pre-filled syringe equipped with a needle has an average gliding force that is 20 N or less. In embodiments, the average gliding force is in the range of 5 to 20 N or 5 to 15 N. In embodiments, the pre-filled syringe has a break loose force of 3 to 12 N, preferably 3 to 9 N.

In certain embodiments, the syringe equipped with a needle and comprising the liquid pharmaceutical formulation according to the first aspect essentially maintains the maximum and/or average gliding force needed to eject it from a syringe during storage. In certain embodiments, following storage at 5° C. for 36 months, the maximum gliding force of the syringe pre-filled with the liquid formulation is 14 N or lower, 12 N or lower or 10 N or lower, and/or the maximum gliding force does not increase by more than 5 N or more than 4 N or more than 3 N. In certain embodiments, following storage at 5° C. for 24 months, the maximum gliding force of the syringe pre-filled with the liquid formulation is 14 N or lower, 12 N or lower, 10 N or lower or 8 N or lower, and/or the maximum gliding force does not increase by more than 3 N or more than 2 N or more than 1.5 N. In certain embodiments, following storage at 5° C. for 9 months, the maximum gliding force is 9 N or lower or 8 N or lower, and/or the maximum gliding force does not increase by more than 2 N or more than 1.5 N or more than 1 N. In certain embodiments, following storage at 5° C. for 3 months, the maximum gliding force is 8 N or lower or 7.5 N or lower, and/or the maximum gliding force does not increase by more than 1.5 N or more than 1 N. In certain embodiments, following storage at 25° C. for 3 months, the maximum gliding force is 10 N or lower or 8 N or lower, and/or the maximum gliding force does not increase by more than 3 N or more than 2 N or more than 1.5 N. In certain embodiments, following storage at 25° C. for 1 month, the maximum gliding force of the pre-filled syringe equipped with a needle is 8 N or lower or 7.5 N or lower, and/or the maximum gliding force does not increase by more than 1.5 N or more than 1 N. In certain embodiments, following storage at 40° C. for 1 month, the maximum gliding force is 16 N or lower or 13 N or lower, and/or the maximum gliding force does not increase by more than 10 N or more than 8 N or more than 6 N.

In certain embodiments, following storage at 5° C. for 36 months, the average gliding force of the syringe prefilled with the liquid formulation according to the first aspect and equipped with a needle is 14 N or lower, 12 N or lower, 10 N or lower or 9 N or lower, and/or the average gliding force does not increase by more than 5 N or more than 4 N or more than 3 N. In certain embodiments, following storage at 5° C. for 24 months, the average gliding force of the syringe prefilled with the liquid formulation according to the first aspect and equipped with a needle is 14 N or lower, 12 N or lower, 10 N or lower or 8 N or lower, and/or the average gliding force does not increase by more than 3 N or more than 2 N or more than 1.5 N. In certain embodiments, following storage at 5° C. for 9 months, the average gliding force of the pre-filled syringe equipped with the needle is 9 N or lower or 7.5 N or lower, and/or the average gliding force does not increase by more than 2 N or more than 1.5 N or more than 1 N. In certain embodiments, following storage at 5° C. for 3 months, the average gliding force is 8 N or lower or 7 N or lower, and/or the average gliding force does not increase by more than 1.5 N or more than 1 N or no more than 0.5 N. In certain embodiments, following storage at 25° C. for 12 months, the average gliding force is 15 N or lower or 13 N or lower, and/or the average gliding force does not increase by more than 9 N or more than 8 N or more than 7 N. In certain embodiments, following storage at 25° C. for 3 months, the average gliding force is 9 N or lower or 8 N or lower, and/or the average gliding force does not increase by more than 3 N or more than 2 N or more than 1.5 N. In certain embodiments, following storage at 25° C. for 1 month, the average gliding force is 8 N or lower or 7 N or lower, and/or the average gliding force does not increase by more than 1.5 N or more than 1 N or no more than 0.5 N. In certain embodiments, following storage at 40° C. for 3 month, the average gliding force is 18 N or lower or 15 N or lower, and/or the average gliding force does not increase by more than 12 N or more than 10 N or more than 9 N. In certain embodiments, following storage at 40° C. for 1 month, the average gliding force is 13 N or lower or 10 N or lower, and/or the average gliding force does not increase by more than 7 N or more than 5 N or more than 3 N.

The increase of the maximum or relative gliding force is calculated for the indicated storage time and temperature and in particular determined by comparing the maximum or relative gliding force at the beginning and at the end of the indicated storage. In particular embodiments, the measurements are performed as is described in the examples.

The maximum gliding force of the formulation refers to the maximum mechanical force needed to eject the formulation from a syringe. The average gliding force of the formulation refers to the average mechanical force needed to eject the formulation from a syringe. In some embodiments, the gliding force is determined according to industrial norms such as ISO 7886, ISO 11040 and ISO 11499. In certain embodiments, the maximum and average gliding force of the formulation is determined using a tensile and compression testing machine such as a Zwick 2.5TS/N of Zwick (Germany), in particular as described in the examples. The measurement may be performed using a 1 ml syringe with a 27 gauge×12 inch needle, such as a Neopak 1 ml syringe of Becton Dickinson (USA), in particular a syringe equipped with a needle as used in the examples. The measurement may be performed using a speed of about 300 to 500 mm/min such as about 380 mm/min, in particular 379.2 mm/min, for example for 5 seconds.

A further aspect according to the present disclosure pertains to the liquid pharmaceutical formulations or the lyophilized pharmaceutical formulations according to the first aspect or the container according to the further aspect disclosed herein for therapeutic treatment of a human subject. The disease to be treated is a disease that can be treated with an anti-IL-23p19 antibody and such diseases are known in the art. The disease may be selected from the group consisting of inflammatory diseases, autoimmune diseases, respiratory diseases, metabolic disorders and cancer. In embodiments, the disease is a chronic disease. The disease to be treated may be selected from psoriasis and inflammatory bowel disease. In further embodiments, the disease to be treated may be selected from psoriatic arthritis and Crohn's disease. Administering the high concentration 150 mg/ml liquid pharmaceutical formulation according to the present disclosure to the patient for therapy is advantageous for the reasons discussed herein.

Further Embodiments of the 150 mg/ml Formulation

In the following, further specific contemplated embodiments of the 150 mg/ml antibody formulation according to the first aspect are disclosed:
1. A liquid pharmaceutical formulation comprising
a) 150 mg/ml of an anti-IL-23p19 antibody, wherein the antibody comprises a light chain amino acid sequence according to SEQ ID NO: 18 and a heavy chain amino acid sequence according to SEQ ID NO: 19;
b) a polyol; and
c) a surfactant.
2. The formulation according to embodiment 1, comprising
d) a buffer.
3. The formulation according to embodiment 1 or 2, wherein the antibody is risankizumab.
4. The formulation according to any one of embodiments 1 to 3, wherein the antibody has been recombinantly produced in a mammalian cell.
5. The formulation according to embodiment 4, wherein the antibody has been recombinantly produced in a CHO cell.
6. The formulation according to one or more of embodiments 1 to 5, wherein the polyol is selected from a sugar, a sugar alcohol and combinations thereof.
7. The formulation according to embodiment 6, wherein the polyol is selected from trehalose, sucrose, sorbitol, mannitol and combinations thereof.
8. The formulation according to embodiment 6, wherein the polyol is a sugar, optionally selected from trehalose and sucrose.
9. The formulation according to embodiment 6, comprising trehalose as polyol.
10. The formulation according to embodiment 6, wherein the polyol is selected from sorbitol and mannitol.
11. The formulation according to embodiment 6, comprising mannitol as polyol.
12. The formulation according to any one of embodiments 1 to 11, wherein the liquid pharmaceutical formulation does not comprise sorbitol.
13. The formulation according to one or more of embodiments 1 to 9, wherein the formulation does not comprise a sugar alcohol.
14. The formulation according to one or more of embodiments 1 to 13, having one or more of the following characteristics:
(i) the concentration of the polyol in the formulation is at least 95 mM;
(ii) the concentration of the polyol in the formulation is at least 125 mM;
(iii) the concentration of the polyol in the formulation is at least 150 mM;
(iv) the concentration of the polyol in the formulation is ≤500 mM, ≤450 mM or ≤400 mM; (v) the concentration of the polyol in the formulation is ≤350 mM, ≤300 mM or ≤275 mM; and/or
(vi) the concentration of the polyol in the formulation lies in the range of 95 mM to 450 mM or 125 mM to 400 mM;
optionally wherein the polyol is a sugar and/or a sugar alcohol.
15. The formulation according to one or more of embodiments 1 to 13, wherein the concentration of the polyol in the formulation is in the range of 95 mM to 250 mM, optionally wherein the polyol is a sugar.
16. The formulation according to one or more of embodiments 1 to 13, wherein the concentration of the polyol in the formulation is in the range of 125 mM to 225 mM, optionally wherein the polyol is a sugar such as trehalose.
17. The formulation according to one or more of embodiments 1 to 13, wherein the concentration of the polyol is in the range of 145 mM to 225 mM, optionally wherein the polyol is a sugar such as trehalose.
18. The formulation according to one or more of embodiments 1 to 13, wherein the polyol is a sugar and wherein the concentration of the sugar is in the range of 150 mM to 200 mM, optionally wherein the sugar is trehalose.
19. The formulation according to one or more of embodiments 1 to 13, wherein the polyol is a sugar and wherein the concentration of the sugar is in the range of 160 mM to 200 mM, optionally wherein the sugar is trehalose.
20. The formulation according to one or more of embodiments 1 to 13, wherein the polyol is a sugar and wherein the concentration of the sugar is in the range of 170 mM to 200 mM, optionally wherein the sugar is trehalose.
21. The formulation according to one or more of embodiments 1 to 13, comprising 185 mM trehalose as polyol.
22. The formulation according to one or more of embodiments 1 to 21, wherein the surfactant is a non-ionic surfactant.
23. The formulation according to embodiment 22, wherein the surfactant is a polysorbate.
24. The formulation according to embodiment 22 or 23, wherein the non-ionic surfactant is selected from polysorbate 20 and/or polysorbate 80.
25. The formulation according to one or more of embodiments 1 to 24, wherein the surfactant is polysorbate 20.
26. The formulation according to one or more of embodiments 1 to 25, in particular any one of embodiments 23 to 25, wherein the concentration of the surfactant in the formulation is at least 0.05 mg/ml, optionally at least 0.075 mg/ml.
27. The formulation according to one or more of embodiments 1 or 26, in particular any one of embodiments 23 to 25, wherein the concentration of the surfactant in the formulation is in a range of 0.05 mg/ml to 0.75 mg/ml.
28. The formulation according to one or more of embodiments 1 or 27, in particular any one of embodiments 23 to 25, wherein the concentration of the surfactant in the formulation is in the range of 0.05 mg/ml to 0.5 mg/ml or 0.075 mg/ml to 0.3 mg/ml.
29. The formulation according to embodiment 25, wherein the formulation comprises 0.2 mg/ml polysorbate 20 as surfactant.
30. The formulation according to embodiment 29, wherein the polyol is a sugar and wherein the concentration of the sugar is in the range of 145 mM to 225 mM, optionally wherein the sugar is trehalose.
31. The formulation according to one or more of embodiments 1 to 30, wherein the pH of the liquid pharmaceutical formulation is in the range of pH 5.0 to 7.5 or pH 5.0 to 7.0.
32. The formulation according to one or more of embodiments 1 to 30, wherein the pH of the liquid pharmaceutical formulation is in a range of 5.2 to 6.5 or 5.2 to 6.2.
33. The formulation according to one or more of embodiments 1 to 30, wherein the pH of the liquid pharmaceutical formulation is in a range of 5.5 to 6.5 or 5.5 to 6.2.
34. The formulation according to one or more of embodiments 1 to 30, wherein the pH of the liquid pharmaceutical formulation is in a range of 5.5 to 5.9.
35. The formulation according to one or more of embodiments 1 to 30, wherein the pH of the liquid pharmaceutical formulation is in a range of 5.6 to 5.8.
36. The formulation according to one or more of embodiments 1 to 30, wherein the pH of the liquid pharmaceutical formulation is 5.7 or 6.2.
37. The formulation according to one or more of embodiments 2 to 36, wherein the buffer has a pKa within 1.5 or one pH unit of the final pH of the liquid pharmaceutical formulation at 25° C., optionally wherein the buffer has a pKa within the range of pH 4.2 to 7.2 or pH 4.5 to 7 at 25° C.
38. The formulation according to one or more of embodiments 2 to 37, wherein the buffer is an organic buffer, which is optionally selected from an acetate buffer and a succinate buffer.
39. The formulation according to embodiment 38, wherein the buffer is an acetate buffer, optionally wherein the acetate buffer comprises sodium acetate and acetic acid.
40. The formulation according to one or more of embodiments 2 to 37, wherein the buffer is a histidine buffer or wherein the formulation fulfills at least one of the following characteristics: (i) it comprises a carboxylic acid buffer; (ii) it does not comprise a succinate buffer.
41. The formulation according to one or more of embodiments 2 to 40, comprising at least 1 mM, at least 2 mM or at least 3 mM buffer, optionally comprising at least 4 mM, at least 4.5 mM or at least 5 mM buffer.
42. The formulation according to one or more of embodiments 2 to 41, wherein the buffer concentration is 100 mM or less, 75 mM or less or 50 mM or less.
43. The formulation according to one or more of embodiments 2 to 41, wherein the buffer concentration is 20 mM or less or 15 mM or less.
44. The formulation according to one or more of embodiments 2 to 41, wherein the buffer concentration is in the range of 4 mM to 50 mM.
45. The formulation according to one or more of embodiments 2 to 41, wherein the buffer concentration is in the range of 5 mM to 25 mM or 5 mM to 20 mM.
46. The formulation according to one or more of embodiments 2 to 41, wherein the buffer concentration is in the range of 5 mM to 15 mM or 7 mM to 12 mM.
47. The formulation according to one or more of embodiments 2 to 41, wherein the buffer concentration is 10 mM.
48. The formulation according to one or more of embodiments 2 to 47, wherein the formulation comprises a single buffer, optionally an acetate buffer.
49. The formulation according to any one of claim 1 or 3 to 36, wherein the formulation does not comprise a buffer.
50. The formulation according to one or more of embodiments 1 to 49, wherein the formulation is an aqueous formulation.
51. The formulation according to any one of embodiments 2 to 48 or 50, comprising
a) 150 mg/ml of the antibody;
b) a sugar, optionally wherein the concentration of the sugar is in the range of 95 mM to 250 mM or 145 mM to 225 mM;
c) a non-ionic surfactant, optionally wherein the concentration of the non-ionic surfactant is in the range of 0.05 mg/ml to 0.5 mg/ml or 0.075 mg/ml to 0.3 mg/ml; and
d) a buffer.
52. The formulation according any one of embodiments 2 to 48 or 50 to 51, comprising
a) 150 mg/ml of the antibody;
b) trehalose, optionally wherein the concentration of trehalose is in the range of 95 mM to 250 mM or 145 mM to 225 mM;
c) a polysorbate, optionally wherein the concentration of the polysorbate is in the range of 0.05 mg/ml to 0.5 mg/ml or 0.075 mg/ml to 0.3 mg/ml; and
d) a buffer.
53. The formulation according to embodiment 51 or 52, wherein the buffer is an acetate buffer or a succinate buffer, optionally wherein the buffer concentration is in the range of 5 mM to 25 mM.
54. The formulation according to embodiment 52 or 53, wherein the polysorbate is polysorbate 20.
55. The formulation according to any one of embodiments 51 to 54, wherein the pH of the formulation is in a range of pH 5.2 to pH 6.5, optionally wherein the pH is in the range of 5.2 to 6.2 or 5.5 to 6.2 or is 5.7.
56. The formulation according any one of embodiments 2 to 48 or 50 to 55, comprising
a) 150 mg/ml of the antibody;
b) 170 mM to about 200 mM trehalose;
c) 0.1 mg/ml to 0.3 mg/ml polysorbate, optionally polysorbate 20; and
d) a buffer, optionally wherein the buffer is an acetate buffer.
57. The liquid pharmaceutical formulation according to one or more of embodiments 1, 3 to 36 or 49 to 50, comprising
a) 150 mg/ml of the antibody;
b) a polyol, optionally wherein the polyol is a sugar or sugar alcohol; and
c) a non-ionic surfactant, optionally a polysorbate;
d) no buffer.
58. The formulation according to embodiment 57, wherein the pH of the formulation is in a range of pH 5.2 to pH 6.5, optionally wherein the pH in the range of 5.2 to 6.2 or 5.5 to 6.2.
59. The formulation according to embodiment 58, wherein the pH is 5.7.
60. The formulation according to any one of embodiments 57 to 59, comprising 80 mM to 250 mM polyol, optionally wherein the polyol is trehalose.
61. The formulation according to any one of embodiments 57 to 60, wherein the concentration of the non-ionic surfactant is in the range of 0.05 mg/ml to 0.5 mg/ml, 0.075 mg/ml to 0.4 mg/ml or 0.1 mg/ml to 0.3 mg/ml.
62. The formulation according to embodiment 61, wherein the non-ionic surfactant is a polysorbate, optionally polysorbate 20.
63. The formulation according to one or more of embodiments 1 to 62, further comprising an amino acid as additive.
64. The formulation according to embodiment 63, wherein the amino acid has a charged side chain, optionally a positive-charged side chain such as L-arginine.
65. The formulation according to embodiment 63 or 64, wherein the amino acid is present in the formulation as a salt, optionally a hydrochloride (HCl) salt.
66. The formulation according to embodiment 63, wherein the amino acid is methionine.
67. The formulation according to embodiment 63, wherein the amino acid is L-proline.
68. The formulation according to one or more of embodiments 1 to 67, wherein the formulation has one or more of the following characteristics
(i) it does not comprise arginine;
(ii) it does not comprise an amino acid with a positive-charged side chain;
(iii) it does not comprise an amino acid with a charged side chain;
(iv) it does not comprise methionine; and/or
(v) it does not comprise an amino acid as additive.
69. The liquid pharmaceutical formulation according to any one of embodiments 2 to 68, comprising
a) 150 mg/ml of the antibody;
b) 185 mM trehalose;
c) 0.2 mg/ml polysorbate 20; and
d) 10 mM acetate buffer;
wherein the pH is in the range of 5.2 to 6.2 and optionally is 5.7.
70. The formulation according to any one of embodiments 1 to 69, wherein the formulation is stable.
71. The formulation according to embodiment 70, fulfilling one or more of the following stability characteristics:
(i) following storage at 5° C. for 36 months, at least 94%, at least 95% or at least 96% of the antibody is present as a monomer as measured by UP-SEC, and/or the relative monomer content of the antibody does not decrease by more than 3%, more than 2.5%, more than 2% or more than 1.5%;
(ii) following storage at 5° C. for 24 months, at least 94%, at least 95% or at least 96% of the antibody is present as a monomer as measured by UP-SEC, and/or the relative monomer content of the antibody does not decrease by more than 3%, more than 2.5%, more than 2%, more than 1.5% or more than 1%;
(iii) following storage at 5° C. for 9 months, at least 96% or at least 96.5% of the antibody is present as a monomer as measured by UP-SEC, and/or the relative monomer content of the antibody does not decrease by more than 1.5% or more than 1%;
(iv) following storage at 5° C. for 3 months, at least 96% or at least 97% of the antibody is present as a monomer as measured by UP-SEC, and/or the relative monomer content of the antibody does not decrease by more than 1% or more than 0.7% or more than 0.5%;
(v) following storage at 25° C. for 12 months, at least 90% or at least 92% of the antibody is present as a monomer as measured by UP-SEC, and/or the relative monomer content of the antibody does not decrease by more than 7% or more than 6% or more than 5%;
(vi) following storage at 25° C. for 3 months, at least 95% of the antibody is present as a monomer as measured by UP-SEC, and/or the relative monomer content of the antibody does not decrease by more than 3% or more than 2%;
(vii) following storage at 25° C. for 1 month, at least 96% of the antibody is present as a monomer as measured by UP-SEC, and/or the relative monomer content of the antibody does not decrease by more than 2% or more than 1%;
(viii) following storage at 40° C. for 3 months, at least 87% or at least 88% of the antibody is present as a monomer as measured by UP-SEC, and/or the relative monomer content of the antibody does not decrease by more than 10% or more than 9% or more than 8%; and/or
(ix) following storage at 40° C. for 1 month, at least 93% or at least 94% of the antibody is present as a monomer as measured by UP-SEC, and/or the relative monomer content of the antibody does not decrease by more than 5% or more than 4%.
72. The formulation according to embodiment 70 or 71, fulfilling one or more of the following stability characteristics:
(i) following storage at 5° C. for at least 36 months, the formulation has an opalescence of 12 FNU (Formazin Nephelometry Units) or less or 10 FNU or less, and/or the opalescence does not increase by more than 5 FNU or more than 3 FNU;
(ii) following storage at 5° C. for at least 3, 6, 9, 12, 18 or 24 months, the formulation has an opalescence of 12 FNU (Formazin Nephelometry Units) or less or 10 FNU or less, and/or the opalescence does not increase by more than 5 FNU or more than 3 FNU;
(iii) following storage at 25° C. for at least 1, 3, 6, 9 or 12 months, the formulation has an opalescence of 12 FNU or less or 10 FNU or less, and/or the opalescence does not increase by more than 7 FNU or more than 5 FNU;
(iv) following storage at 40° C. for at least 1 or 3 months, the formulation has an opalescence of 12 FNU or less or 10 FNU or less, and/or the opalescence does not increase by more than 5 FNU or more than 3 FNU; and/or
(v) following shaking at 25° C. for 21 days, the formulation has an opalescence of 12 FNU or less or 10 FNU or less, and/or the opalescence of the formulation does not increase by more than 3 FNU or more than 2 FNU.
73. The formulation according to any one of embodiments 70 to 72, fulfilling one or both of the following stability characteristics:
(i) following shaking at 25° C. for 21 days, at least 95% or at least 96% of the antibody is present as a monomer as measured by UP-SEC, and/or the relative monomer content of the antibody does not decrease by more than 2% or more than 1%; and/or
(ii) following shaking at 25° C. for 21 days, less than 3% or less than 2% of the antibody is present as high molecular weight (HMW) species as measured by UP-SEC, and/or the relative HMW content of the antibody does not increase by more than 2% or more than 1.5% or more than 1%.
74. The formulation according to any one of embodiments 70 to 73, fulfilling one or more of the following stability characteristics:
(i) following storage at 5° C. for 36 months, less than 4% or less than 3% of the antibody is present as high molecular weight (HMW) species as measured by UP-SEC, and/or the relative HMWS content of the antibody does not increase by more than 2% or more than 1.5%;
(ii) following storage at 5° C. for 24 months, less than 4% or less than 3% of the antibody is present as high molecular weight (HMW) species as measured by UP-SEC, and/or the relative HMW content of the antibody does not increase by more than 2% or more than 1.5% or more than 1%;
(iii) following storage at 5° C. for 9 months, less than 4% or less than 3% or less than 2.5% of the antibody is present as high molecular weight (HMW) species as measured by UP-SEC, and/or the relative HMW content of the antibody does not increase by more than 1% or more than 0.8% or more than 0.6%;
(iv) following storage at 5° C. for 3 months, less than 4% or less than 3% or less than 2.5% of the antibody is present as high molecular weight (HMW) species as measured by UP-SEC, and/or the relative HMW content of the antibody does not increase by more than 1% or more than 0.8% or more than 0.6%;
(v) following storage at 40° C. for 3 months, less than 6.5% or less than 6% or less than 5.5% of the antibody is present as a high molecular weight (HMW) species as measured by UP-SEC, and/or the relative HMW content of the antibody does not increase by more than 5% or more than 4%; and/or
(vi) following storage at 40° C. for 1 month, less than 5% or less than 4.5% or less than 4% of the antibody is present as high molecular weight (HMW) species as measured by UP-SEC, and/or the relative HMW content of the antibody does not increase by more than 2.5% or more than 2%.
75. The formulation according to any one of embodiments 70 to 74, fulfilling one or more of the following stability characteristics:
(i) following storage at 25° C. for 12 months, less than 5% or less than 4% of the antibody is present as high molecular weight (HMW) species as measured by UP-SEC, and/or the relative HMW content of the antibody does not increase by more than 3% or more than 2.5% or more than 2%;
(ii) following storage at 25° C. for 3 months, less than 4% or less than 3.5% or less than 3.2% of the antibody is present as high molecular weight (HMW) species as measured by UP-SEC, and/or the relative HMW content of the antibody does not increase by more than 2% or more than 1.5%; and/or
(iii) following storage at 25° C. for 1 month, less than 4% or less than 3.5% or less than 3% of the antibody is present as high molecular weight (HMW) species as measured by UP-SEC, and/or the relative HMW content of the antibody does not increase by more than 1.5% or more than 1%.
76. The formulation according to any one of embodiments 70 to 75, fulfilling one or more of the following stability characteristics:
(i) following storage at 5° C. for 36 months, less than 2% or less than 1.5% of the antibody is present as low molecular weight (LMW) species as measured by UP-SEC, and/or the relative LMWS content of the antibody does not increase by more than 1.5% or more than 1.5% or more than 0.5%;
(ii) following storage at 5° C. for 24 months, less than 2% or less than 1.5% of the antibody is present as low molecular weight (LMW) species as measured by UP-SEC, and/or the relative LMW content of the antibody does not increase by more than 1.5% or more than 1.5% or more than 0.5%;
(iii) following storage at 5° C. for 9 months, less than 2% or less than 1.5% of the antibody is present as a low molecular weight (LMW) species as measured by UP-SEC, and/or the relative LMW content of the antibody does not increase by more than 1.5% or more than 1.5% or more than 0.5%;
(iv) following storage at 5° C. for 3 months, less than 2% or less than 1.5% or less than 1% of the antibody is present as a low molecular weight (LMW) species as measured by UP-SEC, and/or the relative LMW content of the antibody does not increase by more than 1% or more than 0.5% or more than 0.25%;
(v) following storage at 40° C. for 3 months, less than 8% or less than 7% or less than 6% of the antibody is present as a low molecular weight (LMW) species as measured by UP-SEC, and/or the relative LMW content of the antibody does not increase by more than 8% or more than 7% or more than 6%; and/or
(vi) following storage at 40° C. for 1 month, less than 4% or less than 3.5% or less than 3% of the antibody is present as a low molecular weight (LMW) species as measured by UP-SEC, and/or the relative LMW content of the antibody does not increase by more than 3% or more than 2.5% or more than 2.2%.
77. The formulation according to any one of embodiments 70 to 76, fulfilling one or more of the following stability characteristics:
(i) following storage at 25° C. for 12 months, less than 6% or less than 5% or less than 4.5% of the antibody is present as a low molecular weight (LMW) species as measured by UP-SEC, and/or the relative LMW content of the antibody does not increase by more than 5% or more than 4% or more than 3%;
(ii) following storage at 25° C. for 3 months, less than 3% or less than 2% or less than 1.8% of the antibody is present as a low molecular weight (LMW) species as measured by UP-SEC, and/or the relative LMW content of the antibody does not increase by more than 2% or more than 1.5% or more than 1%; and/or
(iii) following storage at 25° C. for 1 month, less than 2% or less than 1.5% or less than 1.2% of the antibody is present as a low molecular weight (LMW) species as measured by UP-SEC, and/or the relative LMW content of the antibody does not increase by more than 1% or more than 0.6% or more than 0.4%.
78. The formulation according to any one of embodiments 70 to 77, fulfilling one or more of the following stability characteristics:
(i) following storage at 5° C. for 36 months, at least 55%, at least 60% or at least 65% of the antibody is present as main peak variants, and/or the relative content of main peak variants of the antibody does not decrease by more than 8% or more than 7% or more than 5%, as determined by ion exchange chromatography (IEC);
(ii) following storage at 5° C. for 24 months, at least 55%, at least 60% or at least 65% of the antibody is present as main peak variants, and/or the relative content of main peak variants of the antibody does not decrease by more than 8% or more than 7% or more than 5%, as determined by ion exchange chromatography (IEC);
(iii) following storage at 5° C. for 6 months, at least 60% or at least 65% of the antibody is present as main peak variants, and/or the relative content of main peak variants of the antibody does not decrease by more than 5% or more than 4%, as determined by ion exchange chromatography (IEC);
(iv) following storage at 5° C. for 3 months, at least 60% or at least 65% of the antibody is present as main peak variants, and/or the relative content of main peak variants of the antibody does not decrease by more than 4% or more than 3% or more than 2%, as determined by ion exchange chromatography (IEC);
(v) following storage at 25° C. for 12 months, at least 35% or at least 40% or at least 45% of the antibody is present as main peak variants, and/or the relative content of main peak variants of the antibody does not decrease by more than 35% or more than 30% or more than 25%, as determined by ion exchange chromatography (IEC);
(vi) following storage at 25° C. for 3 months, at least 55% or at least 60% of the antibody is present as main peak variants, and/or the relative content of main peak variants of the antibody does not decrease by more than 15% or more than 10%, as determined by ion exchange chromatography (IEC); and/or
(vii) following storage at 25° C. for 1 month, at least 60% or at least 65% of the is present as main peak variants, and/or the relative content of main peak variants of the antibody does not decrease by more than 10% or more than 5%, as determined by ion exchange chromatography (IEC).
79. The formulation according to any one of embodiments 70 to 78, fulfilling one or more of the following stability characteristics:
(i) following storage at 5° C. for 36 months, less than 30% or less than 28% of the antibody is present as acidic peak group variants, and/or the relative content of acidic peak group variants of the antibody does not increase by more than 4% or more than 3% or more than 2%, as determined by ion exchange chromatography (IEC);
(ii) following storage at 5° C. for 24 months, less than 30% or less than 28% of the antibody is present as acidic peak group variants, and/or the relative content of acidic peak group variants of the antibody does not increase by more than 4% or more than 3% or more than 2%, as determined by ion exchange chromatography (IEC);
(iii) following storage at 5° C. for 6 months, less than 30% or less than 28% of the antibody is present as acidic peak group variants, and/or the relative content of acidic peak group variants of the antibody does not increase by more than 4% or more than 3% or more than 2%, as determined by ion exchange chromatography (IEC);
(iv) following storage at 5° C. for 3 months, less than 30% or less than 28% of the antibody is present as acidic peak group variants, and/or the relative content of acidic peak group variants of the antibody does not increase by more than 3% or more than 2% or more than 1%, as determined by ion exchange chromatography (IEC);
(v) following storage at 25° C. for 12 months, less than 50%, less than 45% or less than 40% of the antibody is present as acidic peak group variants, and/or the relative content of acidic peak group variants of the antibody does not increase by more than 30% or more than 25% or more than 20%, as determined by ion exchange chromatography (IEC);
(vi) following storage at 25° C. for 3 months, less than 40% or less than 35% or less than 30% of the antibody is present as acidic peak group variants, and/or the relative content of acidic peak group variants of the antibody does not increase by more than 10% or more than 8% or more than 6%, as determined by ion exchange chromatography (IEC); and/or
(vii) following storage at 25° C. for 1 month, less than 35% or less than 30% or less than 28% of the antibody is present as acidic peak group variants, and/or the relative content of acidic peak group variants of the antibody does not increase by more than 4% or more than 3%, as determined by ion exchange chromatography (IEC).
80. The formulation according to any one of embodiments 70 to 79, fulfilling one or more of the following stability characteristics:
(i) following storage at 5° C. for 36 months, less than 20%, less than 17%, less than 15% or less than 13% of the antibody is present as basic peak group variants, and/or the relative content of basic peak group variants of the antibody does not increase by more than 10% or more than 8% or more than 6%, as determined by ion exchange chromatography (IEC);
(ii) following storage at 5° C. for 24 months, less than 20%, less than 17%, less than 15% or less than 13% of the antibody is present as basic peak group variants, and/or the relative content of basic peak group variants of the antibody does not increase by more than 10% or more than 8% or more than 6%, as determined by ion exchange chromatography (IEC);
(iii) following storage at 5° C. for 6 months, less than 15% or less than 10% of the antibody is present as basic peak group variants, and/or the relative content of basic peak group variants of the antibody does not increase by more than 4% or more than 3% or more than 2%, as determined by ion exchange chromatography (IEC);
(iv) following storage at 5° C. for 3 months, less than 15% or less than 10% of the antibody is present as basic peak group variants, and/or the relative content of basic peak group variants of the antibody does not increase by more than 3% or more than 2%, as determined by ion exchange chromatography (IEC);
(v) following storage at 25° C. for 12 months, less than 30% or less than 25% or less than 22% of the antibody is present as basic peak group variants, and/or the relative content of basic peak group variants of the antibody does not increase by more than 25% or more than 20% or more than 15%, as determined by ion exchange chromatography (IEC);
(vi) following storage at 25° C. for 3 months, less than 20% or less than 15% or less than 12% of the antibody is present as basic peak group variants, and/or the relative content of basic peak group variants of the antibody does not increase by more than 9% or more than 7% or more than 5%, as determined by ion exchange chromatography (IEC); and/or
(vii) following storage at 25° C. for 1 month, less than 15% or less than 10% or less than 9% of the antibody is present as basic peak group variants, and/or the relative content of basic peak group variants of the antibody does not increase by more than 3% or more than 2%, as determined by ion exchange chromatography (IEC).
81. The formulation according to any one of embodiments 70 to 80, fulfilling one or more of the following stability characteristics
(i) following storage at 5° C. for 36 months, at least 95% or at least 97% of the specific binding activity to IL-23 is measured compared to a reference antibody, wherein the reference antibody has not been stored;
(ii) following storage at 5° C. for 4, 6, 9, 12, 18 or 24 months, at least 95% or at least 97% of the specific binding activity to IL-23 is measured compared to a reference antibody, wherein the reference antibody has not been stored;
(iii) following storage at 25° C. for 2, 3, 4, 6, 9, 12, or 18 months, at least 93% or at least 96% of the specific binding activity to IL-23 is measured compared to a reference antibody, wherein the reference antibody has not been stored; and/or
(iv) following storage at 40° C. for 3, 4 or 6 months, at least 90% or at least 95% of the specific binding activity to IL-23 is measured compared to a reference antibody, wherein the reference antibody has not been stored.
82. The formulation according to one or more of embodiments 1 to 81, wherein the dynamic viscosity measured at 20° C. is ≤30 mPas (mPa·s), ≤25 mPas or ≤20 mPas.
83. The formulation according to one or more of embodiments 1 to 82, wherein the formulation has a conductivity in a range of 0.8 to 5 mS/cm, optionally in a range of 1 to 2 mS/cm or 1.2 to 1.8 mS/cm.
84. The formulation according to one or more of embodiments 1 to 83, wherein the formulation has an osmolality in a range of 225 mOsm/kg to 375 mOsm/kg, such as 250 mOsm/kg to 350 mOsm/kg, 275 mOsm/kg to 330 mOsm/kg or 290 mOsm/kg to 320 mOsm/kg.
85. The formulation according to one or more of embodiments 1 to 84, wherein the formulation is an injectable formulation.
86. The formulation according to embodiment 85, wherein the formulation is suitable for subcutaneous injection.
87. The formulation according to one or more of embodiments 1 to 86, wherein the formulation is not and has not been subjected to a reconstitution step before administration.
88. The formulation according to any one of embodiments 1 to 86, prepared by reconstitution from a lyophilized formulation.
89. A lyophilized formulation of an anti-IL-23p19 antibody, wherein the antibody comprises a light chain amino acid sequence according to SEQ ID NO: 18 and a heavy chain amino acid sequence according to SEQ ID NO: 19, made by lyophilizing the liquid formulation as defined in any one of embodiments 1 to 69, optionally wherein the liquid formulation is an aqueous solution.
90. A lyophilized formulation of an anti-IL-23p19 antibody, wherein the antibody comprises a light chain amino acid sequence according to SEQ ID NO: 18 and a heavy chain amino acid sequence according to SEQ ID NO: 19, providing upon reconstitution the liquid formulation as defined in any one of embodiments 1 to 69 or 82 to 86.
91. A lyophilized formulation comprising
a) an anti-IL-23p19 antibody, wherein the antibody comprises a light chain amino acid sequence according to SEQ ID NO: 18 and a heavy chain amino acid sequence according to SEQ ID NO: 19, in an amount that upon reconstitution provides an antibody concentration of 150 mg/ml;
b) a polyol;
c) a surfactant; and
d) optionally a buffer.
92. The lyophilized formulation according to embodiment 91, wherein the antibody is risankizumab.
93. The lyophilized formulation according to embodiment 91 or 92, wherein the polyol has one or more of the characteristics as defined in any one of embodiments 6 to 13, optionally wherein the polyol is a sugar, optionally selected from trehalose and sucrose.
94. The lyophilized formulation according to any one of embodiments 91 to 93, wherein the surfactant has one or more of the characteristics as defined in any one of embodiments 22 to 25, optionally wherein the surfactant is a polysorbate.
95. The lyophilized formulation according to any one of embodiments 91 to 94, comprising d) a buffer, wherein the buffer has one or more of the characteristics as defined in any one of embodiments 37 to 40.
96. The lyophilized formulation according to any one of embodiments 91 to 95, wherein the formulation has a pH as defined in any one of embodiments 31 to 36 when reconstituted.
97. A sealed container, optionally, a vial or pre-filled syringe, containing the liquid pharmaceutical formulation according to any one of embodiments 1 to 87.
98. A container, optionally, a sealed vial, containing the lyophilized formulation according to any one of embodiments 88 to 96.
99. The product according to embodiment 97, wherein the container contains 2 ml or less of the liquid formulation, optionally 1.5 ml or less or 1 ml or less of the liquid formulation.
100. The product according to any one of embodiments 97 to 99, comprising a single dose of 150 mg antibody.
101. The formulation according to any one of embodiments 1 to 96 or the product according to any one of embodiments 97 to 100 for therapeutic treatment of a human subject.
102. The formulation according to any one of embodiments 1 to 96 or the product according to any one of embodiments 97 to 100 for use in the treatment of a disease selected from psoriasis and inflammatory bowel disease.
103. The formulation according to any one of embodiments 1 to 96 or the product according to any one of embodiments 97 to 100 for use in the treatment of a disease selected from psoriatic arthritis and Crohn's disease.
104. A stable liquid pharmaceutical formulation comprising
a) 150 mg/ml of an anti-IL-23p19 antibody, wherein the antibody comprises a light chain amino acid sequence according to SEQ ID NO: 18 and a heavy chain amino acid sequence according to SEQ ID NO: 19;
b) a tonicity modifier; and
c) a surfactant,
wherein the formulation has a pH of 5.5-5.9 and the formulation is isotonic.
105. The stable formulation according to embodiment 104, wherein the antibody is risankizumab.
106. The stable formulation according to embodiment 104 or 105, wherein tonicity modifier is a polyol.
107. The stable formulation according to any one of embodiments 104 to 106, wherein the tonicity modifier is a polyol as defined in any one of the above embodiments 6 to 11.
108. The stable formulation according to any one of embodiments 104 to 107, wherein the concentration of the tonicity modifier has one or more of the characteristics as defined for the polyol in any one of the above embodiments 14 to 21, optionally wherein the tonicity modifier is a polyol as defined therein, optionally a sugar and/or sugar alcohol, optionally selected from trehalose and sucrose.
109. The stable formulation according to any one of embodiments 104 to 108, wherein the surfactant has one or more of the characteristics as defined in any one of the above embodiments 22 to 25, optionally wherein the surfactant is a polysorbate, optionally selected from polysorbate 20 and polysorbate 80.
110. The stable formulation according to any one of embodiments 104 to 109, wherein the concentration of the surfactant in the formulation is as defined in any one of the above embodiments 26 to 30.
111. The stable formulation according to any one of embodiments 104 to 110, wherein the formulation has a pH of in the range of 5.6 to 5.8, optionally wherein the pH of the formulation is 5.7.
112. The stable formulation according to any one of embodiments 104 to 111, comprising d) a buffer, optionally wherein the buffer has one or more of the characteristics as defined in any one of embodiments 37 to 40 and 48.
113. The stable formulation according to embodiment 112, wherein the buffer has a concentration as defined in any one of embodiments 41 to 47.
114. The stable formulation according to any one of embodiments 104 to 113, wherein the formulation is an aqueous formulation.
115. The stable formulation according to any one of embodiments 104 to 114, fulfilling one or more of the stability characteristics as defined in any one of embodiments 71 to 81.
116. The stable formulation according to any one of embodiments 104 to 111 or 114 to 115, wherein the formulation is buffer-free.
117. The stable formulation according to any one of embodiments 104 to 116, wherein the formulation has an osmolality of 290-320 mOsm/kg.
118. The stable formulation according to any one of embodiments 104 to 117, having at least one or at least two of the following characteristics:
(i) the surfactant is a non-ionic surfactant;
(ii) the surfactant is a polysorbate, optionally selected from polysorbate 20 and polysorbate 80;
(iii) wherein the concentration of the surfactant in the formulation is in a range of 0.05 mg/ml to 0.5 mg/ml, optionally within a range of 0.075 mg/ml to 0.4 mg/ml or 0.1 mg/ml to 0.3 mg/ml; and/or
(iv) it has any one of the characteristics as defined in embodiments 12, 13, or 63 to 68.
119. The stable formulation according to any one of embodiments 104 to 118, prepared by reconstitution from a lyophilized formulation.
120. A lyophilized formulation of an anti-IL-23p19 antibody, wherein the antibody comprises a light chain amino acid sequence according to SEQ ID NO: 18 and a heavy chain amino acid sequence according to SEQ ID NO: 19, made by lyophilizing the liquid formulation as defined in any one of embodiments 104 to 118, optionally wherein the stable liquid formulation is an aqueous solution.
121. A lyophilized formulation of an anti-IL-23p19 antibody, wherein the antibody comprises a light chain amino acid sequence according to SEQ ID NO: 18 and a heavy chain amino acid sequence according to SEQ ID NO: 19, providing upon reconstitution the stable liquid formulation as defined in any one of embodiments 104 to 118.

### Methods of Treatment

In one aspect, the present invention provides methods for the treatment of IL-23A related diseases. In one aspect, the present invention provides methods for treating a disease, for example an inflammatory disease, in particular methods comprising administering an anti-IL-23A antibody to a patient in certain amounts and/or at certain intervals. In one aspect, a method of the present invention is for the treatment of psoriasis or psoriatic arthritis. In one aspect, a method of the present invention is for the treatment of Ankylosing Spondylitis (AS). In one aspect, a method of the present invention is for the treatment axial (spinal) spondyloarthritis (ax-SpA), for example non-radiographic ax-SpA.

In one aspect, the present invention provides an anti-IL-23A antibody for use in the treatment of a disease, for example an inflammatory disease, for example psoriasis, psoriatic arthritis or axial (spinal) spondyloarthritis (ax-SpA), including Ankylosing Spondylitis and non-radiographic ax-SpA, by administration in certain amounts and/or at certain intervals as described herein.

In one aspect, the present invention provides for the use of an anti-IL-23A antibody for the preparation of a medicament for the treatment of a disease, for example an inflammatory disease, for example psoriasis, psoriatic arthritis or axial (spinal) spondyloarthritis (ax-SpA), including Ankylosing Spondylitis and non-radiographic ax-SpA, by administration in certain amounts and/or at certain intervals as described herein.

In one aspect, a method of the present invention comprises administering to the patient an initial dose of the anti-IL-23A antibody followed by the administration of one or more maintenances doses of the anti-IL-23A antibody. Optionally, a loading dose of the anti-IL-23A antibody is administered to the patient between the administration of the initial dose and the administration of the first maintenance dose.

In one aspect, in a method of the instant invention the interval between the administration of the initial dose and the first maintenance dose is 4 to 24 weeks, for example 4 to 16 weeks, for example 4 to 12 weeks, for example 4, 6, 8, 12, 16, 20 or 24 weeks. In another aspect, the interval between the administration of the first maintenance dose and the subsequent maintenance doses is 4 to 24 weeks, for example 4 to 16 weeks, for example 4 to 12 weeks, for example 4, 6, 8, 12, 16, 20 or 24 weeks.

In one aspect, in a method of the instant invention the interval between the administration of the initial dose and the first maintenance dose is 8 to 24 weeks, for example 8 to 16 weeks, for example 8 to 12 weeks, for example 6 to 24 weeks, for example 6 to 16 weeks, for example 6 to 12 weeks, for example 6, 8, 12, 16, 20 or 24 weeks. In another aspect, the interval between the administration of the first maintenance dose and the subsequent maintenance doses is 8 to 24 weeks, for example 8 to 16 weeks, for example 8 to 12 weeks, for example 6 to 24 weeks, for example 6 to 16 weeks, for example 6 to 12 weeks, for example 6, 8, 12, 16, 20 or 24 weeks.

In one aspect, in a method of the instant invention, the interval between the administration of the initial dose and the first maintenance dose and the interval between the administration of the first maintenance dose and the subsequent maintenance doses are the same, for example 4 to 24 weeks, for example 4 to 16 weeks, for example 4 to 12 weeks, for example 4, 6, 8, 12, 16, 20 or 24 weeks.

In one aspect, in a method of the instant invention, the interval between the administration of the initial dose and the first maintenance dose and the interval between the administration of the first maintenance dose and the subsequent maintenance doses are the same, for example 8 to 24 weeks, for example 8 to 16 weeks, for example 8 to 12 weeks, for example 6 to 24 weeks, for example 6 to 16 weeks, for example 6 to 12 weeks, for example 6, 8, 12, 16, 20 or 24 weeks.

In one aspect, in a method of the instant invention, a loading dose of the anti-IL-23A antibody is administered to the patient between the administration of the initial dose and the administration of the first maintenance doses.

In one aspect, the loading dose is administered to the patient 1, 2, 3, 4, 5 or 6 weeks after the administration of the initial dose. In one aspect, in a method of the instant invention the interval between the administration of the loading dose and the first maintenance dose is 8 to 24 weeks, for example 8 to 16 weeks, for example 8 to 12 weeks, for example 6 to 24 weeks, for example 6 to 16 weeks, for example 6 to 12 weeks, for example 6, 8, 12, 16, 20 or 24 weeks. In another aspect, the interval between the administration of the first maintenance dose and the subsequent maintenance doses is 8 to 24 weeks, for example 8 to 16 weeks, for example 8 to 12 weeks, for example 6 to 24 weeks, for example 6 to 16 weeks, for example 6 to 12 weeks, for example 6, 8, 12, 16, 20 or 24 weeks.

In one aspect, in a method of the instant invention, the interval between the administration of the loading dose and the first maintenance dose and the interval between the administration of the first maintenance dose and the subsequent maintenance doses are the same, for example 8 to 24 weeks, for example 8 to 16 weeks, for example 8 to 12 weeks, for example 6 to 24 weeks, for example 6 to 16 weeks, for example 6 to 12 weeks, for example 6, 8, 12, 16, 20 or 24 weeks.

In one aspect, the amount of anti-IL-23A antibody in the initial dose and in the maintenance dose is the same. In one aspect, the amount of anti-IL-23A antibody in the initial dose and in the loading dose is the same. In one aspect, the amount of anti-IL-23A antibody in the loading dose and in the maintenance dose is the same. In one aspect, the amount of anti-IL-23A antibody in the initial dose, in the loading dose and in the maintenance dose is the same.

In one aspect, the amount of anti-IL-23A antibody in the maintenance doses is lower than the amount of anti-IL-23A antibody in the initial dose and/or loading dose. In one aspect, the amount of anti-IL-23A antibody in the initial dose is twice the amount of anti-IL-23A antibody in the maintenance dose, for example in the absence of a loading dose. In one aspect, the amount of anti-IL-23A antibody in the initial dose and in the loading dose is twice the amount of anti-IL-23A antibody in the maintenance dose.

In one aspect, in a method of the present invention, the initial dose comprises 15 to 300 mg, for example 15 to 250 mg of the anti-IL-23A antibody. In another aspect, the maintenance doses comprise 15 to 300 mg, for example 15 to 250 mg of the anti-IL-23A antibody. In another aspect, if a loading dose is administered, such loading dose comprises 15 to 300 mg, for example 15 to 250 mg of the anti-IL-23A antibody.

In a further aspect, an initial dose, a loading dose or a maintenance dose according to the present invention comprises 70 to 90 mg, 80 to 100 mg, 90 to 110 mg, 100 to 120 mg, 110 to 130 mg, 120 to 140 mg, 130 to 150 mg, 140 to 160 mg, 150 to 170 mg, 160 to 180 mg, 170 to 190 mg, 180 to 200 mg, 190 to 210 mg, 200 to 220 mg, 210 to 230 mg, 220 to 240 mg, 230 to 250 mg, 240 to 260 mg, 250 to 270 mg, 260 to 280 mg, 270 to 290 mg or 280 to 300 mg of the anti-IL-23A antibody.

In a further aspect, an initial dose, a loading dose or a maintenance dose according to the present invention comprises 18, 20, 30, 40, 50, 60, 70, 80, 90, 100, 110, 120, 130, 140, 150, 160, 170, 180, 190, 200, 210, 220, 230, 240, 250, 260, 270, 280, 290 or 300 mg of the anti-IL-23A antibody. In a further aspect, an initial dose, a loading dose or a maintenance dose according to the present invention comprises 75 mg of the anti-IL-23A antibody.

In a further aspect, the present invention provides a method for treating an inflammatory disease, said method comprising administering to a patient 15 to 250 mg of an anti-IL-23A antibody. In one aspect, 70 to 90 mg, 80 to 100 mg, 90 to 110 mg, 100 to 120 mg, 110 to 130 mg, 120 to 140 mg, 130 to 150 mg, 140 to 160 mg, 150 to 170 mg, 160 to 180 mg, 170 to 190 mg, 180 to 200 mg, 190 to 210 mg, 200 to 220 mg, 210 to 230 mg, 220 to 240 mg, 230 to 250 mg, 240 to 260 mg, 250 to 270 mg, 260 to 280 mg, 270 to 290 mg or 280 to 300 mg of the anti-IL-23A antibody is administered to the patient. In one aspect, 18, 20, 30, 40, 50, 60, 70, 80, 90, 100, 110, 120, 130, 140, 150, 160, 170, 180, 190, 200, 210, 220, 230, 240, 250, 260, 270, 280, 290 or 300 mg of the anti-IL-23A antibody is administered to the patient. In one aspect, 75 mg of the anti-IL-23A antibody is administered to the patient. In one aspect, the disease is psoriasis or psoriatic arthritis. In one aspect, the disease is axial (spinal) spondyloarthritis (ax-SpA), including ankylosing spondylitis and non-radiographic ax-SpA.

In one aspect, in a method of the present invention, 90-180 mg of the anti-IL-23 antibody is administered to the patient every 6-12 weeks, for example every 8-12 weeks, with or without a loading dose.

Representative examples of doses and dose regimens according to the present invention are disclosed in Table A.

TABLE A
doses and dose regimens

| | Loading | | | |
|---|---|---|---|---|
| | dose (mg, | | | |
| | 4 weeks | | | Alternative |
| Initial | after | Maintenance | Frequency of | frequencies of |
| dose | initial | dose | maintenance | maintenance |
| (mg) | dose) | (mg) | doses | doses |
| 90 | x | 90 | Every 12 weeks | Every 6, 8, 16, |
| | | | | 20 or 24 weeks |
| 90 | 90 | 90 | Every 12 weeks | Every 6, 8, 16, |
| | | | | 20 or 24 weeks |
| 180 | x | 180 | Every 12 weeks | Every 6, 8, 16, |
| | | | | 20 or 24 weeks |
| 180 | 180 | 180 | Every 12 weeks | Every 6, 8, 16, |
| | | | | 20 or 24 weeks |
| 150 | x | 150 | Every 12 weeks | Every 6, 8, 16, |
| | | | | 20 or 24 weeks |
| 150 | 150 | 150 | Every 12 weeks | Every 6, 8, 16, |
| | | | | 20 or 24 weeks |
| 140 | x | 140 | Every 12 weeks | Every 6, 8, 16, |
| | | | | 20 or 24 weeks |
| 140 | 140 | 140 | Every 12 weeks | Every 6, 8, 16, |
| | | | | 20 or 24 weeks |
| 130 | x | 130 | Every 12 weeks | Every 6, 8, 16, |
| | | | | 20 or 24 weeks |
| 130 | 130 | 130 | Every 12 weeks | Every 6, 8, 16, |
| | | | | 20 or 24 weeks |
| 120 | x | 120 | Every 12 weeks | Every 6, 8, 16, |
| | | | | 20 or 24 weeks |
| 120 | 120 | 120 | Every 12 weeks | Every 6, 8, 16, |
| | | | | 20 or 24 weeks |
| 110 | x | 110 | Every 12 weeks | Every 6, 8, 16, |
| | | | | 20 or 24 weeks |
| 110 | 110 | 110 | Every 12 weeks | Every 6, 8, 16, |
| | | | | 20 or 24 weeks |
| 100 | x | 100 | Every 12 weeks | Every 6, 8, 16, |
| | | | | 20 or 24 weeks |
| 100 | 100 | 100 | Every 12 weeks | Every 6, 8, 16, |
| | | | | 20 or 24 weeks |
| 160 | x | 160 | Every 12 weeks | Every 6, 8, 16, |
| | | | | 20 or 24 weeks |
| 160 | 160 | 160 | Every 12 weeks | Every 6, 8, 16, |
| | | | | 20 or 24 weeks |
| 170 | x | 170 | Every 12 weeks | Every 6, 8, 16, |
| | | | | 20 or 24 weeks |
| 170 | 170 | 170 | Every 12 weeks | Every 6, 8, 16, |
| | | | | 20 or 24 weeks |
| 190 | x | 190 | Every 12 weeks | Every 6, 8, 16, |
| | | | | 20 or 24 weeks |
| 190 | 190 | 190 | Every 12 weeks | Every 6, 8, 16, |
| | | | | 20 or 24 weeks |
| 200 | x | 200 | Every 12 weeks | Every 6, 8, 16, |
| | | | | 20 or 24 weeks |
| 200 | 200 | 200 | Every 12 weeks | Every 6, 8, 16, |
| | | | | 20 or 24 weeks |
| 210 | 210 | 210 | Every 12 weeks | Every 6, 8, 16, |
| | | | | 20 or 24 weeks |
| 220 | 220 | 220 | Every 12 weeks | Every 6, 8, 16, |
| | | | | 20 or 24 weeks |
| 230 | 230 | 230 | Every 12 weeks | Every 6, 8, 16, |
| | | | | 20 or 24 weeks |
| 240 | 240 | 240 | Every 12 weeks | Every 6, 8, 16, |
| | | | | 20 or 24 weeks |
| 250 | 250 | 250 | Every 12 weeks | Every 6, 8, 16, |
| | | | | 20 or 24 weeks |
| 260 | 260 | 260 | Every 12 weeks | Every 6, 8, 16, |
| | | | | 20 or 24 weeks |
| 270 | 270 | 270 | Every 12 weeks | Every 6, 8, 16, |
| | | | | 20 or 24 weeks |
| 280 | 280 | 280 | Every 12 weeks | Every 6, 8, 16, |
| | | | | 20 or 24 weeks |
| 290 | 290 | 290 | Every 12 weeks | Every 6, 8, 16, |
| | | | | 20 or 24 weeks |
| 300 | 300 | 300 | Every 12 weeks | Every 6, 8, 16, |
| | | | | 20 or 24 weeks |

x: no loading dose administered.

In Table A when a loading dose is administered, it is indicated as being administered 4 weeks after the initial dose.

It is also within the scope of the instant invention to administer the loading dose 1 week after the initial dose for each of the doses and frequencies of maintenance doses identified in the above table.

It is also within the scope of the instant invention to administer the loading dose 2 week after the initial dose for each of the doses and frequencies of maintenance doses identified in the above table.

It is also within the scope of the instant invention to administer the loading dose 3 week after the initial dose for each of the doses and frequencies of maintenance doses identified in the above table.

It is also within the scope of the instant invention to administer the loading dose 5 week after the initial dose for each of the doses and frequencies of maintenance doses identified in the above table.

It is also within the scope of the instant invention to administer the loading dose 6 week after the initial dose for each of the doses and frequencies of maintenance doses identified in the above table.

For example, in the context of the present invention, if no loading dose is administered to the patient, and with a frequency of administration of the maintenance doses of 12 weeks, the initial dose is administered to the patient at week 0, followed by a further administration at week 12 (first maintenance dose), then at week 24 (second maintenance dose) and so on at dosing intervals of 12 weeks. In one aspect, the initial dose and the maintenance doses comprise 150 mg of the anti-IL-23A antibody. In one aspect, the initial dose and the maintenance doses comprise 75 mg of the anti-IL-23A antibody.

For example, in the context of the present invention, if a loading dose is administered to the patient, and with a frequency of administration of the maintenance doses of 12 weeks, the initial dose is administered to the patient at week 0, followed by a further administration at week 4 (loading dose), then at week 16 (first maintenance dose), at week 28 (second maintenance dose) and so on at dosing intervals of 12 weeks. In one aspect, the initial dose, the loading dose and the maintenance doses comprise 150 mg of the anti-IL-23A antibody. In one aspect, the initial dose and the loading dose comprise 300 mg of the anti-IL-23A antibody and the maintenance doses comprise 150 mg of the anti-IL-23A antibody. In one aspect, the initial dose and the loading dose comprise 180 mg of the anti-IL-23A antibody and the maintenance doses comprise 90 mg of the anti-IL-23A antibody. In one aspect, the initial dose, the loading dose and the maintenance doses comprise 75 mg of the anti-IL-23A antibody.

For example, in the context of the present invention, if no loading dose is administered to the patient, and with a frequency of administration of the maintenance doses of 8 weeks, the initial dose is administered to the patient at week 0, followed by a further administration at week 8 (first maintenance dose), then at week 16 (second maintenance dose) and so on at dosing intervals of 8 weeks. In one aspect, the initial dose and the maintenance doses comprise 90 mg of the anti-IL-23A antibody. In one aspect, the initial dose and the maintenance doses comprise 150 mg of the anti-IL-23A antibody.

For example, in the context of the present invention, if a loading dose is administered to the patient, and with a frequency of administration of the maintenance doses of 8 weeks, the initial dose is administered to the patient at week 0, followed by a further administration at week 4 (loading dose), then at week 12 (first maintenance dose), at week 20 (second maintenance dose) and so on at dosing intervals of 8 weeks. In one aspect, the initial dose, the loading dose and the maintenance doses comprise 90 mg of the anti-IL-23A antibody. In one aspect, the initial dose, the loading dose and the maintenance doses comprise 150 mg of the anti-IL-23A antibody.

For example, in the context of the present invention, if no loading dose is administered to the patient, and with a frequency of administration of the maintenance doses of 16 weeks, the initial dose is administered to the patient at week 0, followed by a further administration at week 16 (first maintenance dose), then at week 32 (second maintenance dose) and so on at dosing intervals of 16 weeks. In one aspect, the initial dose and the maintenance doses comprise 150 mg of the anti-IL-23A antibody.

For example, in the context of the present invention, if a loading dose is administered to the patient, and with a frequency of administration of the maintenance doses of 16 weeks, the initial dose is administered to the patient at week 0, followed by a further administration at week 4 (loading dose), then at week 20 (first maintenance dose), at week 36 (second maintenance dose) and so on at dosing intervals of 16 weeks. In one aspect, the initial dose, the loading dose and the maintenance doses comprise 150 mg of the anti-IL-23A antibody.

For example, in the context of the present invention, if no loading dose is administered to the patient, and with a frequency of administration of the maintenance doses of 6 weeks, the initial dose is administered to the patient at week 0, followed by a further administration at week 6 (first maintenance dose), then at week 12 (second maintenance dose) and so on at dosing intervals of 6 weeks. In one aspect, the initial dose and the maintenance doses comprise 150 mg of the anti-IL-23A antibody.

For example, in the context of the present invention, if a loading dose is administered to the patient, and with a frequency of administration of the maintenance doses of 6 weeks, the initial dose is administered to the patient at week 0, followed by a further administration at week 4 (loading dose), then at week 10 (first maintenance dose), at week 16 (second maintenance dose) and so on at dosing intervals of 6 weeks. In one aspect, the initial dose, the loading dose and the maintenance doses comprise 150 mg of the anti-IL-23A antibody.

For example, in the context of the present invention, if the frequency of administration of the maintenance doses is 4 weeks, the initial dose is administered to the patient at week 0, followed by a further administration at week 4 (first maintenance dose), then at week 8 (second maintenance dose), at week 12 (third maintenance dose) and so on at dosing intervals of 4 weeks. In one aspect, the initial dose and the maintenance doses comprise 150 mg of the anti-IL-23A antibody.

In a further aspect, in a method according to the present invention, an initial dose of an anti-IL-23 antibody is administered to the patient. A second dose of the anti-IL-23 antibody is not administered to the patient as long as the therapeutic benefit of the initial dose is maintained, for example as assessed by PASI score, for example PASI 90, PASI 75, PASI 100 or PASI 50, but a second dose is administered to the patient when the disease severity increases above a certain level, for example when the patient no longer maintains a certain PASI score, e.g. PASI 90, PASI 75, PASI 100 or PASI 50. The initial dose and the second dose of the anti-IL-23 antibody are for example doses as described herein. In one aspect, the patient is monitored after the initial dose is administered, for example by assessing the PASI score of the patient. In one aspect, a third dose of the anti-IL-23 antibody is administered to the patient when the patient no longer maintains a certain PASI score, e.g. PASI 90, PASI 75, PASI 100 or PASI 50, after the administration of the second dose. The third dose of the anti-IL-23 antibody is for example a dose as described herein, for example the same dose as the second dose. In one aspect, the patient is monitored after the second dose is administered, for example by assessing the PASI score of the patient. In a further aspect, at least one additional dose is administered to the patient in such a way.

In a further aspect, in a method according to the present invention, a loading dose of the anti-IL-23 antibody is administered to the patient after the administration of the initial dose, for example after a time period as described herein. A third dose of the anti-IL-23 antibody is not administered to the patient as long as the therapeutic benefit of the initial dose is maintained, for example as assessed by PASI score, for example PASI 90, PASI 75, PASI 100 or PASI 50, but a third dose is administered to the patient when the disease severity increases above a certain level, for example when the patient no longer maintains a certain PASI score, e.g. PASI 90, PASI 75, PASI 100 or PASI 50. In one aspect, the loading dose of the anti-IL-23 antibody is a dose as described herein. In one aspect, a fourth dose of the anti-IL-23 antibody is administered to the patient when the patient no longer maintains a certain PASI score, e.g. PASI 90, PASI 75, PASI 100 or PASI 50, after administration of the third dose. The fourth dose of the anti-IL-23 antibody is for example a dose as described herein, for example the same dose as the third dose. In one aspect, the patient is monitored after the third dose is administered, for example by assessing the PASI score of the patient. In a further aspect, at least one additional dose is administered to the patient in such a way.

Accordingly, In one embodiment, the present invention further provides a method for treating an inflammatory disease, in one aspect for treating psoriasis or psoriatic arthritis, comprising administering to a patient an anti-IL-23A antibody, said method comprising:
a) administering an initial dose of said anti-IL-23A antibody to the patient; and
b) administering a second dose of said anti-IL-23A antibody to the patient when the patient no longer maintains PASI 90, PASI 75, PASI 100 or PASI 50.

In one embodiment, the present invention further provides a method for treating an inflammatory disease, in one aspect for treating psoriasis or psoriatic arthritis, comprising administering to a patient an anti-IL-23A antibody, said method comprising:
a) administering an initial dose of said anti-IL-23A antibody to the patient; and
b) administering a second dose of said anti-IL-23A antibody to the patient when the patient no longer maintains a sPGA score of clear (score of 0) or almost clear (score of 1).

In one aspect, in a method of the present invention, a first maintenance dose is administered to the patient up to 32 weeks after the administration of the initial dose. In one aspect, in a method of the present invention, the first maintenance dose is administered to the patient 28 weeks or 32 weeks after the administration of the initial dose.

In one aspect, in a method of the present invention, a first maintenance dose is administered to the patient up to 32 weeks after the administration of the loading dose. In one aspect, in a method of the present invention, the first maintenance dose is administered to the patient 28 weeks or 32 weeks after the administration of the loading dose.

In one aspect, in a method of the present invention, at least one additional maintenance dose is administered to the patient up to 32 weeks after the administration of the first maintenance dose. In one aspect, in a method of the present invention, at least one additional maintenance dose is administered to the patient 28 weeks or 32 weeks after the administration of the first maintenance dose.

In one aspect, the amount of anti-IL-23A antibody in the second and following doses is lower than the amount of anti-IL-23A antibody in the initial dose. In one aspect, the amount of anti-IL-23A antibody in the initial dose is twice the amount of anti-IL-23A antibody in the second and following doses.

In one embodiment, the present invention further provides a method for treating an inflammatory disease, in one aspect for treating psoriasis or psoriatic arthritis, comprising administering to a patient an anti-IL-23A antibody, said method comprising:
a) administering an initial dose of said anti-IL-23A antibody to the patient;
b) administering a loading dose of said anti-IL-23A antibody to the patient 1 to 6 weeks, for example 4 weeks, after said initial dose is administered; and
c) administering a third dose of said anti-IL-23A antibody to the patient when the patient no longer maintains PASI 90, PASI 75, PASI 100 or PASI 50.

In one aspect, the amount of anti-IL-23A antibody in the third and following doses is lower than the amount of anti-IL-23A antibody in the initial dose and/or loading dose. In one aspect, the amount of anti-IL-23A antibody in the initial dose is twice the amount of anti-IL-23A antibody in the third and following doses. In one aspect, the amount of anti-IL-23A antibody in the initial dose and in the loading dose is twice the amount of anti-IL-23A antibody in the third and following doses.

In one embodiment, an anti-IL-23A antibody in any one of the methods above is disclosed herein.

In one aspect, in any one of the methods above, a pharmaceutical composition comprising an anti-IL-23A antibody is administered to the patient. In one aspect, formulation 2 disclosed in Example 4 comprising an anti-IL-23A antibody, for example Antibody A, Antibody B, Antibody C or Antibody D is administered to the patient. In one aspect, formulation 3 disclosed in Example 4 comprising an anti-IL-23A antibody, for example Antibody A, Antibody B, Antibody C or Antibody D is administered to the patient.

In one aspect, the anti-IL-23A antibody is a humanized antibody. In one aspect, the anti-IL-23A antibody is a monoclonal antibody. In one aspect, the anti-IL-23A antibody is a full length antibody. In one aspect, the anti-IL-23A antibody is a humanized monoclonal antibody, for example a full length humanized monoclonal antibody.

An antibody described herein recognizes specific “IL-23A antigen epitope” or “IL-23A epitope”. As used herein these terms refer to a molecule (e.g., a peptide) or a fragment of a molecule capable of immunoreactivity with an anti-IL-23A antibody and, for example, include an IL-23A antigenic determinant recognized by any of the antibodies having a light chain/heavy chain sequence combination of SEQ ID NO:11/14, 11/15, 10/14 or 10/15.

The generalized structure of antibodies or immunoglobulin is well known to those of skill in the art. These molecules are heterotetrameric glycoproteins, typically of about 150,000 daltons, composed of two identical light (L) chains and two identical heavy (H) chains and are typically referred to as full length antibodies. Each light chain is covalently linked to a heavy chain by one disulfide bond to form a heterodimer, and the heterotrimeric molecule is formed through a covalent disulfide linkage between the two identical heavy chains of the heterodimers. Although the light and heavy chains are linked together by one disulfide bond, the number of disulfide linkages between the two heavy chains varies by immunoglobulin isotype. Each heavy and light chain also has regularly spaced intrachain disulfide bridges. Each heavy chain has at the amino-terminus a variable domain (V_{H}), followed by three or four constant domains (C_{H1}, C_{H2}, C_{H3}, and C_{H4}), as well as a hinge region between C_{H1} and C_{H2}. Each light chain has two domains, an amino-terminal variable domain (V_{L}) and a carboxy-terminal constant domain (CL). The V_{L} domain associates non-covalently with the V_{H} domain, whereas the CL domain is commonly covalently linked to the C_{H1} domain via a disulfide bond. Particular amino acid residues are believed to form an interface between the light and heavy chain variable domains (Chothia et al., 1985, J. Mol. Biol. 186:651-663). Variable domains are also referred herein as variable regions.

Certain domains within the variable domains differ extensively between different antibodies i.e., are “hypervariable.” These hypervariable domains contain residues that are directly involved in the binding and specificity of each particular antibody for its specific antigenic determinant. Hypervariability, both in the light chain and the heavy chain variable domains, is concentrated in three segments known as complementarity determining regions (CDRs) or hypervariable loops (HVLs). CDRs are defined by sequence comparison in Kabat et al., 1991, In: Sequences of Proteins of Immunological Interest, 5^{th} Ed. Public Health Service, National Institutes of Health, Bethesda, Md., whereas HVLs (also referred herein as CDRs) are structurally defined according to the three-dimensional structure of the variable domain, as described by Chothia and Lesk, 1987, J. Mol. Biol. 196: 901-917. These two methods result in slightly different identifications of a CDR. As defined by Kabat, CDR-L1 is positioned at about residues 24-34, CDR-L2, at about residues 50-56, and CDR-L3, at about residues 89-97 in the light chain variable domain; CDR-H1 is positioned at about residues 31-35, CDR-H2 at about residues 50-65, and CDR-H3 at about residues 95-102 in the heavy chain variable domain. The exact residue numbers that encompass a particular CDR will vary depending on the sequence and size of the CDR. Those skilled in the art can routinely determine which residues comprise a particular CDR given the variable region amino acid sequence of the antibody. The CDR1, CDR2, CDR3 of the heavy and light chains therefore define the unique and functional properties specific for a given antibody.

The three CDRs within each of the heavy and light chains are separated by framework regions (FR), which contain sequences that tend to be less variable. From the amino terminus to the carboxy terminus of the heavy and light chain variable domains, the FRs and CDRs are arranged in the order: FR1, CDR1, FR2, CDR2, FR3, CDR3, and FR4. The largely 3-sheet configuration of the FRs brings the CDRs within each of the chains into close proximity to each other as well as to the CDRs from the other chain. The resulting conformation contributes to the antigen binding site (see Kabat et al., 1991, NIH Publ. No. 91-3242, Vol. I, pages 647-669), although not all CDR residues are necessarily directly involved in antigen binding.

FR residues and Ig constant domains are not directly involved in antigen binding, but contribute to antigen binding and/or mediate antibody effector function. Some FR residues are thought to have a significant effect on antigen binding in at least three ways: by noncovalently binding directly to an epitope, by interacting with one or more CDR residues, and by affecting the interface between the heavy and light chains. The constant domains are not directly involved in antigen binding but mediate various Ig effector functions, such as participation of the antibody in antibody dependent cellular cytotoxicity (ADCC), complement dependent cytotoxicity (CDC) and antibody dependent cellular phagocytosis (ADCP).

The light chains of vertebrate immunoglobulins are assigned to one of two clearly distinct classes, kappa (κ) and lambda (λ), based on the amino acid sequence of the constant domain. By comparison, the heavy chains of mammalian immunoglobulins are assigned to one of five major classes, according to the sequence of the constant domains: IgA, IgD, IgE, IgG, and IgM. IgG and IgA are further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively. The subunit structures and three-dimensional configurations of the classes of native immunoglobulins are well known.

The terms, “antibody”, “anti-IL-23A antibody”, “anti-IL-23p19 antibody”, “humanized anti-IL-23A antibody”, “humanized anti-IL-23p19 antibody”, “humanized anti-IL-23A epitope antibody”, humanized anti-IL-2319 epitope antibody”, “variant humanized anti-IL-23A epitope antibody” and “variant humanized anti-IL-23p19 epitope antibody” specifically encompass monoclonal antibodies (including full length monoclonal antibodies), polyclonal antibodies, and antibody fragments such as variable domains and other portions of antibodies that exhibit a desired biological activity, e.g., IL-23A binding. The term “monoclonal antibody” (mAb) refers to an antibody that is highly specific, being directed against a single antigenic determinant, an “epitope”. Therefore, the modifier “monoclonal” is indicative of antibodies directed to the identical epitope and is not to be construed as requiring production of the antibody by any particular method. It should be understood that monoclonal antibodies can be made by any technique or methodology known in the art; including e.g., the hybridoma method (Kohler et al., 1975, Nature 256:495), or recombinant DNA methods known in the art (see, e.g., U.S. Pat. No. 4,816,567), or methods of isolation of monoclonal recombinantly produced using phage antibody libraries, using techniques described in Clackson et al., 1991, Nature 352: 624-628, and Marks et al., 1991, J. Mol. Biol. 222: 581-597.

The term “monomer” refers to a homogenous form of an antibody. For example, for a full-length antibody, monomer means a monomeric antibody having two identical heavy chains and two identical light chains.

Chimeric antibodies consist of the heavy and light chain variable regions of an antibody from one species (e.g., a non-human mammal such as a mouse) and the heavy and light chain constant regions of another species (e.g., human) antibody and can be obtained by linking the DNA sequences encoding the variable regions of the antibody from the first species (e.g., mouse) to the DNA sequences for the constant regions of the antibody from the second (e.g. human) species and transforming a host with an expression vector containing the linked sequences to allow it to produce a chimeric antibody. Alternatively, the chimeric antibody also could be one in which one or more regions or domains of the heavy and/or light chain is identical with, homologous to, or a variant of the corresponding sequence in a monoclonal antibody from another immunoglobulin class or isotype, or from a consensus or germline sequence. Chimeric antibodies can include fragments of such antibodies, provided that the antibody fragment exhibits the desired biological activity of its parent antibody, for example binding to the same epitope (see, e.g., U.S. Pat. No. 4,816,567; and Morrison et al., 1984, Proc. Natl. Acad. Sci. USA 81: 6851-6855).

The terms, “antibody fragment”, “anti-IL-23A antibody fragment”, “anti-IL-23A epitope antibody fragment”, “humanized anti-IL-23A antibody fragment”, “humanized anti-IL-23A epitope antibody fragment”, “variant humanized anti-IL-23A epitope antibody fragment” refer to a portion of a full length anti-IL-23A antibody, in which a variable region or a functional capability is retained, for example, specific IL-23A epitope binding. Examples of antibody fragments include, but are not limited to, a Fab, Fab′, F(ab′)₂, Fd, Fv, scFv and scFv-Fc fragment.

Full length antibodies can be treated with enzymes such as papain or pepsin to generate useful antibody fragments. Papain digestion is used to produces two identical antigen-binding antibody fragments called “Fab” fragments, each with a single antigen-binding site, and a residual “Fc” fragment. The Fab fragment also contains the constant domain of the light chain and the C_{H1} domain of the heavy chain. Pepsin treatment yields a F(ab′)₂ fragment that has two antigen-binding sites and is still capable of cross-linking antigen.

Fab′ fragments differ from Fab fragments by the presence of additional residues including one or more cysteines from the antibody hinge region at the C-terminus of the C_{H1} domain. F(ab′)₂ antibody fragments are pairs of Fab′ fragments linked by cysteine residues in the hinge region. Other chemical couplings of antibody fragments are also known.

“Fv” fragment contains a complete antigen-recognition and binding site consisting of a dimer of one heavy and one light chain variable domain in tight, non-covalent association. In this configuration, the three CDRs of each variable domain interact to define an antigen-biding site on the surface of the V_{H}-V_{L} dimer. Collectively, the six CDRs confer antigen-binding specificity to the antibody.

A “single-chain Fv” or “scFv” antibody fragment is a single chain Fv variant comprising the V_{H} and V_{L} domains of an antibody where the domains are present in a single polypeptide chain. The single chain Fv is capable of recognizing and binding antigen. The scFv polypeptide may optionally also contain a polypeptide linker positioned between the V_{H} and V_{L} domains in order to facilitate formation of a desired three-dimensional structure for antigen binding by the scFv (see, e.g., Pluckthun, 1994, In The Pharmacology of monoclonal Antibodies, Vol. 113, Rosenburg and Moore eds., Springer-Verlag, New York, pp. 269-315).

A “humanized antibody” or a “humanized antibody fragment” is a specific type of chimeric antibody which includes an immunoglobulin amino acid sequence variant, or fragment thereof, which is capable of binding to a predetermined antigen and which, comprises one or more FRs having substantially the amino acid sequence of a human immunoglobulin and one or more CDRs having substantially the amino acid sequence of a non-human immunoglobulin. This non-human amino acid sequence often referred to as an “import” sequence is typically taken from an “import” antibody domain, particularly a variable domain. In general, a humanized antibody includes at least the CDRs or HVLs of a non-human antibody, inserted between the FRs of a human heavy or light chain variable domain. The present invention describes specific humanized anti-IL-23A antibodies which contain CDRs derived from the mouse monoclonal antibodies or humanized CDRs shown in Tables 1 and 2 inserted between the FRs of human germline sequence heavy and light chain variable domains. It will be understood that certain mouse FR residues may be important to the function of the humanized antibodies and therefore certain of the human germline sequence heavy and light chain variable domains residues are modified to be the same as those of the corresponding mouse sequence.

In another aspect, a humanized anti-IL-23A antibody comprises substantially all of at least one, and typically two, variable domains (such as contained, for example, in Fab, Fab′, F(ab′)₂, Fabc, and Fv fragments) in which all, or substantially all, of the CDRs correspond to those of a non-human immunoglobulin, and specifically herein, all of the CDRs are mouse or humanized sequences as detailed in Tables 1 and 2 herein below and all, or substantially all, of the FRs are those of a human immunoglobulin consensus or germline sequence. In another aspect, a humanized anti-IL-23A antibody also includes at least a portion of an immunoglobulin Fc region, typically that of a human immunoglobulin. Ordinarily, the antibody will contain both the light chain as well as at least the variable domain of a heavy chain. The antibody also may include one or more of the C_{H1}, hinge, C_{H2}, C_{H3}, and/or C_{H4} regions of the heavy chain, as appropriate.

A humanized anti-IL-23A antibody can be selected from any class of immunoglobulins, including IgM, IgG, IgD, IgA and IgE, and any isotype, including IgG₁, IgG₂, IgG₃, IgG₄, IgA₁ and IgA₂. For example, the constant domain can be a complement fixing constant domain where it is desired that the humanized antibody exhibit cytotoxic activity, and the isotype is typically IgG₁. Where such cytotoxic activity is not desirable, the constant domain may be of another isotype, e.g., IgG₂. An alternative humanized anti-IL-23A antibody can comprise sequences from more than one immunoglobulin class or isotype, and selecting particular constant domains to optimize desired effector functions is within the ordinary skill in the art. In specific embodiments, the present invention provides antibodies that are IgG1 antibodies and more particularly, are IgG1 antibodies in which there is a knock-out of effector functions.

The FRs and CDRs, or HVLs, of a humanized anti-IL-23A antibody need not correspond precisely to the parental sequences. For example, one or more residues in the import CDR, or HVL, or the consensus or germline FR sequence may be altered (e.g., mutagenized) by substitution, insertion or deletion such that the resulting amino acid residue is no longer identical to the original residue in the corresponding position in either parental sequence but the antibody nevertheless retains the function of binding to IL-23A. Such alteration typically will not be extensive and will be conservative alterations. Usually, at least 75% of the humanized antibody residues will correspond to those of the parental consensus or germline FR and import CDR sequences, more often at least 90%, and most frequently greater than 95%, or greater than 98% or greater than 99%.

Immunoglobulin residues that affect the interface between heavy and light chain variable regions (“the V_{L}-V_{H} interface”) are those that affect the proximity or orientation of the two chains with respect to one another. Certain residues that may be involved in interchain interactions include V_{L} residues 34, 36, 38, 44, 46, 87, 89, 91, 96, and 98 and V_{H} residues 35, 37, 39, 45, 47, 91, 93, 95, 100, and 103 (utilizing the numbering system set forth in Kabat et al., Sequences of Proteins of Immunological Interest (National Institutes of Health, Bethesda, Md., 1987)). U.S. Pat. No. 6,407,213 also discusses that residues such as V_{L} residues 43 and 85, and V_{H} residues 43 and 60 also may be involved in this interaction. While these residues are indicated for human IgG only, they are applicable across species. Important antibody residues that are reasonably expected to be involved in interchain interactions are selected for substitution into the consensus sequence.

The terms “consensus sequence” and “consensus antibody” refer to an amino acid sequence which comprises the most frequently occurring amino acid residue at each location in all immunoglobulins of any particular class, isotype, or subunit structure, e.g., a human immunoglobulin variable domain. The consensus sequence may be based on immunoglobulins of a particular species or of many species. A “consensus” sequence, structure, or antibody is understood to encompass a consensus human sequence as described in certain embodiments, and to refer to an amino acid sequence which comprises the most frequently occurring amino acid residues at each location in all human immunoglobulins of any particular class, isotype, or subunit structure. Thus, the consensus sequence contains an amino acid sequence having at each position an amino acid that is present in one or more known immunoglobulins, but which may not exactly duplicate the entire amino acid sequence of any single immunoglobulin. The variable region consensus sequence is not obtained from any naturally produced antibody or immunoglobulin. Kabat et al., 1991, Sequences of Proteins of Immunological Interest, 5th Ed. Public Health Service, National Institutes of Health, Bethesda, Md., and variants thereof. The FRs of heavy and light chain consensus sequences, and variants thereof, provide useful sequences for the preparation of humanized anti-IL-23p19 antibodies. See, for example, U.S. Pat. Nos. 6,037,454 and 6,054,297.

Human germline sequences are found naturally in the human population. A combination of those germline genes generates antibody diversity. Germline antibody sequences for the light chain of the antibody come from conserved human germline kappa or lambda v-genes and j-genes. Similarly the heavy chain sequences come from germline v-, d- and j-genes (LeFranc, M-P, and LeFranc, G, “The Immunoglobulin Facts Book” Academic Press, 2001).

As used herein, “variant”, “anti-IL-23A variant”, “humanized anti-IL-23A variant”, or “variant humanized anti-IL-23A” each refers to a humanized anti-IL-23A antibody having at least a light chain variable murine CDR from any of the sequences as shown in Table 1 or a heavy chain murine CDR sequence derived from the murine monoclonal antibody as shown in Table 2. Variants include those having one or more amino acid changes in one or both light chain or heavy chain variable domains, provided that the amino acid change does not substantially impair binding of the antibody to IL-23A. Exemplary antibodies produced herein include those designated as Antibody A, Antibody B, Antibody C and Antibody D, and the various light chains and heavy chains of the same are shown in SEQ ID Nos:18 and 21, and SEQ ID Nos:19 and 20, respectively.

An “isolated” antibody is one that has been identified and separated and/or recovered from a component of its natural environment. Contaminant components of the antibody's natural environment are those materials that may interfere with diagnostic or therapeutic uses of the antibody, and can be enzymes, hormones, or other proteinaceous or nonproteinaceous solutes. In one aspect, the antibody will be purified to at least greater than 95% isolation by weight of antibody.

An isolated antibody includes an antibody in situ within recombinant cells in which it is produced, since at least one component of the antibody's natural environment will not be present. Ordinarily however, an isolated antibody will be prepared by at least one purification step in which the recombinant cellular material is removed.

The term “antibody performance” refers to factors that contribute to antibody recognition of antigen or the effectiveness of an antibody in vivo. Changes in the amino acid sequence of an antibody can affect antibody properties such as folding, and can influence physical factors such as initial rate of antibody binding to antigen (kₐ), dissociation constant of the antibody from antigen (k_{d}), affinity constant of the antibody for the antigen (Kd), conformation of the antibody, protein stability, and half-life of the antibody.

The term “epitope tagged” when used herein, refers to an anti-IL-23A antibody fused to an “epitope tag”. An “epitope tag” is a polypeptide having a sufficient number of amino acids to provide an epitope for antibody production, yet is designed such that it does not interfere with the desired activity of the anti-IL-23A antibody. The epitope tag is usually sufficiently unique such that an antibody raised against the epitope tag does not substantially cross-react with other epitopes. Suitable tag polypeptides generally contain at least 6 amino acid residues and usually contain about 8 to 50 amino acid residues, or about 9 to 30 residues. Examples of epitope tags and the antibody that binds the epitope include the flu HA tag polypeptide and its antibody 12CA5 (Field et al., 1988 Mol. Cell. Biol. 8: 2159-2165; c-myc tag and 8F9, 3C7, 6E10, G4, B7 and 9E10 antibodies thereto (Evan et al., 1985, Mol. Cell. Biol. 5(12):3610-3616; and Herpes simplex virus glycoprotein D (gD) tag and its antibody (Paborsky et al. 1990, Protein Engineering 3(6): 547-553). In certain embodiments, the epitope tag is a “salvage receptor binding epitope”. As used herein, the term “salvage receptor binding epitope” refers to an epitope of the Fc region of an IgG molecule (such as IgG₁, IgG₂, IgG₃, or IgG₄) that is responsible for increasing the in vivo serum half-life of the IgG molecule.

For diagnostic as well as therapeutic monitoring purposes, the antibodies of the invention also may be conjugated to a label, either a label alone or a label and an additional second agent (prodrug, chemotherapeutic agent and the like). A label, as distinguished from the other second agents refers to an agent that is a detectable compound or composition and it may be conjugated directly or indirectly to an antibody of the present invention. The label may itself be detectable (e.g., radioisotope labels or fluorescent labels) or, in the case of an enzymatic label, may catalyze chemical alteration of a substrate compound or composition that is detectable. Labeled anti-IL-23A antibody can be prepared and used in various applications including in vitro and in vivo diagnostics.

In various aspects of the present invention one or more domains of the antibodies will be recombinantly expressed. Such recombinant expression may employ one or more control sequences, i.e., polynucleotide sequences necessary for expression of an operably linked coding sequence in a particular host organism. The control sequences suitable for use in prokaryotic cells include, for example, promoter, operator, and ribosome binding site sequences. Eukaryotic control sequences include, but are not limited to, promoters, polyadenylation signals, and enhancers. These control sequences can be utilized for expression and production of anti-IL-23A antibody in prokaryotic and eukaryotic host cells.

A nucleic acid sequence is “operably linked” when it is placed into a functional relationship with another nucleic acid sequence. For example, a nucleic acid presequence or secretory leader is operably linked to a nucleic acid encoding a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading frame. However, enhancers are optionally contiguous. Linking can be accomplished by ligation at convenient restriction sites. If such sites do not exist, synthetic oligonucleotide adaptors or linkers can be used.

As used herein, the expressions “cell”, “cell line”, and “cell culture” are used interchangeably and all such designations include the progeny thereof. Thus, “transformants” and “transformed cells” include the primary subject cell and cultures derived therefrom without regard for the number of transfers.

The term “mammal” for purposes of treatment refers to any animal classified as a mammal, including humans, domesticated and farm animals, and zoo, sports, or pet animals, such as dogs, horses, cats, cows, and the like. Preferably, the mammal is human.

A “disorder”, as used herein, is any condition that would benefit from treatment with an anti-IL-23A antibody described herein. This includes chronic and acute disorders or diseases including those pathological conditions that predispose the mammal to the disorder in question.

As used herein, the term “IL-23-associated disorder” or “IL-23-associated disease” refers to a condition in which IL-23 activity contributes to the disease and typically where IL-23 is abnormally expressed. An IL-23-associated disorder includes diseases and disorders of the immune system, such as autoimmune disorders and inflammatory diseases. Such conditions include psoriasis, inflammatory bowel disease, for example ulcerative colitis or Crohn's disease, and spondyloarthritis, for example ankylosing spondylitis, non-radiographic axial spondyloarthritis, peripheral spondyloarthritis or psoriatic arthritis.

The term “intravenous infusion” refers to introduction of an agent into the vein of an animal or human patient over a period of time greater than approximately 15 minutes, generally between approximately 30 to 90 minutes.

The term “intravenous bolus” or “intravenous push” refers to drug administration into a vein of an animal or human such that the body receives the drug in approximately 15 minutes or less, generally 5 minutes or less.

The term “subcutaneous administration” refers to introduction of an agent under the skin of an animal or human patient, preferable within a pocket between the skin and underlying tissue, by relatively slow, sustained delivery from a drug receptacle. Pinching or drawing the skin up and away from underlying tissue may create the pocket.

The term “subcutaneous infusion” refers to introduction of a drug under the skin of an animal or human patient, preferably within a pocket between the skin and underlying tissue, by relatively slow, sustained delivery from a drug receptacle for a period of time including, but not limited to, 30 minutes or less, or 90 minutes or less. Optionally, the infusion may be made by subcutaneous implantation of a drug delivery pump implanted under the skin of the animal or human patient, wherein the pump delivers a predetermined amount of drug for a predetermined period of time, such as 30 minutes, 90 minutes, or a time period spanning the length of the treatment regimen.

The term “subcutaneous bolus” refers to drug administration beneath the skin of an animal or human patient, where bolus drug delivery is less than approximately 15 minutes; in another aspect, less than 5 minutes, and in still another aspect, less than 60 seconds. In yet even another aspect, administration is within a pocket between the skin and underlying tissue, where the pocket may be created by pinching or drawing the skin up and away from underlying tissue.

The term “therapeutically effective amount” is used to refer to an amount of an active agent that relieves or ameliorates one or more of the symptoms of the disorder being treated. In another aspect, the therapeutically effective amount refers to a target serum concentration that has been shown to be effective in, for example, slowing disease progression. Efficacy can be measured in conventional ways, depending on the condition to be treated.

The terms “treatment” and “therapy” and the like, as used herein, are meant to include therapeutic as well as prophylactic, or suppressive measures for a disease or disorder leading to any clinically desirable or beneficial effect, including but not limited to alleviation or relief of one or more symptoms, regression, slowing or cessation of progression of the disease or disorder. Thus, for example, the term treatment includes the administration of an agent prior to or following the onset of a symptom of a disease or disorder thereby preventing or removing one or more signs of the disease or disorder. As another example, the term includes the administration of an agent after clinical manifestation of the disease to combat the symptoms of the disease. Further, administration of an agent after onset and after clinical symptoms have developed where administration affects clinical parameters of the disease or disorder, such as the degree of tissue injury or the amount or extent of metastasis, whether or not the treatment leads to amelioration of the disease, comprises “treatment” or “therapy” as used herein. Moreover, as long as the compositions of the invention either alone or in combination with another therapeutic agent alleviate or ameliorate at least one symptom of a disorder being treated as compared to that symptom in the absence of use of the anti-IL-23A antibody composition, the result should be considered an effective treatment of the underlying disorder regardless of whether all the symptoms of the disorder are alleviated or not.

The term “package insert” is used to refer to instructions customarily included in commercial packages of therapeutic products, that contain information about the indications, usage, administration, contraindications and/or warnings concerning the use of such therapeutic products.

### Antibodies

The CDRs of selected antibodies used in the context of the present invention are shown in Table 1 and 2. The variable regions of selected antibodies used in the context of the present invention are shown in Table 3 and 4.

TABLE 1
LIGHT CHAIN CDR sequences

| | L-CDR1 | L-CDR2 | L-CDR3 |
|---|---|---|---|
| 6B8 | KASRDVAIAVA | WASTRHT | HQYSSYPFT |
| | (SEQ ID NO: 1) | (SEQ ID NO: 2) | (SEQ ID NO: 3) |

TABLE 2
HEAVY CHAIN CDR sequences

| | H-CDR1 | H-CDR2 | H-CDR3 |
|---|---|---|---|
| 6B8 | GNTFTDQTIH | YIYPRDDSPKY | PDRSGYAWFIY |
| | (SEQ ID | NENFKG (SEQ | (SEQ ID |
| | NO: 4) | ID NO: 5) | NO: 6) |
| Hu_6B8-2 | GYTFTDQTIH | YIYPRDDSPKY | PDRSGYAWFIY |
| | (SEQ ID | NENFKG (SEQ | (SEQ ID |
| | NO: 7) | ID NO: 5) | NO: 6) |
| Hu_6B8-5 | GFTFTDQTIH | YIYPRDDSPKY | PDRSGYAWFIY |
| | (SEQ ID | NENFKG (SEQ | (SEQ ID |
| | NO: 8) | ID NO: 5) | NO: 6) |
| Hu_6B8- | GGTFTDQTIH | YIYPRDDSPKY | PDRSGYAWFIY |
| 36/65 | (SEQ ID | NENFKG (SEQ | (SEQ ID |
| | NO: 9) | ID NO: 5) | NO: 6) |

TABLE 3
Humanized 6B8-VK Sequences

| | |
|---|---|
| 6B8CVK-65 | DIQMTQSPSSLSASVGDRVTITCKASRDVAIAVAWYQQ |
| | KPGKVPKLLLFWASTRHTGVPDRFSGSGSGTDFTLTIS |
| | SLQPEDLADYYCHQYSSYPFTFGQGTKLEIK |
| | (SEQ ID NO: 10) |
| 6B8CVK-66 | DIQMTQSPSSLSASVGDRVTITCKASRDVAIAVAWYQQ |
| | KPGKVPKLLIYWASTRHTGVPSRFSGSGSRTDFTLTIS |
| | SLQPEDVADYFCHQYSSYPFTFGSGTKLEIK |
| | (SEQ ID NO: 11) |
| 6B8CVK-67 | DIQMTQSPSSLSASVGDRVTITCKASRDVAIAVAWYQQ |
| | KPGKVPKLLLYWASTRHTGVPSRFSGSGSRTDFTLTIS |
| | SLQPEDVATYYCHQYSSYPFTFGSGTKLEIK |
| | (SEQ ID NO: 12) |
| 6B8CVK-78 | DIQMTQSPSSLSASVGDRVTITCKASRDVAIAVAWYQQ |
| | KPGKVPKLLLFWASTRHTGVPDRFSGSGSRTDFTLTIS |
| | SLQPEDLADYYCHQYSSYPFTFGSGTKLEIK |
| | (SEQ ID NO: 13) |

TABLE 4
Humanized 6B8-VH Sequence

| | |
|---|---|
| 6B8CVH-02 | QVQLVQSGAEVKKPGSSVKVSCKASGYTFTDQTIHWMR |
| | QAPGQGLEWIGYIYPRDDSPKYNENFKGKVTITADKST |
| | STAYMELSSLRSEDTAVYYCAIPDRSGYAWFIYWGQGT |
| | LVTVSS (SEQ ID NO: 14) |
| 6B8CVH-05 | QVQLVQSGAEVKKPGSSVKVSCKASGFTFTDQTIHWVR |
| | QAPGQGLEWMGYIYPRDDSPKYNENFKGKVTLTADKST |
| | STAYMELSSLRSEDTAVYYCAIPDRSGYAWFIYWGQGT |
| | LVTVSS (SEQ ID NO: 15) |
| 6B8CVH-36 | QVQLVQSGAEVKKPGSSVKTSCKASGGTFTDQTIHWVR |
| | QRPGQGLEWMGYIYPRDDSPKYNENFKGRVTITADKST |
| | STAYMELSSLRSEDTAVYYCAIPDRSGYAWFIYWGQGT |
| | LVTVSS (SEQ ID NO: 16) |
| 6B8CVH-65 | QVQLVQSGAEVKKPGSSVKVSCKASGGTFTDQTIHWVR |
| | QAPGQGLEWMGYIYPRDDSPKYNENFKGRVTLTADKST |
| | STAYMELSSLRSEDTAVYFCARPDRSGYAWFIYWGQGT |
| | LVTVSS (SEQ ID NO: 17) |

Selected combination of humanized light chain and heavy chain variable regions derived from mouse antibody 6B8 resulted in Antibodies A, B, C and D:
Antibody A: 6B8-IgG1KO-2 with IgK-66 (heavy chain variable region 6B8CVH-02 and light chain variable region 6B8CVK-66);
Antibody B: 6B8-IgG1KO-5 with IgK-66 (heavy chain variable region 6B8CVH-05 and light chain variable region 6B8CVK-66);
Antibody C: 6B8-IgG1KO-2 with IgK-65 (heavy chain variable region 6B8CVH-02 and light chain variable region 6B8CVK-65);
Antibody D: 6B8-IgG1KO-5 with IgK-65 (heavy chain variable region 6B8CVH-05 and light chain variable region 6B8CVK-65).

Antibodies A, B, C and D have the heavy and light chain sequences shown in Table 5.

TABLE 5
Heavy and Light Chain DNA
and Amino Acid Sequences
for Antibodies A, B, C, and D

| | | |
|---|---|---|
| Antibody A | IgK light | DIQMTQSPSSLSASVGDRVTITCKA |
| | Chain #66 | SRDVAIAVAWYQQKPGKVPKLLIYW |
| | | ASTRHTGVPSRFSGSGSRTDFTLTI |
| | | SSLQPEDVADYFCHQYSSYPFTFGS |
| | | GTKLEIKRTVAAPSVFIFPPSDEQL |
| | | KSGTASVVCLLNNFYPREAKVQWKV |
| | | DNALQSGNSQESVTEQDSKDSTYSL |
| | | SSTLTLSKADYEKHKVYACEVTHQG |
| | | LSSPVTKSFNRGEC |
| | | (SEQ ID NO: 18) |
| | IgG1 KO | QVQLVQSGAEVKKPGSSVKVSCKAS |
| | Heavy | GYTFTDQTIHWMRQAPGQGLEWIGY |
| | Chain #2 | IYPRDDSPKYNENFKGKVTITADKS |
| | | TSTAYMELSSLRSEDTAVYYCAIPD |
| | | RSGYAWFIYWGQGTLVTVSSASTKG |
| | | PSVFPLAPSSKSTSGGTAALGCLVK |
| | | DYFPEPVTVSWNSGALTSGVHTFPA |
| | | VLQSSGLYSLSSVVTVPSSSLGTQT |
| | | YICNVNHKPSNTKVDKRVEPKSCDK |
| | | THTCPPCPAPEAAGGPSVFLFPPKP |
| | | KDTLMISRTPEVTCVVVDVSHEDPE |
| | | VKFNWYVDGVEVHNAKTKPREEQYN |
| | | STYRVVSVLTVLHQDWLNGKEYKCK |
| | | VSNKALPAPIEKTISKAKGQPREPQ |
| | | VYTLPPSREEMTKNQVSLTCLVKGF |
| | | YPSDIAVEWESNGQPENNYKTTPPV |
| | | LDSDGSFFLYSKLTVDKSRWQQGNV |
| | | FSCSVMHEALHNHYTQKSLSLSPG |
| | | (SEQ ID NO: 19) |
| Antibody B | IgK light | (SEQ ID NO: 18) |
| | Chain #66 | |
| | IgG1KO | QVQLVQSGAEVKKPGSSVKVSCKAS |
| | Heavy | GFTFTDQTIHWVRQAPGQGLEWMGY |
| | Chain #5 | IYPRDDSPKYNENFKGKVTLTADKS |
| | | TSTAYMELSSLRSEDTAVYYCAIPD |
| | | RSGYAWFIYWGQGTLVTVSSASTKG |
| | | PSVFPLAPSSKSTSGGTAALGCLVK |
| | | DYFPEPVTVSWNSGALTSGVHTFPA |
| | | VLQSSGLYSLSSVVTVPSSSLGTQT |
| | | YICNVNHKPSNTKVDKRVEPKSCDK |
| | | THTCPPCPAPEAAGGPSVFLFPPKP |
| | | KDTLMISRTPEVTCVVVDVSHEDPE |
| | | VKFNWYVDGVEVHNAKTKPREEQYN |
| | | STYRVVSVLTVLHQDWLNGKEYKCK |
| | | VSNKALPAPIEKTISKAKGQPREPQ |
| | | VYTLPPSREEMTKNQVSLTCLVKGF |
| | | YPSDIAVEWESNGQPENNYKTTPPV |
| | | LDSDGSFFLYSKLTVDKSRWQQGNV |
| | | FSCSVMHEALHNHYTQKSLSLSPG |
| | | (SEQ ID NO: 20) |
| Antibody C | IgK light | DIQMTQSPSSLSASVGDRVTITCKA |
| | Chain #65 | SRDVAIAVAWYQQKPGKVPKLLLFW |
| | | ASTRHTGVPDRFSGSGSGTDFTLTI |
| | | SSLQPEDLADYYCHQYSSYPFTFGQ |
| | | GTKLEIKRTVAAPSVFIFPPSDEQL |
| | | KSGTASVVCLLNNFYPREAKVQWKV |
| | | DNALQSGNSQESVTEQDSKDSTYSL |
| | | SSTLTLSKADYEKHKVYACEVTHQG |
| | | LSSPVTKSFNRGEC |
| | | (SEQ ID NO: 21) |
| | IgG1KO | (SEQ ID NO: 19) |
| | Heavy | |
| | Chain #2 | |
| Antibody D | IgK light | (SEQ ID NO: 21) |
| | Chain #65 | |
| | IgG1KO | (SEQ ID NO: 20) |
| | Heavy | |
| | Chain #5 | |

Light chains and heavy chain variable regions of Antibodies A, B, C, and D are underlined in Table 5 above.

In one embodiment, an anti-IL-23A antibody comprises the light chain sequence of SEQ ID NO:18 and the heavy chain sequence of SEQ ID NO:19. In one embodiment, an anti-IL-23A antibody comprises the light chain sequence of SEQ ID NO:18 and the heavy chain sequence of SEQ ID NO:20. In one embodiment, an anti-IL-23A antibody comprises the light chain sequence of SEQ ID NO:21 and the heavy chain sequence of SEQ ID NO:19. In one embodiment, an anti-IL-23A antibody comprises the light chain sequence of SEQ ID NO:21 and the heavy chain sequence of SEQ ID NO:20.

In one embodiment, an anti-IL-23A antibody consists of the light chain sequence of SEQ ID NO:18 and the heavy chain sequence of SEQ ID NO:19. In one embodiment, an anti-IL-23A antibody consists of the light chain sequence of SEQ ID NO:18 and the heavy chain sequence of SEQ ID NO:20. In one embodiment, an anti-IL-23A antibody consists of the light chain sequence of SEQ ID NO:21 and the heavy chain sequence of SEQ ID NO:19. In one embodiment, an anti-IL-23A antibody consists of the light chain sequence of SEQ ID NO:21 and the heavy chain sequence of SEQ ID NO:20.

In a further embodiment, an anti-IL-23A antibody binds to human IL-23A at an epitope consisting of amino acid residues 108 to 126 and amino acid residues 137 to 151 of SEQ ID NO: 22.

In a further embodiment, an anti-IL-23A antibody competitively binds to human IL-23A with an antibody of the present invention, for example Antibody A, Antibody B, Antibody C or Antibody D described herein. The ability of an antibody to competitively bind to IL-23A can be measured using competitive binding assays known in the art.

In some embodiments, an anti-IL-23A antibody comprises light chain variable region sequences having the amino acid sequence set forth in of SEQ ID NO:10, 11, 12 or 13. In some embodiments, an anti-IL-23A antibody comprises heavy chain variable region sequences having the amino acid sequence set forth in of SEQ ID NO:14, 15, 16 or 17 (see Tables 3 and 4 above). The CDR sequences of these antibodies are shown in Tables 1 and 2. For example, anti-IL-23A antibodies are monoclonal antibodies with the combinations of light chain variable and heavy chain variable regions of SEQ ID NO: 11/14, 11/15, 10/14 or 10/15. Such variable regions can be combined with human constant regions.

### Polynucleotides, Vectors, Host Cells, and Recombinant Methods

Other embodiments encompass isolated polynucleotides that comprise a sequence encoding an anti-IL-23A antibody, vectors, and host cells comprising the polynucleotides, and recombinant techniques for production of the humanized antibody. The isolated polynucleotides can encode any desired form of the anti-IL-23A antibody including, for example, full length monoclonal antibodies, Fab, Fab′, F(ab′)₂, and Fv fragments.

The polynucleotide(s) that comprise a sequence encoding an anti-IL-23A antibody can be fused to one or more regulatory or control sequence, as known in the art, and can be contained in suitable expression vectors or host cell as known in the art. Each of the polynucleotide molecules encoding the heavy or light chain variable domains can be independently fused to a polynucleotide sequence encoding a constant domain, such as a human constant domain, enabling the production of intact antibodies. Alternatively, polynucleotides, or portions thereof, can be fused together, providing a template for production of a single chain antibody.

For recombinant production, a polynucleotide encoding the antibody is inserted into a replicable vector for cloning (amplification of the DNA) or for expression. Many suitable vectors for expressing the recombinant antibody are available. The vector components generally include, but are not limited to, one or more of the following: a signal sequence, an origin of replication, one or more marker genes, an enhancer element, a promoter, and a transcription termination sequence.

The anti-IL-23A antibodies can also be produced as fusion polypeptides, in which the antibody is fused with a heterologous polypeptide, such as a signal sequence or other polypeptide having a specific cleavage site at the amino terminus of the mature protein or polypeptide. The heterologous signal sequence selected is typically one that is recognized and processed (i.e., cleaved by a signal peptidase) by the host cell. For prokaryotic host cells that do not recognize and process the anti-IL-23A antibody signal sequence, the signal sequence can be substituted by a prokaryotic signal sequence. The signal sequence can be, for example, alkaline phosphatase, penicillinase, lipoprotein, heat-stable enterotoxin II leaders, and the like. For yeast secretion, the native signal sequence can be substituted, for example, with a leader sequence obtained from yeast invertase alpha-factor (including Saccharomyces and Kluyveromyces α-factor leaders), acid phosphatase, C. albicans glucoamylase, or the signal described in WO90/13646. In mammalian cells, mammalian signal sequences as well as viral secretory leaders, for example, the herpes simplex gD signal, can be used. The DNA for such precursor region is ligated in reading frame to DNA encoding the anti-IL-23A antibody.

Expression and cloning vectors contain a nucleic acid sequence that enables the vector to replicate in one or more selected host cells. Generally, in cloning vectors this sequence is one that enables the vector to replicate independently of the host chromosomal DNA, and includes origins of replication or autonomously replicating sequences. Such sequences are well known for a variety of bacteria, yeast, and viruses. The origin of replication from the plasmid pBR322 is suitable for most Gram-negative bacteria, the 2-1 plasmid origin is suitable for yeast, and various viral origins (SV40, polyoma, adenovirus, VSV, and BPV) are useful for cloning vectors in mammalian cells. Generally, the origin of replication component is not needed for mammalian expression vectors (the SV40 origin may typically be used only because it contains the early promoter).

Expression and cloning vectors may contain a gene that encodes a selectable marker to facilitate identification of expression. Typical selectable marker genes encode proteins that confer resistance to antibiotics or other toxins, e.g., ampicillin, neomycin, methotrexate, or tetracycline, or alternatively, are complement auxotrophic deficiencies, or in other alternatives supply specific nutrients that are not present in complex media, e.g., the gene encoding D-alanine racemase for Bacilli.

One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid, and hygromycin. Common selectable markers for mammalian cells are those that enable the identification of cells competent to take up a nucleic acid encoding an anti-IL-23A antibody, such as DHFR (dihydrofolate reductase), thymidine kinase, metallothionein-I and -II (such as primate metallothionein genes), adenosine deaminase, ornithine decarboxylase, and the like. Cells transformed with the DHFR selection gene are first identified by culturing all of the transformants in a culture medium that contains methotrexate (Mtx), a competitive antagonist of DHFR. An appropriate host cell when wild-type DHFR is employed is the Chinese hamster ovary (CHO) cell line deficient in DHFR activity (e.g., DG44).

Alternatively, host cells (particularly wild-type hosts that contain endogenous DHFR) transformed or co-transformed with DNA sequences encoding an anti-IL-23A antibody, wild-type DHFR protein, and another selectable marker such as aminoglycoside 3′-phosphotransferase (APH), can be selected by cell growth in medium containing a selection agent for the selectable marker such as an aminoglycosidic antibiotic, e.g., kanamycin, neomycin, or G418. See, e.g., U.S. Pat. No. 4,965,199.

Where the recombinant production is performed in a yeast cell as a host cell, the TRP1 gene present in the yeast plasmid YRp7 (Stinchcomb et al., 1979, Nature 282: 39) can be used as a selectable marker. The TRP1 gene provides a selection marker for a mutant strain of yeast lacking the ability to grow in tryptophan, for example, ATCC No. 44076 or PEP4-1 (Jones, 1977, Genetics 85:12). The presence of the trp1 lesion in the yeast host cell genome then provides an effective environment for detecting transformation by growth in the absence of tryptophan. Similarly, Leu2p-deficient yeast strains such as ATCC 20,622 and 38,626 are complemented by known plasmids bearing the LEU2 gene.

In addition, vectors derived from the 1.6 m circular plasmid pKD1 can be used for transformation of Kluyveromyces yeasts. Alternatively, an expression system for large-scale production of recombinant calf chymosin was reported for K. lactis (Van den Berg, 1990, Bio/Technology 8:135). Stable multi-copy expression vectors for secretion of mature recombinant human serum albumin by industrial strains of Kluyveromyces have also been disclosed (Fleer et al., 1991, Bio/Technology 9:968-975).

Expression and cloning vectors usually contain a promoter that is recognized by the host organism and is operably linked to the nucleic acid molecule encoding an anti-IL-23p19 antibody or polypeptide chain thereof. Promoters suitable for use with prokaryotic hosts include phoA promoter, β-lactamase and lactose promoter systems, alkaline phosphatase, tryptophan (trp) promoter system, and hybrid promoters such as the tac promoter. Other known bacterial promoters are also suitable. Promoters for use in bacterial systems also will contain a Shine-Dalgarno (S.D.) sequence operably linked to the DNA encoding the anti-IL-23A antibody.

Many eukaryotic promoter sequences are known. Virtually all eukaryotic genes have an AT-rich region located approximately 25 to 30 bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the poly A tail to the 3′ end of the coding sequence. All of these sequences are suitably inserted into eukaryotic expression vectors.

Examples of suitable promoting sequences for use with yeast hosts include the promoters for 3-phosphoglycerate kinase or other glycolytic enzymes, such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Inducible promoters have the additional advantage of transcription controlled by growth conditions. These include yeast promoter regions for alcohol dehydrogenase 2, isocytochrome C, acid phosphatase, derivative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657. Yeast enhancers also are advantageously used with yeast promoters.

Anti-IL-23A antibody transcription from vectors in mammalian host cells is controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, or from heat-shock promoters, provided such promoters are compatible with the host cell systems.

The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment. A system for expressing DNA in mammalian hosts using the bovine papilloma virus as a vector is disclosed in U.S. Pat. No. 4,419,446. A modification of this system is described in U.S. Pat. No. 4,601,978. See also Reyes et al., 1982, Nature 297:598-601, disclosing expression of human p-interferon cDNA in mouse cells under the control of a thymidine kinase promoter from herpes simplex virus. Alternatively, the Rous sarcoma virus long terminal repeat can be used as the promoter.

Another useful element that can be used in a recombinant expression vector is an enhancer sequence, which is used to increase the transcription of a DNA encoding an anti-IL-23A antibody by higher eukaryotes. Many enhancer sequences are now known from mammalian genes (e.g., globin, elastase, albumin, α-fetoprotein, and insulin). Typically, however, an enhancer from a eukaryotic cell virus is used. Examples include the SV40 enhancer on the late side of the replication origin (bp 100-270), the cytomegalovirus early promoter enhancer, the polyoma enhancer on the late side of the replication origin, and adenovirus enhancers. See also Yaniv, 1982, Nature 297:17-18 for a description of enhancing elements for activation of eukaryotic promoters. The enhancer may be spliced into the vector at a position 5′ or 3′ to the anti-IL-23A antibody-encoding sequence, but is preferably located at a site 5′ from the promoter.

Expression vectors used in eukaryotic host cells (yeast, fungi, insect, plant, animal, human, or nucleated cells from other multicellular organisms) can also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding anti-IL-23A antibody. One useful transcription termination component is the bovine growth hormone polyadenylation region. See WO94/11026 and the expression vector disclosed therein. In some embodiments, humanized anti-IL-23p19 antibodies can be expressed using the CHEF system. (See, e.g., U.S. Pat. No. 5,888,809; the disclosure of which is incorporated by reference herein.)

Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41 P disclosed in DD 266,710 published Apr. 12, 1989), Pseudomonas such as P. aeruginosa, and Streptomyces. One preferred E. coli cloning host is E. coli 294 (ATCC 31,446), although other strains such as E. coli B, E. coli X1776 (ATCC 31,537), and E. coli W3110 (ATCC 27,325) are suitable. These examples are illustrative rather than limiting.

In addition to prokaryotes, eukaryotic microbes such as filamentous fungi or yeast are suitable cloning or expression hosts for anti-IL-23A antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastors (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated anti-IL-23A antibody are derived from multicellular organisms. Examples of invertebrate cells include plant and insect cells, including, e.g., numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruit fly), and Bombyx mori (silk worm). A variety of viral strains for transfection are publicly available, e.g., the L-1 variant of Autographa californica NPV and the Bm-5 strain of Bombyx mori NPV, and such viruses may be used, particularly for transfection of Spodoptera frugiperda cells.

Plant cell cultures of cotton, corn, potato, soybean, petunia, tomato, and tobacco can also be utilized as hosts.

In another aspect, expression of anti-IL-23A antibodies is carried out in vertebrate cells. The propagation of vertebrate cells in culture (tissue culture) has become routine procedure and techniques are widely available. Examples of useful mammalian host cell lines are monkey kidney CV1 line transformed by SV40 (COS-7, ATCC CRL 1651), human embryonic kidney line (293 or 293 cells subcloned for growth in suspension culture, (Graham et al., 1977, J. Gen Virol. 36: 59), baby hamster kidney cells (BHK, ATCC CCL 10), Chinese hamster ovary cells/-DHFR1 (CHO, Urlaub et al., 1980, Proc. Natl. Acad. Sci. USA 77: 4216; e.g., DG44), mouse sertoli cells (TM4, Mather, 1980, Biol. Reprod. 23:243-251), monkey kidney cells (CV1 ATCC CCL 70), African green monkey kidney cells (VERO-76, ATCC CRL-1587), human cervical carcinoma cells (HELA, ATCC CCL 2), canine kidney cells (MDCK, ATCC CCL 34), buffalo rat liver cells (BRL 3A, ATCC CRL 1442), human lung cells (W138, ATCC CCL 75), human liver cells (Hep G2, HB 8065), mouse mammary tumor (MMT 060562, ATCC CCL51), TR1 cells (Mather et al., 1982, Annals N.Y. Acad. Sci. 383: 44-68), MRC 5 cells, FS4 cells, and human hepatoma line (Hep G2).

Host cells are transformed with the above-described expression or cloning vectors for anti-IL-23A antibody production and cultured in conventional nutrient media modified as appropriate for inducing promoters, selecting transformants, or amplifying the genes encoding the desired sequences.

The host cells used to produce an anti-IL-23A antibody described herein may be cultured in a variety of media. Commercially available media such as Ham's F10 (Sigma-Aldrich Co., St. Louis, Mo.), Minimal Essential Medium ((MEM), (Sigma-Aldrich Co.), RPMI-1640 (Sigma-Aldrich Co.), and Dulbecco's Modified Eagle's Medium ((DMEM), Sigma-Aldrich Co.) are suitable for culturing the host cells. In addition, any of the media described in one or more of Ham et al., 1979, Meth. Enz. 58: 44, Barnes et al., 1980, Anal. Biochem. 102: 255, U.S. Pat. Nos. 4,767,704, 4,657,866, 4,927,762, 4,560,655, 5,122,469, WO 90/103430, and WO 87/00195 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as gentamicin), trace elements (defined as inorganic compounds usually present at final concentrations in the micromolar range), and glucose or an equivalent energy source. Other supplements may also be included at appropriate concentrations that would be known to those skilled in the art. The culture conditions, such as temperature, pH, and the like, are those previously used with the host cell selected for expression, and will be apparent to the ordinarily skilled artisan.

When using recombinant techniques, the antibody can be produced intracellularly, in the periplasmic space, or directly secreted into the medium. If the antibody is produced intracellularly, the cells may be disrupted to release protein as a first step. Particulate debris, either host cells or lysed fragments, can be removed, for example, by centrifugation or ultrafiltration. Carter et al., 1992, Bio/Technology 10:163-167 describes a procedure for isolating antibodies that are secreted to the periplasmic space of E. coli. Briefly, cell paste is thawed in the presence of sodium acetate (pH 3.5), EDTA, and phenylmethylsulfonylfluoride (PMSF) over about 30 minutes. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants. A variety of methods can be used to isolate the antibody from the host cell.

The antibody composition prepared from the cells can be purified using, for example, hydroxylapatite chromatography, gel electrophoresis, dialysis, and affinity chromatography, with affinity chromatography being a typical purification technique. The suitability of protein A as an affinity ligand depends on the species and isotype of any immunoglobulin Fc domain that is present in the antibody. Protein A can be used to purify antibodies that are based on human gamma1, gamma2, or gamma4 heavy chains (see, e.g., Lindmark et al., 1983 J. Immunol. Meth. 62:1-13). Protein G is recommended for all mouse isotypes and for human gamma3 (see, e.g., Guss et al., 1986 EMBO J. 5:1567-1575). A matrix to which an affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a C_{H3} domain, the Bakerbond ABX™ resin (J. T. Baker, Phillipsburg, N.J.) is useful for purification. Other techniques for protein purification such as fractionation on an ion-exchange column, ethanol precipitation, reverse phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, typically performed at low salt concentrations (e.g., from about 0-0.25M salt).

### Therapeutic Uses

In another embodiment, an anti-IL-23A antibody disclosed herein is useful in the treatment of various disorders associated with the expression of IL-23p19 as described herein. In one aspect, a method for treating an IL-23 associated disorder comprises administering a therapeutically effective amount of an anti-IL-23A antibody to a subject in need thereof.

The anti-IL-23A antibody is administered by any suitable means, including parenteral, subcutaneous, intraperitoneal, intrapulmonary, and intranasal, and, if desired for local immunosuppressive treatment, intralesional administration (including perfusing or otherwise contacting the graft with the antibody before transplantation). The anti-IL-23A antibody or agent can be administered, for example, as an infusion or as a bolus. Parenteral infusions include intramuscular, intravenous, intraarterial, intraperitoneal, or subcutaneous administration. In addition, the anti-IL-23A antibody is suitably administered by pulse infusion, particularly with declining doses of the antibody. In one aspect, the dosing is given by injections, most preferably intravenous or subcutaneous injections, depending in part on whether the administration is brief or chronic. In one aspect, the dosing of the anti-IL-23 antibody is given by subcutaneous injections.

For the prevention or treatment of disease, the appropriate dosage of antibody will depend on a variety of factors such as the type of disease to be treated, as defined above, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments.

The term “suppression” is used herein in the same context as “amelioration” and “alleviation” to mean a lessening of one or more characteristics of the disease.

The antibody is formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “therapeutically effective amount” of the antibody to be administered will be governed by such considerations.

The antibody may optionally be formulated with one or more agents currently used to prevent or treat the disorder in question. The effective amount of such other agents depends on the amount of anti-IL-23A antibody present in the formulation, the type of disorder or treatment, and other factors discussed above.

### IL-23-Associated Disorders

The anti-IL-23p19 antibodies or agents are useful for treating or preventing an immunological disorder characterized by abnormal expression of IL-23, e.g., by inappropriate activation of immune cells (e.g., lymphocytes or dendritic cells). Such abnormal expression of IL-23 can be due to, for example, increased IL-23 protein levels.

Immunological diseases that are characterized by inappropriate activation of immune cells and that can be treated or prevented by the methods described herein can be classified, for example, by the type(s) of hypersensitivity reaction(s) that underlie the disorder. These reactions are typically classified into four types: anaphylactic reactions, cytotoxic (cytolytic) reactions, immune complex reactions, or cell-mediated immunity (CMI) reactions (also referred to as delayed-type hypersensitivity (DTH) reactions). (See, e.g., Fundamental Immunology (William E. Paul ed., Raven Press, N.Y., 3rd ed. 1993).) Immunological diseases include inflammatory diseases and autoimmune diseases.

Examples of immunological diseases include the following: psoriasis, inflammatory bowel disease, for example ulcerative colitis or Crohn's disease, and spondyloarthritis, for example ankylosing spondylitis, non-radiographic axial spondyloarthritis, peripheral spondyloarthritis or psoriatic arthritis.

In one aspect, in the context of the present invention, the immunological disease is psoriasis. Psoriasis is a chronic inflammatory disease of the skin characterized by dysfunctional keratinocyte differentiation and hyperproliferation and marked accumulation of inflammatory T cells and dendritic cells. For example, the immunological disease includes plaque psoriasis, for example chronic plaque psoriasis, for example moderate to severe chronic plaque psoriasis, for example in patients who are candidates for systemic therapy or phototherapy. In one aspect, the immunological disease is moderate to severe plaque psoriasis and the patient is a patient who failed to respond to, or who has a contraindication to, or is intolerant to other systemic therapy including ciclosporin, methotrexate, psoralen or ultraviolet-A light (PUVA).

For example, the immunological disease includes palmar pustular psoriasis, guttate psoriasis, inverse psoriasis, pustular psoriasis or erythodermic psoriasis (also known as psoriatic erythroderma). For example, the immunological disease includes generalized pustular psoriasis (GPP).

For psoriasis, disease severity can be characterized by body surface area (BSA) involvement with <5% being considered mild, 5-10% moderate and >10% severe. In some cases, disease status is measured using the Psoriasis Area and Severity Index (PASI), a composite measure of erythema, induration, desquamation and BSA affected by psoriasis with a range of scores from 0 to 72. In one aspect, the percentage of patients reaching PASI₇₅ (PASI 75), a 75% reduction in score from baseline at a certain time (for example 12 or 16 weeks) is used as a primary endpoint in psoriasis treatment, for example in psoriasis treatment trials. In one aspect, the percentage of patients reaching a PASI₉₀ (PASI 90), a 90% reduction in score from baseline at a certain time (for example at week 12 or 16) is used as primary endpoint in psoriasis treatment, for example in psoriasis treatment trials.

In one aspect, the percentage of patients reaching a PASI₉₀ at week 12 pursuant to a method according to the present invention, for example using Antibody A, is at least 60%, at least 65% or at least 70%.

In one aspect, the percentage of patients reaching a sPGA score of clear (score of 0) or almost clear (score of 1) at a certain time (for example at week 12 or 16) is used as primary endpoint in psoriasis treatment, for example in psoriasis treatment trials.

In one aspect, a patient treated within the context of the instant invention is a patient with stable moderate to severe chronic plaque-type psoriasis with or without psoriatic arthritis involving 10% or more body surface area, with disease severity PASI equal or greater than 12 and/or sPGA score of moderate and above (score of at least 3). In one aspect, a patient treated within the context of the instant invention is a patient with a psoriasis disease duration of at least 6 months prior to initiating the treatment.

In one aspect, in the context of the present invention, the immunological disease is psoriatic arthritis. For psoriatic arthritis, a patient has psoriatic skin lesions or a history of psoriasis in combination with nail dystrophy, inflammation of the digits (dactylitis) and a characteristic radiographic appearance (ill-defined ossification near joint margins) with a negative serum test for rheumatoid factor. Efficacy of treatment of psoriatic arthritis in clinical trials is frequently gauged using the percentage of patients achieving the American College of Rheumatology (ACR) composite measure of efficacy, as has been used prominently in trials in rheumatoid arthritis. The ACR20 indicates achievement of at least a 20% improvement in the number of swollen and tender joint counts as well as 20% improvement in 3 of 5 ACR core set measures when comparing baseline to some point in time after initiation of treatment e.g., at 24 weeks.

In one aspect, the percentage of ACR20 responders (20% improvement in American College of Rheumatology response criteria), for example at 24 weeks, is used as primary endpoint in psoriatic arthritis treatment, for example in psoriatic arthritis treatment trials.

In one aspect, immunological disease is psoriatic arthritis, for example active psoriatic arthritis, and the anti-IL-23A antibody is used alone or in combination with one or more non-biologic DMARDs (Disease-Modifying Antirheumatic Drug), for example to reduce signs and symptoms. In one aspect, the anti-IL-23A antibody is used or indicated to inhibit the progression of structural damage, and/or improve physical function.

In one aspect, the anti-IL-23A antibody is used alone or in combination with methotrexate (MTX) for the treatment of psoriatic arthritis, for example active psoriatic arthritis, for example when the response to previous non-biological DMARD therapy has been inadequate. In one aspect, the anti-IL-23A antibody is used to reduce the rate of progression of peripheral joint damage as measured by X-ray and/or to improve physical function.

In one aspect, in the context of the present invention, the immunological disease is axial (spinal) spondyloarthritis (ax-SpA), including ankylosing spondylitis (AS, also called radiographic ax-SpA) and non-radiographic ax-SpA.

In one aspect, in the context of the present invention, the immunological disease is Ankylosing Spondylitis. Ankylosing Spondylitis (AS) is an inflammatory disease involving primarily the axial skeleton and sacroiliac joints. Other musculoskeletal manifestations of the disease include peripheral arthritis and enthesitis. Extra-articular disease includes anterior uveitis, osteoporosis, cardiac disease with primarily valvular involvement, renal disease, lung disease, gastrointestinal disease, and skin disease. AS has a male predominance with a male-to-female ratio of 3:1. The peak age of onset is typically in the second or third decade of life. Estimates of the prevalence of AS worldwide range from approximately 0.5% to 0.07%. Patients with AS are likely to lose their physical function and ability to work, which is likely to have a significant impact on the quality of life unless an appropriate treatment is administered to the patients.

For ankylosing spondylitis, a patient has inflammatory back pain, asymmetrical peripheral arthritis, inflammation of the tendons (enthesitis) and extra-articular features, such as uveitis, and a strong genetic association with human leucocyte antigen B27. In one aspect, the percentage Ankylosing Spondylitis Disease Activity Score 40 (ASDAS 40) responders, for example at 24 weeks is used as primary endpoint in ankylosing spondylitis treatment, for example in ankylosing spondylitis treatment trials.

In one aspect, in the context of the present invention, the immunological disease is non-radiographic axial spondyloarthritis (ax-SpA). Non-radiographic axial spondyloarthritis, a more recently defined entity than AS, is considered to represent an earlier manifestation of the same pathologic processes as AS, although it is increasingly recognized that some patients (particularly females) may not progress to radiographic disease, and may therefore be considered to have a distinct subtype of this disease.

### Pharmaceutical Compositions and Administration Thereof

A composition comprising an anti-IL-23A antibody can be administered to a subject having or at risk of having an immunological disorder. The term “subject” as used herein means any mammalian patient to which an anti-IL-23A antibody can be administered, including, e.g., humans and non-human mammals, such as primates, rodents, and dogs. Subjects specifically intended for treatment using the methods described herein include humans. The antibodies can be administered either alone or in combination with other compositions in the prevention or treatment of the immunological disorder.

Anti-IL-23A antibodies for use in such pharmaceutical compositions are described herein, for example Antibody A, Antibody B, Antibody C or Antibody D.

Various delivery systems are known and can be used to administer the anti-IL-23A antibody. Methods of introduction include but are not limited to intradermal, intramuscular, intraperitoneal, intravenous, subcutaneous, intranasal, epidural, and oral routes. The anti-IL-23A antibody can be administered, for example by infusion, bolus or injection, and can be administered together with other biologically active agents such as chemotherapeutic agents. Administration can be systemic or local. In one embodiment, the administration is by subcutaneous injection. Formulations for such injections may be prepared in for example prefilled syringes that may be administered once every other week.

In specific embodiments, the anti-IL-23A antibody is administered by injection, by means of a catheter, by means of a suppository, or by means of an implant, the implant being of a porous, non-porous, or gelatinous material, including a membrane, such as a sialastic membrane, or a fiber. Typically, when administering the composition, materials to which the anti-IL-23A antibody or agent does not absorb are used.

In other embodiments, the anti-IL-23A antibody is delivered in a controlled release system. In one embodiment, a pump may be used (see, e.g., Langer, 1990, Science 249:1527-1533; Sefton, 1989, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985, Science 228:190; During et al., 1989, Ann. Neurol. 25:351; Howard et al., 1989, J. Neurosurg. 71:105.) Other controlled release systems are discussed, for example, in Langer, supra.

An anti-IL-23p19 antibody is typically administered as pharmaceutical compositions comprising a therapeutically effective amount of the antibody and one or more pharmaceutically compatible ingredients.

In typical embodiments, the pharmaceutical composition is formulated in accordance with routine procedures as a pharmaceutical composition adapted for intravenous or subcutaneous administration to human beings. Typically, compositions for administration by injection are solutions in sterile isotonic aqueous buffer. Where necessary, the pharmaceutical can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical is administered by injection, an ampoule of sterile water for injection or saline can be provided so that the ingredients can be mixed prior to administration.

Further, the pharmaceutical composition can be provided as a pharmaceutical kit comprising (a) a container containing an anti-IL-23A antibody in lyophilized form and (b) a second container containing a pharmaceutically acceptable diluent (e.g., sterile water) for injection. The pharmaceutically acceptable diluent can be used for reconstitution or dilution of the lyophilized anti-IL-23A antibody. Optionally associated with such container(s) can be a notice in the form prescribed by a governmental agency regulating the manufacture, use or sale of pharmaceuticals or biological products, which notice reflects approval by the agency of manufacture, use or sale for human administration.

Examples of pharmaceutical compositions used in the context of the present invention are disclosed in Example 4 hereinbelow.

Numeric ranges are inclusive of the numbers defining the range. The headings provided herein are not limitations of the various aspects or embodiments of this disclosure which can be read by reference to the specification as a whole.

As used in the subject specification, the singular forms “a”, “an” and “the” include plural referents unless the context clearly dictates otherwise. The terms “include,” “have,” “comprise” and their variants are used synonymously and to be construed as non-limiting. Throughout the specification, where compositions are described as comprising components or materials, it is contemplated that the compositions can in embodiments also consist essentially of, or consist of, any combination of the recited components or materials, unless described otherwise. The technology illustratively disclosed herein suitably may be practiced in the absence of any element which is not specifically disclosed herein.

The invention is further described in the following examples, which are not intended to limit the scope of the invention.

### EXAMPLES

### Example 1: Clinical Studies

The results shown in Examples 1a, 1b and 1c are derived from the same clinical study.

### Example 1a: Study

This study assessed the efficacy and safety of Antibody A vs ustekinumab in patients with moderate-to-severe plaque psoriasis.

166 patients were randomly assigned to one of three dose regimens of Antibody A (18 mg single injection; 90 or 180 mg at weeks 0 and 4) or ustekinumab (45 or 90 mg). PASI (Psoriasis Area and Severity Index), sPGA (Static Physician Global Assessment) and safety parameters were assessed. Primary endpoint was achievement of PASI 90 (achievement of ≥90% reduction from baseline PASI score) at week 12, with primary analysis comparing the two highest doses of Antibody A (pooled) with ustekinumab.

The primary analysis showed Antibody A to be superior to ustekinumab (PASI 90 response rate of 77.1% for Antibody A vs 40% for ustekinumab [p<0.0001]). sPGA scores of 0 (clear) or 1 (almost clear) were achieved by 90.4% of Antibody A patients compared with 67.5% of ustekinumab patients. Complete clearing of lesions (PASI 100) was achieved in 45.8% of Antibody A patients and 17.5% of ustekinumab patients. AEs were similar across treatment groups with no drug-related severe or serious AEs (adverse events).

Selective blockade of IL-23p19 with Antibody A is associated with superior clinical response to ustekinumab in patients with moderate-to-severe plaque psoriasis.

### Example 1b: Study

Objectives: The efficacy and safety of the selective IL-23p19 antagonist monoclonal antibody Antibody A were assessed and compared with ustekinumab in patients with moderate-to-severe plaque psoriasis (including those with and without concurrent psoriatic arthritis (PsA)).

Methods: In this Phase 2 study, 166 patients were randomly assigned (1:1:1:1 ratio) to receive subcutaneous injections of one of three dose regimens of Antibody A (18 mg single dose at week 0; 90 or 180 mg at weeks 0, 4 and 16) or ustekinumab (45 or 90 mg at weeks 0, 4 and 16). Skin lesions were assessed using the Psoriasis Area and Severity Index (PASI) with the primary endpoint of PASI 90 (90% improvement from baseline) at 12 weeks. In patients who had concurrent PsA (either diagnosed by rheumatologist or suspected), the pain was assessed using the Visual Analogue Scale (pain-VAS) at baseline and at weeks 4, 12 and 24. In this interim analysis, all patients had completed the week 12 visit; there is incomplete data after week 12.

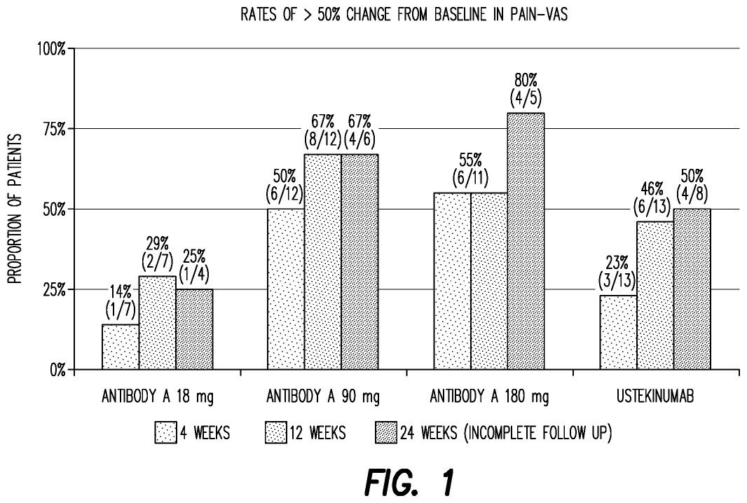

Results: The primary endpoint of PASI 90 response at week 12 was achieved by 32.6% ( 14/43), 73.2% ( 30/41) and 81.0% ( 34/42) of Antibody A patients in the 18, 90 and 180 mg groups, respectively, and 40.0% ( 16/40) of ustekinumab patients. A two-sided Cochran-Mantel-Haenszel test of PASI 90 response at week 12 between the 18, 90 and 180 mg groups of Antibody A and ustekinumab gave p-values of 0.4337, 0.0013 and <0.0001, respectively. Of 166 patients, 46 (27.7%) had concurrent PsA. Median percentage decrease from baseline in pain-VAS at week 12 were 31.9%, 70.2% and 58.4%, respectively, in patients in Antibody A 18, 90 and 180 mg dose groups compared with 57.3% in ustekinumab-treated patients. More than 50% decrease in pain-VAS (defined post hoc) at week 12 was achieved in 29% ( 2/7), 73% ( 8/11) and 50% ( 6/12) of patients in Antibody A 18, 90 and 180 mg dose groups, respectively, compared with 54% ( 7/13) in ustekinumab-treated patients. FIG. 1 demonstrates that reductions in pain-VAS score were observed as early as 4 weeks and maintained through 24 weeks (incomplete data). Adverse events (AEs) were similar across treatment groups with no drug-related severe or serious AEs.

Conclusions: Selective blockade of IL-23p19 with Antibody A is associated with superior clinical response compared to ustekinumab on skin lesions in patients with moderate-to-severe plaque psoriasis. Numeric improvement over ustekinumab in pain-VAS was also observed in patients with PsA.

PASI 50, PASI 75 and PASI 100 responses at week 12 were also determined in the above study. They showed proportions of patients achieving PASI 50 at week 12 of 93%, 95% and 100% of Antibody A patients in the 18, 90 and 180 mg groups, respectively, and 87% of ustekinumab patients. Achievement of PASI 50 over time is shown in FIG. 2.

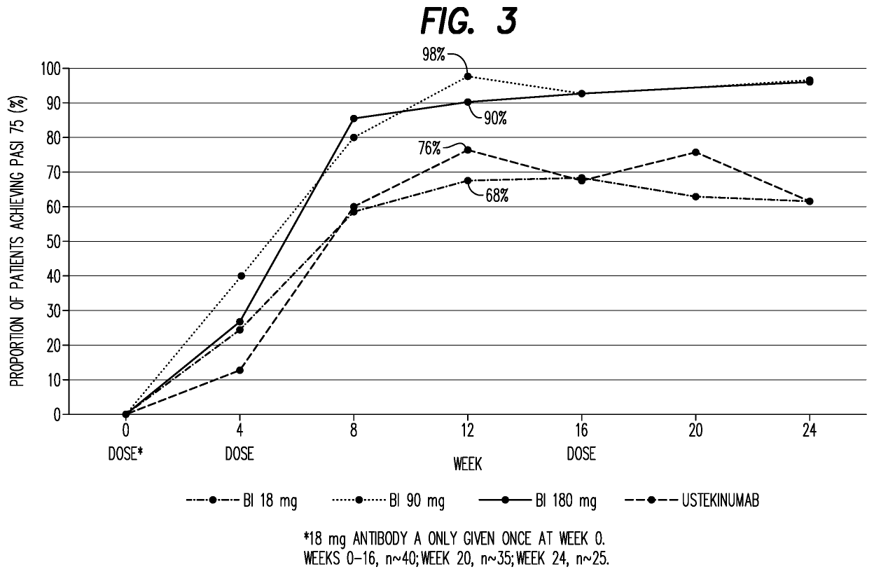

They also showed proportions of patients achieving PASI 75 at week 12 of 76%, 90% and 98% of Antibody A patients in the 18, 90 and 180 mg groups, respectively, and 68% of ustekinumab patients. Achievement of PASI 75 over time is shown in FIG. 3.

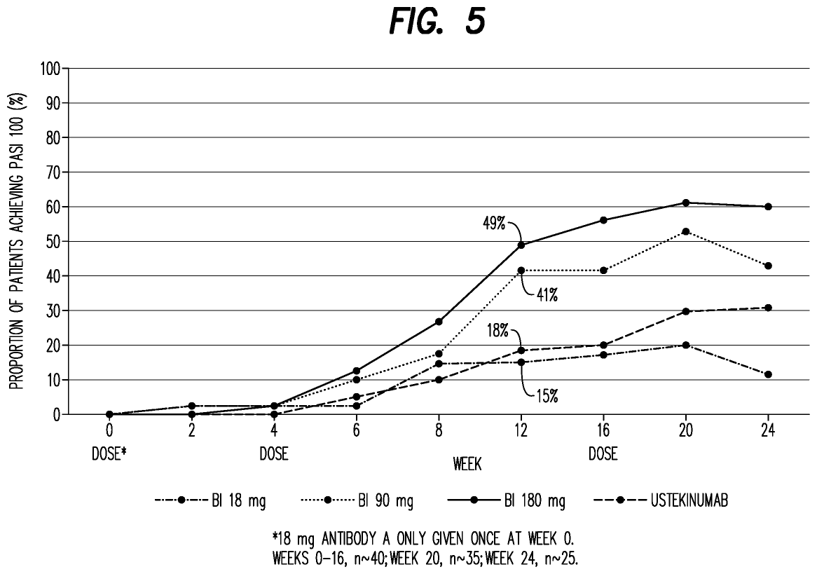

They also showed proportions of patients achieving PASI 100 at week 12 of 18%, 41% and 49% of Antibody A patients in the 18, 90 and 180 mg groups, respectively, and 15% of ustekinumab patients. Achievement of PASI 100 over time is shown in FIG. 5.

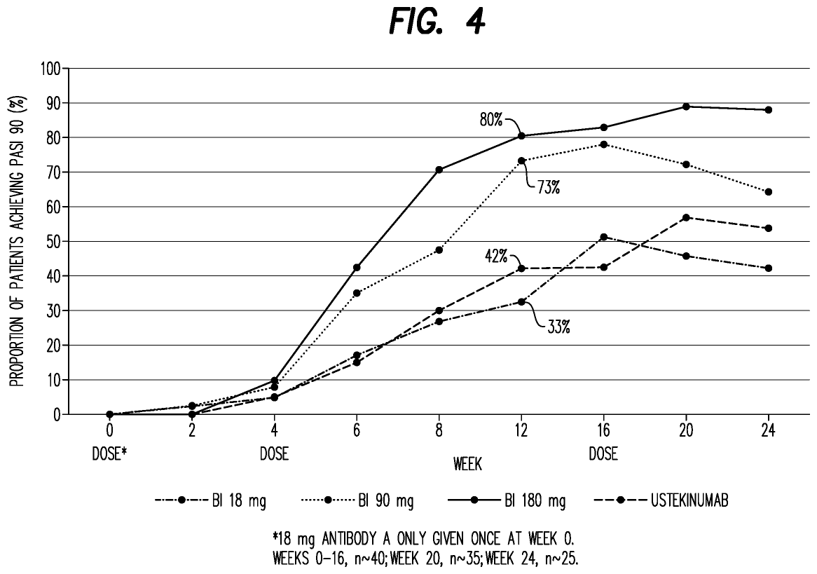

Achievement of PASI 90 over time is shown in FIG. 4.

### Example 1c: Study

This study assessed the onset and duration of clinical response following treatment with a selective IL-23p19 inhibitor (Antibody A) compared with ustekinumab in patients with moderate-to-severe chronic plaque psoriasis.

Materials & Methods: In this multi-center, randomized, double-blind (within Antibody A dose groups) Phase II study, 166 patients with moderate-to-severe chronic plaque psoriasis were assigned to receive subcutaneous injections of either Antibody A at three different dose regimens (18 mg single injection; 90 or 180 mg at Weeks 0, 4 and 16) or ustekinumab (45 or 90 mg, based on weight, at Weeks 0, 4 and 16). PASI was assessed at Weeks 0, 1, 2, 4, 6 and 8, followed by every 4 weeks for a total of 48 weeks. Safety was assessed at all visits by recording adverse events (AEs).

Results: At Week 24, PASI 90 was achieved by 66% ( 27/41) and 86% ( 36/42) of patients in the 90 mg and 180 mg Antibody A arms, respectively, compared with 55% ( 22/40) of patients receiving ustekinumab. Complete clearing of lesions (PASI 100) was achieved in 41% ( 17/41) and 57% ( 24/42) of patients in the 90 and 180 mg Antibody A arms, compared with 28% of patients receiving ustekinumab. The time to onset of PASI 90 was approximately twice as fast for patients in the 90 and 180 mg Antibody A arms compared with those in the ustekinumab arm (median days to onset of PASI 90=57 days [in both Antibody A dose groups; ~8 weeks] vs 113 days [~16 weeks], respectively, p=0.0016 [90 mg] and p<0.0001 [180 mg]). Patients in the 90 and 180 mg Antibody A arms who achieved PASI 90 maintained that response longer than those receiving ustekinumab (days to 50% of patients having first loss of PASI 90=337 days [in both Antibody A dose groups; ~48 weeks] vs 253 days [~36 weeks], respectively). AEs were similar between treatment arms and there was no dose response relationship for any AE. Seven patients reported serious AEs (four in the 18 mg and two in the 90 mg Antibody A arms and one in the ustekinumab arm); all were considered unrelated to study medication.

The same study showed that Antibody A had a rapid onset of effect with 69% and 66% of patients (180 mg and 90 mg, respectively) achieving PASI 50 by Week 4, vs 45% of ustekinumab patients. By Week 8, 83% and 80% of Antibody A patients (180 mg and 90 mg, respectively) had achieved PASI 75 vs 60% of ustekinumab patients. By Week 12, the proportion of patients achieving PASI 90 (primary endpoint) was 81% and 73% for 180 mg and 90 mg Antibody A arms vs 40% for ustekinumab. Body weight had little impact on efficacy for Antibody A. At Week 20, 90% and 76% of Antibody A patients (180 mg and 90 mg, respectively) achieved PASI 90 vs 55% for ustekinumab; and 62% and 51% of Antibody A patients had complete clearing of lesions (PASI 100) vs 25% of ustekinumab patients. Clinical responses were sustained at high levels beyond Week 36 for the 180 mg and 90 mg Antibody A dose groups. In patients with PsA, there was a greater decrease in pain at Week 12 observed with Antibody A (180 mg and 90 mg, pooled) vs ustekinumab (68% vs 57%). In patients with psoriatic arthritis, pain was assessed by visual analog scale and expressed as median percentage change from baseline.

Conclusions: Selective blockade of IL-23 by Antibody A demonstrates superior efficacy, a more rapid onset and longer duration of response compared with blockade of IL-12 and IL-23 with ustekinumab.

In the same study, Dermatology Life Quality Index (DLQI) and the EuroQol-5D (EQ-5D) were completed at Weeks 0, 12, 24, and 48. The median percentage changes from baseline in DLQI total score (analyzed descriptively and by the van Elteren test) were compared with ustekinumab at Weeks 12 and 24. The proportion of patients achieving a DLQI score of 0 or 1 was also assessed. The EQ-5D index score was analyzed descriptively. Antibody A results are reported for the pooled 90 mg and 180 mg arms.

At baseline, the median DLQI score was 13.0 for patients randomized to Antibody A, compared with 16.0 for those randomized to ustekinumab. Mean EQ-5D index score was 0.7 in both treatment arms. At Week 12, the median percentage improvement in DLQI score was greater for patients receiving Antibody A than for those receiving ustekinumab (100% vs 90.6%, respectively; p=0.0304). The proportion of patients with a DLQI score of 0 or 1 at Week 24 was 80% for Antibody A patients compared with 61% for ustekinumab patients. At Week 12, Antibody A patients were observed to have a 50% greater improvement in mean EQ-5D score compared with ustekinumab patients (0.3 vs 0.2, respectively). This was sustained at Week 24.

In patients with moderate-to-severe plaque psoriasis, selective blockade of IL-23 by Antibody A demonstrated superior efficacy and provides significant improvement in QoL outcomes compared with ustekinumab.

In the same study, the efficacy of Antibody A was assessed for the treatment of scalp, palmoplantar and nail psoriasis.

Psoriasis Scalp Severity Index (PSSI) and Palmoplantar Psoriasis Area and Severity Index (PPASI) were used to evaluate scalp and palmoplantar psoriasis, respectively. Nail Psoriasis Severity Index (NAPSI) was assessed on the hands only. Assessments were performed at baseline and (in those who had symptoms at baseline) at Weeks 1, 2, 4, 6, 8, 12 and every 4 weeks through Week 48. Patients who were documented to have no signs or symptoms were assigned a total score of zero for that visit. Median percentage improvements from baseline for Antibody A (90 mg and 180 mg, pooled) were compared with those for ustekinumab.

One hundred and fifty four (92.8%) patients had scalp psoriasis, 42 (25.3%) patients had palmoplantar psoriasis and 96 (57.8%) patients had nail psoriasis. Scalp and palmoplantar disease responded rapidly; by Week 2, Antibody A-treated patients showed improvements from baseline in PSSI and PPASI of 50% and 51%, compared with 37% and 30% for ustekinumab-treated patients. By Week 6, patients in both Antibody A and ustekinumab groups had complete clearance of scalp psoriasis, which were maintained throughout the study. For PPASI, complete clearance was seen at Week 6 for Antibody A and Week 16 for ustekinumab and maintained throughout the study. Nail disease resolved more slowly, with 30% improvement in NAPSI for Antibody A at Week 6 compared to 0% for ustekinumab. By Week 12, improvements in NAPSI were 41% and 36% for Antibody A and ustekinumab, respectively, and by Week 24, the improvements in NAPSI were 61% and 67%, respectively.

Selective blockade of IL-23 by Antibody A demonstrates promising efficacy in the treatment of scalp, palmoplantar, and nail psoriasis, and suggests a more rapid improvement may be achieved with Antibody A compared with ustekinumab.

### Example 1d: Study

Clinical responses following drug withdrawal and retreatment or switching from ustekinumab to Antibody A are tested.

Materials & Methods: Of the 166 patients with moderate-to-severe plaque psoriasis in the above Phase II study, 109 patients who successfully completed 48 weeks, or whose PASI response fell below 50% prior to completing the trial, entered an open-label extension (OLE) study and were treated with Antibody A 90 mg every 12 weeks. Efficacy (PASI 90 and 100) and safety (AEs) were assessed. Non-response imputation was used for missing efficacy data.

Results: At the initiation of the OLE, PASI 90 rates were 0% (0/22), 53.6% ( 15/28), 50.0% ( 16/32) and 14.8% ( 4/27) of patients from the Phase II Antibody A (18 mg, 90 mg and 180 mg) and ustekinumab arms, respectively. Twelve weeks after receiving a single injection of 90 mg Antibody A, 59.1% ( 13/22), 71.4% ( 20/28), 68.8% ( 22/32) and 66.7% ( 18/27) of patients in each arm achieved PASI 90. Among all non-responders (PASI improvement <50%) from the Phase II study, 48.6% ( 17/35) achieved PASI 90 response 12 weeks after a single injection of 90 mg Antibody A. In addition, all patients who entered the OLE with a PASI 90 response maintained PASI 90 responses. Similarly, PASI 100 rates at initiation of the OLE were 0% (0/22), 39.3% ( 11/28), 28.1% ( 9/32) and 3.7% ( 1/27) for the 4 Phase II arms, respectively. These increased to 31.8% ( 7/22), 42.9% ( 12/28), 40.6% ( 13/32) and 37.0% ( 10/27) 12 weeks after a single injection of 90 mg Antibody A. AEs were similar to the preceding Phase II trial. One patient experienced serious AEs (18 mg Antibody A arm) that were considered unrelated to study medication.

Conclusions: Patients who had been previously treated with ustekinumab responded well to Antibody A. In addition, re-treatment with Antibody A maintained or improved upon PASI 90 response rates.

### Example 2: Assessment of Efficacy

Antibody A is administered to a patient at a dose and dose regimen described herein. The efficacy of Antibody A is assessed by one or more of the following endpoints, which are determined using known methods. Endpoints are measured for example at one or more of week 4, 6, 8, 12, 16, 20, 24, 28, 32, 36, 40, 44, 48, 52, 76 or 104 after administration or as set forth below.

### Psoriasis:

Achievement of ≥90% reduction from baseline PASI score (PASI₉₀), for example at week 12.
Achievement of ≥75% reduction from baseline in PASI score (PASI₇₅), for example at week 12 and 24.
Achievement of 100% reduction from baseline in PASI score (PASI₁₀₀), for example at week 12.
Achievement of ≥50% reduction from baseline in PASI score (PASI₅₀), for example at week 12.
Achievement of PASI₉₀, for example at week 24.
Achievement of sPGA clear or almost clear, for example at week 12.
Percentage of PASI reduction from baseline, for example at week 12.
Time to loss of PASI₅₀ response. This endpoint is calculated from the first treatment to first <50% reduction of PASI score compared with baseline after the response has been achieved.
   Other endpoints are as follows:
Achievement of PASI₉₀ and PASI₇₅ for example at week 4, 6, 8, 16, 20, 28, 32, 36, 40, 44, 48.
Achievement of PASI₁₀₀, PASI₅₀ and percentage of PASI reduction from baseline, for example at week 4, 6, 8, 16, 20, 24, 28, 32, 36, 40, 44, 48.
Achievement of sPGA clear or almost clear, for example at week 4, 6, 8, 16, 20, 24, 28, 32, 36, 40, 44, 48.
Time to onset of PASI₅₀ response (from the first treatment to first ≥50% reduction of PASI score compared with baseline).
Percent change from baseline in NAPSI (Nail Psoriasis Severity Index), PSSI (Psoriasis Scalp Severity Index) and PPASI (Palmoplantar Psoriasis Area Severity Index), for example at week 4, 6, 8, 12, 16, 20, 24, 28, 32, 36, 40, 44, 48.
Change from baseline in PGAR (Patient's Global Assessment Rank) and PAI (Patient's Assessment of Itch), for example at week 4, 12, 24, 36, 48.
Change from baseline in Pain-VAS, for example at week 12 (in subgroup of patients with psoriatic arthritis only).
Change from baseline in Dermatology Life Quality Index (DLQI), for example at weeks 4, 6, 8, 12, 16, 20, 24, 48.
Change from baseline in EQ-5D (EuroQoL Group Questionnaire) VAS score (Visual Analog Scale), for example at week 12.
Change from baseline in EQ-5D index score, for example at week 12.
Proportion of patients achieving Dermatology Life Quality Index (DLQI) scores of 0 or 1, for example at week 12.
Change from baseline in psoriasis symptoms evaluated using the total score on the PSS (Psoriasis Symptoms Scale), for example at week 16.
Achievement of total score on the PSS of 0, for example at week 16.
   Psoriatic arthritis (responses assessed for example at one or more of week 4, 6, 8, 12, 16, 20, 24, 28, 32, 36, 40, 44, 48, 52, 76, 104):
Psoriatic Arthritis Screening and Evaluation (PASE) Questionnaire, for example at week 4, 6, 8, 12, 16, 20, 24, 28, 32, 36, 40, 44, 48.
20% improvement in American College of Rheumatology response criteria (ACR 20), for example at week 4, 6, 8, 12, 16, 20, 24, 28, 32, 36, 40, 44, 48.
50% and 70% improvement in American College of Rheumatology response criteria (ACR 50 and ACR 70).
Clinical Disease Activity Index (CDAI).
Disease Activity Score for 28-joint counts (DAS28).
Dactylitis (swelling of the whole digit), with assessment of the number of all 20 digits that are affected (ranges from 0 to 20 digits (fingers and toes) as present or absent for each digit).
Enthesitis (inflammation of tendon and ligament insertions, as assessed by the presence or absence of tenderness at the six sites of the Leeds Enthesitis Index (LEI)).
Individual ACR components including the number of tender or painful joints, the number of swollen joints.
The patient's global assessment of disease activity and joint pain.
The physician's global assessment of disease activity.
Responses of the Health Assessment Questionnaire-Disability Index (HAQ-DI), Health Assessment Questionnaire-Disability Index (HAQ-DI).
C-reactive protein levels.
Erythrocyte sedimentation rate.
Patient-reported outcome measures included the Bath Ankylosing Spondylitis Disease Activity Index (BASDAI). Scores on the Bath Ankylosing Spondylitis Disease Activity Index (BASDAI).
Version 2 of the Medical Outcomes Study 36-Item Short-Form Health Survey (SF-36).
Psoriasis Symptom Inventory.
The modified total Sharp score (mTSS), for example at week 24, 52, 104.
The tender-joint count.
The swollen-joint count.
Patient's assessment of pain,
Patient's assessment of Level of disability.
Acute-phase reactant (C-reactive protein or erythrocyte sedimentation rate),
Patient's global assessment of disease.
Physician's global assessment of disease.
Minimal Disease Activity (MDA).
Composite Psoriatic Disease Activity Index (CPDAI).
Psoriasis Area and Severity Index (PASI) percentage change from baseline (PASI 75/90/100).
Modified Nail Psoriasis Severity Index (mNAPSI).
Psoriatic Arthritis Quality of Life (PsAQOL).
Functional Assessment of Chronic Illness Therapy-Fatigue (FACIT-F).
Psoriatic Arthritis Magnetic Resonance Imaging Scoring System (PsAMRIS), for example at week 24, 52 and 104.
   For Ankylosing Spondylitis and non-radiographic ax-SpA (responses assessed for example at one or more of week 4, 6, 8, 12, 16, 20, 24, 28, 32, 36, 40, 44, 48, 52, 76, 104):
Patient-reported inflammatory symptoms.
Acute-phase reactants (erythrocyte sedimentation rate [ESR] or CRP).
Bath Ankylosing Spondylitis Disease Activity Index (BASDAI). Change in BASDAI score as compared to baseline.
Ankylosing Spondylitis Disease Activity Score (ASDAS). Change in ASDAS score compared to baseline.
Bath Ankylosing Spondylitis Function Index (BASFI). Change in BASFI score as compared to baseline.
Bath Ankylosing Spondylitis Metrology Index (BASMI). Change in spinal mobility assessed by BASMI score as compared to baseline.
Magnetic Resonance Imaging (MRI), for example at week 24, 52, 104.
ASAS 40 response.
ASAS 5/6 response.
ASAS partial remission criteria.
ASAS 20 response
Change in peripheral joint count (TJC and SJC) as compared to baseline.
Change in entheseal score (MASES) at as compared to baseline.
Change in Ankylosing Spondylitis Disease Activity Score (ASDAS) score as compared to baseline.
Time to loss of maintenance.
Time to post-treatment flare.
Time to loss of maintenance.
Ankylosing Spondylitis Quality of Life scale (ASQoL).

### Example 3: Pharmacokinetic Endpoint(s)

In order to determine the pharmacokinetic parameters of an anti-IL-23A antibody, such as Antibody A, the following parameters are evaluated using methods known in the art:
maximum measured plasma concentration (Cmax)
time from dosing to the maximum plasma concentration (tmax)
area under the plasma concentration-time curve over the time interval of treatment (AUC0-t)
area under the plasma concentration-time curve over the time interval from zero extrapolated to infinity (AUC0-∞)
terminal half-life (t½)
population pharmacokinetics.

### Example 4: Pharmaceutical Compositions

Examples of formulations suitable for an antibody of the present invention are shown below. Antibodies used in the formulations below are for example Antibody A, Antibody B, Antibody C or Antibody D.

### Formulation 1:

| | | | Nominal Amount |
|---|---|---|---|
| | Concentration | Concentration | [mg/vial] |
| Components | [mmol/L] | [g/l] | V = 10.0 ml |
| Antibody | | 10.0 | 100.0 |
| Succinic acid | 0.7 | 0.083 | 0.8 |
| Disodium | 24.3 | 6.564 | 65.6 |
| succinate | | | |
| hexahydrate | | | |
| Sodium chloride | 125 | 7.305 | 73.1 |
| Polysorbat 20 | 0.16 | 0.20 | 0.20 |
| Water for Injection | — | Ad 1 L | Ad 1 mL |

The pH of formulation 1 is typically in the range of pH 6.0 to 7.0, for example pH 6.5. This formulation is particularly suitable for intravenous administration.

Molecular weight (MW in g/mol) of used excipients: Disodium succinate hexahydrate=270.14 g/mol; Succinic acid=118.09 g/mol; Sodium chloride=58.44 g/mol.

The osmolarity of the formulation is 300+/−30 mOsmol/kg, as determined using an Osmomat 030 (Gonotec GmbH, Berlin, Germany). The density at 20° C. of the formulation is approximately 1.0089 g/cm³, as determined using a measuring unit DMA 4500 (Anton Paar GmbH, Ostfildern-Schamnhausen, Germany).

### Formulation 2:

| | | | Nominal Amount |
|---|---|---|---|
| | Concentration | Concentration | [mg/syringe] |
| Components | [mmol/L] | [g/l] | V = 1.0 ml |
| Antibody | 0.6 | 90.0 | 90.0 |
| Succinic acid | 0.5 | 0.059 | 0.059 |
| Disodium | 3.9 | 1.054 | 1.054 |
| succinate | | | |
| hexahydrate | | | |
| Sorbitol | 225 | 41.00 | 41.00 |
| Polysorbat 20 | 0.16 | 0.20 | 0.20 |
| Water for Injection | — | Ad 1 L | Ad 1 mL |

The pH of formulation 2 is typically in the range of pH 5.5 to 6.5, for example 5.5 to 6.1, for example the pH is 5.8. This formulation is particularly suitable for subcutaneous administration.

Molecular weight (MW in g/mol) of used excipients:
MVW: Succinic acid (C₄H₆O₄)=118.09 g/mol
MVW: Disodium succinate hexahydrate (C₄O₄Na₂H₄×6H₂O)=270.14 g/mol
MW: Sorbitol=182.17 g/mol
MW: Polysorbate 20=1227.72 g/mol

The osmolarity of the formulation is 300+/−30 mOsmol/kg, as determined using an Osmomat 030 (Gonotec GmbH, Berlin, Germany). The density at 20° C. of the formulation is approximately 1.040 g/cm³, as determined using a measuring unit DMA 4500 (Anton Paar GmbH, Ostfildern-Scharnhausen, Germany).

### Formulation 3:

| | | | Nominal Amount |
|---|---|---|---|
| | Concentration | Concentration | [mg/syringe] |
| Components | [mmol/L] | [g/l] | V = 1.0 ml |
| Antibody | 0.6 | 90.0 | 90.0 |
| Sorbitol | 240 | 43.733 | 43.733 |
| Polysorbat 20 | 0.16 | 0.20 | 0.20 |
| Water for Injection | — | Ad 1 L | Ad 1 mL |

The pH of formulation 3 is typically in the range of pH 5.5 to 6.5, for example 5.5 to 6.1, for example the pH is 5.8. This formulation is particularly suitable for subcutaneous administration.

Molecular weight (MW in g/mol) of used excipients:
MW: Sorbitol=182.17 g/mol
MW: Polysorbate 20=1227.72 g/mol.

The osmolarity of the formulation is 300+/−30 mOsmol/kg, as determined using an Osmomat 030 (Gonotec GmbH, Berlin, Germany).

### Example 5: Molecular and Histopathological Profile

The molecular and histopathological profile in skin lesions from psoriasis patients treated with Antibody A vs ustekinumab were compared.

Materials & Methods: Lesional skin biopsies were obtained in two clinical trials of moderate-to-severe psoriasis patients, a Ph I study of single doses of Antibody A (0.01 mg/kg to 5 mg/kg) vs placebo and the Ph II study described above in Examples 1a to 1c comparing treatment with Antibody A (18 mg single injection; 90 mg or 180 mg at Wks 0, 4 and 16) vs ustekinumab (45 mg or 90 mg at Wks 0, 4 and 16). Samples were collected from 39 patients in the Ph I study (at baseline and Wk 8) and 60 patients in the Ph II study (at baseline and Wk 4) for histopathology and transcriptome-wide RNA profiling. Univariate associations were assessed using linear regression. Effect size, p-values and FDR were calculated for significant transcripts.

Results: As early as Wk 4, Antibody A treatment decreased expression of histopathology biomarkers including K16, Ki67, CD3, neutrophil gelatinase lipocalin, CD11c, DC-LAMP, b-defensin 2 and S100A7. In the Ph II study, 50% ( 7/14) and 64% ( 9/14) of patients on 90 or 180 mg Antibody A, respectively, were graded as excellent improvement (based on a global histopathology score) compared with 27% ( 4/15) for ustekinumab. Expression of a selected set of genes was significantly decreased in lesions at Wk 4 in those patient samples that were graded as excellent improvement vs other categories. The pathways altered included type I IFN, S-100, keratinocyte and late cornified envelope family. Treatment with Antibody A resulted in significant decreases (p<0.005) from baseline to Wk 4 in the expression of 1714 unique genes, including IL-23R, CCL20, IL-360, β-defensin 4B, and type I IFN pathway and late cornified envelope family genes, in patients who received Antibody A but not ustekinumab. Expression of a set of 1709 genes was significantly decreased by both treatments. At 4 and 8 wks, Antibody A strongly downregulated expression of genes associated with keratinocytes and epidermal cells and, to a lesser extent, those associated with monocytes, macrophages, dendritic cells and neutrophils. Table 7 shows selected genes decreased by Antibody A (Bonferroni correction was applied for multiple comparisons).

Conclusions: The superior clinical efficacy of Antibody A was associated with early changes in the molecular and histopathological profile of skin lesions in psoriasis patients compared with ustekinumab.

Skin biopsies were collected from 58 patients at baseline and at Week 4. Biomarkers associated with psoriatic disease and the IL-23 pathway were assessed in skin biopsies, pre- and post-treatment, by immunohistochemistry. Biopsy results were classified as excellent, good, slight, no change, or worsening (see Table 6A for classification criteria). A summary of global histopathologic scores by treatment group in a subset of patients who had lesional biopsies collected at baseline and at Week 4 is shown in Table 6B. The scores are based on the classifications in Table 6A. Only 52 biopsies were assessed, because some of the biopsies were either not assessable or missing at baseline or Week 4 and thus excluded from the analysis.

Additionally, transcriptome-wide RNA sequencing was performed on biopsy samples.

Global transcriptome-wide sequencing of RNA from lesion skin biopsy samples from a subset of 58 patients was achieved using the Illumina Hi-Seq 2000 (Illumina Inc., San Diego, CA). A generalized linear model of the RNA seq data for each gene was fitted using empirical Bayes estimates of the dispersion (variance). The model included the change in log gene expression as the dependent variable and treatment group as the independent variable. This model yielded estimates of the adjusted log fold-change associated with treatment and an associated p-value for each RNA transcript. P-values of <0.005 were considered significant. The experiment-wise false discovery rate was computed according to standard methods using Bonferroni correction applied for multiple comparisons.

TABLE 6A
Immunohistochemistry Grading Classification Criteria

| Grading | Classification Criteria |
|---|---|
| Excellent | Disease reversal or complete improvement by histological |
| improvement | criteria. Epidermal acanthosis is reversed, terminal |
| | differentiation abnormalities (lack of granular layer, |
| | parakeratosis) are reversed, keratin 16 is no longer |
| | expressed by supra basal keratinocytes (thus regenerative |
| | epidermal hyperplasia is eliminated), and tissue patterning |
| | no longer has psoriasis-like features. Histologically, the |
| | treated lesion looks like non-lesional skin. If examined by |
| | histopathology, psoriasis would not be diagnosed as an |
| | underlying disease. |
| Good | Strong attenuation of psoriasis features, but there is focal |
| improvement | keratin 16 expression, indicating that some regions of the |
| | epidermis maintain the regenerative epidermal hyperplasia |
| | phenotype. Epidermal acanthosis will be reduced, epidermal |
| | differentiation improved, and the overall biopsy will have |
| | weak psoriasis-like features. If examined by histopathology, |
| | a clear diagnosis of psoriasis would not be given. The |
| | biopsy will probably not look like non-lesional skin, but |
| | will retain focal features of the psoriatic process. |
| Slight | Partial attenuation of psoriasis features. Epidermis will |
| improvement | be thinner than the pre-treatment biopsy and some |
| | improvement in epidermal differentiation will be evident, |
| | but the biopsy will retain uniform keratin 16 staining in |
| | supra basal keratinocytes, so the epidermis is displaying |
| | regenerative epidermal hyperplasia. |
| No change | No histological change from pre-treatment biopsy. |
| Worsening | Increased acanthosis of the epidermis (more epidermal |
| | hyperplasia than baseline). |

TABLE 6B
Summary of global histopathologic scores

| | Antibody A | | | | | | Ustekinumab | |
|---|---|---|---|---|---|---|---|---|
| | 18 mg | | 90 mg | | 180 mg | | 45/90 mg | |
| N (%) | (N = 12) | | (N = 13) | | (N = 13) | | (N = 14) | |
| Excellent | 3 | (25) | 7 | (54) | 9 | (69) | 4 | (29) |
| improvement | | | | | | | | |
| Good | 4 | (33) | 2 | (15) | 3 | (23) | 2 | (14) |
| improvement | | | | | | | | |
| Slight | 4 | (33) | 1 | (8) | 1 | (8) | 6 | (43) |
| improvement | | | | | | | | |

| | | | | | | | |
|---|---|---|---|---|---|---|---|
| No change | 1 | (8) | 3 | (23) | 0 | 1 | (7) |

| | | | | | |
|---|---|---|---|---|---|
| Worsening | 0 | 0 | 0 | 1 | (7) |

Table 6C shows the numbers of patients in PASI response over time with a mean decrease in each of 3423 significant genes at Week 4, the expression of which was significantly decreased after treatment with Antibody A (1714 genes) and after treatment with Antibody A and treatment with ustekinumab (1709 genes).

Further analysis showed that the expression of 440 genes was significantly decreased by Antibody A at week 4 with a >2 fold change (baseline to week 12). Only 89 of these genes showed decreased expression with treatment with Antibody A, while reductions in the expression of 351 overlapping genes between treatment Antibody A and treatment with ustekinumab was observed. The 89 genes are listed in Table 6D.

TABLE 6C
Numbers of patients in PASI response over time with a mean
decrease in each of 3423 significant genes at Week 4

| PASI response | Week 16 | Week 24 | Week 48 |
|---|---|---|---|
| PASI 75 | 39/41 (95.1%) | 38/41 (92.7%) | 29/41 (70.7%) |
| PASI 90 | 35/41 (85.4%) | 31/41 (75.6%) | 20/41 (48.8%) |
| PASI 100 | 22/41 (53.7%) | 22/41 (53.7%) | 11/41 (26.8%) |

The key pathways modulated by Antibody A versus ustekinumab at week 4 are as follows (p-value<0.005 & fold change >1.5 for the genes included in these pathways):

Stronger enrichment/modulation for Antibody A:
Hypoxia, myogenesis, UV response DN, angiogenesis, coagulation, xenobiotic metabolism, estrogen response late, G2M checkpoint, estrogen response early, epithelial mesenchymal transition.

Stronger enrichment/modulation for ustekinumab:
IL2 STAT5 signaling, Interferon_Alpha_response, Interferon Gamma response, IL6 JAIK STAT3 signaling, inflammatory response, TNFA signaling via NFKB.

TABLE 6D

| | |
|---|---|
| NPC1L1 | NPC1-like 1 [Source: HGNC Symbol; Acc: 7898] |
| ARSF | arylsulfatase F [Source: HGNC Symbol; Acc: 721] |
| CHI3L2 | chitinase 3-like 2 [Source: HGNC Symbol; Acc: 1933] |
| PITX1 | paired-like homeodomain 1 [Source: HGNC Symbol; Acc: 9004] |
| CNGB1 | cyclic nucleotide gated channel beta 1 [Source: HGNC Symbol; |
| | Acc: 2151] |
| SLC12A3 | solute carrier family 12 (sodium/chloride transporters), member 3 |
| | [Source: HGNC Symbol; Acc: 10912] |
| ATP12A | ATPase, H+/K+ transporting, nongastric, alpha polypeptide |
| | [Source: HGNC Symbol; Acc: 13816] |
| ARG2 | arginase, type II [Source: HGNC Symbol; Acc: 664] |
| VNN3 | vanin 3 [Source: HGNC Symbol; Acc: 16431] |
| SERPIND1 | serpin peptidase inhibitor, clade D (heparin cofactor), member 1 |
| | [Source: HGNC Symbol; Acc: 4838] |
| OLFM4 | olfactomedin 4 [Source: HGNC Symbol; Acc: 17190] |
| DKK4 | dickkopf homolog 4 (Xenopus laevis) [Source: HGNC Symbol; |
| | Acc: 2894] |
| SIGLEC5 | sialic acid binding Ig-like lectin 5 [Source: HGNC Symbol; |
| | Acc: 10874] |
| HYAL4 | hyaluronoglucosaminidase 4 [Source: HGNC Symbol; Acc: 5323] |
| GNAT1 | guanine nucleotide binding protein (G protein), alpha transducing |
| | activity polypeptide 1 [Source: HGNC Symbol; Acc: 4393] |
| CCL20 | chemokine (C-C motif) ligand 20 [Source: HGNC Symbol; Acc: 10619] |
| C11orf9 | chromosome 11 open reading frame 9 [Source: HGNC Symbol; |
| | Acc: 1181] |
| SRMS | src-related kinase lacking C-terminal regulatory tyrosine and |
| | N-terminal |
| | myristylation sites [Source: HGNC Symbol; Acc: 11298] |
| TGM3 | transglutaminase 3 (E polypeptide, protein-glutamine-gamma- |
| | glutamyltransferase) [Source: HGNC Symbol; Acc: 11779] |
| TEX101 | testis expressed 101 [Source: HGNC Symbol; Acc: 30722] |
| ACRV1 | acrosomal vesicle protein 1 [Source: HGNC Symbol; Acc: 127] |
| EHF | ets homologous factor [Source: HGNC Symbol; Acc: 3246] |
| NKX2-8 | NK2 homeobox 8 [Source: HGNC Symbol; Acc: 16364] |
| REN | renin [Source: HGNC Symbol; Acc: 9958] |
| ANKRD31 | ankyrin repeat domain 31 [Source: HGNC Symbol; Acc: 26853] |
| GBAP1 | glucosidase, beta, acid pseudogene 1 [Source: HGNC Symbol; |
| | Acc: 4178] |
| PGLYRP2 | peptidoglycan recognition protein 2 [Source: HGNC Symbol; |
| | Acc: 30013] |
| IL23R | interleukin 23 receptor [Source: HGNC Symbol; Acc: 19100] |
| LCE3D | late cornified envelope 3D [Source: HGNC Symbol; Acc: 16615] |
| PPBP | pro-platelet basic protein (chemokine (C-X-C motif) ligand 7) |
| | [Source: HGNC Symbol; Acc: 9240] |
| BMPER | BMP binding endothelial regulator [Source: HGNC Symbol; |
| | Acc: 24154] |
| CLEC3A | C-type lectin domain family 3, member A [Source: HGNC Symbol; |
| | Acc: 2052] |
| A2ML1 | alpha-2-macroglobulin-like 1 [Source: HGNC Symbol; Acc: 23336] |
| TGM6 | transglutaminase 6 [Source: HGNC Symbol; Acc: 16255] |
| MTNR1A | melatonin receptor 1A [Source: HGNC Symbol; Acc: 7463] |
| MZB1 | marginal zone B and B1 cell-specific protein [Source: HGNC |
| | Symbol; Acc: 30125] |
| CEACAM3 | carcinoembryonic antigen-related cell adhesion molecule 3 |
| | [Source: HGNC Symbol; Acc: 1815] |
| REG3A | regenerating islet-derived 3 alpha [Source: HGNC Symbol; |
| | Acc: 8601] |
| ABCG4 | ATP-binding cassette, sub-family G (WHITE), member 4 [Source: |
| | HGNC Symbol; Acc: 13884] |
| SLC22A13 | solute carrier family 22 (organic anion transporter), member 13 |
| | [Source: HGNC Symbol; Acc: 8494] |
| MYO1H | myosin IH [Source: HGNC Symbol; Acc: 13879] |
| GCNT4 | glucosaminyl (N-acetyl) transferase 4, core 2 [Source: HGNC |
| | Symbol; Acc: 17973] |
| HMGN2P46 | high mobility group nucleosomal binding domain 2 pseudogene 46 |
| | [Source: HGNC Symbol; Acc: 26817] |
| ALOX12B | arachidonate 12-lipoxygenase, 12R type [Source: HGNC Symbol; |
| | Acc: 430] |
| MRGPRX3 | MAS-related GPR, member X3 [Source: HGNC Symbol; Acc: 17980] |
| FAM181B | family with sequence similarity 181, member B [Source: HGNC |
| | Symbol; Acc: 28512] |
| MAFA | v-maf musculoaponeurotic fibrosarcoma oncogene homolog A |
| | (avian) [Source: HGNC Symbol; Acc: 23145] |
| TREX2 | three prime repair exonuclease 2 [Source: HGNC Symbol; |
| | Acc: 12270] |
| ADAP2 | ArfGAP with dual PH domains 2 [Source: HGNC Symbol; |
| | Acc: 16487] |
| KRT76 | keratin 76 [Source: HGNC Symbol; Acc: 24430] |
| LCE3E | late cornified envelope 3E [Source: HGNC Symbol; Acc: 29463] |
| CYP4F2 | cytochrome P450, family 4, subfamily F, polypeptide 2 [Source: |
| | HGNC Symbol; Acc: 2645] |
| KLK12 | kallikrein-related peptidase 12 [Source: HGNC Symbol; Acc: 6360] |
| TMPRSS11A | transmembrane protease, serine 11A [Source: HGNC Symbol; |
| | Acc: 27954] |
| CCK | cholecystokinin [Source: HGNC Symbol; Acc: 1569] |
| ERC2 | ELKS/RAB6-interacting/CAST family member 2 [Source: HGNC |
| | Symbol; Acc: 31922] |
| RNF222 | ring finger protein 222 [Source: HGNC Symbol; Acc: 34517] |
| ZFP92 | zinc finger protein 92 homolog (mouse) [Source: HGNC Symbol; |
| | Acc: 12865] |
| HIST1H3C | histone cluster 1, H3c [Source: HGNC Symbol; Acc: 4768] |
| HIST1H2BH | histone cluster 1, H2bh [Source: HGNC Symbol; Acc: 4755] |
| FCHSD1 | FCH and double SH3 domains 1 [Source: HGNC Symbol; |
| | Acc: 25463] |
| MUC2 | mucin 2, oligomeric mucus/gel-forming [Source: HGNC Symbol; |
| | Acc: 7512] |
| TRIM40 | tripartite motif containing 40 [Source: HGNC Symbol; Acc: 18736] |
| PTCHD2 | patched domain containing 2 [Source: HGNC Symbol; Acc: 29251] |
| CTXN3 | cortexin 3 [Source: HGNC Symbol; Acc: 31110] |
| MFSD2B | major facilitator superfamily domain containing 2B [Source: HGNC |
| | Symbol; Acc: 37207] |
| C22orf42 | chromosome 22 open reading frame 42 [Source: HGNC Symbol; |
| | Acc: 27160] |
| TRAV8-4 | T cell receptor alpha variable 8-4 [Source: HGNC Symbol; |
| | Acc: 12149] |
| IGHE | immunoglobulin heavy constant epsilon [Source: HGNC Symbol; |
| | Acc: 5522] |
| IGHV1-3 | immunoglobulin heavy variable 1-3 [Source: HGNC Symbol; |
| | Acc: 5552] |
| KLK9 | kallikrein-related peptidase 9 [Source: HGNC Symbol; Acc: 6370] |
| CPLX3 | complexin 3 [Source: HGNC Symbol; Acc: 27652] |
| C3orf43 | chromosome 3 open reading frame 43 [Source: HGNC Symbol; |
| | Acc: 27407] |
| AZGP1P2 | alpha-2-glycoprotein 1, zinc-binding pseudogene 2 [Source: HGNC |
| | Symbol; Acc: 912] |
| PLIN5 | perilipin 5 [Source: HGNC Symbol; Acc: 33196] |
| SMARCE1P6 | SWI/SNF related, matrix associated, actin dependent regulator of |
| | chromatin, subfamily e, member 1 pseudogene 6 [Source: HGNC |
| | Symbol; Acc: 39737] |
| ZNF812 | zinc finger protein 812 [Source: HGNC Symbol; Acc: 33242] |
| RPL26P34 | ribosomal protein L26 pseudogene 34 [Source: HGNC Symbol; |
| | Acc: 36249] |
| CYP4A22-AS1 | CYP4A22 antisense RNA 1 [Source: HGNC Symbol; Acc: 43715] |
| HK2P1 | hexokinase 2 pseudogene 1 [Source: HGNC Symbol; Acc: 4924] |
| LINC00659 | long intergenic non-protein coding RNA 659 [Source: HGNC |
| | Symbol; Acc: 44316] |
| SERPINH1P1 | serpin peptidase inhibitor, clade H1, pseudogene 1 [Source: HGNC |
| | Symbol; Acc: 19917] |
| CSF2RBP1 | colony stimulating factor 2 receptor, beta, low-affinity (granulocyte- |
| | macrophage) pseudogene 1 [Source: HGNC Symbol; Acc: 2437] |
| OR5H8P | olfactory receptor, family 5, subfamily H, member 8 pseudogene |
| | [Source: HGNC Symbol; Acc: 14773] |
| C8orf56 | chromosome 8 open reading frame 56 [Source: HGNC Symbol; |
| | Acc: 28595] |
| FABP5P11 | fatty acid binding protein 5 pseudogene 11 [Source: HGNC Symbol; |
| | Acc: 19328] |
| UGT1A8 | UDP glucuronosyltransferase 1 family, polypeptide A8 [Source: |
| | HGNC Symbol; Acc: 12540] |
| AOX2P | aldehyde oxidase 2 pseudogene [Source: HGNC Symbol; |
| | Acc: 18450] |
| LCE3C | late cornified envelope 3C [Source: HGNC Symbol; Acc: 16612] |

TABLE 7
Select Genes Decreased by Antibody A at 4
Weeks Post Treatment as Assessed by RNAseq

| | | Antibody A treated | |
|---|---|---|---|
| | | (pooled 90 and 180 mg) | |
| Differentially | | Mean Log₂ | |
| expressed gene | | fold change | Adjusted |
| by RNAseq | Protein name | (vs. baseline) | P-value |

IL-23/IL-17 pathway related

| | | | |
|---|---|---|---|
| IL-23R | Interleukin 23 receptor | −0.05 | 0.001 |

Epidermal hyperplasia

| | | | |
|---|---|---|---|
| IL-36B | Interleukin 36 β | −0.26 | 0.003 |
| DEFB4B | β-defensin 4B | −1.89 | 1.02 × 10⁻⁷ |

Epithelial cell differentiation

| | | | |
|---|---|---|---|
| | Late cornified | | |
| | envelope proteins | | |
| LCE3A | 3A | −2.7 | 1.85 × 10⁻¹⁰ |
| LCE3B | 3B | −0.96 | 1.65 × 10⁻⁵ |
| LCE3C | 3C | −1.84 | 3.85 × 10⁻⁶ |
| LCE3D | 3D | −1.53 | 7.08 × 10⁻¹⁰ |
| LCE3E | 3E | −1.68 | 1.04 × 10⁻¹⁰ |

Immune-related pathways

| | | | |
|---|---|---|---|
| IFI27 | IFN α-inducible | −0.87 | 1.53 × 10⁻⁸ |
| | protein 27 | | |
| IFI27L2 | IFN α-inducible | −0.12 | 0.007 |
| | protein 27-like | | |
| | protein 2 | | |
| IFI44L | IFN-induced protein | −1.01 | 6.63 × 10⁻⁶ |
| | 44-like | | |
| IFIT1 | IFN-induced protein | −0.90 | 2.38 × 10⁻⁶ |
| | with tetratricopeptide | | |
| | repeats 1 | | |
| IRF9 | IFN regulatory factor 9 | −0.22 | 8.0 × 10⁻⁵ |

### Example 6: Immunogenicity Assessments

Immunogenicity of Antibody A was assessed using a multi-tiered approach. All samples were first analyzed in the anti-drug antibody (ADA) screening assay. Samples found to be putative positive in the ADA screening assay were then assessed in the ADA confirmatory assay. Samples that were confirmed positive were then titrated and further characterized in the neutralizing anti-drug antibody (Nab) screening assay. Samples that were found to be putative positive in the Nab screening assay were then evaluated in the Nab specificity assay.

The ADA screening assay began with acid dissociation of the sample, followed by a validated drug-bridging electrochemiluminescence (ECL) assay. Samples and controls were treated with 300 mM acetic acid to dissociate the Antibody A ADA complex. The acid-treated samples were then incubated and neutralized with 1 M Tris pH 9.5 in a mixture of biotinylated-Antibody A and ruthenium-Antibody A. The neutralized samples were then added to the blocked Meso Scale Discovery® (MSD) streptavidin plate and incubated. After washing, the drug-ADA complex was detected by the addition of MSD read buffer to the plate and subsequent excitation of the ruthenium via an electrochemical reaction to generate Ru(bpy)₃²⁺ luminescence that was read by the MSD Sector Imager 6000. The ECL signal intensity correlated with the level of anti-Antibody A antibodies present in the sample. A monoclonal antibody raised against Antibody A was used as the positive control (PC). The sensitivity assessed during validation was 0.177 ng/mL in 100% human K3EDTA plasma. The drug tolerance was such that 100 ng/mL of PC in 100% human K3EDTA plasma could be detected in the presence of 100 g/mL or less of Antibody A.

The validated Nab assay measured neutralizing antibodies to Antibody A in human plasma through assessment of STAT3 phosphorylation. Plasma samples were incubated with a fixed concentration of Antibody A and IL-23. The human large B cell lymphoma cell line, DB, used in the assay displayed increased phosphorylated STAT3 in the presence of IL-23. Phosphorylated-STAT3 was evaluated using the PathScan® Phospho-Stat3 (Tyr705) sandwich ELISA that was read using a SpectraMax Plus³⁸⁴ Absorbance Microplate Reader. In the absence of neutralizing antibodies, Antibody A bound to IL-23 resulted in decreased IL-23R activation and reduced STAT3 phosphorylation. In the presence of neutralizing antibodies, increased IL-23-mediated IL-23R activation and consequent STAT3 phosphorylation occurred.

A NAb specificity assay was also used in order to confirm that a reduction in signal observed in the NAb screening assay was due to antibodies and not due to non-specific matrix components. A monoclonal antibody raised against Antibody A was used as the PC. The relative assay sensitivity assessed during validation was 125 ng/mL in 100% human K3EDTA plasma. The drug tolerance was such that 250 ng/mL of PC in 100% human K3EDTA plasma could be detected in the presence of 70 ng/mL or less of Antibody A.

### Material and Methods for Examples 7-10

### 1. Preparation of the Starting Material

The starting material of risankizumab, produced in CHO cells and purified, was adjusted to pH 5.9, where necessary, prior to UF/DF process. At the end, the solution was concentrated and the concentrated starting material was used for preparing formulations according to the subsequent examples.

### 2. Syringes

Storage of formulations in syringes was essentially performed by using Neopak syringes with rubber stopper of Becton Dickinson (USA). The break loose force, as well as the maximum and average gliding force were measured using such syringes. In embodiments, 1 ml Neopak syringes with a 27 gauge 12 inch needle and a rubber stopper of Becton Dickinson (USA) were used.

### Example 7: Characterization of the Starting Material

### 1. Influence of the pH on the Antibody

RALS (right-angle light scattering) measurements were performed for risankizumab at different pH values in a buffer mixture of 10 mM acetate, 10 mM citrate, 10 mM phosphate, 115 mM NaCl. The results are shown in Table 8.

TABLE 8
Results of the RALS measurements for different pH values.

| pH value | | 4.5 | 5.0 | 5.5 | 6.0 | 6.5 | 7.0 |
|---|---|---|---|---|---|---|---|
| RALS | Tₒₙₛₑₜ/° C. | 70/71 | 72/72 | 75/75 | 76/76 | 76/75 | 76/76 |

The results show that the onset of unfolding increases when increasing the pH until a plateau is reached. The highest onset temperatures indicating high stability were measured above pH of 5.0 to 7. Therefore, the pH should not be too acidic (<5).

### 2. Determination of the Buffering Capacity of the Starting Material

The buffer capacity of the antibody starting material was determined inter alia in order to facilitate the pH adjustment of the formulation solutions of the subsequent stability studies and to avoid protein damage. The titration to determine the buffer capacity of the antibody was performed at the following concentrations: 150 mg/mL; 100 mg/mL; 50 mg/mL (twice) and 20 mg/mL.

The dilution was carried out in beakers according to Table 9.

TABLE 9
Dilution scheme for determination of the buffering capacity of the starting material.

| Target concentration | Volume of initial solution | Volume | Final volume |
|---|---|---|---|
| [mg/mL] | [mL] | MilliQ [mL] | [mL] |
| 150 | 4.67 | 1.33 | 5.00 |
| 100 | 3.11 | 2.89 | 5.00 |
| 50 | 1.56 | 4.44 | 5.00 |
| 50 | 2.6 | 7.40 | 10.00 |

After dilution, 5 mL of the respective solution was transferred into a 10R glass vial using a volumetric pipette and titrated. 0.2 M NaOH solution was selected for titration (stirring speed 250 rpm). For each antibody concentration the titration was performed and the amount of added NaOH was calculated. The slope and the reciprocal value of the slope as shown in Table 10 were calculated using Excel.

TABLE 10
Results of the calculation of the slope and the
corresponding reciprocal values.

| Protein concentration | | |
|---|---|---|
| (mg/mL) | Slope | Reciprocal of the slope |
| 20.47 | 1.090000 | 0.92 |
| 50.74 | 0.434821 | 2.30 |
| 50.77 | 0.432378 | 2.31 |
| 102.33 | 0.201703 | 4.96 |
| 152.30 | 0.133120 | 7.51 |

The concentration dependent buffer capacity levels were obtained by drawing the concentration against the reciprocal values of the slope. As a result, a straight line with a slope of 0.0505 and a y-axis intercept of −0.2007 was obtained. The results and the below examples show that risankizumab itself has significant buffering capacity allowing to prepare 150 mg/ml buffer-free formulations according to the present disclosure that do not comprise any further/additional buffer substance.

### Example 8: Analysis of Different pH Values and Buffer Substances

1. Comparison of Acetate and Succinate Buffer Systems with Different pH Values for the Assessment of Stability of 150 mg/mL Formulations

### 1.1. Preparation of Formulations

The formulations shown in Table 11 were prepared and analyzed:

TABLE 11
Composition of the analyzed formulations.

| | | Protein | | | | |
|---|---|---|---|---|---|---|
| | | concentration | | Excipient | Excipient | Excipient |
| Formulation | pH | mg/mL | buffer | 1 | 2 | 3 |
| F1 | 6.2 | 150 | 4.4 mM | 200 mM | — | 0.02% |
| | | | succinate | sorbitol | | PS20 |
| F2 | 5.9 | 150 | 4.4 mM | 200 mM | — | 0.02% |
| | | | succinate | sorbitol | | PS20 |
| F3 | 5.7 | 150 | 10 mM | 190 mM | — | 0.02% |
| | | | acetate | sorbitol | | PS20 |
| F4 | 5.7 | 150 | 10 mM | 115 mM | 50 mM | 0.02% |
| | | | acetate | sorbitol | L-arginine | PS20 |
| | | | | | HCl | |
| F5 | 5.7 | 150 | 10 mM | 150 mM | 50 mM | 0.02% |
| | | | acetate | sorbitol | L-proline | PS20 |

0.02% PS20 corresponds to 0.2 mg/mL PS20.

Samples were taken within a period of 18 months (0, 3, 6, 8, 12 and 18 months). Storage conditions were: SOC and 25° C./60% relative humidity. Each formulation was filled into Neopak syringes. The filling within a laminar flow was set to a volume of 1.1 mL. The syringes were closed with a stopper and analyzed visually for particles before storage. Afterwards, the syringes were stored hanging in syringe-trays at the respective temperature. In parallel, the buffer solutions were stored as controls. After preparing the formulations, each solution was sterile filtered and the prepared formulations were stored in syringes (Neopak syringes of Becton Dickinson (USA).

### 1.2. Analytics

For analysis of the samples, inter alia high pressure size exclusion chromatography (HP-SEC) and ultra-performance size exclusion chromatography (UP-SEC) were performed and the turbidity at 860 nm (also referred to as opalescence) was measured. The syringeability of the formulations stored in syringes (Neopak) was analyzed by measuring the mechanical forces that are required to release/inject the formulation. A pressure test was performed with a speed of 379.2 mm/min (5 sec). The viscosity of the formulations was measured at 20° C. using the HAAKE RheoStress 600 with a C35/1 rotor. Double measurements were performed.

Further details on the utilized analysis methods are described below.

### 1.3. Results

### 1.3.1. Measurement of the Monomer Content

For determining the monomer content the samples were analyzed by HP-SEC and UP-SEC.

### HP-SEC Analysis

The results obtained by HP-SEC analysis at a storage temperature of 25° C. and 5° C. are shown in Table 12:

TABLE 12
Results HP-SEC monomer in % analysis over 18 months
storage at different temperatures in syringes.

| | | | F1 in | F2 in | F3 in | F4 in | F5 in |
|---|---|---|---|---|---|---|---|
| Time | | Temperature | % | % | % | % | % |
| Initial | | 25° C. | 95.8 | 96.4 | 96.6 | 96.7 | 96.5 |
| 3 | months | | 94.5 | 95.1 | 95.4 | 96.1 | 95.6 |
| 6 | months | | 94.4 | 94.9 | 95.1 | 95.8 | 95.4 |
| 8 | months | | 93.7 | 94.3 | 94.6 | 95.3 | 94.9 |
| 12 | months | | 92.9 | 93.5 | 93.9 | 94.8 | 94.3 |
| 18 | months | | 91.1 | 92.4 | 92.8 | 94.0 | 93.4 |
| Initial | | 5° C. | 95.8 | 96.4 | 96.6 | 96.7 | 96.5 |
| 6 | months | | 95.6 | 96.2 | 96.4 | 96.6 | 96.4 |
| 8 | months | | 95.2 | 95.9 | 96.1 | 96.4 | 96.2 |
| 18 | months | | 93.4 | 94.7 | 95.1 | 95.8 | 95.4 |

Over a storage time of 18 months at 25° C. the monomer content was between 91-94%. The strongest decrease was measured for F1 with −5%; the lowest decrease was measured with −3% for formulation 4 and 5 tested in this example. Over a storage time of 18 months at 5° C. the monomer content was between 93-96%. The strongest decrease was measured for F1 with −2.4%; the lowest decrease was measured with −0.9% and −1.1% for formulation 4 and 5.

### UP-SEC Analysis

The results of the UP-SEC analysis were analogous to those of the HP-SEC and thus confirm these results.

TABLE 12a
Results UP-SEC monomer in % analysis over 18 months
storage at different temperatures in syringes.

| | | | F1 in | F2 in | F3 in | F4 in | F5 in |
|---|---|---|---|---|---|---|---|
| Time | | Temperature | % | % | % | % | % |
| Initial | | 25° C. | 94.9 | 95.4 | 95.5 | 95.7 | 95.4 |
| 3 | months | | 93.5 | 94.2 | 94.4 | 95.0 | 94.6 |
| 4 | months | | 93.6 | 92.7 | 93.0 | 93.6 | 93.3 |
| 5 | months | | 90.6 | 91.2 | 91.4 | 92.1 | 91.7 |
| 6 | months | | 88.4 | 89.4 | 89.8 | 90.6 | 90.3 |
| Initial | | 5° C. | 94.9 | 95.4 | 95.5 | 95.7 | 95.4 |
| 8 | months | | 94.6 | 95.3 | 95.5 | 95.7 | 95.7 |
| 18 | months | | 92.7 | 93.8 | 94.2 | 94.7 | 94.4 |

Over a storage time of 18 months at 25° C. the monomer content (UP-SEC) was between 88-91%. The strongest decrease was measured for F1 with −6.4%; the lowest decrease was measured with −5.1% and −5.4% for formulation 4 and 5. Over a storage time of 18 months at 5° C. the monomer content (UP-SEC) was between 93-95%. The strongest decrease was measured for F1 with −2.2%; the lowest decrease was measured with −1% for formulation 4 and 5.

### Results and Discussion

All tested formulations were overall stable in view of the monomer content, showing that these formulations are stable at 5° C. and 25° C. over long storage times up to 18 months.

### 1.3.2. Measurement of the HMW Content

For determining the HMW content the samples were analyzed by HP-SEC and UP-SEC.

### HP-SEC Analysis

The results obtained by HP-SEC analysis at a storage temperature of 25° C. and 5° C. are shown in Table 13.

TABLE 13
Results HP-SEC HMW content in % at different storage
temperatures over 18 months in syringes.

| | | | F1 in | F2 in | F3 in | F4 in | F5 in |
|---|---|---|---|---|---|---|---|
| Time | | Temperature | % | % | % | % | % |
| Initial | | 25° C. | 4.0 | 3.4 | 3.2 | 3.1 | 3.3 |
| 3 | months | | 5.1 | 4.5 | 4.3 | 3.6 | 4.0 |
| 6 | months | | 5.1 | 4.6 | 4.4 | 3.6 | 4.1 |
| 8 | months | | 5.7 | 5.1 | 4.8 | 4.0 | 4.5 |
| 12 | months | | 6.2 | 5.5 | 5.2 | 4.2 | 4.8 |
| 18 | months | | 7.6 | 6.4 | 5.9 | 4.7 | 5.3 |
| Initial | | 5° C. | 4.0 | 3.4 | 3.2 | 3.1 | 3.3 |
| 6 | months | | 4.2 | 3.6 | 3.4 | 3.2 | 3.4 |
| 8 | months | | 4.6 | 3.9 | 3.7 | 3.4 | 3.7 |
| 18 | months | | 6.2 | 5.0 | 4.5 | 3.9 | 4.3 |

Over a storage time of 18 months at 25° C. the HMW species were increased by 2-4%. The strongest increase was measured for F1 with +3.6%; the lowest increase was measured with +1.6% and +2.0% for formulation 4 and 5. Over a storage time of 18 months at 5° C. the HMW species were increased by 1-2%. The strongest increase was measured for F1 with +2.2%; the lowest increase was measured with +0.8% and +1.0% for formulation 4 and 5.

### UP-SEC Analysis

The results of the UP-SEC analysis were analogous to those of the HP-SEC and thus confirm these results.

TABLE 13a
Results UP-SEC HMW content in % at different storage
temperatures over 18 months in syringes.

| | | | F1 in | F2 in | F3 in | F4 in | F5 in |
|---|---|---|---|---|---|---|---|
| Time | | Temperature | % | % | % | % | % |
| Initial | | 25° C. | 3.9 | 3.4 | 3.2 | 3.1 | 3.3 |
| 3 | months | | 5.1 | 4.5 | 4.2 | 3.6 | 3.9 |
| 8 | months | | 4.0 | 5.0 | 4.8 | 4.0 | 4.4 |
| 12 | months | | 6.0 | 5.4 | 5.0 | 4.2 | 4.7 |
| 18 | months | | 7.4 | 6.4 | 6.0 | 4.9 | 5.5 |
| Initial | | 5° C. | 3.9 | 3.4 | 3.2 | 3.1 | 3.3 |
| 8 | months | | 4.4 | 3.7 | 3.5 | 3.3 | 3.5 |
| 18 | months | | 5.8 | 4.7 | 4.3 | 3.8 | 4.1 |

Over a storage time of 18 months at 25° C. the HMW were increased by 2-3.5%. The strongest increase was measured for F1 with +3.5%; the lowest increase was measured with +1.8% and +2.2% for formulation 4 and 5. Over a storage time of 18 months at 5° C. the HMW were increased by 0.7-1.9%. The strongest increase was measured for F1 with +1.9%; the lowest increase was measured with +0.7% and +0.8% for formulation 4 and 5.

### Results and Discussion

All tested formulations were overall stable in view of the HVMW content, showing that various formulations according to the present disclosure are stable at 5° C. and 25° C. over long storage times up to 18 months.

### 1.3.3. Turbidity Measurements

TABLE 14
Results of the turbidity at 860 nm in FNU over 18 months
at different storage temperatures in syringes.

| | | | F1 in | F2 in | F3 in | F4 in | F5 in |
|---|---|---|---|---|---|---|---|
| Time | | Temperature | FNU | FNU | FNU | FNU | FNU |
| Initial | | 25° C. | 6 | 6 | 5 | 8 | 5 |
| 3 | months | | 6 | 6 | 6 | 8 | 5 |
| 6 | months | | 6 | 5 | 4 | 7 | 5 |
| 8 | months | | 7 | 7 | 6 | 9 | 7 |
| 12 | months | | 8 | 7 | — | 9 | 6 |
| 18 | months | | 9 | 7 | 7 | 10 | 7 |
| Initial | | 5° C. | 6 | 6 | 5 | 8 | 5 |
| 6 | months | | 6 | 5 | 4 | 8 | 4 |
| 8 | months | | 6 | 5 | 4 | 7 | 4 |
| 18 | months | | 7 | 6 | 5 | 9 | 5 |

The turbidity was measured at a wavelength of 860 nm, showing an increase in Formazine Nephelometric Units (FNU) of 1 to 3 during the storage time of 18 months at 25° C. Formulation 1 had the strongest increase in 3 FNU and formulation 2 the smallest increase in 1 FNU. Formulation 4 containing L-arginine had the highest turbidity from the beginning on.

The turbidity was measured at a wavelength of 860 nm, showing an increase of 0-1 FNU during the storage time of 18 months at 5° C.

### Results and Discussion

The turbidity measurement show that storage in a fridge over 18 months does not result in relative changes in turbidity. Formulation 4 containing L-arginine had the highest turbidity rendering arginine-free formulations more advantageous.

### 1.3.4. Conductivity Measurements

The conductivity of the formulations was measured.

TABLE 15
Results of the conductivity measurements in mS/cm at
a storage temperature at 25° C. for up to 18 months.

| Time | | Temperature | F1 | F2 | F3 | F4 | F5 |
|---|---|---|---|---|---|---|---|
| Initial | | 25° C. | 1.25 | 1.37 | 1.53 | 3.96 | 1.54 |
| 3 | months | | 1.08 | 1.27 | 1.40 | 3.71 | 1.41 |
| 6 | months | | 1.29 | 1.34 | 1.52 | 3.85 | 1.52 |
| 8 | months | | 1.14 | 1.22 | 1.35 | 3.66 | 1.42 |
| 12 | months | | 1.18 | 1.23 | 1.43 | 3.61 | 1.43 |
| 18 | months | | 1.15 | 1.26 | 1.39 | 4.05 | 1.38 |
| Initial | | 5° C. | 1.25 | 1.37 | 1.53 | 3.96 | 1.54 |
| 6 | months | | 1.25 | 1.35 | 1.48 | 3.92 | 1.54 |
| 8 | months | | 1.12 | 1.18 | 1.36 | 3.67 | 1.38 |
| 18 | months | | 1.07 | 1.14 | 1.29 | 3.99 | 1.36 |

### Results and Discussion

The conductivity remained constant for all 5 formulations over a storage time of 18 months and at a temperature of 5° C. and 25° C. While the conductivity of F1, F2, F3 and F5 has a value between 1 to 2 mS/cm, the L-arginine containing formulation F4 had a relatively high conductivity of just below 4 mS/cm.

### 1.4. Further Analytics and Results

In addition, further analytics were performed for the five formulations tested (storage times and temperature were as described above) with the following results.
The pH value remained essentially constant over the storage time of 18 months and at the different storage temperatures tested. Measured pH values were thus in a range from 5.7-6.3.
The osmolality remained essentially constant over the storage time of 18 months and at the different storage temperatures tested. The tested values ranged from 296-333 mOsm/kg.
The dynamic viscosity at 20° C. remained essentially constant over the storage time of 18 months and at the different storage temperatures tested. The dynamic viscosity was in a range of 10-14 mPas.
The protein concentration remained essentially constant over the storage time of 18 months and at the different storage temperatures tested. Small deviations of protein concentration are due to analytical variations, leading to ranges of 148-159 mg/mL.
The HP-SEC fragments contents remained essentially constant over the storage time of 18 months and at the different storage temperatures tested. The fragments content was in a range of 0.2-1.4%. The UP-SEC LMW contents remained essentially constant over the storage time of 18 months and at the different storage temperatures tested. In particular, a low increase of 3% was measured for 25° C. and 0.1-0.3% for 5° C. over 18 months. The LMW content was in a range of 1.0-4.5%.
The weak cation exchange (WCX) main peak, acidic peak group (APG) and basic peak group (BPG) content remained constant over the storage time of 18 months at 5° C. No differences between the formulations were observed regarding the main peak, APG and BPG.
The hydrophobic interaction chromatography (HIC) main peak content remained essentially constant over the storage time of 12 months at 25° C. or 8 months at 5° C. The post peak increased by about 2-3% over at the storage temperature and time. The pre-peak was slightly increased by 5-7% over 12 months at 25° C. and did not increase at 5° C. over 8 months. No differences between the formulations were observed regarding the main peak, post peak and pre-peak.
The specific binding activity remained essentially constant over the storage time of 18 months and at the different storage temperatures tested with only minimal reduction of 3% and 1-2% for 25° C. and 5° C., respectively, for 18 months. The specific binding activity was in a range of 96-101%.
No visible particles were observed.

### 1.5. Summary of Results

All 5 formulations were stable over 18 months storage time at 25° C. and 5° C. Formulation 4, however, comprises an additional auxiliary agent. Formulation 3 was stable and did not have an additional auxiliary agent in contrast to the arginine-containing formulation F4. At a pH of about 5.7 less aggregate formation was observed.

2. Comparison of Acetate and Succinate Buffer Systems with Different pH Values in Free/Thaw Experiments

It was analyzed whether freezing and thawing of a 150 mg/mL formulation has an influence on the product quality of risankizumab. Therefore, 3 formulations were filled in mini bags having a volume of 10 mL or 14 mL for the initial value, followed by freezing at −40° C. Furthermore, one bag was stored at 2-8° C. The storage time for both conditions (−40° C. and 2-8° C.) was 3 weeks,

The freezing was performed by a controlled freezing process. Afterwards, the bags were transferred into a −40° C. freezer and kept frozen for the indicated storage times.

### 2.1. Preparation of Formulations

Each freeze/thaw cycle comprised freezing at −40° C. in a lyophilizer and subsequent transfer into a −40° C. freezer and thawing in a lyophilizer at a maximal thawing rate of 20° C./min until room temperature after 3 weeks. The tested formulations are shown in Table 16.

TABLE 16
Composition of the formulations.

| | | Protein | | | |
|---|---|---|---|---|---|
| | | concen- | | | |
| Formula- | | tration | | | |
| tion | pH | mg/mL | buffer | Excipient 1 | Excipient 2 |
| F1 | 6.2 | 150 | 10 mM succinate | 185 mM | 0.02 w % PS20 |
| | | | | sorbitol | |
| F2 | 5.7 | 150 | 10 mM acetate | 185 mM | 0.02 w % PS20 |
| | | | 6.5 mM succinate | sorbitol | |
| F3 | 5.7 | 150 | 10 mM acetate | 160 mM | 0.02 w % PS20 |
| | | | 6.5 mM succinate | trehalose | |

The starting material was stored at 2-8° C. in a fridge until use. The bags (“Mini Flexboy Bags”) having a sample volume of 10 mL were frozen using a lyophilizer with a controlled freezing rate of 0.5° C./min until reaching a temperature of −40° C.

### 2.2. Analytics

The samples were directly before analysis thawed in a controlled manner using a lyophilizer. To measure the protein stability HP-SEC analysis was performed and the binding activity was measured. The turbidity was measured at 860 nm and at 400-600 nm. Further details on the utilized analysis methods are described below.

### 2.3. Results

### 2.3.1. Measurement of the Monomer and HMW Content

For determining the stability of the formulations, HP-SEC and UP-SEC analyses were performed, showing the monomer and HMVW contents. Following results were obtained:

TABLE 17
Results HP-SEC monomer content in % after 3 weeks storage (frozen
at −40° C. and at 2-8° C.) and initial values.

| | F1- | F1- | F2- | F2- | F3- | F3- |
|---|---|---|---|---|---|---|
| | Bag 1 | Bag 2 | Bag 1 | Bag 2 | Bag 1 | Bag 2 |
| | % | % | % | % | % | % |
| Initial | 98.7 | 98.6 | 98.9 | 98.9 | 98.9 | 98.9 |
| 3 weeks | 98.3 | 98.3 | 98.7 | 98.7 | 98.7 | 98.7 |
| 5° C. | | | | | | |
| 3 weeks | 98.6 | 98.6 | 98.9 | 98.9 | 98.9 | 98.9 |
| −40° C. | | | | | | |

The UP-SEC measurements confirm the results of the HP-SEC measurement.

TABLE 17a
Results UP-SEC monomer content in % after 3 weeks storage (frozen
at −40° C. and at 2-8° C.) and initial values.

| | F1- | F1- | F2- | F2- | F3- | F3- |
|---|---|---|---|---|---|---|
| | Bag 1 | Bag 2 | Bag 1 | Bag 2 | Bag 1 | Bag 2 |
| | % | % | % | % | % | % |
| Initial | 97.0 | 97.0 | 97.4 | 97.5 | 97.4 | 97.4 |
| 3 weeks | 97.0 | 97.0 | 97.4 | 97.4 | 97.3 | 97.3 |
| 5° C. | | | | | | |
| 3 weeks | 97.0 | 97.0 | 97.4 | 97.5 | 97.4 | 97.4 |
| −40° C. | | | | | | |

The remaining antibody content missing to 10000 was present as HMN/W species.

### Results and Discussion

Overall, the results of the HP-SEC analysis demonstrate stability for all formulations, showing that the formulations allow for freeze/thaw cycle. Some formulations showed better results regarding a higher monomer content and less HMW species.

### 2.3.2. Measurement of the Binding Activity

The binding activity against rhIL-23 was measured utilizing surface plasmon resonance (Biacore) measurements. The binding activity is overall the same in all formulations, demonstrating applicability of the formulations. In particular, the binding activity was measured to be in a range between 95 to 110% and the specific binding activity was about 100%. The 3 weeks of storage in a frozen state or at 2-8° C. did not change the binding activity.

### 2.3.3. Measurement of the Viscosity

Another important parameter of protein formulations is the viscosity, which preferably is not too high in order to allow the formulation to be injected (e.g. pass a needle without excessive use of force). Therefore, the dynamic viscosity was measured.

The dynamic viscosity is very similar for F1, F2 and F3 being in a range of 8.7 to 10 mPas.

### 2.4. Further Analytics and Results

In addition, further analytics were performed for the three formulations tested with the following results.
The sub-visible particle content (≥25 μm, ≥10 μm, ≥5 μm) remained essentially constant over 3 weeks at 5° C. or at −40° C. including a freeze/thaw cycle. The sum of counted particles was essentially the same for all three formulations.
The osmolality remained essentially constant over 3 weeks at 5° C. or at −40° C. including a freeze/thaw cycle. The tested values ranged from 299-321 mOsm/kg for 150 mg/mL formulations.
The turbidity at 860 nm and 400-600 nm remained essentially constant over 3 weeks at 5° C. or at −40° C. including a freeze/thaw cycle. The tested values ranged from 2-7 FNU at 860 nm and 4-13 FNU at 400-600 nm.
The pH value remained essentially constant over 3 weeks at 5° C. or at −40° C. including a freeze/thaw cycle. Measured pH values were thus in a range from 5.7-6.2.
The conductivity remained essentially constant over 3 weeks at 5° C. or at −40° C. including a freeze/thaw cycle. Measured conductivity were thus in a range from 1.3-2.5 mS.
The protein concentration remained essentially constant over 3 weeks at 5° C. or at −40° C. including a freeze/thaw cycle. Small deviations of protein concentration are due to analytical variations, leading to ranges of 149-157 mg/mL for initial protein contents of 150 mg/mL.
The hydrophobic interaction chromatography (HIC) main peak content remained essentially constant over 3 weeks at 5° C. or at −40° C. including a freeze/thaw cycle. The HIC main peak values ranged from 97.1-97.7%. The post peak and pre peak remained essentially constant over 3 weeks at 5° C. or at −40° C. including a freeze/thaw cycle.
The weak cation exchange (WCX) chromatography main peak content remained essentially constant over 3 weeks at 5° C. or at −40° C. including a freeze/thaw cycle. The WCX main peak values range from 72.5-73.8%. The acidic peak group (APG) and basic peak group (BPG) remained essentially constant over 3 weeks at 5° C. or at −40° C. including a freeze/thaw cycle. No differences between the formulations were observed regarding main peak, APG and BGP.
The capillary gel electrophoresis (CGE) analysis showed essentially constant values over 3 weeks at 5° C. or at −40° C. including a freeze/thaw cycle. The non-reduced main peak contents ranged from 96.7-97.6%.

### 2.5. Summary of the Results

The results of the present example demonstrate stability of risankizumab provided in different 150 mg/mL formulations over freeze/thaw cycle. The single freeze/thaw cycle with a storage time of 3 weeks at −40° C. or at 2-8° C. for 3 weeks had no influence on the product quality of risankizumab. Therefore, 150 mg/mL formulations are a suitable concentration. A pH of 5.7 appeared to have performed slightly better compared to other pH values.

3. Comparison of Acetate and Succinate Buffer Systems with Different pH Values

A particularly suitable pH is in the range of 5.2 to 6.2 such as 5.5 to 6.2 or about 5.7. A higher pH can lead to increased protein aggregation, measured by SEC. A lower pH can lead to chemical degradation. In prior studies, sorbitol was used to adjust the tonicity. In the present example, trehalose and mannitol were used instead of sorbitol to adjust the tonicity. Seven sorbitol-free formulations were chosen and tested at following three conditions (i) 5° C. for 18 months, r.h. not monitored; (ii) 25° C./60 m r.h. for 18 months and (iii) 40° C./75% r.h. for 6 months.

The compositions of formulation 1-7 are depicted below in Table 18.

TABLE 18
Composition of the analyzed formulations.

| Formulation | Composition |
|---|---|
| F1 | 150 mg/mL risankizumab, 10 mM acetate + 100 mM trehalose + 100 mM |
| | mannitol + 0.2 mg/mL PS20, pH 5.7 |
| F2 | 150 mg/mL risankizumab, 10 mM acetate + 200 mM trehalose + 0.2 mg/mL |
| | PS20, pH 5.7 |
| F3 | 150 mg/mL risankizumab, 10 mM acetate + 100 mM trehalose + 50 mM L- |
| | arginine HCI + 0.2 mg/mL PS20, pH 5.7 |
| F4 | 150 mg/mL risankizumab, 4.4 mM succinate + 200 mM trehalose + 0.2 |
| | mg/mL PS20, pH 6.0 |
| F5 | 150 mg/mL risankizumab, 4.4 mM succinate + 100 mM trehalose + 50 mM |
| | L-arginine HC1 + 0.2 mg/mL PS20, pH 6.0 |
| F6 | 150 mg/mL risankizumab, 4.4 mM succinate + 100 mM trehalose + 100 |
| | mM mannitol + 0.2 mg/mL PS20, pH 6.0 |
| F7 | 150 mg/mL risankizumab, 200 mM trehalose + 0.2 mg/mL PS20, pH 5.7 |
| | (buffer free) |

The formulations and formulation buffers were sterile-filtered (filter type 0.22 μm) and filled under laminar flow with a fill volume of 1.04 mL in syringes (Neopak). The formulations were prepared by mixing the starting material with a concentration solution comprising the auxiliary agents (excipients, buffer, etc.). The filled syringes were stored horizontally in rondotrays protected from light using cardboard boxes at 2-8° C. Following packing materials were used:
Neopak syringe (1 ml syringe with 27 gauge 12 inch needle)
Rubber stopper
Rondotray

### 3.1. Analytics

For analysis of the samples, HP-SEC and UP-SEC were performed and the turbidity (also referred to as opalescence) was measured. Following devices were used for analysis:
UPLC, UPSEC: UPLC 29/31 Waters ACQUITY, Waters, MA
HPLC, WCX/SEC: HPLC 82/83/107 Waters ALLIACE, Waters, MA
Particle count/size by MFI: Micro Flow Imagine, 5200 BOT A/B (Roboter), Protein Simple, GER
Osmometer: Osmomat 3000 Gonotec GmbH, GER
pH-meter: SevenGo, Mettler Toledo, GER
Turbidity photometer: 2100AN Turbidimeter, Hach-Lange GmbH, GER
Protein concentration by Solo VPE: Solo VPE, C. Technologies, Inc., NJ
Biacore: Biacore T200, GE Healthcare Life Science, UK
Tensile and Compression testing machine: Zwick 2.5TS/N 21159574 Zwick, Germany

Further details on the utilized analysis methods are described below.

### 3.2. Results

### 3.2.1. Measurement of the Monomer Content

UP-SEC and HP-SEC were used to determine the loss of monomer content. Monomer content is a key quality attribute of protein stability and quality during stress-induced storage. The following table shows the results of the UP-SEC measurement.

TABLE 19
Monomer content measured by UP-SEC [%] of seven formulations
stored at 5° C., 25° C. and 40° C.

| | Storage | | | | | | | |
|---|---|---|---|---|---|---|---|---|
| Storage | time, | | | | | | | |
| condition | months | F1 | F2 | F3 | F4 | F5 | F6 | F7 |
| 5° C. | 0 | 98.2 | 98.2 | 98.2 | 97.9 | 98.1 | 97.9 | 98.1 |
| | 4 | 97.8 | 97.8 | 98.0 | 97.5 | 97.8 | 97.5 | 97.8 |
| | 6 | 97.6 | 97.6 | 97.8 | 97.3 | 97.7 | 97.3 | 97.6 |
| | 9 | 97.5 | 97.5 | 97.7 | 97.0 | 97.5 | 97.0 | 97.4 |
| | 12 | 97.5 | 97.5 | 97.8 | 97.1 | 97.5 | 97.1 | 97.5 |
| | 18 | — | 97.3 | — | — | — | — | — |
| 25° C. | 0 | 98.2 | 98.2 | 98.2 | 97.9 | 98.1 | 97.9 | 98.1 |
| | 2 | 97.1 | 97.1 | 97.4 | 96.8 | 97.3 | 96.8 | 97.1 |
| | 4 | 96.5 | 96.5 | 96.9 | 96.3 | 96.8 | 96.2 | 96.5 |
| | 6 | 95.8 | 95.8 | 96.2 | 95.6 | 96.2 | 95.5 | 95.8 |
| | 9 | 95.1 | 95.1 | 95.5 | 94.9 | 95.5 | 94.9 | 95.2 |
| | 12 | 94.7 | 94.7 | 95.0 | 94.5 | 95.1 | 94.5 | 94.7 |
| | 18 | — | 93.3 | — | — | — | — | — |
| 40° C. | 0 | 98.2 | 98.2 | 98.2 | 97.9 | 98.1 | 97.9 | 98.1 |
| | 2 | 92.6 | 92.7 | 92.4 | 92.6 | 92.4 | 92.4 | 92.8 |
| | 4 | 88.4 | 88.5 | 87.7 | 88.6 | 88.2 | 88.5 | 88.8 |
| | 6 | 84.3 | 84.4 | 83.6 | 84.9 | 84.3 | 84.7 | 84.7 |

HP-SEC confirms the results of UP-SEC. Compared to UP-SEC, no additional information were generated by HP-SEC.

### Results and Discussion

All analyzed formulations were stable at the tested conditions.

### 3.2.2. Measurement of the HMW Level

UP-SEC and HP-SEC were used to determine levels of HMW formation. The following table shows the results of the UP-SEC measurement. The HVW content correlates with the monomer content. Loss of monomer leads to increase of HMW.

TABLE 20
HMW measured by UP-SEC [%] of seven formulations stored
at 5° C., 25° C. and 40° C.

| | Storage | | | | | | | |
|---|---|---|---|---|---|---|---|---|
| Storage | time, | | | | | | | |
| condition | months | F1 | F2 | F3 | F4 | F5 | F6 | F7 |
| 5° C. | 0 | 1.3 | 1.3 | 1.2 | 1.6 | 1.4 | 1.6 | 1.4 |
| | 4 | 1.5 | 1.6 | 1.4 | 1.9 | 1.5 | 1.9 | 1.6 |
| | 6 | 1.6 | 1.7 | 1.5 | 2.0 | 1.6 | 2.0 | 1.7 |
| | 9 | 1.8 | 1.8 | 1.6 | 2.3 | 1.8 | 2.3 | 1.9 |
| | 12 | 1.9 | 1.9 | 1.6 | 2.3 | 1.8 | 2.3 | 1.9 |
| | 18 | — | 2.0 | — | — | — | — | — |
| 25° C. | 0 | 0 | 1.3 | 1.3 | 1.2 | 1.6 | 1.4 | 1.6 |
| | 2 | 2.0 | 2.0 | 1.7 | 2.4 | 1.8 | 2.4 | 2.0 |
| | 4 | 2.2 | 2.2 | 1.8 | 2.5 | 1.9 | 2.5 | 2.2 |
| | 6 | 2.4 | 2.4 | 2.0 | 2.7 | 2.1 | 2.8 | 2.4 |
| | 9 | 2.7 | 2.7 | 2.2 | 3.0 | 2.3 | 3.0 | 2.7 |
| | 12 | 2.8 | 2.8 | 2.3 | 3.2 | 2.4 | 3.2 | 2.9 |
| | 18 | — | 3.2 | — | — | — | — | — |
| 40° C. | 0 | 0 | 1.3 | 1.3 | 1.2 | 1.6 | 1.4 | 1.6 |
| | 2 | 3.7 | 3.6 | 3.4 | 3.9 | 3.4 | 4.0 | 3.5 |
| | 4 | 5.2 | 5.1 | 5.1 | 5.3 | 4.8 | 5.4 | 4.9 |
| | 6 | 6.7 | 6.7 | 6.5 | 6.8 | 6.2 | 6.8 | 6.4 |

UP-SEC confirms the results of UP-SEC. Compared to UP-SEC, no additional information were generated by UP-SEC.

### Results and Discussion

Overall, all formulations were stable with low amounts of HMW species even after storage at 40° C.

### 3.2.3. Measurement of the LMW Level

UP-SEC was used to determine levels of LMW formation. The following table shows the results of the UP-SEC measurement.

TABLE 21
LMW content measured by UP-SEC [%] of seven formulations stored
at 5° C., 25° C. and 40° C.

| | Storage | | | | | | | |
|---|---|---|---|---|---|---|---|---|
| Storage | time, | | | | | | | |
| condition | months | F1 | F2 | F3 | F4 | F5 | F6 | F7 |
| 5° C. | 0 | 0.5 | 0.5 | 0.5 | 0.5 | 0.5 | 0.6 | 0.6 |
| | 4 | 0.6 | 0.6 | 0.7 | 0.6 | 0.7 | 0.7 | 0.7 |
| | 6 | 0.7 | 0.7 | 0.7 | 0.7 | 0.7 | 0.7 | 0.7 |
| | 9 | 0.7 | 0.7 | 0.7 | 0.7 | 0.7 | 0.7 | 0.7 |
| | 12 | 0.6 | 0.6 | 0.6 | 0.6 | 0.6 | 0.6 | 0.6 |
| | 18 | — | 0.7 | — | — | — | — | — |
| 25° C. | 0 | 0.5 | 0.5 | 0.5 | 0.5 | 0.5 | 0.6 | 0.6 |
| | 2 | 0.9 | 0.9 | 1.0 | 0.9 | 0.9 | 0.9 | 0.9 |
| | 4 | 1.3 | 1.3 | 1.4 | 1.3 | 1.3 | 1.3 | 1.3 |
| | 6 | 1.8 | 1.7 | 1.8 | 1.7 | 1.7 | 1.7 | 1.7 |
| | 9 | 2.2 | 2.2 | 2.4 | 2.1 | 2.2 | 2.1 | 2.2 |
| | 12 | 2.4 | 2.4 | 2.6 | 2.3 | 2.5 | 2.4 | 2.4 |
| | 18 | — | 3.6 | — | — | — | — | — |
| 40° C. | 0 | 0.5 | 0.5 | 0.5 | 0.5 | 0.5 | 0.6 | 0.6 |
| | 2 | 3.7 | 3.7 | 4.3 | 3.5 | 4.1 | 3.5 | 3.7 |
| | 4 | 6.4 | 6.4 | 7.2 | 6.1 | 7.0 | 6.1 | 6.4 |
| | 6 | 8.9 | 8.9 | 9.9 | 8.4 | 9.5 | 8.5 | 8.9 |

### Results and Discussion

Overall, all formulations were stable with low amounts of LMW species even after storage at 40° C.

### 3.2.4. Measurement of the LMW Level

For assessing the stability of the formulations, the LMW content was also measured by HP-SEC analysis. The results of this analysis are shown below.

TABLE 22
LMW content measured by HP-SEC [%] of seven formulations stored
at 5° C., 25° C. and 40° C.

| | Storage | | | | | | | |
|---|---|---|---|---|---|---|---|---|
| Storage | time, | | | | | | | |
| condition | months | F1 | F2 | F3 | F4 | F5 | F6 | F7 |
| 5° C. | 0 | 0.2 | 0.2 | 0.2 | 0.2 | 0.2 | 0.2 | 0.2 |
| | 4 | 0.3 | 0.3 | 0.3 | 0.3 | 0.3 | 0.3 | 0.3 |
| | 6 | 0.3 | 0.3 | 0.3 | 0.3 | 0.3 | 0.3 | 0.3 |
| | 9 | 0.3 | 0.3 | 0.3 | 0.3 | 0.3 | 0.3 | 0.3 |
| | 12 | 0.4 | 0.3 | 0.3 | 0.3 | 0.3 | 0.3 | 0.3 |
| | 18 | — | 0.3 | — | — | — | — | — |
| 25° C. | 0 | 0.2 | 0.2 | 0.2 | 0.2 | 0.2 | 0.2 | 0.2 |
| | 2 | 0.4 | 0.4 | 0.4 | 0.4 | 0.4 | 0.4 | 0.4 |
| | 4 | 0.5 | 0.5 | 0.6 | 0.5 | 0.5 | 0.5 | 0.6 |
| | 6 | 0.6 | 0.6 | 0.6 | 0.6 | 0.6 | 0.6 | 0.6 |
| | 9 | 0.8 | 0.8 | 0.8 | 0.8 | 0.8 | 0.8 | 0.8 |
| | 12 | 1.0 | 1.0 | 1.0 | 1.0 | 1.0 | 1.0 | 1.0 |
| | 18 | — | 1.2 | — | — | — | — | — |
| 40° C. | 0 | 0.2 | 0.2 | 0.2 | 0.2 | 0.2 | 0.2 | 0.2 |
| | 2 | 1.3 | 1.3 | 1.4 | 1.3 | 1.4 | 1.2 | 1.3 |
| | 4 | 2.2 | 2.2 | 2.4 | 2.1 | 2.3 | 2.1 | 2.2 |
| | 6 | 3.0 | 3.0 | 3.3 | 2.8 | 3.1 | 2.8 | 3.0 |

### Results and Discussion

The LMW content increases only slightly over time but all tested formulations generally resulted in low levels of fragmentation.

### 3.2.5. Turbidity Measurements

The results of opalescence are summarized below. No changes in opalescence were observed over the time of storage at the different conditions. The formulations containing L-arginine HCl (F3 and F5) showed the highest opalescence. However, there was no increasing opalescence in the course of the study for F3 and F5. In none of the tested formulations visible particles were observed.

TABLE 23
Opalescence [FNU] of seven formulations stored at
5° C., 25° C. and 40° C.

| | Storage | | | | | | | |
|---|---|---|---|---|---|---|---|---|
| Storage | time, | | | | | | | |
| condition | months | F1 | F2 | F3 | F4 | F5 | F6 | F7 |
| 5° C. | 0 | 1 | 5 | 11 | 7 | 15 | 6 | 4 |
| | 4 | 5 | 5 | 11 | 7 | 13 | 7 | 4 |
| | 6 | 6 | 6 | 12 | 7 | 14 | 8 | 4 |
| | 9 | 5 | 5 | 11 | 7 | 15 | 8 | 6 |
| | 12 | 6 | 6 | 12 | 7 | 12 | 8 | 3 |
| | 18 | — | 6 | — | — | — | — | — |
| 25° C. | 0 | 5 | 5 | 11 | 7 | 15 | 6 | 4 |
| | 2 | 6 | 5 | 12 | 7 | 13 | 7 | 4 |
| | 4 | 5 | 5 | 13 | 7 | 13 | 7 | 3 |
| | 6 | 7 | 6 | 12 | 8 | 14 | 8 | 4 |
| | 9 | 8 | 7 | 13 | 9 | 14 | 8 | 4 |
| | 12 | 6 | 6 | 12 | 8 | 14 | 8 | 4 |
| | 18 | — | 7 | — | — | — | — | — |

### Results and Discussion

L-arginine HCL containing formulations showed increased opalescence.

### 3.3. Further Analytics and Results

In addition, further analytics were performed for the seven formulations tested with the following results (storage times and temperature were as described above).
The protein concentration remained essentially constant over the storage time of 18 months and at the different storage temperatures tested.
The pH value remained essentially constant over the storage time of 18 months and at the different storage temperatures tested.
The osmolality remained essentially constant over the storage time of 18 months and at the different storage temperatures tested. The tested values ranged from 298-326 mOsm/kg.
No visible particles were observed.
The IEC/WCX measurements of formulations at 5° C. for the tested storage times showed constant levels of main peak (69.7-72.2%), APG (18-20%) and BPG (8-13%). At 25° C. and 40° C. the main peak decreases and APG level increases for all formulations in a similar range. At 25° C. and 40° C. BPG level of formulations with pH 6.0 is lightly lower (up to 2%) than formulations with pH 5.7.
The particle content measured by MFI remained essentially constant over the storage time of 18 months and at the different storage temperatures tested. In particular, for particles ≥10 μm and ≥25 μm no relevant increasing particle count could be observed for all formulations over time of storage at 5° C. and 25° C./60% r.h. For particles ≥2 μm, the particle count at 25° C. was increased. The increase was for all formulations in a similar range.
The specific binding activity remained essentially constant over the storage time of 18 months and at the different storage temperatures tested. The specific binding activity was in a range of 95-100%.
The break loose and gliding forces remained essentially constant over the storage time of 18 months and at 5° C. At 25° C. the maximum gliding force, average gliding force and break loose force increased over time of storage for all formulations. No differences between the formulations were observed. Maximum gliding forces ranged from 7.1-23.6 N, average gliding forces from 6.7-20.5 N and break loose force from 3.4-7.1 N.
The dynamic viscosity measured at 20° C. remained essentially constant over the storage time of 18 months and at the different storage temperatures tested. The dynamic viscosity was in a range of 8.3-10.7 mPas.

### 3.4. Summary of Results

The present example describes the storage stability of seven different 150 mg/mL risankizumab formulations. Trehalose and mannitol instead of sorbitol were used to adjust the tonicity. F2 was additionally analyzed after 18 months.

UP-SEC showed that a pH of 6.0 compared to pH 5.7 leads in the tested formulations to a slightly larger degradation of risankizumab in form of lower monomer content and higher HMW. L-arginine HCl containing formulations showed slightly lower degradation measured by UP-SEC but increased opalescence and slightly increased LMW contents. IEC showed no relevant differences between the formulations and were therefore not a decisive factor. The same applies to pH, protein concentration, osmolality, viscosity, break loose and gliding force, subvisible particles and visible particles. No differences between the formulations were seen by this data.

4. Comparison of Acetate and Succinate Buffer Systems with Different pH Values Over Long Term Storage

The present example analyzes the storage stability of seven different 150 mg/mL risankizumab formulations in Neopak syringes to analyze the storage stability of the formulations and identify advantageous formulations. In comparison to the seven formulations of the prior example, the tonicity was slightly modified. Three conditions were again tested (as identified above). The formulations analyzed are summarized in Table 24.

TABLE 24
Composition of the analyzed formulations.

| Formulation | Composition |
|---|---|
| F1 | 150 mg/mL risankizumab, 10 mM acetate + 95 mM trehalose + 95 mM |
| | mannitol + 0.2 mg/mL PS20, pH 5.7 |
| F2 | 150 mg/mL risankizumab, 10 mM acetate + 185 mM trehalose + 0.2 mg/mL |
| | PS20, pH 5.7 |
| F3 | 150 mg/mL risankizumab, 10 mM acetate + 110 mM trehalose + 50 mM L- |
| | arginine HCl + 0.2 mg/mL PS20, pH 5.7 |
| F4 | 150 mg/mL risankizumab, 4.4 mM succinate + 180 mM trehalose + 0.2 |
| | mg/mL PS20, pH 6.0 |
| F5 | 150 mg/mL risankizumab, 4.4 mM succinate + 110 mM trehalose + 50 mM |
| | L-arginine HCl + 0.2 mg/mL PS20, pH 6.0 |
| F6 | 150 mg/mL risankizumab, 4.4 mM succinate + 95 mM trehalose + 95 mM |
| | mannitol + 0.2 mg/mL PS20, pH 6.0 |
| F7 | 150 mg/mL risankizumab, 200 mM trehalose + 0.2 mg/mL PS20, pH 5.7 |
| | (buffer free) |

The formulations were prepared by mixing the starting material with a concentrated spike solution (comprising the auxiliary agents, i.e. the excipients and buffers).

### 4.1. Analytics

For analysis of the samples, inter alia UP-SEC was performed and the opalescence was measured. Further details on the utilized analysis methods are described below.

### 4.2. Results

### 4.2.1. Measurement of the HMV Content

TABLE 25
Measured HMW content in % of the UP-SEC analysis for long-term
storage at 5° C., 25° C. and 40° C.

| | Storage | | | | | | | |
|---|---|---|---|---|---|---|---|---|
| Storage | time, | F1 in | F2 in | F3 in | F4 in | F5 in | F6 in | F7 in |
| condition | months | % | % | % | % | % | % | % |
| 5° C. | 0 | 1.3 | 1.3 | 1.2 | 1.6 | 1.4 | 1.6 | 1.4 |
| | 4 | 1.5 | 1.6 | 1.4 | 1.9 | 1.5 | 1.9 | 1.6 |
| | 6 | 1.6 | 1.7 | 1.5 | 2.0 | 1.6 | 2.0 | 1.7 |
| | 9 | 1.8 | 1.8 | 1.6 | 2.3 | 1.8 | 2.3 | 1.9 |
| | 12 | 1.9 | 1.9 | 1.6 | 2.3 | 1.8 | 2.3 | 1.9 |
| | 18 | — | 2.0 | — | — | — | — | — |
| 25° C. | 0 | 0 | 1.3 | 1.3 | 1.2 | 1.6 | 1.4 | 1.6 |
| | 2 | 2.0 | 2.0 | 1.7 | 2.4 | 1.8 | 2.4 | 2.0 |
| | 4 | 2.2 | 2.2 | 1.8 | 2.5 | 1.9 | 2.5 | 2.2 |
| | 6 | 2.4 | 2.4 | 2.0 | 2.7 | 2.1 | 2.8 | 2.4 |
| | 9 | 2.7 | 2.7 | 2.2 | 3.0 | 2.3 | 3.0 | 2.7 |
| | 12 | 2.8 | 2.8 | 2.3 | 3.2 | 2.4 | 3.2 | 2.9 |
| | 18 | — | 3.2 | — | — | — | — | — |
| 40° C. | 0 | 0 | 1.3 | 1.3 | 1.2 | 1.6 | 1.4 | 1.6 |
| | 2 | 3.7 | 3.6 | 3.4 | 3.9 | 3.4 | 4.0 | 3.5 |
| | 4 | 5.2 | 5.1 | 5.1 | 5.3 | 4.8 | 5.4 | 4.9 |
| | 6 | 6.7 | 6.7 | 6.5 | 6.8 | 6.2 | 6.8 | 6.4 |

### Results and Discussion

The HMV content remained overall low in all tested formulations indicating that the used formulations can stabilize risankizumab at high concentrations. The UP-SEC results further show that a pH of 6.0 compared to pH 5.7 leads to a larger degradation of risankizumab in form of lower monomer contents and higher HMV levels. Therefore, a pH of 5.7 is particularly advantageous for formulations according to the present disclosure. Nevertheless, also the formulations having a higher pH value of 6.0 such as F4, F5 and F6 showed overall good performance in view of the UP-SEC analysis results.

### 4.2.2. Measurement of the LMWV Content

TABLE 26
Measured LMW content in % of the UP-SEC analysis for long-term
storage at 5° C., 25° C. and 40° C.

| | | Storage | | | | | | | |
|---|---|---|---|---|---|---|---|---|---|
| Storage | | time, | F1 in | F2 in | F3 in | F4 in | F5 in | F6 in | F7 in |
| condition | | months | % | % | % | % | % | % | % |
| 5° | C. | 0 | 0.5 | 0.5 | 0.5 | 0.5 | 0.5 | 0.6 | 0.6 |
| | | 4 | 0.6 | 0.6 | 0.7 | 0.6 | 0.7 | 0.7 | 0.7 |
| | | 6 | 0.7 | 0.7 | 0.7 | 0.7 | 0.7 | 0.7 | 0.7 |
| | | 9 | 0.7 | 0.7 | 0.7 | 0.7 | 0.7 | 0.7 | 0.7 |
| | | 12 | 0.6 | 0.6 | 0.6 | 0.6 | 0.6 | 0.6 | 0.6 |
| | | 18 | — | 0.7 | — | — | — | — | — |
| 25° | C. | 0 | 0.5 | 0.5 | 0.5 | 0.5 | 0.5 | 0.6 | 0.6 |
| | | 2 | 0.9 | 0.9 | 1.0 | 0.9 | 0.9 | 0.9 | 0.9 |
| | | 4 | 1.3 | 1.3 | 1.4 | 1.3 | 1.3 | 1.3 | 1.3 |
| | | 6 | 1.8 | 1.7 | 1.8 | 1.7 | 1.7 | 1.7 | 1.7 |
| | | 9 | 2.2 | 2.2 | 2.4 | 2.1 | 2.2 | 2.1 | 2.2 |
| | | 12 | 2.4 | 2.4 | 2.6 | 2.3 | 2.5 | 2.4 | 2.4 |
| | | 18 | — | 3.6 | — | — | — | — | — |
| 40° | C. | 0 | 0.5 | 0.5 | 0.5 | 0.5 | 0.5 | 0.6 | 0.6 |
| | | 2 | 3.7 | 3.7 | 4.3 | 3.5 | 4.1 | 3.5 | 3.7 |
| | | 4 | 6.4 | 6.4 | 7.2 | 6.1 | 7.0 | 6.1 | 6.4 |
| | | 6 | 8.9 | 8.9 | 9.9 | 8.4 | 9.5 | 8.5 | 8.9 |

### Results and Discussion

The tested formulations were stable over the measurement time for all tested temperatures. Therefore, high protein concentrations of 150 mg/mL risankizumab were effectively stabilized using the tested formulations. At higher storage temperature, a slight increase of LMW content was observed for L-arginine HCl containing formulations. This is a surprising finding, as L-arginine containing formulations typically are known to further stabilize formulations. Hence, a formulation of 150 mg/mL risankizumab is different from other protein formulations in this respect. A formulation according to the present disclosure without arginine is therefore preferred.

### 4.2.3. Measurement of the Opalescence

TABLE 27
Measured opalescence in FNU for long-term storage at
5° C., 25° C. and 40° C.

| | | Storage | | | | | | | |
|---|---|---|---|---|---|---|---|---|---|
| Storage | | time, | F1 in | F2 in | F3 in | F4 in | F5 in | F6 in | F7 in |
| condition | | months | FNU | FNU | FNU | FNU | FNU | FNU | FNU |
| 5° | C. | 0 | 1 | 5 | 11 | 7 | 15 | 6 | 4 |
| | | 4 | 5 | 5 | 11 | 7 | 13 | 7 | 4 |
| | | 6 | 6 | 6 | 12 | 7 | 14 | 8 | 4 |
| | | 9 | 5 | 5 | 11 | 7 | 15 | 8 | 6 |
| | | 12 | 6 | 6 | 12 | 7 | 12 | 8 | 3 |
| | | 18 | — | 6 | — | — | — | — | — |
| 25° | C. | 0 | 5 | 5 | 11 | 7 | 15 | 6 | 4 |
| | | 2 | 6 | 5 | 12 | 7 | 13 | 7 | 4 |
| | | 4 | 5 | 5 | 13 | 7 | 13 | 7 | 3 |
| | | 6 | 7 | 6 | 12 | 8 | 14 | 8 | 4 |
| | | 9 | 8 | 7 | 13 | 9 | 14 | 8 | 4 |
| | | 12 | 6 | 6 | 12 | 8 | 14 | 8 | 4 |
| | | 18 | — | 7 | — | — | — | — | — |

### Results and Discussion

Overall, no or only a slight increase in opalescence was observed over time, indicating stability of all tested formulations. For formulations which contain L-arginine HCl (F3 and F5) a higher opalescence was observed.

### 4.3. Summary of Results

The measured parameters show that all formulations are suitable for preparing high risankizumab concentrations of 150 mg/mL in a stable manner. The long-term stability revealed some differences between the formulations:
UP-SEC showed that a pH of 6.0 compared to pH 5.7 leads to a larger degradation of risankizumab in form of lower monomer content and higher HMW.
L-arginine HCl containing formulations showed lower degradation measured by UP-SEC but increased opalescence and slight increase of LMW.

F2 was additionally analyzed after 18 months.

Noteworthy, also formulations F1 and F7 were stable. Buffer-free formulations and formulations comprising more than one type of tonicity agent were also stable and are thus suitable to provide a formulation comprising 150 mg/mL risankizumab.

In summary, formulation F2 was found to be particularly stable in respect to the measured LMW and HMW content, as well as opalescence, indicating a superior stability. This result was very surprising, as typically risankizumab is known to be used at higher pH values. Hence, the particularly high concentration of risankizumab shifts the optimal pH value to about 5.7, which was unexpected. Moreover, it was surprising that L-arginine HCl did not result in further stabilization but in fact reduced the stability of the formulations, supported by the higher opalescence and measured LMW content. Consequently, the particular properties of risankizumab at a high concentration, e.g. 150 mg/mL, necessitate different optimal conditions than formerly known formulations of risankizumab.

5. Comparison of Acetate and Succinate Buffer Systems with Different pH Values while Shaking

The goal of the present example was to evaluate the influence of shaking stress on the product quality of different formulations at 150 mg/mL risankizumab. Different formulations were tested regarding their ability to stabilize risankizumab against shaking stress. Therefore, the formulation was exposed to different shaking stresses at the antibody concentration of 150 mg/mL.

In total eleven formulations that differ in pH, buffering and tonicity agent were filled into 6R vials and 1 mL Neopak syringes with 27 gauge 12 inch needles and were shaken for 21 days at room temperature. Corresponding buffer solutions without protein were shaken, stored and analyzed as well. Shaking conditions:
Shaking temperature: room temperature (approx. 25° C.)
Shaking time: 21 days
Type of shaking: Horizontal shaker (vial), rocking shaker (syringe); the shaking was conducted protected from light

To exclude the influence of temperature as an additional stress on product quality further vials and syringes were stored at room temperature without shaking.

### 5.1. Preparation of Formulations

11 risankizumab test formulations were prepared (see table 28) and subjected to:
a) shaking of vials for 1, 5, 7, 14, 21 days in a horizontal shaker with 300 U/min (protected from light);
b) shaking of syringes for 1, 5, 7, 14, 21 days in a rocking shaker, movement adjusted to respective viscosity to ensure air bubble movement (protected from light); and
c) room temperature (25° C.) for 1, 5, 7, 14, 21 days (protected from light).

TABLE 28
Formulations chosen for shaking study.

| | |
|---|---|
| F1 | 10 mM acetate + 6.5 mM succinate + 185 mM sorbitol + 0.02 % PS20, pH |
| | 5.7 |
| F2 | 10 mM acetate + 200 mM sorbitol + 0.02 % PS20, pH 5.7 |
| F3 | 10 mM acetate + 50 mM L-arginine HC1 + 110 mM sorbitol + 0.02 % PS20, |
| | pH 5.7 |
| F4 | 10 mM succinate + 185 mM sorbitol + 0.02 % PS20, pH 6.2 |
| F5 | 10 mM acetate + 95 mM mannitol + 95 mM trehalose + 0.02 % PS20, pH |
| | 5.7 |
| F6 | 10 mM acetate + 185 mM trehalose + 0.02 % PS20, pH 5.7 |
| F7 | 10 mM acetate + 50 mM L-arginine HCl + 110 mM trehalose + 0.02 % |
| | PS20, pH 5.7 |
| F8 | 4.4 mM succinate + 185 mM trehalose + 0.02 % PS20, pH 6.0 |
| F9 | 4.4 mM succinate + 50 mM L-arginine HCl + 110 mM trehalose + 0.02 % |
| | PS20, pH 6.0 |
| F10 | 4.4 mM succinate + 95 mM mannitol + 95 mM trehalose + 0.02 % PS20, |
| | pH 6.0 |
| F11 | Buffer-free, 200 mM trehalose, 0.02 % PS20, pH 5.7 |

As a packaging material the formulations were added to vials (Schott) or Neopak syringes. The sterile filtered protein solutions were filled under laminar flow into the sterilized primary packaging materials. The filling volume for the vials was defined to be 3.6 mL. The syringes were filled with 1.04 mL each. All vials and syringes were inspected for visual particles and results were recorded.

### 5.2. Analytics

The analytics at each analytical time point were performed directly after sampling except for the chromatographic assays like SEC for which samples were stored at −70° C. until measurement. Following devices were used for analysis:
UV-Vis spectrophotometer Solo VPE: Conc. at 280 nm, baseline correction at 320 nm, extinction coefficient: 1.52; C Technologies, Inc., NJ, USA
Opalescence meter: HACH Lange opalescence meter; filter: 400-600 nm; Hach Lange GmbH, Dusseldorf, Germany
Ultra performance size exclusion chromatography (UP-SEC): UPLC26, H-Class UV-detection at 280 nm Waters, Milford, MA
Charge heterogeneity by weak cation exchange chromatography (WCX): HPLC75; Fluorescence detection Extinction: 278 nm, Emission: 350 nm; Waters, Milford, MA
IL-23 binding activity: Biacore T200 Chip: CM5 GE Healthcare, Chalfont St Giles, UK
pH-meter: SevenGo—Mettler Toledo, Columbus, OH
Particel sizer: Micro Flow Imaging™ Flow Microscope; By micro flow imaging (MFI); Brightwell Technologies Inc, Ottawa, ON, Canada
Osmometer: Osmomat 030 By freezing point depression, Gonotec GmbH, Berlin, Germany

Further details on the utilized analysis methods are described below.

### 5.3. Results

### 5.3.1. Measurement of the Monomer Content

Monomer content is a key quality attribute of protein stability and quality during stress-induced storage. HP-SEC and UP-SEC were used to measure the monomer content of the formulations.

### UP-SEC Analysis

TABLE 29
UP-SEC-Monomer in % of syringe and vial: Initial values and values after
1/5/7/14/21 days of shaking and after 21 days without movement at 25° C.

| | | | F1 | F2 | F3 | F4 | F5 | F6 | F7 | F8 | F9 | F10 | F11 |
|---|---|---|---|---|---|---|---|---|---|---|---|---|---|
| Time | | Packing | (%) | (%) | (%) | (%) | (%) | (%) | (%) | (%) | (%) | (%) | (%) |
| Initial | | Syringe | 97.9 | 97.9 | 97.9 | 97.5 | 97.8 | 97.8 | 97.9 | 97.4 | 97.5 | 97.5 | 97.7 |
| 1 | day | | 97.6 | 97.6 | 97.7 | 97.0 | 97.6 | 97.6 | 97.7 | 97.3 | 97.5 | 97.3 | 97.6 |
| 5 | days | | 97.5 | 97.5 | 97.6 | 96.9 | 97.5 | 97.5 | 97.6 | 97.2 | 97.4 | 97.2 | 97.5 |
| 7 | days | | 97.4 | 97.4 | 97.5 | 96.7 | 97.4 | 97.3 | 97.5 | 97.0 | 97.3 | 96.9 | 97.3 |
| 14 | days | | 97.3 | 97.2 | 97.4 | 96.6 | 97.2 | 97.2 | 97.4 | 96.9 | 97.2 | 96.8 | 97.2 |
| 21 | days | | 97.3 | 97.3 | 97.4 | 96.7 | 97.2 | 97.3 | 97.4 | 96.9 | 97.2 | 96.9 | 97.3 |
| 21 | days* | | 97.3 | 97.3 | 97.5 | 96.8 | 97.3 | 97.3 | 97.5 | 97.1 | 97.4 | 97.0 | 97.4 |
| Initial | | Vial | 97.9 | 97.8 | 97.9 | 97.5 | 97.8 | 97.7 | 97.9 | 97.6 | 97.8 | 97.7 | 97.9 |
| 1 | day | | 97.7 | 97.7 | 97.8 | 97.3 | 97.7 | 97.7 | 97.8 | 97.5 | 97.7 | 97.5 | 97.7 |
| 5 | days | | 97.5 | 97.5 | 97.6 | 97.0 | 97.5 | 97.5 | 97.6 | 97.3 | 97.5 | 97.3 | 97.6 |
| 7 | days | | 97.5 | 97.5 | 97.6 | 97.0 | 97.5 | 97.5 | 97.6 | 97.2 | 97.5 | 97.3 | 97.4 |
| 14 | days | | 97.3 | 97.3 | 97.4 | 96.7 | 97.2 | 97.2 | 97.4 | 97.0 | 97.3 | 97.0 | 97.3 |
| 21 | days | | 97.2 | 97.2 | 97.4 | 96.7 | 97.2 | 97.2 | 97.4 | 96.9 | 97.2 | 97.0 | 97.3 |
| 21 | days* | | 97.3 | 97.3 | 97.4 | 96.7 | 97.2 | 97.2 | 97.4 | 97.0 | 97.3 | 97.0 | 97.3 |

*corresponds to no movement, whereas the other samples were shaken for the indicated amount of time.

### HP-SEC Analysis

The trends of the HP-SEC analysis are analogous to those of the UP-SEC and thus confirm these results. Monomer values ranging from 97.4-98.8% were obtained.

### Results and Discussion

Overall, the measurements in syringes and vials show similar trends and all formulations proved to be stable with only slight reductions in monomer content.

### 5.3.2. Measurement of the HMW Content

HP-SEC and UP-SEC were used to measure the monomer content of the formulations. HP-SEC was used to determine levels of aggregate (HMW) formation during shaking of the syringes and vials.

### UP-SEC Analysis

The results of the UP-SEC analysis are shown below. The data show similar results as the HP-SEC analysis that the formation of aggregates is mainly driven by the pH. When comparing the initial values it is obvious that formulations at pH 6.0 or 6.2 show a slightly increased HMW content from 0.2 to 0.5% in comparison to solutions formulated at pH 5.7. This trend could also be seen after 21 days of shaking with HMW contents of approx. 1.6% in formulations ≥pH 6.0 and 1.3% in formulations at pH 5.7.

Formulations containing L-arginine like F3 and F7 showed the lowest level of aggregation after 21 days of shaking. The differences in monomer content for the eleven formulations tested in this study observed in UP-SEC and HP-SEC are not significant. The loss of monomer content was in an acceptable range for all formulations being tested. Generally, it can be summarized that the shaking does not significantly increase the HMW content in comparison to the results after 21 days without movement.

The data obtained using the UP-SEC is summarized in Table 30.

TABLE 30
UP-SEC HMW content in % of syringe and vial: Initial values and values after
1/5/7/14/21 days of shaking and after 21 days without movement at 25° C.

| | | | F1 | F2 | F3 | F4 | F5 | F6 | F7 | F8 | F9 | F10 | F11 |
|---|---|---|---|---|---|---|---|---|---|---|---|---|---|
| Time | | Packing | (%) | (%) | (%) | (%) | (%) | (%) | (%) | (%) | (%) | (%) | (%) |
| Initial | | Syringe | 0.9 | 0.9 | 0.8 | 1.3 | 0.9 | 0.9 | 0.8 | 1.1 | 1.0 | 1.0 | 0.9 |
| 1 | day | | 1.0 | 1.0 | 1.0 | 1.6 | 1.0 | 1.0 | 0.9 | 1.3 | 1.1 | 1.3 | 1.0 |
| 5 | days | | 1.1 | 1.1 | 1.1 | 1.8 | 1.2 | 1.2 | 1.0 | 1.4 | 1.2 | 1.4 | 1.1 |
| 7 | days | | 1.2 | 1.3 | 1.1 | 2.0 | 1.3 | 1.3 | 1.2 | 1.7 | 1.4 | 1.7 | 1.3 |
| 14 | days | | 1.3 | 1.4 | 1.2 | 2.0 | 1.4 | 1.4 | 1.2 | 1.7 | 1.4 | 1.8 | 1.4 |
| 21 | days | | 1.4 | 1.4 | 1.3 | 2.0 | 1.5 | 1.5 | 1.2 | 1.7 | 1.5 | 1.8 | 1.4 |
| 21 | days* | | 1.4 | 1.3 | 1.2 | 1.9 | 1.4 | 1.4 | 1.2 | 1.6 | 1.3 | 1.6 | 1.3 |
| Initial | | Vial | 0.9 | 0.9 | 0.8 | 1.3 | 0.9 | 0.9 | 0.8 | 1.1 | 0.9 | 1.1 | 0.9 |
| 1 | day | | 1.0 | 1.0 | 0.9 | 1.4 | 1.0 | 1.0 | 0.9 | 1.2 | 1.0 | 1.2 | 1.0 |
| 5 | days | | 1.1 | 1.1 | 1.0 | 1.6 | 1.1 | 1.1 | 1.0 | 1.4 | 1.2 | 1.3 | 1.1 |
| 7 | days | | 1.2 | 1.2 | 1.1 | 1.7 | 1.2 | 1.2 | 1.0 | 1.4 | 1.1 | 1.4 | 1.2 |
| 14 | days | | 1.4 | 1.4 | 1.2 | 1.9 | 1.4 | 1.4 | 1.2 | 1.7 | 1.3 | 1.7 | 1.3 |
| 21 | days | | 1.4 | 1.4 | 1.3 | 1.9 | 1.4 | 1.4 | 1.2 | 1.7 | 1.4 | 1.7 | 1.4 |
| 21 | days* | | 1.4 | 1.4 | 1.3 | 2.0 | 1.5 | 1.5 | 1.2 | 1.7 | 1.4 | 1.7 | 1.4 |

*corresponds to no movement, whereas the other samples were shaken for the indicated amount of time.

### HP-SEC Analysis

The trend of the data of the UP-SEC analysis are analogous to those of the UP-SEC and thus confirm these results.

### Results and Discussion

Overall, all tested formulations proved to be stable. Similar results were obtained for vials and syringes.

### 5.3.3. Measurement of the Opalescence and Further Parameters

Opalescence, osmolality, pH-values and protein concentrations of all tested formulations remained essentially unchanged following 21 days of shaking for syringes on a horizontal shaker as well as for vials on a rocking shaker (see subsequent data). The lowest level of opalescence was observed in the buffer-free formulation F11 without any additional buffer like acetate or succinate. No observations could be made with regard to visual inspection. No significant difference can be observed by comparing the generated data of vials and syringes.

The increased opalescence level of F6 after 1 day and F10 after 5 days could not be confirmed by the results of the following sampling time points. Therefore, it is likely that a measuring error occurred and these results do not have relevance for the interpretation of the results.

TABLE 31
Shaking of formulations in syringe and vial: Initial values, values after 1/5/7/14/21
days of shaking and values after 21 days without (w/o) movement of opalescence,
osmolality, pH-values and protein conc. (shaking at room temperature).

| | | | Opalescence (FNU) | | Osmolality (mOsm/kg) | | pH | | Protein (mg/mL) | |
|---|---|---|---|---|---|---|---|---|---|---|
| Formulation | Time | | Syringe | Vial | Syringe | Vial | Syringe | Vial | Syringe | Vial |
| F1 | Initial | | 9.0 | 8.1 | 307 | 306 | 5.6 | 5.7 | 150 | 152 |
| | 1 | day | 8.1 | 8.3 | 304 | 305 | 5.7 | 5.7 | 151 | 151 |
| | 5 | days | 7.6 | 9.2 | 307 | 305 | 5.7 | 5.8 | 151 | 151 |
| | 7 | days | 8.5 | 9.0 | 307 | 306 | 5.7 | 5.7 | 148 | 150 |
| | 14 | days | 8.6 | 8.3 | 305 | 307 | 5.7 | 5.7 | 152 | 151 |
| | 21 | days | 8.8 | 8.6 | 304 | 306 | 5.8 | 5.7 | 150 | 149 |
| | 21 | days* | 9.6 | 8.0 | 308 | 304 | 5.7 | 5.7 | 151 | 149 |
| F2 | Initial | | 5.6 | 5.4 | 306 | 307 | 5.6 | 5.6 | 151 | 152 |
| | 1 | day | 5.9 | 5.7 | 304 | 305 | 5.7 | 5.7 | 149 | 152 |
| | 5 | days | 5.2 | 5.6 | 305 | 305 | 5.7 | 5.7 | 149 | 149 |
| | 7 | days | 5.7 | 5.8 | 304 | 305 | 5.7 | 5.7 | 149 | 149 |
| | 14 | days | 5.4 | 5.4 | 306 | 307 | 5.7 | 5.7 | 151 | 151 |
| | 21 | days | 6.5 | 7.0 | 304 | 304 | 5.7 | 5.7 | 151 | 150 |
| | 21 | days* | 5.7 | 4.8 | 309 | 305 | 5.7 | 5.7 | 150 | 151 |
| F3 | Initial | | 12.8 | 11.3 | 298 | 289 | 5.6 | 5.7 | 154 | 152 |
| | 1 | day | 13.0 | 12.2 | 289 | 282 | 5.7 | 5.8 | 151 | 149 |
| | 5 | days | 12.7 | 12.7 | 291 | 286 | 5.6 | 5.7 | 153 | 151 |
| | 7 | days | 12.2 | 13.3 | 292 | 291 | 5.7 | 5.7 | 153 | 149 |
| | 14 | days | 12.2 | 11.9 | 293 | 291 | 5.7 | 5.7 | 152 | 151 |
| | 21 | days | 12.7 | 12.2 | 291 | 290 | 5.7 | 5.7 | 153 | 151 |
| | 21 | days* | 13.0 | 11.3 | 293 | 292 | 5.7 | 5.7 | 152 | 154 |
| F4 | Initial | | 13.4 | 11.1 | 301 | 294 | 6.1 | 6.2 | 151 | 150 |
| | 1 | day | 11.4 | 11.4 | 296 | 297 | 6.0 | 6.3 | 151 | 152 |
| | 5 | days | 11.9 | 12.4 | 297 | 296 | 6.0 | 6.2 | 150 | 148 |
| | 7 | days | 11.7 | 12.8 | 299 | 295 | 6.2 | 6.2 | 153 | 149 |
| | 14 | days | 11.9 | 11.2 | 298 | 296 | 6.2 | 6.2 | 152 | 153 |
| | 21 | days | 12.2 | 12.3 | 301 | 298 | 6.2 | 6.2 | 151 | 151 |
| | 21 | days* | 12.9 | 11.5 | 297 | 299 | 6.2 | 6.2 | 153 | 152 |
| F5 | Initial | | 5.9 | 4.9 | 310 | 306 | 5.6 | 5.7 | 156 | 154 |
| | 1 | day | 5.6 | 5.3 | 306 | 303 | 5.7 | 5.7 | 154 | 153 |
| | 5 | days | 5.7 | 5.8 | 305 | 305 | 5.6 | 5.7 | 154 | 154 |
| | 7 | days | 5.5 | 6.0 | 309 | 304 | 5.7 | 5.7 | 153 | 151 |
| | 14 | days | 4.9 | 5.0 | 306 | 305 | 5.7 | 5.7 | 156 | 157 |
| | 21 | days | 5.4 | 7.5 | 306 | 308 | 5.7 | 5.7 | 155 | 154 |
| | 21 | days* | 6.3 | 4.7 | 307 | 305 | 5.6 | 5.7 | 154 | 153 |
| F6 | Initial | | 5.7 | 4.9 | 313 | 311 | 5.7 | 5.7 | 153 | 151 |
| | 1 | day | 11.0 | 6.1 | 304 | 307 | 5.6 | 5.8 | 152 | 151 |
| | 5 | days | 6.0 | 6.1 | 308 | 305 | 5.6 | 5.7 | 150 | 149 |
| | 7 | days | 5.6 | 5.9 | 307 | 308 | 5.7 | 5.7 | 151 | 151 |
| | 14 | days | 5.2 | 4.9 | 307 | 309 | 5.7 | 5.7 | 150 | 151 |
| | 21 | days | 5.7 | 6.5 | 311 | 310 | 5.7 | 5.7 | 152 | 152 |
| | 21 | days* | 5.5 | 4.8 | 311 | 310 | 5.7 | 5.7 | 153 | 150 |
| F7 | Initial | | 12.3 | 10.8 | 307 | 304 | 5.6 | 5.7 | 153 | 153 |
| | 1 | day | 12.3 | 11.3 | 302 | 302 | 5.6 | 5.7 | 153 | 154 |
| | 5 | days | 12.1 | 12.5 | 301 | 302 | 5.6 | 5.7 | 151 | 153 |
| | 7 | days | 11.4 | 12.6 | 302 | 302 | 5.7 | 5.7 | 153 | 154 |
| | 14 | days | 12.2 | 10.9 | 304 | 303 | 5.7 | 5.7 | 153 | 152 |
| | 21 | days | 11.5 | 12.1 | 306 | 304 | 5.7 | 5.7 | 153 | 152 |
| | 21 | days* | 12.6 | 10.6 | 305 | 303 | 5.6 | 5.7 | 153 | 153 |
| F8 | Initial | | 7.0 | 6.1 | 295 | 290 | 5.9 | 6.0 | 151 | 151 |
| | 1 | day | 6.7 | 7.3 | 290 | 287 | 5.9 | 6.0 | 151 | 150 |
| | 5 | days | 6.9 | 7.6 | 289 | 286 | 5.9 | 6.0 | 150 | 150 |
| | 7 | days | 6.5 | 7.1 | 293 | 297 | 6.0 | 6.0 | 150 | 151 |
| | 14 | days | 6.5 | 6.6 | 294 | 289 | 6.0 | 6.0 | 151 | 149 |
| | 21 | days | 7.0 | 6.5 | 293 | 292 | 6.0 | 6.0 | 150 | 150 |
| | 21 | days* | 7.5 | 6.3 | 291 | 288 | 6.0 | 6.0 | 151 | 147 |
| F9 | Initial | | 13.9 | 12.4 | 298 | 291 | 5.9 | 6.0 | 149 | 149 |
| | 1 | day | 12.5 | 12.7 | 292 | 293 | 5.9 | 6.0 | 151 | 148 |
| | 5 | days | 13.6 | 13.5 | 291 | 292 | 5.9 | 6.0 | 150 | 148 |
| | 7 | days | 12.8 | 13.7 | 295 | 296 | 6.0 | 6.0 | 150 | 153 |
| | 14 | days | 12.7 | 12.3 | 295 | 294 | 6.0 | 6.0 | 149 | 150 |
| | 21 | days | 13.1 | 12.9 | 295 | 296 | 6.0 | 6.0 | 150 | 150 |
| | 21 | days* | 13.8 | 12.4 | 294 | 297 | 6.0 | 6.0 | 152 | 149 |
| F10 | Initial | | 7.1 | 6.3 | 301 | 295 | 6.0 | 6.0 | 148 | 149 |
| | 1 | day | 6.7 | 6.5 | 295 | 293 | 5.9 | 6.0 | 149 | 148 |
| | 5 | days | 11.1 | 8.3 | 292 | 291 | 5.9 | 6.0 | 146 | 147 |
| | 7 | days | 6.5 | 7.5 | 296 | 296 | 6.0 | 6.0 | 149 | 148 |
| | 14 | days | 6.1 | 6.1 | 297 | 298 | 6.0 | 6.0 | 151 | 148 |
| | 21 | days | 6.6 | 6.5 | 295 | 295 | 6.0 | 6.0 | 149 | 149 |
| | 21 | days* | 7.4 | 5.9 | 295 | 295 | 6.0 | 6.0 | 150 | 148 |
| F11 | Initial | | 4.9 | 3.3 | 306 | 298 | 5.6 | 5.6 | 148 | 146 |
| | 1 | day | 3.8 | 4.0 | 299 | 300 | 5.6 | 5.7 | 147 | 144 |
| | 5 | days | 4.9 | 4.2 | 300 | 297 | 5.6 | 5.7 | 145 | 147 |
| | 7 | days | 3.7 | 4.2 | 300 | 300 | 5.7 | 5.7 | 147 | 146 |
| | 14 | days | 3.7 | 3.2 | 301 | 301 | 5.7 | 5.7 | 147 | 148 |
| | 21 | days | 3.7 | 3.3 | 303 | 301 | 5.6 | 5.7 | 148 | 146 |
| | 21 | days* | 5.2 | 3.2 | 302 | 298 | 5.6 | 5.7 | 146 | 147 |

*corresponds to no movement, whereas the other samples were shaken for the indicated amount of time.

### Binding Activity

The results of the SPR (Biacore) measurements showed that the shaking does not influence the binding activity of the molecule for the syringes on a rocking shaker as well as for the vials on a horizontal shaker. Overall, the binding activity remained high in a range of 91-111% and the specific binding activity in a range of 98-107%.

### Results and Discussion

The opalescence depends on the formulation composition and ranges from 5 FNU in an excipient-free or buffer-free respectively to 14 FNU, but did not significantly increase over time.

The pH value, osmolality, opalescence and protein concentrations as well as binding activity against IL-23 remained unchanged for all formulations over the entire study period.

### 5.4. Further Analytics and Results

In addition, further analytics were performed for the eleven formulations tested with the following results. Shaking type and times, storage and used syringes and vials were as described above.
The HP-SEC fragments contents remained essentially constant over the shaking time in vials and syringes. The fragments content was in a range of 0.3-0.5%. The UP-SEC LMW contents remained essentially constant over the shaking time in vials and syringes. The fragments content was in a range of 1.3-1.4%.
The weak cation exchange chromatography (WCX) showed that the percentage of the distribution of the main peak, APG and BPG remained at a constant level for all formulations tested during the study. The levels of the main peak, APG and BPG did not change significantly over shaking time. No differences between the formulations were observed.
The particle content measured by Micro Flow Imaging (MFI) remained essentially constant over the 21 days shaking time.

### 5.5. Summary

All in all, only minor differences in stability-indicating parameters were detected between formulations following exposure to shaking stress. For example, the formulations F3 and F7 showed highest monomer content (HP-SEC & UP-SEC) but they also showed the highest level of opalescence. It can be summarized that the formulations being tested in this study would be a viable formulation.

6. Comparison of Acetate and Succinate Buffer Systems with Different pH Values in Multiple Freeze/Thaw Cycles

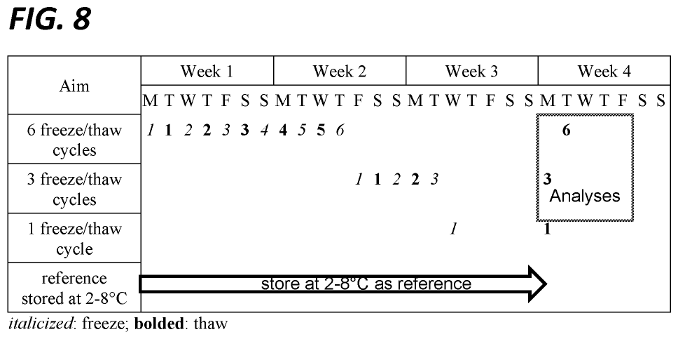

The freeze and thaw behavior of different formulations at 150 mg/mL risankizumab and its influence on product quality was evaluated. Therefore, the formulations were exposed to freezing and thawing stresses in mini bags at the intended target concentration of 150 mg/mL and a fill volume of 12 mL to simulate the conditions of bag-freezing in pilot or large scale. An overview of the experimental schedule is provided in FIG. 8.

In total eleven formulations that differ in pH, buffering strategy and tonicity agent plus an intermediate storage bulk were filled into mini-bags with a filling volume of 12 mL followed by a controlled freezing step to −40° C. Additionally bags were stored at 5° C. Note, the eleven formulations correspond to the ones tested in the previous example.

The freezing step was performed using a freeze-thaw setup provided by a classical freeze-dryer. Here, the mini-bags with a sample volume of 12 mL were frozen controlled with a freezing ramp of 0.5° C./min to −40° C. At this point the temperature remained constant over sixteen hours to ensure complete freezing of the sample volume. The thawing step was performed according to the freezing step with a heating rate of 0.5° C./min. The hold time at room temperature was set to four hours.

A complete freeze/thaw cycle (1×F/T) is defined as follows:
1. Freezing from room temperature to −40° (0.5° C./min)
2. Hold time of 16 hours at −40° C.
3. Thawing from −40° C. to room temperature (0.5° C./min)
4. Hold time of 4 hours at room temperature

This procedure was conducted for 1×F/T, 3×F/T and 6×F/T. After completion of the final process cycle the bags were transferred into a freezer at −40° C. and stored until the samples were thawed and analyzed simultaneously.

### 6.1. Preparation of Formulations

The formulations were prepared as described in the prior example (see Table 28). A further formulation F12 was prepared which is excipient-free containing 0.02% PS20, pH 5.7. 12 mL sterile filtered protein solutions were filled under laminar flow into the sterilized primary packaging materials, which are Mini Flexboy bags having a volume of 15 mL. All bags were inspected for visual particles and results were recorded.

In each freeze-thaw run twelve bags were put on each plate within the freeze-dryer. In total 36 bags were freeze/thawed each run including three bags per formulation. The bags were distributed in a defined scheme to eliminate influences of the bag position within the freeze-dryer.

### 6.2. Analytics

After conducting all cycles according to the experimental plan above, the bags were thawed in an additional thawing step successively. This procedure had the advantage that the samples could be analyzed altogether. The bags were transferred into the precooled freeze-dryer at −40° C. followed by the thawing step. HP-SEC, UP-SEC, osmolality, pH, protein concentration, opalescence, binding activity and sub-visible particle measurements were performed. Following devices were used for analysis:
UV-Vis spectrophotometer Solo VPE: Conc. at 280 nm, baseline correction at 320 nm, extinction coefficient: 1.52; C Technologies, Inc., NJ, USA
Opalescence: HACH Lange opalescence meter; filter: 400-600 nm; Hach Lange GmbH, Dusseldorf, Germany
Ultra performance size exclusion chromatography (UP-SEC): UPLC26, H-Class UV-detection at 280 nm Waters, Milford, MA
Charge heterogeneity by weak cation exchange chromatography (WCX): HPLC75; Fluorescence detection Extinction: 278 nm, Emission: 350 nm; Waters, Milford, MA
IL-23 binding activity: Biacore T200 Chip: CM5 GE Healthcare, Chalfont St Giles, UK
pH-meter: SevenGo—Mettler Toledo, Columbus, OH
Particel sizer: Micro Flow Imaging™ Flow Microscope; By micro flow imaging (MFI); Brightwell Technologies Inc, Ottawa, ON, Canada
Osmometer: Osmomat 030 By freezing point depression, Gonotec GmbH, Berlin, Germany

Further details on the utilized analysis methods are described below.

### 6.3. Results

### 6.3.1. Measurement of the Monomer and HMW Content

Monomer content is a key quality attribute of protein stability and quality during stress-induced storage. HP-SEC and UP-SEC was used to determine levels of aggregate formation during freeze/thaw of the mini-bags. The subsequent table summarizes the results of the HP-SEC & UP-SEC analytic.

TABLE 32
HP-SEC/UP-SEC: Initial values and values after
1/3/6 F/T cycles and three weeks at 5° C. in %.

| | | Aggregate | Monomer | Fragment | Aggregate | Monomer | Fragment |
|---|---|---|---|---|---|---|---|
| | Sampling | HP-SEC | HP-SEC | HP-SEC | UP-SEC | UP-SEC | UP-SEC |
| Formulation | time point | % | % | % | % | % | % |
| F1 | initial | 1.4 | 98.3 | 0.4 | 1.3 | 97.2 | 1.5 |
| | 1 × F/T | 1.5 | 98.1 | 0.4 | 1.4 | 97.3 | 1.3 |
| | 3 × F/T | 1.5 | 98.2 | 0.4 | 1.4 | 97.4 | 1.2 |
| | 6 × F/T | 1.5 | 98.1 | 0.4 | 1.4 | 97.4 | 1.3 |
| | 5° C. | 1.6 | 98.1 | 0.4 | 1.4 | 97.3 | 1.3 |
| F2 | initial | 1.4 | 98.3 | 0.3 | 1.3 | 97.2 | 1.5 |
| | 1 × F/T | 1.5 | 98.1 | 0.4 | 1.5 | 97.2 | 1.3 |
| | 3 × F/T | 1.4 | 98.2 | 0.4 | 1.4 | 97.3 | 1.2 |
| | 6 × F/T | 1.5 | 98.2 | 0.4 | 1.4 | 97.3 | 1.3 |
| | 5° C. | 1.5 | 98.1 | 0.4 | 1.4 | 97.3 | 1.3 |
| F3 | initial | 1.2 | 98.4 | 0.3 | 1.2 | 97.3 | 1.5 |
| | 1 × F/T | 1.3 | 98.3 | 0.4 | 1.3 | 97.4 | 1.3 |
| | 3 × F/T | 1.3 | 98.4 | 0.4 | 1.3 | 97.5 | 1.3 |
| | 6 × F/T | 1.4 | 98.3 | 0.4 | 1.3 | 97.5 | 1.3 |
| | 5° C. | 1.4 | 98.3 | 0.4 | 1.3 | 97.5 | 1.3 |
| F4 | initial | 1.6 | 98.0 | 0.3 | 1.8 | 96.8 | 1.5 |
| | 1 × F/T | 2.1 | 97.6 | 0.4 | 2.0 | 96.8 | 1.2 |
| | 3 × F/T | 1.9 | 97.7 | 0.4 | 1.9 | 96.8 | 1.3 |
| | 6 × F/T | 2.1 | 97.5 | 0.4 | 1.9 | 96.8 | 1.3 |
| | 5° C. | 2.0 | 97.6 | 0.4 | 1.9 | 96.9 | 1.3 |
| F5 | initial | 1.6 | 98.0 | 0.3 | 1.3 | 97.2 | 1.5 |
| | 1 × F/T | 1.5 | 98.1 | 0.4 | 1.4 | 97.3 | 1.3 |
| | 3 × F/T | 1.4 | 98.2 | 0.4 | 1.4 | 97.3 | 1.3 |
| | 6 × F/T | 1.5 | 98.2 | 0.4 | 1.4 | 97.3 | 1.3 |
| | 5° C. | 1.5 | 98.2 | 0.4 | 1.4 | 97.3 | 1.3 |
| F6 | initial | 1.3 | 98.3 | 0.3 | 1.3 | 97.2 | 1.5 |
| | 1 × F/T | 1.5 | 98.1 | 0.4 | 1.4 | 97.3 | 1.3 |
| | 3 × F/T | 1.4 | 98.2 | 0.4 | 1.4 | 97.3 | 1.3 |
| | 6 × F/T | 1.5 | 98.2 | 0.4 | 1.4 | 97.3 | 1.3 |
| | 5° C. | 1.5 | 98.1 | 0.4 | 1.4 | 97.3 | 1.3 |
| F7 | initial | 1.3 | 98.4 | 0.3 | 1.2 | 97.3 | 1.5 |
| | 1 × F/T | 1.4 | 98.3 | 0.4 | 1.3 | 97.4 | 1.3 |
| | 3 × F/T | 1.3 | 98.3 | 0.4 | 1.3 | 97.5 | 1.3 |
| | 6 × F/T | 1.4 | 98.3 | 0.4 | 1.2 | 97.5 | 1.3 |
| | 5° C. | 1.4 | 98.3 | 0.4 | 1.3 | 97.5 | 1.3 |
| F8 | initial | 1.5 | 98.2 | 0.3 | 1.5 | 97.0 | 1.4 |
| | 1 × F/T | 1.8 | 97.8 | 0.4 | 1.7 | 97.0 | 1.3 |
| | 3 × F/T | 1.7 | 97.9 | 0.4 | 1.6 | 97.1 | 1.3 |
| | 6 × F/T | 1.7 | 97.9 | 0.4 | 1.6 | 97.1 | 1.3 |
| | 5° C. | 1.7 | 97.9 | 0.4 | 1.6 | 97.1 | 1.3 |
| F9 | initial | 1.5 | 98.2 | 0.3 | 1.3 | 97.2 | 1.5 |
| | 1 × F/T | 1.5 | 98.1 | 0.4 | 1.4 | 97.3 | 1.3 |
| | 3 × F/T | 1.4 | 98.2 | 0.4 | 1.4 | 97.3 | 1.3 |
| | 6 × F/T | 1.5 | 98.1 | 0.4 | 1.4 | 97.3 | 1.3 |
| | 5° C. | 1.5 | 98.1 | 0.4 | 1.4 | 97.3 | 1.3 |
| F10 | initial | 1.6 | 98.1 | 0.3 | 1.5 | 97.0 | 1.5 |
| | 1 × F/T | 1.7 | 97.9 | 0.4 | 1.6 | 97.1 | 1.3 |
| | 3 × F/T | 1.7 | 98.0 | 0.4 | 1.6 | 97.1 | 1.3 |
| | 6 × F/T | 1.7 | 98.0 | 0.4 | 1.6 | 97.1 | 1.3 |
| | 5° C. | 1.7 | 97.9 | 0.4 | 1.5 | 97.3 | 1.3 |
| F11 | initial | 1.4 | 98.3 | 0.3 | 1.3 | 97.3 | 1.5 |
| | 1 × F/T | 1.4 | 98.2 | 0.4 | 1.4 | 97.3 | 1.3 |
| | 3 × F/T | 1.4 | 98.2 | 0.4 | 1.4 | 97.3 | 1.3 |
| | 8 × F/T | 1.4 | 98.2 | 0.4 | 1.4 | 97.3 | 1.3 |
| | 5° C. | 1.4 | 98.2 | 0.4 | 1.4 | 97.3 | 1.3 |
| F12 | initial | 1.5 | 98.2 | 0.3 | 1.3 | 97.2 | 1.5 |
| | 1 × F/T | 1.7 | 98.0 | 0.4 | 1.6 | 97.1 | 1.3 |
| | 3 × F/T | 1.8 | 97.8 | 0.4 | 1.7 | 97.0 | 1.3 |
| | 6 × F/T | 2.0 | 97.6 | 0.4 | 1.9 | 96.8 | 1.3 |
| | 5° C. | 1.5 | 98.1 | 0.4 | 1.3 | 97.4 | 1.3 |

The data show that the formation of aggregates is mainly driven by the pH. By comparing the results after six F/T cycles it is obvious that formulations at pH 6.0 or 6.2 show a slightly increased HMW content of 0.2-0.6% in comparison to solutions formulated at pH 5.7. Formulations containing L-arginine like F3 and F7 showed the lowest level of aggregation after six F/T cycles. Generally, it can be summarized that the freeze/thaw stress does not significantly increase the HMW content in comparison to the results after 21 days at 5° C.

### 6.3.2. Measurement of the Opalescence and Further Parameters

Opalescence, osmolality, pH-values and protein concentrations of all tested formulations remained unchanged following 6 F/T cycles and after storage at 5° C. for three weeks. The lowest level of opalescence was observed in buffer-free formulations (F11 & F12).

TABLE 33
Initial values and values after 1/3/6 F/T cycles of opalescence in
FNU, osmolality in mOsm/kg, pH-values and protein conc. in g/L.

| | | | | | | Protein |
|---|---|---|---|---|---|---|
| | | Sampling | Opalescence | Osmolality | | conc. |
| Formulation | Components | time point | FNU | mOsmol · kg⁻¹ | pH | g · L⁻¹ |
| F1 | 10 mM acetate + | initial | 7.7 | 306 | 5.6 | 153 |
| | 6.5 mM succinate + | 1 × F/T | 8.0 | 307 | 5.6 | 150 |
| | 185 mM sorbitol + | 3 × F/T | 8.1 | 308 | 5.7 | 151 |
| | 0.02% PS20, | 6 × F/T | 8.1 | 307 | 5.7 | 153 |
| | pH 5.7 | 5° C. | 8.8 | 306 | 5.7 | 151 |
| F2 | 10 mM acetate + | initial | 4.8 | 304 | 5.7 | 153 |
| | 200 mM sorbitol + | 1 × F/T | 5.2 | 305 | 5.6 | 152 |
| | 0.02% PS20, | 3 × F/T | 4.8 | 306 | 5.6 | 153 |
| | pH 5.7 | 6 × F/T | 5.4 | 306 | 5.7 | 152 |
| | | 5° C. | 5.6 | 306 | 5.7 | 150 |
| F3 | 10 mM acetate + | initial | 11.1 | 292 | 5.7 | 153 |
| | 50 mM L-arginine HCl + | 1 × F/T | 11.8 | 292 | 5.6 | 152 |
| | 110 mM sorbitol + | 3 × F/T | 12.1 | 292 | 5.6 | 152 |
| | 0.02% PS20, | 6 × F/T | 12.2 | 291 | 5.6 | 153 |
| | pH 5.7 | 5° C. | 11.5 | 292 | 5.6 | 150 |
| F4 | 10 mM succinate + | initial | 10.6 | 306 | 6.1 | 154 |
| | 185 mM sorbitol + | 1 × F/T | 11.1 | 296 | 6.1 | 150 |
| | 0.02% PS20, | 3 × F/T | 12.1 | 300 | 6.1 | 151 |
| | pH 6.2 | 6 × F/T | 11.3 | 301 | 6.1 | 151 |
| | | 5° C. | 11.3 | 300 | 6.1 | 151 |
| F5 | 10 mM acetate + | initial | 4.7 | 307 | 5.6 | 154 |
| | 95 mM mannitol + | 1 × F/T | 4.9 | 302 | 5.6 | 151 |
| | 95 mM trehalose + | 3 × F/T | 5.4 | 305 | 5.6 | 153 |
| | 0.02% PS20, | 6 × F/T | 5.1 | 305 | 5.6 | 153 |
| | pH 5.7 | 5° C. | 4.9 | 306 | 5.6 | 150 |
| F6 | 10 mM acetate + | initial | 4.5 | 309 | 5.6 | 152 |
| | 185 mM trehlaose + | 1 × F/T | 4.7 | 306 | 5.6 | 152 |
| | 0.02% PS20, | 3 × F/T | 5.0 | 309 | 5.6 | 152 |
| | pH 5.7 | 6 × F/T | 4.8 | 309 | 5.6 | 151 |
| | | 5° C. | 4.7 | 309 | 5.6 | 150 |
| F7 | 10 mM acetate + | initial | 10.6 | 307 | 5.7 | 152 |
| | 50 mM L-arginine HCl + | 1 × F/T | 11.2 | 303 | 5.6 | 151 |
| | 110 mM trehalose + | 3 × F/T | 11.8 | 301 | 5.6 | 151 |
| | 0.02% PS20, | 6 × F/T | 11.0 | 305 | 5.6 | 153 |
| | pH 5.7 | 5° C. | 12.0 | 304 | 5.6 | 152 |
| F8 | 4.4 mM succinate + | initial | 6.2 | 300 | 5.9 | 148 |
| | 185 mM trehalose + | 1 × F/T | 6.4 | 291 | 5.9 | 150 |
| | 0.02% PS20, | 3 × F/T | 6.7 | 296 | 5.9 | 151 |
| | pH 6.0 | 6 × F/T | 6.3 | 295 | 5.9 | 152 |
| | | 5° C. | 6.2 | 296 | 5.9 | 151 |
| F9 | 4.4 mM succinate + | initial | 11.9 | 303 | 5.9 | 153 |
| | 50 mM L arginine HCl + | 1 × F/T | 12.4 | 294 | 5.9 | 151 |
| | 110 mM trehalose + | 3 × F/T | 12.9 | 296 | 5.8 | 153 |
| | 0.02% PS20, | 6 × F/T | 12.3 | 295 | 5.8 | 153 |
| | pH 6.0 | 5° C. | 11.8 | 295 | 5.9 | 150 |
| F10 | 4.4 mM succinate + | initial | 6.0 | 314 | 5.9 | 153 |
| | 85 mM mannitol + | 1 × F/T | 5.8 | 298 | 5.9 | 151 |
| | 95 mM trehalose + | 3 × F/T | 6.6 | 298 | 5.8 | 150 |
| | 0.02% PS20, | 6 × F/T | 6.1 | 298 | 5.8 | 153 |
| | pH 6.0 | 5° C. | 5.9 | 301 | 5.9 | 150 |
| F11 | Buffer-free, | initial | 3.4 | 322 | 5.6 | 154 |
| | 200 mM trehalose, | 1 × F/T | 3.3 | 305 | 5.6 | 150 |
| | 0.02% PS20, | 3 × F/T | 3.7 | 306 | 5.6 | 151 |
| | pH 5.7 | 6 × F/T | 3.6 | 305 | 5.7 | 153 |
| | | 5° C. | 3.6 | 306 | 5.6 | 152 |
| F12 | Excipient-free, | initial | 3.5 | 29 | 5.6 | 151 |
| | 0.02% PS20, | 1 × F/T | 3.8 | 25 | 5.6 | 152 |
| | pH 5.7 | 3 × F/T | 4.0 | 25 | 5.6 | 151 |
| | | 6 × F/T | 3.8 | 25 | 5.6 | 151 |
| | | 5° C. | 3.6 | 27 | 5.6 | 151 |

### Binding Activity

The results of the SPR (Biacore) measurements of the IL23 binding activity show that the freeze/thaw cycles do not influence the binding activity of the molecule. The binding activity ranges between 96-117%.

### Results and Discussion

The measured opalescence depended on the formulation. The pH value, osmolality, opalescence and protein concentrations as well as binding of IL-23 essentially remained unchanged and thus stable for all formulations over the entire investigation period regardless of the stress condition (F/T & hold time at 5° C.).

### 6.3.3. Measurement of Particles

The subsequent table summarizes the number of particles for each STP. No clear trends could be observed for all formulations being tested. The formulations F3, F7 and F9 show a slightly increased amount of SVP in comparison to other formulations being tested. This observation was mainly seen for the SVP ≥2 μm and ≥10 μm.

TABLE 34
Subvisible Particles - MFI: Initial values and values after
one, three and six F/T cycles and three weeks at 5° C.

| | Particle | | Number of measured particles | | | | | | | | | | | |
|---|---|---|---|---|---|---|---|---|---|---|---|---|---|---|
| Treatment | size | | F1 | F2 | F3 | F4 | F5 | F6 | F7 | F8 | F9 | F10 | F11 | F12 |
| Initial | ≥2 | μm | 505 | 1104 | 1604 | 723 | 508 | 1046 | 878 | 1259 | 622 | 1255 | 1314 | 673 |
| | ≥10 | μm | 44 | 32 | 50 | 34 | 31 | 38 | 18 | 71 | 19 | 38 | 41 | 36 |
| | ≥25 | μm | 2 | 2 | 2 | 2 | 2 | 3 | 2 | 7 | 2 | 1 | 2 | 2 |
| 1 × F/T | ≥2 | μm | 1443 | 660 | 2426 | 831 | 710 | 883 | 2784 | 1260 | 2557 | 1070 | 925 | 1759 |
| | ≥10 | μm | 49 | 19 | 639 | 18 | 38 | 21 | 683 | 28 | 995 | 45 | 35 | 63 |
| | ≥25 | μm | 1 | 0 | 6 | 2 | 5 | 2 | 1 | 2 | 8 | 4 | 1 | 2 |
| 3 × F/T | ≥2 | μm | 1485 | 836 | 2764 | 1440 | 717 | 656 | 3501 | 698 | 2708 | 901 | 520 | 1513 |
| | ≥10 | μm | 79 | 42 | 898 | 56 | 49 | 44 | 604 | 34 | 950 | 60 | 30 | 59 |
| | ≥25 | μm | 6 | 6 | 10 | 3 | 2 | 3 | 10 | 5 | 3 | 5 | 5 | 3 |
| 6 × F/T | ≥2 | μm | 1392 | 1008 | 4551 | 1214 | 714 | 1121 | 3265 | 1051 | 2752 | 810 | 925 | 1868 |
| | ≥10 | μm | 81 | 41 | 1089 | 31 | 48 | 69 | 518 | 38 | 1209 | 60 | 43 | 54 |
| | ≥25 | μm | 7 | 10 | 41 | 4 | 9 | 6 | 17 | 1 | 5 | 7 | 3 | 3 |
| 5° C. | ≥2 | μm | 1780 | 1259 | 2222 | 1341 | 555 | 784 | 2148 | 879 | 2825 | 1343 | 983 | 2131 |
| | ≥10 | μm | 121 | 37 | 439 | 54 | 32 | 35 | 599 | 17 | 815 | 50 | 38 | 80 |
| | ≥25 | μm | 4 | 1 | 6 | 6 | 3 | 4 | 2 | 1 | 2 | 6 | 3 | 7 |

### Results and Discussion

While for ≥2 m particles similar trends are observed, a slight increase of particles having a size of ≥10 m was observed for F3, F7 and F9 compared to others. For ≥25 m particles, a slight increase for F3 after 6×F/T was observed. Overall, particle formation was no major issue during F/T for all tested formulations.

### 6.4. Further Analytics and Results

In addition, further analytics were performed for the twelve formulations tested with the following results (freeze/thaw cycles were as described above).
The weak cation exchange chromatography (WCX) showed that the percentage of the distribution of the main peak, APG and BPG remained at a constant level for all formulations tested during the study. The levels of the main peak, APG and BPG did not change significantly over 6 freeze/thaw cycles. The main peak ranged from 65-67%, the APG content from 21-23% the BPG content from about 11-14%. No differences between the formulations were observed.

### 6.5. Summary of Results

The results can be summarized as follows:
Visual inspection: After six F/T cycles, no observations could be made during visual inspection for all formulations.
SVP: Regarding the sub-visible particle level no major issues could be observed. There was a slight increase for F3, F7 and F9 compared to other formulations but significantly below specifications of Pharmacopeia.
HP-SEC & UP-SEC: For the test methods with a focus on protein integrity like HP-SEC and UP-SEC F3 turned out to be the most stable formulation and F4 as the least stable. F12 without any buffer or excipients exhibited an acceptable stability during freeze/thaw cycles.
IEC: For the results of the IEC no discrimination for any formulation could be observed. The F/T-cycles do not negatively influence the contribution of APG and BPG.
Opalescence: The opalescence depends on the formulation and ranges from 4 FNU in an excipient-free or buffer-free formulation to 13 FNU.
The pH value, osmolality, opalescence and protein concentrations as well as binding remained unchanged for all formulations over the entire investigation period regardless of the stress condition (F/T & hold time at 5° C.).

It can be summarized that most of the formulations being tested in this example are viable formulations for a 150 mg/mL formulation. Only minor effects of the freeze/thaw stress on protein stability were observed. Due to medical concern with formulations containing sorbitol, these formulations may be found less advantageous in order to also address fructose-intolerant patients. Nevertheless, for other patients sorbitol-containing solutions may be found useful. All in all, only minor differences in stability-indicating parameters could be detected between formulations following exposure to F/T. For example the formulation F3 was most stable in monomer content (HP-SEC) but contrarily an increased level of subvisible particles could be detected.

### 7. Impact of the pH on the Formulation Stability

The impact of the pH value on the stability of 150 mg/mL risankizumab formulations were tested with the formulations shown in Table 35.

TABLE 35
Composition of formulations.

| Formulation | pH | Acetate | Trehalose | Polysorbate 20 |
|---|---|---|---|---|
| F1 | 5.0 | 10 mM | 185 mM | 0.2 mg/mL |
| F2 | 5.2 | | | |
| F3 | 5.5 | | | |
| F4 | 5.7 | | | |
| F5 | 5.9 | | | |
| F6 | 6.2 | | | |

### 7.1. Preparation of Formulations

The formulations were prepared as described above.

### 7.2. Analytics

Measurements of the samples were performed at 1, 3, 6, 9, 12, 18, 24 and 36 months storage, as well as initially before storage. Various methods for analysis were used, including HIC, UP-SEC, IEC, as well as viscosity, break loose and gliding force and binding specificity measurements. Further details on the utilized analysis methods are described below.

### 7.3. Results

### 7.3.1. Measurement of the Monomer Content

The monomer content was measured as in the previous examples using UP-SEC analysis, the results are shown in Table 36.

TABLE 36
UP-SEC-Monomer-measurements in % of the
formulations having different pH-values.

| | | Storage | | | | | | |
|---|---|---|---|---|---|---|---|---|
| Storage | | time, | | | | | | |
| condition | | months | F1 | F2 | F3 | F4 | F5 | F6 |
| initial | | 0 | 98.3 | 98.3 | 98.2 | 98.0 | 97.8 | 97.3 |
| 5° | C. | 3 | 98.1 | 98.1 | 97.9 | 97.7 | 97.4 | 96.8 |
| 5° | C. | 6 | 98.0 | 98.0 | 97.8 | 97.6 | 97.4 | 96.9 |
| 5° | C. | 9 | 97.5 | 97.5 | 97.3 | 97.2 | 97.0 | 96.5 |
| 5° | C. | 12 | 97.5 | 97.5 | 97.4 | 97.2 | 97.0 | 96.5 |
| 5° | C. | 18 | 97.6 | 97.6 | 97.5 | 97.4 | 97.2 | 96.8 |
| 5° | C. | 24 | 97.3 | 97.4 | 97.3 | 97.2 | 97.0 | 96.6 |
| 5° | C. | 36 | 97.1 | 97.0 | 97.0 | 96.9 | 96.8 | 96.0 |
| 25° | C. | 1 | 97.6 | 97.6 | 97.4 | 97.2 | 97.0 | 96.4 |
| 25° | C. | 3 | 96.4 | 96.6 | 96.5 | 96.4 | 96.1 | 95.7 |
| 25° | C. | 6 | 95.1 | 95.4 | 95.7 | 95.7 | 95.5 | 95.1 |
| 25° | C. | 9 | 93.4 | 94.0 | 94.4 | 94.5 | 94.4 | 94.0 |
| 25° | C. | 12 | 92.3 | 93.1 | 93.8 | 93.9 | 93.9 | 93.5 |
| 40° | C. | 1 | 94.2 | 94.6 | 94.9 | 94.8 | 94.7 | 94.3 |
| 40° | C. | 3 | 87.6 | 89.0 | 90.3 | 90.6 | 90.8 | 90.7 |

### Results and Discussion

The monomer content measurements show that the tested formulations were stable over the tested range of pH values from pH 5.0 to 6.2. Therefore, a broad range of pH values is applicable in order to obtain high stability formulations of 150 mg/mL risankizumab. High monomer values were obtained for pH values around 5.7, whereas relatively low monomer contents were measured for more acidic conditions of about pH 5.0 (see for instance last measurement points of pH 5.0 at 25° C. or 40° C.). Hence, formulations of high concentrations of risankizumab (here 150 mg/mL) having a pH of about 5.7 proved to be particularly advantageous, in particular in the provided formulations of the present example.

### 7.3.2. Measurement of the HMW Content

Also the HMW content of the formulations was determined using UP-SEC, whereby following results were obtained:

TABLE 37
UP-SEC-HMW measurements in % of the formulations
having different pH-values.

| | Storage | | | | | | |
|---|---|---|---|---|---|---|---|
| Storage | time, | | | | | | |
| condition | months | F1 | F2 | F3 | F4 | F5 | F6 |
| initial | 0 | 0.8 | 0.9 | 1.1 | 1.2 | 1.4 | 1.9 |

| | | | | | | | | |
|---|---|---|---|---|---|---|---|---|
| 5° | C. | 3 | 1.0 | 1.1 | 1.3 | 1.4 | 1.8 | 2.4 |
| 5° | C. | 6 | 1.1 | 1.2 | 1.4 | 1.6 | 1.8 | 2.3 |
| 5° | C. | 9 | 1.3 | 1.4 | 1.6 | 1.7 | 1.9 | 2.4 |
| 5° | C. | 12 | 1.4 | 1.4 | 1.6 | 1.7 | 2.0 | 2.4 |
| 5° | C. | 18 | 1.4 | 1.5 | 1.6 | 1.7 | 1.9 | 2.3 |
| 5° | C. | 24 | 1.8 | 1.8 | 1.9 | 2.1 | 2.3 | 2.7 |
| 5° | C. | 36 | 1.8 | 1.9 | 2.0 | 2.1 | 2.3 | 2.8 |
| 25° | C. | 1 | 1.3 | 1.4 | 1.6 | 1.8 | 2.1 | 2.7 |
| 25° | C. | 3 | 1.7 | 1.8 | 2.1 | 2.3 | 2.6 | 3.0 |
| 25° | C. | 6 | 2.1 | 2.2 | 2.3 | 2.4 | 2.6 | 3.1 |
| 25° | C. | 9 | 2.6 | 2.6 | 2.9 | 2.8 | 3.0 | 3.4 |
| 25° | C. | 12 | 2.9 | 2.9 | 2.8 | 3.0 | 3.1 | 3.5 |
| 40° | C. | 1 | 2.5 | 2.5 | 2.7 | 2.9 | 3.1 | 3.6 |
| 40° | C. | 3 | 4.9 | 4.5 | 4.3 | 4.5 | 4.6 | 4.8 |

### Results and Discussion

The HMW content correlates with the monomer measurements. Overall, the tested formulations were stable over a range of pH values. Particular low increases of the HMW contents were obtained for pH values around 5.7. However, higher pH values (e.g. a pH of 6.2) appear to have led to slightly higher HMW values.

### 7.3.3. Measurement of the LMWV Content

The LMW content was measured by UP-SEC analysis, which revealed following results:

TABLE 38
UP-SEC-LMW-measurements in % of the formulations
having different pH-values.

| | Storage | | | | | | |
|---|---|---|---|---|---|---|---|
| Storage | time, | | | | | | |
| condition | months | F1 | F2 | F3 | F4 | F5 | F6 |
| initial | 0 | 0.8 | 0.8 | 0.8 | 0.8 | 0.8 | 0.8 |

| | | | | | | | | |
|---|---|---|---|---|---|---|---|---|
| 5° | C. | 3 | 0.9 | 0.8 | 0.8 | 0.8 | 0.8 | 0.8 |
| 5° | C. | 6 | 0.9 | 0.8 | 0.9 | 0.9 | 0.8 | 0.8 |
| 5° | C. | 9 | 1.2 | 1.2 | 1.1 | 1.1 | 1.1 | 1.1 |
| 5° | C. | 12 | 1.1 | 1.1 | 1.1 | 1.0 | 1.0 | 1.0 |
| 5° | C. | 18 | 1.0 | 0.9 | 0.9 | 0.9 | 0.9 | 0.9 |
| 5° | C. | 24 | 0.9 | 0.8 | 0.8 | 0.7 | 0.7 | 0.7 |
| 5° | C. | 36 | 1.1 | 1.1 | 1.0 | 1.0 | 0.9 | 1.2 |
| 25° | C. | 1 | 1.2 | 1.1 | 1.0 | 1.0 | 1.0 | 1.0 |
| 25° | C. | 3 | 1.8 | 1.6 | 1.4 | 1.4 | 1.4 | 1.3 |
| 25° | C. | 6 | 2.8 | 2.4 | 2.0 | 1.9 | 1.9 | 1.8 |
| 25° | C. | 9 | 4.1 | 3.5 | 2.7 | 2.8 | 2.7 | 2.6 |
| 25° | C. | 12 | 4.8 | 4.1 | 3.4 | 3.1 | 3.0 | 3.0 |
| 40° | C. | 1 | 3.3 | 2.9 | 2.5 | 2.3 | 2.2 | 2.1 |
| 40° | C. | 3 | 7.5 | 6.5 | 5.4 | 5.0 | 4.7 | 4.5 |

### Results and Discussion

The LMW content correlates with the monomer measurements. Particular low increases of the LMW contents were obtained for pH values around 5.7. However, lower pH values appear to have led to slightly higher LMW values. Overall, the tested formulations were stable over a range of pH values.

### 7.3.4. Measurement of Species by Ion Exchange Chromatography (IEC)

Measurement of the ionic species was performed by ionic exchange chromatography. The results were then sorted into the main peak, acidic peak groups (APG) and basic peak groups (BPG).

TABLE 39
IEC measurements of the main peak in % of the
formulations having different pH-values.

| | Storage | | | | | | |
|---|---|---|---|---|---|---|---|
| Storage | time, | | | | | | |
| condition | months | F1 | F2 | F3 | F4 | F5 | F6 |
| initial | 0 | 72 | 71 | 71 | 72 | 72 | 71 |

| | | | | | | | | |
|---|---|---|---|---|---|---|---|---|
| 5° | C. | 3 | 71 | 71 | 71 | 71 | 70 | 70 |
| 5° | C. | 6 | 70 | 70 | 70 | 70 | 70 | 70 |
| 5° | C. | 12 | 68 | 70 | 71 | 71 | 71 | 71 |
| 5° | C. | 18 | 67 | 69 | 70 | 70 | 71 | 70 |
| 5° | C. | 24 | 65 | 67 | 68 | 69 | 69 | 68 |
| 5° | C. | 36 | 65 | 66 | 68 | 69 | 69 | 69 |
| 25° | C. | 1 | 68 | 68 | 69 | 69 | 69 | 69 |
| 25° | C. | 3 | 61 | 62 | 63 | 64 | 64 | 65 |
| 25° | C. | 6 | 52 | 54 | 57 | 58 | 59 | 60 |
| 25° | C. | 12 | 39 | 43 | 48 | 50 | 51 | 53 |
| 40° | C. | 1 | 46 | 48 | 50 | 52 | 54 | 55 |
| 40° | C. | 3 | 25 | 26 | 28 | 31 | 33 | 35 |

TABLE 40
IEC measurements of APG in % of the formulations
having different pH-values.

| | Storage | | | | | | |
|---|---|---|---|---|---|---|---|
| Storage | time, | | | | | | |
| condition | months | F1 | F2 | F3 | F4 | F5 | F6 |
| initial | 0 | 22 | 22 | 22 | 22 | 22 | 22 |

| | | | | | | | | |
|---|---|---|---|---|---|---|---|---|
| 5° | C. | 3 | 22 | 22 | 23 | 23 | 23 | 23 |
| 5° | C. | 6 | 23 | 23 | 23 | 23 | 23 | 23 |
| 5° | C. | 12 | 20 | 20 | 20 | 20 | 21 | 21 |
| 5° | C. | 18 | 22 | 22 | 23 | 23 | 23 | 23 |
| 5° | C. | 24 | 20 | 21 | 21 | 21 | 22 | 22 |
| 5° | C. | 36 | 21 | 22 | 22 | 22 | 23 | 23 |
| 25° | C. | 1 | 23 | 24 | 24 | 24 | 24 | 24 |
| 25° | C. | 3 | 27 | 28 | 28 | 27 | 28 | 27 |
| 25° | C. | 6 | 32 | 33 | 33 | 33 | 32 | 32 |
| 25° | C. | 12 | 34 | 37 | 39 | 39 | 38 | 38 |
| 40° | C. | 1 | 37 | 38 | 39 | 38 | 37 | 37 |
| 40° | C. | 3 | 55 | 59 | 61 | 60 | 59 | 58 |

TABLE 41
IEC measurements of BPG in % of the formulations
having different pH-values.

| | Storage | | | | | | |
|---|---|---|---|---|---|---|---|
| Storage | time, | | | | | | |
| condition | months | F1 | F2 | F3 | F4 | F5 | F6 |
| initial | 0 | 6 | 6 | 7 | 6 | 7 | 7 |

| | | | | | | | | |
|---|---|---|---|---|---|---|---|---|
| 5° | C. | 3 | 7 | 7 | 7 | 7 | 7 | 7 |
| 5° | C. | 6 | 7 | 7 | 7 | 7 | 7 | 7 |
| 5° | C. | 12 | 12 | 10 | 9 | 9 | 8 | 8 |
| 5° | C. | 18 | 11 | 9 | 8 | 7 | 7 | 6 |
| 5° | C. | 24 | 15 | 13 | 11 | 10 | 9 | 9 |
| 5° | C. | 36 | 15 | 12 | 10 | 9 | 8 | 8 |
| 25° | C. | 1 | 9 | 8 | 8 | 7 | 8 | 8 |
| 25° | C. | 3 | 12 | 11 | 9 | 9 | 8 | 8 |
| 25° | C. | 6 | 16 | 13 | 10 | 9 | 9 | 8 |
| 25° | C. | 12 | 27 | 20 | 14 | 11 | 10 | 9 |
| 40° | C. | 1 | 17 | 14 | 11 | 10 | 9 | 9 |
| 40° | C. | 3 | 20 | 15 | 10 | 9 | 8 | 7 |

### Results and Discussion

The IEC measurements show overall that the tested formulations were stable. All pH values led to high contents of the main peak. Noteworthy, an intermediate pH value of 5.7 and pH values around 5.7 showed a good compromise in comparison to the highest and lowest tested pH values, which showed an increase in APG or BPG species, respectively. Therefore, a pH of approximately 5.7 proved to be advantageous.

### 7.3.5. Measurement of Species by Hydrophobic Interaction Chromatography (HIC)

Measurement of the variants/subspecies of risankizumab was performed by hydrophobic interaction chromatography (HIC). The results were then sorted into the main peak, pre-peaks and post-peaks.

TABLE 42
HIC measurements of the main peak in % of the
formulations having different pH-values.

| | | Storage | | | | | | |
|---|---|---|---|---|---|---|---|---|
| Storage | | time, | | | | | | |
| condition | | months | F1 | F2 | F3 | F4 | F5 | F6 |
| initial | | 0 | 96.8 | 96.7 | 96.7 | 96.7 | 96.4 | 96.0 |
| 5° | C. | 3 | 96.7 | 96.7 | 96.7 | 96.6 | 96.3 | 95.8 |
| 5° | C. | 6 | 96.7 | 96.7 | 96.7 | 96.5 | 96.5 | 95.9 |
| 5° | C. | 12 | 97.2 | 97.2 | 97.1 | 97.1 | 96.9 | 96.6 |
| 5° | C. | 18 | 96.0 | 96.1 | 96.2 | 96.1 | 95.9 | 95.6 |
| 5° | C. | 24 | 96.7 | 96.7 | 96.9 | 96.8 | 96.7 | 96.5 |
| 5° | C. | 36 | 95.3 | 95.4 | 95.7 | 95.7 | 95.7 | 95.4 |
| 25° | C. | 1 | 96.2 | 96.1 | 96.1 | 95.9 | 95.7 | 95.3 |
| 25° | C. | 3 | 95.4 | 95.3 | 95.4 | 95.4 | 95.1 | 94.7 |
| 25° | C. | 6 | 94.7 | 95.0 | 95.1 | 95.1 | 95.1 | 94.8 |
| 25° | C. | 12 | 93.8 | 94.1 | 94.6 | 94.7 | 94.7 | 94.4 |
| 40° | C. | 1 | 93.6 | 94.2 | 94.3 | 94.2 | 94.2 | 93.6 |
| 40° | C. | 3 | 88.5 | 89.8 | 90.4 | 90.8 | 90.7 | 90.7 |

TABLE 43
HIC measurements of pre-peaks in % of the
formulations having different pH-values.

| | Storage | | | | | | |
|---|---|---|---|---|---|---|---|
| Storage | time, | | | | | | |
| condition | months | F1 | F2 | F3 | F4 | F5 | F6 |
| initial | 0 | 1.8 | 1.7 | 1.7 | 1.6 | 1.6 | 1.6 |
| 5º C. | 3 | 1.9 | 1.9 | 1.8 | 1.8 | 1.8 | 1.8 |
| 5º C. | 6 | 1.9 | 1.9 | 1.7 | 1.8 | 1.6 | 1.7 |
| 5º C. | 12 | 1.6 | 1.5 | 1.4 | 1.3 | 1.3 | 1.3 |
| 5º C. | 18 | 2.1 | 2.0 | 1.8 | 1.8 | 1.7 | 1.6 |
| 5º C. | 24 | 1.8 | 1.7 | 1.5 | 1.4 | 1.4 | 1.4 |
| 5º C. | 36 | 2.2 | 2.0 | 1.7 | 1.5 | 1.5 | 1.4 |
| 25° C. | 1 | 2.1 | 2.1 | 2.0 | 1.9 | 2.0 | 1.9 |
| 25° C. | 3 | 2.8 | 2.7 | 2.4 | 2.3 | 2.3 | 2.5 |
| 25° C. | 6 | 3.3 | 3.0 | 2.7 | 2.5 | 2.4 | 2.3 |
| 25° C. | 12 | 4.1 | 3.6 | 3.1 | 2.8 | 2.7 | 2.6 |
| 40° C. | 1 | 3.7 | 3.3 | 3.1 | 3.0 | 2.8 | 3.0 |
| 40° C. | 3 | 7.5 | 6.4 | 6.1 | 5.6 | 5.5 | 5.4 |

TABLE 44
HIC measurements of post-peaks in % of the
formulations having different pH-values.

| | Storage | | | | | | |
|---|---|---|---|---|---|---|---|
| Storage | time, | | | | | | |
| condition | months | F1 | F2 | F3 | F4 | F5 | F6 |
| initial | 0 | 1.5 | 1.6 | 1.6 | 1.7 | 2.0 | 2.4 |
| 5º C. | 3 | 1.4 | 1.4 | 1.6 | 1.7 | 1.9 | 2.5 |
| 5º C. | 6 | 1.4 | 1.4 | 1.6 | 1.7 | 1.9 | 2.3 |
| 5º C. | 12 | 1.2 | 1.2 | 1.4 | 1.6 | 1.7 | 2.1 |
| 5º C. | 18 | 1.9 | 1.9 | 2.0 | 2.1 | 2.4 | 2.8 |
| 5º C. | 24 | 1.5 | 1.6 | 1.6 | 1.7 | 1.9 | 2.1 |
| 5º C. | 36 | 2.5 | 2.6 | 2.6 | 2.8 | 2.8 | 3.2 |
| 25° C. | 1 | 1.8 | 1.8 | 2.0 | 2.2 | 2.3 | 2.9 |
| 25° C. | 3 | 1.8 | 1.9 | 2.2 | 2.2 | 2.6 | 2.9 |
| 25° C. | 6 | 2.1 | 2.1 | 2.3 | 2.4 | 2.5 | 2.9 |
| 25° C. | 12 | 2.0 | 2.1 | 2.2 | 2.4 | 2.5 | 2.9 |
| 40° C. | 1 | 2.7 | 2.6 | 2.6 | 2.8 | 2.9 | 3.5 |
| 40° C. | 3 | 4.1 | 3.8 | 3.5 | 3.6 | 3.8 | 3.9 |

### Results and Discussion

The HIC measurements show overall that the tested formulations were stable. All pH values led to high contents of the main peak. Noteworthy, an intermediate pH value of 5.7 and pH values around 5.7 showed a good compromise in comparison to the highest and lowest tested pH values, which showed an increase in pre- and post-peaks, respectively.

### 7.3.6. Binding Activity

Measurement of the binding activity of risankizumab against IL-23 was performed using the Biacore T200. Following results were obtained:

TABLE 45
Binding activity in % of the
formulations having different pH-values.

| | Storage | | | | | | |
|---|---|---|---|---|---|---|---|
| Storage | time, | | | | | | |
| condition | months | F1 | F2 | F3 | F4 | F5 | F6 |
| initial | 0 | 103 | 103 | 103 | 106 | 105 | 107 |
| 5º C. | 6 | 101 | 104 | 102 | 103 | 104 | 103 |
| 5º C. | 12 | 94 | 97 | 96 | 93 | 97 | 99 |
| 5º C. | 18 | 104 | 102 | 102 | 106 | 105 | 105 |
| 5º C. | 24 | 102 | 96 | 101 | 100 | 97 | 102 |
| 5º C. | 36 | 95 | 97 | 96 | 95 | 96 | 98 |
| 25° C. | 3 | 109 | 107 | 106 | 106 | 103 | 103 |
| 25° C. | 6 | 105 | 99 | 99 | 100 | 103 | 103 |
| 25° C. | 12 | 93 | 101 | 93 | 94 | 92 | 97 |
| 40° C. | 3 | 98 | 100 | 100 | 98 | 97 | 101 |

TABLE 46
Specific binding activity in % of the
formulations having different pH-values.

| | Storage | | | | | | |
|---|---|---|---|---|---|---|---|
| Storage | time, | | | | | | |
| condition | months | F1 | F2 | F3 | F4 | F5 | F6 |
| initial | 0 | 100 | 100 | 100 | 100 | 100 | 100 |
| 5º C. | 6 | 100 | 99 | 99 | 99 | 99 | 99 |
| 5º C. | 12 | 99 | 100 | 100 | 100 | 100 | 100 |
| 5º C. | 18 | 99 | 99 | 99 | 100 | 100 | 100 |
| 5º C. | 24 | 99 | 99 | 99 | 99 | 99 | 99 |
| 5º C. | 36 | 100 | 100 | 100 | 100 | 100 | 100 |
| 25° C. | 3 | 99 | 99 | 99 | 99 | 99 | 99 |
| 25° C. | 6 | 98 | 98 | 99 | 98 | 99 | 98 |
| 25° C. | 12 | 98 | 98 | 98 | 98 | 98 | 98 |
| 40° C. | 3 | 96 | 96 | 97 | 97 | 98 | 98 |

### Results and Discussion

The binding activity measurement show overall high values of the tested formulations. Hence, the tested formulations stabilize risankizumab such that a high binding activity at a pH range from 5.0 to 6.2 was achieved.

### 7.3.7. Measurement of the Opalescence

Furthermore, the opalescence of the formulations was measured. The opalescence slightly changes over the time of analysis but remained overall highly constant ranging from 3 to 11. The results indicate that the formulations are overall stable. Noteworthy, lower pH values in general showed lower opalescence (3 to 6 FNU for pH 5.0) than higher pH values (7 to 11 FNU for pH 6.2). The intermediate pH of 5.7 had a opalescence ranging from 5 to 7 FNU, indicating that providing formulations having a pH of about 5.7 is advantageous, in particular in formulations according to the present example.

### 7.3.8. Measurement of the Viscosity and Syringe Gliding and Break Loose Forces

As further parameters, the viscosity and the syringe forces were measured, which include the average and maximal gliding force, as well as the break loose force. Following results were obtained:

TABLE 47
Measurement of the viscosity in mPas of the tested
formulations having different pH-values over time.

| | Storage | | | | | | |
|---|---|---|---|---|---|---|---|
| Storage | time, | | | | | | |
| condition | months | F1 | F2 | F3 | F4 | F5 | F6 |
| initial | 0 | 8.5 | 8.8 | 9.2 | 9.6 | 9.9 | 10.3 |
| 5º C. | 6 | 8.5 | 8.6 | 9.0 | 9.2 | 9.5 | 10.0 |
| 5º C. | 12 | 8.7 | 8.7 | 8.9 | 9.3 | 9.7 | 10.1 |
| 5º C. | 24 | 8.8 | 9.0 | 9.3 | 9.5 | 9.4 | 10.4 |
| 5º C. | 36 | 8.6 | 8.7 | 8.9 | 9.4 | 9.8 | 10.3 |
| 25° C. | 6 | 8.7 | 8.8 | 8.9 | 9.2 | 9.5 | 10.0 |
| 25° C. | 12 | 8.7 | 8.5 | 9.0 | 9.3 | 9.7 | 10.3 |

TABLE 48
Measurement of the maximal gliding force in N of the
tested formulations having different pH-values over time.

| | Storage | | | | | | |
|---|---|---|---|---|---|---|---|
| Storage | time, | | | | | | |
| condition | months | F1 | F2 | F3 | F4 | F5 | F6 |
| initial | 0 | 6.3 | 5.9 | 6.0 | 6.6 | 6.0 | 7.1 |
| 5º C. | 3 | 6.4 | 6.9 | 6.3 | 6.5 | 6.5 | 7.0 |
| 5º C. | 6 | 8.4 | 7.1 | 6.3 | 6.3 | 6.2 | 6.7 |
| 5º C. | 9 | 7.7 | 8.5 | 6.4 | 6.3 | 7.1 | 6.9 |
| 5º C. | 12 | 9.4 | 7.6 | 6.6 | 6.9 | 7.3 | 6.8 |
| 5º C. | 18 | 9.2 | 10.4 | 8.4 | 6.5 | 6.9 | 7.0 |
| 5º C. | 24 | 9.0 | 8.2 | 7.7 | 7.7 | 6.9 | 6.6 |
| 5º C. | 36 | 9.2 | 8.3 | 7.7 | 7.7 | 6.9 | 7.1 |
| 25° C. | 1 | 7.0 | 8.6 | 7.3 | 6.4 | 6.0 | 5.6 |
| 25° C. | 3 | 11.8 | 10.0 | 8.5 | 7.3 | 8.3 | 6.8 |
| 25° C. | 6 | 12.4 | 11.9 | 11.0 | 11.2 | 8.4 | 7.2 |
| 25° C. | 9 | 12.9 | 13.3 | 13.2 | 10.7 | 11.1 | 8.4 |
| 25° C. | 12 | 13.6 | 16.7 | 13.7 | 11.9 | 12.5 | 10.2 |
| 40° C. | 1 | 10.8 | 10.3 | 11.8 | 8.7 | 7.9 | 7.2 |
| 40° C. | 3 | 18.1 | 20.9 | 20.6 | 18.0 | 17.1 | 13.4 |

TABLE 49
Measurement of the average gliding force in N of the
tested formulations having different pH-values over time.

| Storage | Storage | | | | | | |
|---|---|---|---|---|---|---|---|
| con- | time, | | | | | | |
| dition | months | F1 | F2 | F3 | F4 | F5 | F6 |
| initial | 0 | 5.9 | 5.6 | 5.6 | 6.1 | 5.7 | 6.5 |
| 5º C. | 3 | 5.9 | 6.3 | 5.8 | 6.0 | 6.1 | 6.5 |
| 5º C. | 6 | 7.4 | 6.5 | 5.9 | 5.9 | 5.9 | 6.4 |
| 5º C. | 9 | 6.9 | 7.7 | 6.0 | 5.9 | 6.6 | 6.5 |
| 5º C. | 12 | 8.1 | 6.8 | 6.2 | 6.5 | 6.8 | 6.4 |
| 5º C. | 18 | 8.1 | 9.2 | 7.6 | 6.1 | 6.5 | 6.6 |
| 5º C. | 24 | 7.9 | 7.2 | 7.0 | 7.1 | 6.4 | 6.2 |
| 5º C. | 36 | 8.0 | 7.6 | 7.1 | 7.1 | 6.5 | 6.7 |
| 25° C. | 1 | 6.5 | 7.5 | 6.6 | 6.0 | 5.7 | 5.4 |
| 25° C. | 3 | 9.8 | 8.8 | 7.7 | 6.9 | 7.6 | 6.4 |
| 25° C. | 6 | 10.1 | 9.7 | 9.0 | 9.6 | 7.6 | 6.8 |
| 25° C. | 9 | 10.6 | 11.1 | 10.8 | 8.6 | 9.3 | 7.6 |
| 25° C. | 12 | 10.8 | 13.5 | 10.8 | 9.7 | 9.9 | 8.6 |
| 40° C. | 1 | 9.2 | 8.5 | 9.6 | 7.5 | 7.1 | 6.6 |
| 40° C. | 3 | 14.1 | 15.8 | 14.5 | 12.9 | 12.5 | 10.5 |

TABLE 50
Measurement of the break loose force in N of the
tested formulations having different pH-values over time.

| | Storage | | | | | | |
|---|---|---|---|---|---|---|---|
| Storage | time, | | | | | | |
| condition | months | F1 | F2 | F3 | F4 | F5 | F6 |
| initial | 0 | 3.9 | 3.9 | 4.1 | 4.1 | 4.1 | 4.1 |
| 5º C. | 3 | 4.9 | 4.9 | 5.2 | 5.1 | 5.1 | 5.0 |
| 5º C. | 6 | 4.4 | 4.4 | 4.5 | 4.6 | 4.6 | 4.6 |
| 5º C. | 9 | 4.0 | 4.0 | 4.0 | 4.2 | 4.1 | 4.1 |
| 5º C. | 12 | 4.7 | 4.6 | 4.6 | 4.6 | 4.7 | 4.7 |
| 5º C. | 18 | 4.7 | 4.6 | 4.7 | 4.8 | 4.8 | 4.9 |
| 5º C. | 24 | 4.6 | 4.4 | 4.6 | 4.6 | 4.6 | 4.9 |
| 5º C. | 36 | 4.5 | 4.5 | 4.7 | 4.7 | 4.7 | 4.6 |
| 25° C. | 1 | 4.4 | 4.6 | 4.7 | 5.0 | 4.6 | 4.4 |
| 25° C. | 3 | 5.5 | 5.5 | 5.7 | 5.7 | 5.7 | 5.5 |
| 25° C. | 6 | 5.4 | 5.3 | 5.2 | 5.4 | 5.2 | 5.4 |
| 25° C. | 9 | 5.4 | 5.2 | 5.3 | 5.0 | 5.0 | 5.0 |
| 25° C. | 12 | 5.4 | 5.3 | 5.3 | 5.3 | 5.3 | 5.4 |
| 40° C. | 1 | 5.4 | 5.3 | 5.4 | 5.5 | 5.4 | 5.3 |
| 40° C. | 3 | 7.0 | 6.7 | 6.7 | 6.2 | 6.5 | 6.1 |

### Results and Discussion

The viscosity measurements revealed a slightly higher viscosity for higher pH values. Thus lower pH values, such as pH 5.7, may be found advantageous in order to obtain formulations having a lower viscosity. Note, the mechanical measurements of the gliding and break loose force revealed overall very similar performance.

### 7.4. Further Analytics and Results

In addition, further analytics were performed for the six formulations tested with the following results. Storage times and temperatures were as described above.
The protein concentration remained essentially constant over the storage time of 36, 24, 12 and 3 months at the different storage temperatures tested, respectively 5° C., 25° C. and 40° C. Small deviations of protein concentration (145-155 mg/mL (24 months) and 145-158 mg/mL (36 months)) are due to analytical variations.
The pH value remained essentially constant over the storage time of 36, 24, 12 and 3 months at the different storage temperatures tested, respectively 5° C., 25° C. and 40° C. Measured pH values were thus in a range from 4.9-6.3.
The osmolality remained essentially constant over the storage time of 36, 24, 12 and 3 months at the different storage temperatures tested, respectively 5° C., 25° C. and 40° C. The tested values ranged from 301-323 mOsm/kg.
The protein related particles and foreign particles remained essentially constantly low over the storage time at the different storage temperatures tested.

### 7.5. Summary of Results

The formulations were stable at all tested pH values over long storage times of up to 24 and 36 months. While temperatures appeared to have an impact on the stability (i.e. higher temperatures inducing more instability related effects), all formulations led to sufficient stability even at high temperatures.

In summary, a pH value of 5.7 and values around 5.7 (e.g. 5.5, 5.9) appeared under the used test conditions to result in an advantageous compromise regarding the storage parameters. For instance, the UP-SEC measurements showed intermediate to low HMW and LMW values for pH 5.7, while the highest and lowest pH each showed highest HMW and LMW contents, respectively. Similar results were obtained in the IEC and HIC measurements.

### 8. Impact of the Acetate Concentration on the Formulation Stability

Formulations containing different concentrations of acetate (see Table 51) were stored over different time points at three different temperatures (5° C., 25° C. and 40° C.).

TABLE 51
Composition of formulations.

| Formulation | Acetate/mM | PS20 | Trehalose | pH |
|---|---|---|---|---|
| F1 | 0 | 0.2 mg/mL | 185 mM | 5.7 |
| F2 | 5 | | | |
| F3 | 10 | | | |
| F4 | 15 | | | |
| F5 | 20 | | | |

### 8.1. Preparation of Formulations

The formulations were prepared as described above.

### 8.2. Analytics

Measurements of the samples were performed at 1, 3, 6, 9, 12, 18, 24 and 36 months storage, as well as initially before storage. The storage temperatures were adjusted to 5° C., 25° C. or 40° C. Analytics were performed by measurement using an UP-SEC for monomer, HMW and LMW content and Biacore for the binding activity. Furthermore, the required forces for gliding and break loose force were measured, as well as the osmolality, opalescence and the pH value. Further details on the utilized analysis methods are described below.

### 8.3. Results

### 8.3.1. Measurement of the Monomer Content

UP-SEC analysis was performed to measure the monomer content. Following results were obtained.

TABLE 52
UP-SEC-Monomer-measurements in % of formulations
comprising varying amounts of acetate.

| | Storage | | | | | |
|---|---|---|---|---|---|---|
| Storage | time, | | | | | |
| condition | months | F1 | F2 | F3 | F4 | F5 |
| 5º C. | 0 | 97.9 | 97.9 | 97.9 | 98.0 | 98.0 |
| 5º C. | 3 | 97.6 | 97.6 | 97.7 | 97.7 | 97.7 |
| 5º C. | 6 | 97.5 | 97.5 | 97.6 | 97.6 | 97.5 |
| 5º C. | 9 | 97.2 | — | 97.3 | — | 97.3 |
| 5º C. | 12 | 97.2 | 97.2 | 97.3 | 97.3 | 97.3 |
| 5º C. | 18 | 97.4 | 97.4 | 97.4 | 97.4 | 97.5 |
| 5º C. | 24 | 97.2 | 97.2 | 97.3 | 97.2 | 97.3 |
| 5º C. | 36 | 97.0 | 97.0 | 97.1 | 97.1 | 97.0 |
| 25° C. | 0 | 97.9 | 97.9 | 97.9 | 98.0 | 98.0 |
| 25° C. | 1 | 97.3 | — | 97.3 | — | 97.4 |
| 25° C. | 3 | 96.4 | 96.4 | 96.4 | 96.4 | 96.4 |
| 25° C. | 6 | 95.7 | 95.7 | 95.7 | 95.7 | 95.6 |
| 25° C. | 9 | 94.7 | — | 94.6 | — | 94.6 |
| 25° C. | 12 | 94.1 | 94.1 | 94.1 | 94.1 | 94.1 |
| 40° C. | 0 | 97.9 | 97.9 | 97.9 | 98.0 | 98.0 |
| 40° C. | 1 | 95.2 | — | 95.2 | — | 95.2 |
| 40° C. | 3 | 90.9 | 90.8 | 90.8 | 90.7 | 90.8 |

### Results and Discussion

The monomer measurements show that the formulation is stable over a range of acetate contents, indicating stability for buffer containing and buffer-free formulations with 150 mg/mL risankizumab and formulations according to the present example.

### 8.3.2. Measurement of the HMW Content

Also the HMW content of the formulations was determined via UP-SEC analysis with following results:

TABLE 53
UP-SEC-HMW measurements in % of formulations
comprising varying amounts of acetate.

| | Storage | | | | | |
|---|---|---|---|---|---|---|
| Storage | time, | | | | | |
| condition | months | F1 | F2 | F3 | F4 | F5 |
| 5º C. | 0 | 1.3 | 1.3 | 1.3 | 1.3 | 1.2 |
| 5º C. | 3 | 1.5 | 1.5 | 1.5 | 1.5 | 1.5 |
| 5º C. | 6 | 1.7 | 1.6 | 1.6 | 1.6 | 1.6 |
| 5º C. | 9 | 1.7 | — | 1.6 | — | 1.6 |
| 5º C. | 12 | 1.9 | 1.8 | 1.8 | 1.7 | 1.7 |
| 5º C. | 18 | 1.7 | 1.7 | 1.7 | 1.7 | 1.6 |
| 5º C. | 24 | 2.0 | 2.0 | 2.0 | 2.0 | 2.0 |
| 5º C. | 36 | 2.2 | 2.1 | 2.1 | 2.1 | 2.1 |
| 25° C. | 0 | 1.3 | 1.3 | 1.3 | 1.3 | 1.2 |
| 25° C. | 1 | 1.7 | — | 1.7 | — | 1.7 |
| 25° C. | 3 | 2.3 | 2.2 | 2.2 | 2.2 | 2.2 |
| 25° C. | 6 | 2.5 | 2.5 | 2.4 | 2.5 | 2.5 |
| 25° C. | 9 | 2.7 | — | 2.7 | — | 2.7 |
| 25° C. | 12 | 2.9 | 2.9 | 2.9 | 2.9 | 2.9 |
| 40° C. | 0 | 1.3 | 1.3 | 1.3 | 1.3 | 1.2 |
| 40° C. | 1 | 2.7 | — | 2.6 | — | 2.7 |
| 40° C. | 3 | 4.3 | 4.4 | 4.4 | 4.4 | 4.4 |

### Results and Discussion

The HMW content measurements show that the formulation is stable over a range of acetate contents, indicating stability for buffer containing and buffer-free formulations with 150 mg/mL risankizumab and formulations according to the present example.

### 8.3.3. Measurement of the LMW Content

For LMWV content measurements UP-SEC analysis was performed. Following results were obtained.

TABLE 54
UP-SEC-LMW-measurements in % of formulations
comprising varying amounts of acetate.

| | Storage | | | | | |
|---|---|---|---|---|---|---|
| Storage | time, | | | | | |
| condition | months | F1 | F2 | F3 | F4 | F5 |
| 5º C. | 0 | 0.8 | 0.9 | 0.9 | 0.8 | 0.8 |
| 5º C. | 3 | 0.9 | 0.9 | 0.9 | 0.8 | 0.8 |
| 5º C. | 6 | 0.8 | 0.9 | 0.9 | 0.9 | 0.9 |
| 5º C. | 9 | 1.1 | — | 1.1 | — | 1.1 |
| 5º C. | 12 | 1.0 | 1.0 | 1.0 | 1.0 | 1.0 |
| 5º C. | 18 | 0.9 | 0.9 | 0.9 | 0.9 | 0.9 |
| 5º C. | 24 | 0.8 | 0.8 | 0.8 | 0.8 | 0.8 |
| 5º C. | 36 | 0.9 | 0.9 | 0.8 | 0.9 | 0.9 |
| 25° C. | 0 | 0.8 | 0.9 | 0.9 | 0.8 | 0.8 |
| 25° C. | 1 | 1.0 | — | 1.0 | — | 0.9 |
| 25° C. | 3 | 1.4 | 1.4 | 1.4 | 1.4 | 1.4 |
| 25° C. | 6 | 1.8 | 1.8 | 1.9 | 1.8 | 1.9 |
| 25° C. | 9 | 2.7 | — | 2.7 | — | 2.7 |
| 25° C. | 12 | 3.0 | 3.0 | 3.0 | 3.0 | 3.0 |
| 40° C. | 0 | 0.8 | 0.9 | 0.9 | 0.8 | 0.8 |
| 40° C. | 1 | 2.2 | — | 2.2 | — | 2.2 |
| 40° C. | 3 | 4.8 | 4.8 | 4.8 | 4.8 | 4.9 |

### Results and Discussion

The LMW measurements show that the formulation is stable over a range of acetate contents, indicating stability for buffer containing and buffer-free formulations.

### 8.3.4. Measurement of Binding Activity

In order to analyze whether the acetate content has an influence on the binding activity of risankizumab to IL-23, Biacore analysis was performed. The measurements of the binding activity show high binding activity against human IL-23 for all tested formulations and storage times ranging from 92-105% binding activity and 97-100% specific binding activity. These results support the advantageous stability of the tested formulations and indicate that acetate containing and buffer-free formulations are applicable according to the present disclosure.

### 8.3.5. Measurement of Osmolality

As the acetate content also has an influence on the osmolality of the formulation, this parameter was measured. The results are shown in Table 55.

TABLE 55
Osmolality in mOsm/kg of the formulations at different temperatures and
storage times of formulations comprising varying amounts of acetate.

| | Storage | | | | | |
|---|---|---|---|---|---|---|
| Storage | time, | | | | | |
| condition | months | F1 | F2 | F3 | F4 | F5 |
| 5º C. | 0 | 288 | 293 | 309 | 322 | 335 |
| 5º C. | 3 | 290 | 294 | 310 | 324 | 335 |
| 5º C. | 6 | 291 | 296 | 311 | 322 | 334 |
| 5º C. | 9 | 304 | — | 309 | — | 333 |
| 5º C. | 12 | 292 | 293 | 305 | 320 | 333 |
| 5º C. | 18 | 284 | 287 | 305 | 322 | 330 |
| 5º C. | 24 | 290 | 290 | 306 | 322 | 333 |
| 5º C. | 36 | 288 | 291 | 306 | 322 | 334 |
| 25° C. | 0 | 288 | 293 | 309 | 322 | 335 |
| 25° C. | 1 | 290 | — | 309 | — | 335 |
| 25° C. | 3 | 288 | 293 | 311 | 322 | 334 |
| 25° C. | 6 | 290 | 295 | 313 | 324 | 334 |
| 25° C. | 9 | 295 | — | 311 | — | 333 |
| 25° C. | 12 | 294 | 294 | 309 | 312 | 333 |
| 40° C. | 0 | 288 | 293 | 309 | 322 | 335 |
| 40° C. | 1 | 287 | — | 308 | — | 334 |
| 40° C. | 3 | 291 | 297 | 314 | 324 | 338 |

### Results and Discussion

The measurements show that the osmolality varies from about 290 to 338 mOsm/kg depending on the amount of added acetate. The more acetate was added, the higher the measured osmolality. An osmolality of approximately 310 mOsm/kg is typically desired and an acetate concentration of 10 mM resulted in the tested formulations in a desired osmolality of around 310 mOsm/kg (measured range from 305 to 314 mOsm/kg). In case another concentration of acetate is required or desired, it may be advantageous to modify the content of the other compounds of the formulation (e.g., another excipient such as trehalose) to adjust the osmolality to approximately 310 mOsm/kg.

### 8.3.6. Measurement of Opalescence

The opalescence of the formulations of the present example was also measured to evaluate the stability. The measured opalescence is overall the same for the different formulations ranging between 4-9 FNU. Higher concentrations of acetate led to a slightly higher opalescence of 7-9 FNU for 20 mM acetate than lower concentrations (4-6 FNU for 0 mM acetate). All formulations according to the present example were stable in view of the measured opalescence.

### 8.3.7. Measurement of pH

In order to determine the pH stability of the formulations comprising varying amounts of buffer, i.e. acetate in this example, the pH values were measured over the time of storage at different temperatures. The results are shown in the subsequent table.

TABLE 56
Measured pH value for the formulations containing varying
amounts of acetate at 5, 25 or 40° C. over varying storage times.

| | Storage | | | | | |
|---|---|---|---|---|---|---|
| Storage | time, | | | | | |
| condition | months | F1 | F2 | F3 | F4 | F5 |
| 5º C. | 0 | 5.8 | 5.8 | 5.8 | 5.8 | 5.8 |
| 5º C. | 3 | 5.8 | 5.8 | 5.7 | 5.8 | 5.8 |
| 5º C. | 6 | 5.8 | 5.8 | 5.8 | 5.8 | 5.8 |
| 5º C. | 9 | 5.8 | — | 5.8 | — | 5.8 |
| 5º C. | 12 | 5.7 | 5.8 | 5.8 | 5.7 | 5.8 |
| 5º C. | 18 | 5.7 | 5.8 | 5.8 | 5.8 | 5.7 |
| 5º C. | 24 | 5.8 | 5.8 | 5.8 | 5.8 | 5.8 |
| 5º C. | 36 | 5.8 | 5.8 | 5.8 | 5.8 | 5.8 |
| 25° C. | 0 | 5.8 | 5.8 | 5.8 | 5.8 | 5.8 |
| 25° C. | 1 | 5.8 | — | 5.7 | — | 5.7 |
| 25° C. | 3 | 5.8 | 5.7 | 5.8 | 5.8 | 5.7 |
| 25° C. | 6 | 5.8 | 5.8 | 5.8 | 5.8 | 5.8 |
| 25° C. | 9 | 5.8 | — | 5.8 | — | 5.8 |
| 25° C. | 12 | 5.7 | 5.8 | 5.7 | 5.7 | 5.8 |
| 40° C. | 0 | 5.8 | 5.8 | 5.8 | 5.8 | 5.8 |
| 40° C. | 1 | 5.8 | — | 5.8 | — | 5.7 |
| 40° C. | 3 | 5.7 | 5.7 | 5.7 | 5.8 | 5.7 |

### Results and Discussion

The measurements of the pH value demonstrate that the pH is overall kept constant for the tested formulations according to the present example. Therefore, for all acetate contents, including a formulation comprising no acetate, the formulations were stable regarding the pH value.

### 8.3.8. Measurement of Gliding and Break Loose Forces

The maximal and average gliding force were measured as well as the break loose force for syringes containing the different formulations according to the present example. The results of these measurements are shown below.

TABLE 57
Maximum gliding force in N, initially and after the indicated storage time at
5° C., 25° C. or 40° C. of formulations comprising varying amounts of acetate.

| | Storage | | | | | |
|---|---|---|---|---|---|---|
| Storage | time, | | | | | |
| condition | months | F1 | F2 | F3 | F4 | F5 |
| 5º C. | 0 | 6.9 | 6.3 | 6.6 | 7.1 | 6.7 |
| 5º C. | 3 | 7.3 | 7.0 | 6.3 | 7.4 | 7.6 |
| 5º C. | 6 | 7.5 | 6.9 | 6.6 | 7.0 | 6.4 |
| 5º C. | 9 | 8.1 | — | 7.1 | — | 6.6 |
| 5º C. | 12 | 7.5 | 7.0 | 7.1 | 6.7 | 7.1 |
| 5º C. | 18 | 7.9 | 8.8 | 6.1 | 5.9 | 7.0 |
| 5º C. | 24 | 8.8 | 7.7 | 7.1 | 7.4 | 5.9 |
| 5º C. | 36 | 7.8 | 9.3 | 7.0 | 7.4 | 7.1 |
| 25° C. | 0 | 6.9 | 6.3 | 6.6 | 7.1 | 6.7 |
| 25° C. | 1 | 8.1 | — | 5.9 | — | 5.9 |
| 25° C. | 3 | 9.9 | 8.2 | 6.9 | 7.8 | 7.3 |
| 25° C. | 6 | 12.5 | 12.5 | 8.5 | 8.2 | 7.1 |
| 25° C. | 9 | 13.9 | — | 11.8 | — | 10.5 |
| 25° C. | 12 | 18.4 | 14.7 | 14.5 | 13.1 | 10.8 |
| 40° C. | 0 | 6.9 | 6.3 | 6.6 | 7.1 | 6.7 |
| 40° C. | 1 | 11.8 | — | 11.5 | — | 7.6 |
| 40° C. | 3 | 25.1 | 24.0 | 16.4 | 15.6 | 14.2 |

TABLE 58
Average gliding force in N, initially and after the indicated storage time at
5° C., 25° C. or 40° C. of formulations comprising varying amounts of acetate.

| | Storage | | | | | |
|---|---|---|---|---|---|---|
| Storage | time, | | | | | |
| condition | months | F1 | F2 | F3 | F4 | F5 |
| 5º C. | 0 | 6.3 | 6.0 | 6.2 | 6.6 | 6.2 |
| 5º C. | 3 | 6.5 | 6.4 | 6.0 | 6.8 | 6.9 |
| 5º C. | 6 | 7.0 | 6.5 | 6.2 | 6.6 | 6.1 |
| 5º C. | 9 | 7.2 | — | 6.6 | — | 6.2 |
| 5º C. | 12 | 6.9 | 6.5 | 6.6 | 6.3 | 6.7 |
| 5º C. | 18 | 7.2 | 7.9 | 5.8 | 5.7 | 6.6 |
| 5º C. | 24 | 8.2 | 7.2 | 6.6 | 7.0 | 5.6 |
| 5º C. | 36 | 7.0 | 8.1 | 6.6 | 6.8 | 6.7 |
| 25° C. | 0 | 6.3 | 6.0 | 6.2 | 6.6 | 6.2 |
| 25° C. | 1 | 7.3 | — | 5.5 | — | 5.7 |
| 25° C. | 3 | 8.2 | 7.5 | 6.5 | 6.9 | 6.7 |
| 25° C. | 6 | 10.5 | 10.5 | 7.6 | 7.6 | 6.7 |
| 25° C. | 9 | 11.1 | — | 9.5 | — | 9.1 |
| 25° C. | 12 | 14.0 | 11.9 | 11.5 | 10.2 | 8.9 |
| 40° C. | 0 | 6.3 | 6.0 | 6.2 | 6.6 | 6.2 |
| 40° C. | 1 | 9.5 | — | 8.8 | — | 6.8 |
| 40° C. | 3 | 17.2 | 16.2 | 11.9 | 12.0 | 10.4 |

TABLE 59
Break loose force in N, initially and after the indicated
storage time at 5° C., 25° C. or 40°
C. of formulations comprising varying amounts of acetate.

| | Storage | | | | | |
|---|---|---|---|---|---|---|
| Storage | time, | | | | | |
| condition | months | F1 | F2 | F3 | F4 | F5 |
| 5° C. | 0 | 4.1 | 4.0 | 3.9 | 3.8 | 4.1 |
| 5° C. | 3 | 4.5 | 4.6 | 4.3 | 4.4 | 4.4 |
| 5° C. | 6 | 4.5 | 4.3 | 4.3 | 4.1 | 4.4 |
| 5° C. | 9 | 4.2 | — | 4.5 | — | 4.3 |
| 5° C. | 12 | 4.5 | 4.4 | 4.3 | 4.3 | 4.3 |
| 5° C. | 18 | 4.6 | 4.5 | 4.7 | 4.5 | 4.5 |
| 5° C. | 24 | 4.7 | 4.6 | 4.6 | 4.6 | 4.7 |
| 5° C. | 36 | 4.5 | 4.6 | 4.7 | 4.5 | 4.6 |
| 25° C. | 0 | 4.1 | 4.0 | 3.9 | 3.8 | 4.1 |
| 25° C. | 1 | 4.3 | — | 4.3 | — | 4.7 |
| 25° C. | 3 | 5.0 | 5.4 | 5.0 | 4.8 | 5.2 |
| 25° C. | 6 | 5.1 | 5.4 | 5.1 | 5.0 | 5.2 |
| 25° C. | 9 | 5.2 | — | 5.2 | — | 4.7 |
| 25° C. | 12 | 5.5 | 5.7 | 5.5 | 5.1 | 5.4 |
| 40° C. | 0 | 4.1 | 4.0 | 3.9 | 3.8 | 4.1 |
| 40° C. | 1 | 5.2 | — | 5.0 | — | 5.3 |
| 40° C. | 3 | 6.3 | 6.0 | 6.0 | 5.9 | 6.0 |

### Results and Discussion

The measurements of the gliding force and break loose force show that all formulations are stable and the gliding and break loose forces do not significantly increase over time. Noteworthy, the formulations that do not comprise acetate (F1) or very low concentrations of acetate (F2) show higher forces, indicating that it is useful to add buffer, such as acetate, particularly when aiming at minimizing the required forces applied to a syringe.

### 8.4. Further Analytics and Results

In addition, further analytics were performed for the five formulations tested with the following results. Storage times and temperatures were as described above.
The IEC main peak, APG and BPG content remained constant over 24 and 36 months at 5° C. No differences between the formulations were observed regarding the main peak, APG and BGP.
The HIC main peak content remained constant over 24 months at 5° C. in a range of 96.8-97.2%, as well as 1.4-1.7% pre peak and 1.5-1.9% post peak. Over 36 months at 5° C. HIC main peak contents in a range of 96.3-97.2% were obtained, as well as 1.4-1.7% pre peak and 1.5-2.2% post peak. At 25° C. for up to 12 months storage time main peak contents between 94.3-97.1% were obtained, as well as 1.4-3.0% pre peak and 1.5-2.7% post peak. At 40° C. for up to 3 months storage time main peak contents between 92.0-97.1% were obtained, as well as 1.4-4.6% pre peak and 1.5-3.4% post peak. No differences between the formulations were observed regarding the main peak, pre peak and post peak.
The protein concentration remained essentially constant up to a storage time of 24 and 36 months at the different storage temperatures tested. Small deviations of protein concentration are due to analytical variations, leading to ranges of 147-155 mg/mL (24 months) and 147-157 mg/mL (36 months).
The dynamic viscosity remained essentially constant up to a storage time of 24 and 36 months at the different storage temperatures tested. The dynamic viscosity was in a range of 8.9-10.0 mPas.
The protein related particles and foreign particles remained essentially constantly low over the storage time at the different storage temperatures tested.

### 8.5. Summary of Results

The tested formulations show overall comparable high stability. Therefore, both acetate containing and buffer-free formulations are suitable for the formulations according to the present disclosure. In view of the required force to be applied to the syringes, solutions containing a buffer such as an acetate buffer have proven to be advantageous over buffer-free formulations. Moreover, in order to achieve an osmolality of 310 mOsm/kg, an acetate content of 10 mM has proven to be suitable considering the other compounds present in the formulation according to the present example.

### Example 9: Analysis of Further Excipients

### 1. Influence of the PS20 Content in Shaking Experiments

Formulations were prepared (see Table 60) wherein the PS20 (polysorbate 20) content was varied from 0, 0.05, 0.075, 0.1, 0.2, 0.3 to 0.5 mg/mL and analyzed during shaking times of 0, 1, 5, 7, 14 and 21 days.

TABLE 60
Composition of formulations.

| | PS20/ | | | |
|---|---|---|---|---|
| Formulation | mg/mL | Acetate | Trehalose | pH |
| F1 | 0.0 | 10 mM | 185 mM | 5.7 |
| F2 | 0.05 | | | |
| F3 | 0.075 | | | |
| F4 | 0.1 | | | |
| F5 | 0.2 | | | |
| F6 | 0.3 | | | |
| F7 | 0.5 | | | |

### 1.1. Preparation of Formulations

The formulations were prepared as described above. The formulations were packaged in 2R vials (1.0 mL) or pre-filled syringes (PFS, Neopak, 1.0 mL) for each formulation, as well as for the control and non-shaken formulations.

### 1.2. Analytics

Measurements of the samples were performed on day 0, 1, 5, 7, 14 and 21. Therefore, the total shaking duration was 21 days for both vial and PFS. The shaking was performed at room temperature (25° C.) at 200 U/min for vials (Orbital shaker) and the movement is adjusted to respective viscosity to ensure air bubble movement for PFS (Tilting shaker (Vari Mix Platform Rocker)). All samples were protected from light. The opalescence of the formulations was measured at the indicated measurement points. Further details on the utilized analysis methods are described below.

### 1.3. Results

### Measurement of the Opalescence

To measure the stability of the formulations containing varying amounts of PS20 and subjected to a shaking stress, the opalescence was measured at different time points. The results obtained by the opalescence measurements are shown below:

TABLE 61
Opalescence in FNU of the formulations having
varying amounts of PS20 in syringes.

| | | Shaking time, days, Storage / shaking condition | | | | | | |
|---|---|---|---|---|---|---|---|---|
| | | | 1 | 3 | 7 | 14 | 21 | |
| | PS20, | 0 | rocking | rocking | rocking | rocking | rocking | no |
| Formulation | g/l | initial | shaker | shaker | shaker | shaker | shaker | movement |
| F1_S | 0.00 | 8.18 | 8.26 | 8.48 | 11.00 | 15.20 | 19.95 | 7.13 |
| F2_S | 0.05 | 5.72 | 5.82 | 5.70 | 5.78 | 5.86 | 5.83 | 4.97 |
| F3_S | 0.08 | 5.90 | 5.95 | 5.68 | 8.53 | 6.13 | 5.67 | 6.49 |
| F4_S | 0.10 | 5.61 | 5.19 | 5.94 | 6.12 | 6.10 | 6.01 | 5.47 |
| F5_S | 0.20 | 6.32 | 5.93 | 6.60 | 6.12 | 6.33 | 6.30 | 5.12 |
| F6_S | 0.30 | 6.22 | 6.23 | 5.75 | 5.55 | 5.75 | 5.87 | 5.05 |
| F7_S | 0.50 | 6.10 | 5.77 | 5.61 | 6.01 | 7.33 | 5.78 | 5.32 |

TABLE 62
Opalescence in FNU of the formulations having varying amounts of PS20 in vials.

| | | Shaking time, days, Storage / shaking condition | | | | | | |
|---|---|---|---|---|---|---|---|---|
| | | | 1 | 3 | 7 | 14 | 21 | |
| | PS20, | 0 | horizontal | horizontal | horizontal | horizontal | horizontal | no |
| Formulation | g/l | Initial | shaker | shaker | shaker | shaker | shaker | movement |
| F1_V | 0.00 | 6.83 | 8.29 | 8.37 | 10.72 | 14.52 | 22.50 | 7.96 |
| F2_V | 0.05 | 6.70 | 5.70 | 6.10 | 6.45 | 5.91 | 5.72 | 5.46 |
| F3_V | 0.08 | 5.84 | 5.47 | 6.57 | 6.85 | 6.26 | 6.86 | 6.21 |
| F4_V | 0.10 | 5.67 | 5.10 | 6.24 | 6.39 | 5.75 | 6.28 | 5.81 |
| F5_V | 0.20 | 7.23 | 6.21 | 5.94 | 6.07 | 5.58 | 6.87 | 6.88 |
| F6_V | 0.30 | 6.60 | 5.20 | 5.89 | 5.36 | 5.78 | 5.61 | 5.66 |
| F7_V | 0.50 | 5.70 | 5.62 | 5.61 | 5.81 | 6.64 | 5.48 | 5.89 |

### 1.4. Summary of Results

The shaking study clearly revealed that formulations that do not comprise PS20 significantly increased in opalescence over the shaking time of 21 days. In contrast, all formulations that comprised PS20, i.e. even the lowest amount of 0.05 g/L, showed no increase in opalescence over time. The results substantiate the importance of a surfactant such as the non-ionic surfactant PS20 in formulations according to the present disclosure, in particular formulations comprising 150 mg/mL risankizumab.

### 2. Influence of the PS20 Content During Storage

The prepared formulations (see Table 60) were analyzed over different time points stored at three different temperatures (5° C., 25° C. and 40° C.).

### 2.1. Analytics

Measurements of the samples were performed at 1, 3, 6, 9, 12, 18, 24 and 36 months storage, as well as initially before storage. UP-SEC analysis was performed in order to determine the monomer, HMW and LMW contents. Moreover, sub-visible particle content, gliding force and break loose force were measured. Further details on the utilized analysis methods are described below.

### 2.2. Results

### 2.2.1. Measurement of the Monomer Content

The stability of the formulations was assessed by measuring the monomer content using the UP-SEC analysis. The results are shown below.

TABLE 63
UP-SEC-Monomer-measurements in % of formulations
comprising varying amounts of PS20.

| | Storage | | | | | | | |
|---|---|---|---|---|---|---|---|---|
| Storage | time, | | | | | | | |
| condition | months | F1 | F2 | F3 | F4 | F5 | F6 | F7 |
| initial | 0 | 97.9 | 97.9 | 97.9 | 97.9 | 97.8 | 97.8 | 97.8 |
| 5° C. | 3 | 97.5 | 97.5 | 97.6 | 97.5 | 97.5 | 97.5 | 97.5 |
| 5° C. | 6 | 97.3 | 97.3 | 97.3 | 97.3 | 97.3 | 97.3 | 97.2 |
| 5° C. | 9 | 97.1 | 97.1 | 97.1 | 97.1 | 97.1 | 97.1 | 97.1 |
| 5° C. | 12 | 97.2 | 97.2 | 97.2 | 97.2 | 97.2 | 97.2 | 97.2 |
| 5° C. | 18 | 97.4 | 97.3 | 97.3 | 97.5 | 97.4 | 97.4 | 97.4 |
| 5° C. | 24 | 97.1 | 97.2 | 97.2 | 97.1 | 97.2 | 97.1 | 97.2 |
| 5° C. | 36 | 96.8 | 96.9 | 96.9 | 96.9 | 97.0 | 97.0 | 97.0 |
| 25° C. | 1 | 97.2 | 97.2 | 97.2 | 97.2 | 97.2 | 97.2 | 97.2 |
| 25° C. | 3 | 96.2 | 96.2 | 96.3 | 96.2 | 96.2 | 96.3 | 96.2 |
| 25° C. | 6 | 95.2 | 95.3 | 95.4 | 95.4 | 95.4 | 95.4 | 95.4 |
| 25° C. | 9 | 94.2 | 94.4 | 94.5 | 94.5 | 94.5 | 94.5 | 94.5 |
| 25° C. | 12 | 93.5 | 93.8 | 93.9 | 93.9 | 94.0 | 94.0 | 93.9 |
| 40° C. | 1 | 94.9 | 94.9 | 94.9 | 94.9 | 94.9 | 94.8 | 94.8 |
| 40° C. | 3 | 89.6 | 90.1 | 90.4 | 90.3 | 90.4 | 90.4 | 89.7 |

### Results and Discussion

The monomer measurements show that the formulation is stable over a range of PS20 contents. Particular high monomer values were obtained for PS20 contents around 0.2 mg/mL.

### 2.2.2. Measurement of the HMW Content

The stability of the formulations was further assessed by measuring the HMN/W content, again using UP-SEC. The results are shown below.

TABLE 64
UP-SEC HMW measurements in % of formulations
comprising varying amounts of PS20.

| | Storage | | | | | | | |
|---|---|---|---|---|---|---|---|---|
| Storage | time, | | | | | | | |
| condition | months | F1 | F2 | F3 | F4 | F5 | F6 | F7 |
| initial | 0 | 1.3 | 1.4 | 1.4 | 1.4 | 1.4 | 1.4 | 1.4 |
| 5° C. | 3 | 1.7 | 1.7 | 1.7 | 1.7 | 1.7 | 1.7 | 1.7 |
| 5° C. | 6 | 1.9 | 1.9 | 1.9 | 1.9 | 1.9 | 1.9 | 1.9 |
| 5° C. | 9 | 1.8 | 1.8 | 1.8 | 1.8 | 1.8 | 1.8 | 1.8 |
| 5° C. | 12 | 1.0 | 1.0 | 1.0 | 1.0 | 1.0 | 1.0 | 1.0 |
| 5° C. | 18 | 1.7 | 1.8 | 1.8 | 1.7 | 1.7 | 1.7 | 1.7 |
| 5° C. | 24 | 2.1 | 2.1 | 2.0 | 2.1 | 2.1 | 2.1 | 2.1 |
| 5° C. | 36 | 2.3 | 2.2 | 2.2 | 2.2 | 2.2 | 2.2 | 2.2 |
| 25° C. | 1 | 1.9 | 1.9 | 1.9 | 1.9 | 1.9 | 1.9 | 1.9 |
| 25° C. | 3 | 2.4 | 2.4 | 2.4 | 2.4 | 2.4 | 2.4 | 2.5 |
| 25° C. | 6 | 2.9 | 2.8 | 2.8 | 2.8 | 2.8 | 2.8 | 2.8 |
| 25° C. | 9 | 3.0 | 2.9 | 2.8 | 2.8 | 2.8 | 2.8 | 2.8 |
| 25° C. | 12 | 3.2 | 3.1 | 3.1 | 3.1 | 3.0 | 3.0 | 3.1 |
| 40° C. | 1 | 2.9 | 2.9 | 2.9 | 2.9 | 2.9 | 2.9 | 3.0 |
| 40° C. | 3 | 5.1 | 4.9 | 4.7 | 4.7 | 4.7 | 4.7 | 5.3 |

### Results and Discussion

The HMVW content correlates with the monomer measurements. Overall, the tested formulations were stable over a range of PS20 contents. Particular low increases of the HMW contents were obtained for PS20 contents of 0.2 mg/mL. However, highest and lowest tested PS20 contents appear to have led to slightly higher HMW values.

### 2.2.3. Measurement of the LMW Content

Also the LMWV contents were measured using an UP-SEC and following results were obtained:

TABLE 65
UP-SEC-LMW-measurements in % of formulations
comprising varying amounts of PS20.

| | Storage | | | | | | | |
|---|---|---|---|---|---|---|---|---|
| Storage | time, | | | | | | | |
| condition | months | F1 | F2 | F3 | F4 | F5 | F6 | F7 |
| initial | 0 | 0.8 | 0.8 | 0.8 | 0.8 | 0.8 | 0.8 | 0.8 |
| 5° C. | 3 | 0.8 | 0.8 | 0.8 | 0.8 | 0.8 | 0.8 | 0.8 |
| 5° C. | 6 | 0.8 | 0.8 | 0.8 | 0.8 | 0.8 | 0.8 | 0.8 |
| 5° C. | 9 | 1.1 | 1.1 | 1.1 | 1.1 | 1.1 | 1.1 | 1.1 |
| 5° C. | 12 | 1.9 | 1.8 | 1.8 | 1.8 | 1.8 | 1.8 | 1.8 |
| 5° C. | 18 | 0.8 | 1.0 | 1.0 | 0.8 | 0.9 | 0.9 | 0.9 |
| 5° C. | 24 | 0.8 | 0.8 | 0.8 | 0.8 | 0.8 | 0.8 | 0.8 |
| 5° C. | 36 | 0.9 | 0.9 | 0.9 | 0.9 | 0.9 | 0.9 | 0.9 |
| 25° C. | 1 | 1.0 | 1.0 | 1.0 | 1.0 | 1.0 | 1.0 | 0.9 |
| 25° C. | 3 | 1.4 | 1.3 | 1.3 | 1.4 | 1.4 | 1.3 | 1.3 |
| 25° C. | 6 | 1.9 | 1.9 | 1.8 | 1.8 | 1.8 | 1.8 | 1.8 |
| 25° C. | 9 | 2.8 | 2.7 | 2.7 | 2.7 | 2.7 | 2.7 | 2.7 |
| 25° C. | 12 | 3.3 | 3.1 | 3.1 | 3.1 | 3.0 | 3.0 | 3.0 |
| 40° C. | 1 | 2.2 | 2.3 | 2.2 | 2.2 | 2.2 | 2.3 | 2.3 |
| 40° C. | 3 | 5.3 | 5.1 | 5.0 | 5.0 | 4.9 | 4.9 | 5.0 |

### Results and Discussion

The LMW content correlates with the monomer measurements. Overall, the tested formulations were stable over a range of PS20 contents. Particular low increases of the LMW contents were obtained for PS 20 contents of 0.2 mg/mL. The lowest tested PS20 content (see F1) appears to have led to slightly higher LMW values.

### 2.2.4. Measurement of the Opalescence

Moreover, the opalescence was measured for the formulations containing the varying amounts of PS20. The results are depicted below.

TABLE 66
Opalescence measurements in FNU of formulations
comprising varying amounts of PS20.

| | Storage | | | | | | | |
|---|---|---|---|---|---|---|---|---|
| Storage | time, | | | | | | | |
| condition | months | F1 | F2 | F3 | F4 | F5 | F6 | F7 |
| initial | 0 | 8 | 6 | 6 | 6 | 6 | 6 | 6 |
| 5° C. | 3 | 7 | 6 | 6 | 6 | 7 | 6 | 6 |
| 5° C. | 6 | 6 | 6 | 6 | 6 | 7 | 6 | 6 |
| 5° C. | 9 | 12 | 5 | 7 | 6 | 6 | 7 | 8 |
| 5° C. | 12 | 11 | 10 | 8 | 7 | 7 | 6 | 7 |
| 5° C. | 18 | 19 | 9 | 12 | 8 | 7 | 7 | 6 |
| 5° C. | 24 | 13 | 6 | 7 | 7 | 7 | 7 | 7 |
| 5° C. | 36 | 12 | 6 | 7 | 7 | 7 | 7 | 7 |
| 25° C. | 1 | 6 | 6 | 5 | 6 | 5 | 6 | 6 |
| 25° C. | 3 | 6 | 6 | 6 | 6 | 6 | 6 | 6 |
| 25° C. | 6 | 6 | 6 | 6 | 6 | 7 | 7 | 7 |
| 25° C. | 9 | 6 | 6 | 6 | 7 | 6 | 7 | 8 |
| 25° C. | 12 | 7 | 7 | 7 | 7 | 8 | 7 | 9 |
| 40° C. | 1 | 7 | 6 | 6 | 6 | 6 | 7 | 6 |
| 40° C. | 3 | 7 | 6 | 6 | 7 | 7 | 7 | 8 |

### Results and Discussion

The measurements show that at higher temperatures of 25 and 40° C. all formulations led to no increase in opalescence. However, at a temperature of 5° C. and later storage time points (e.g. 18, 24 and 36 months), formulation F1 (no PS20) showed an increase in opalescence. Hence, incorporating a surfactant such as the non-ionic surfactant PS20 is advantageous.

### 2.2.5. Measurement of the Sub-Visible Particle Content

The formulations were analyzed in regard to their sub-visible particle content (≥2 μm, ≥10 μm and ≥25 μm) over 24 and 36 months stored at 5° C.

TABLE 67
Measurement of the sub-visible particle content of particles with
a size of ≥2, ≥10 and ≥25 μm stored for up to 24 and
36 months at 5° C. of formulations comprising varying amounts of PS20.

| Treat- | Particle | Number of measured particles | | | | | | |
|---|---|---|---|---|---|---|---|---|
| ment | size | F1 | F2 | F3 | F4 | F5 | F6 | F7 |
| Initial | ≥2 μm | 58318 | 1876 | 4832 | 3607 | 7780 | 5121 | 6578 |
| | ≥10 μm | 6061 | 70 | 99 | 88 | 312 | 111 | 278 |
| | ≥25 μm | 183 | 9 | 5 | 2 | 31 | 9 | 26 |
| 5° C., 3 | ≥2 μm | 41237 | 1442 | 4559 | 9475 | 14173 | 10810 | 4201 |
| months | ≥10 μm | 3650 | 48 | 170 | 225 | 177 | 224 | 94 |
| | ≥25 μm | 196 | 2 | 6 | 15 | 1 | 3 | 1 |
| 5° C., 6 | ≥2 μm | 51615 | 4949 | 3580 | 5282 | 11069 | 16838 | 18125 |
| months | ≥10 μm | 3501 | 38 | 48 | 45 | 71 | 105 | 131 |
| | ≥25 μm | 29 | 0 | 2 | 0 | 1 | 6 | 1 |
| 5° C., 9 | ≥2 μm | 70311 | 11911 | 6453 | 6982 | 35316 | 6358 | 10660 |
| months | ≥10 μm | 5195 | 42 | 17 | 60 | 60 | 33 | 79 |
| | ≥25 μm | 122 | 1 | 0 | 2 | 0 | 2 | 9 |
| 5° C., 12 | ≥2 μm | 58322 | 16911 | 9064 | 28517 | 7761 | 15372 | 41782 |
| months | ≥10 μm | 4529 | 121 | 183 | 360 | 141 | 132 | 295 |
| | ≥25 μm | 184 | 2 | 7 | 7 | 1 | 0 | 6 |
| 5° C., 18 | ≥2 μm | 28849 | 4496 | 5760 | 9250 | 7025 | 18772 | 20556 |
| months | ≥10 μm | 2939 | 79 | 84 | 123 | 56 | 181 | 232 |
| | ≥25 μm | 181 | 2 | 6 | 1 | 3 | 2 | 6 |
| 5° C., 24 | ≥2 μm | 61469 | 5862 | 9427 | 7969 | 17688 | 37040 | 3688 |
| months | ≥10 μm | 5407 | 84 | 163 | 197 | 355 | 290 | 148 |
| | ≥25 μm | 66 | 1 | 9 | 9 | 6 | 3 | 5 |
| 5° C., 36 | ≥2 μm | 17157 | 4306 | 7984 | 12335 | 16377 | 21911 | 14799 |
| months | ≥10 μm | 2255 | 49 | 54 | 146 | 172 | 136 | 65 |
| | ≥25 μm | 257 | 1 | 2 | 8 | 5 | 2 | 3 |

The measurements of the sub-visible particle content show that all formulations are stable at 5° C. for up to 24 and 36 months. Only formulations without PS20 (F1) appeared to result in some particle formation, substantiating that it is advantageous to add a surfactant such as the non-ionic surfactant PS20 to the formulations according to the present disclosure.

### 2.2.6. Measurement of the Gliding Force and Break Loose Force

The maximal and average gliding force as well as the break loose force were measured for the formulations comprising varying amounts of PS20. The results of the measurements are shown below.

TABLE 68
Maximal gliding force in N. The formulations
comprised varying amounts of PS20.

| | Storage | | | | | | | |
|---|---|---|---|---|---|---|---|---|
| Storage | time, | | | | | | | |
| condition | months | F1 | F2 | F3 | F4 | F5 | F6 | F7 |
| initial | 0 | 7.2 | 7.1 | 6.2 | 6.5 | 6.4 | 6.3 | 6.9 |
| 5° C. | 3 | 7.8 | 7.4 | 8.1 | 6.8 | 6.8 | 6.5 | 6.4 |
| 5° C. | 6 | 7.7 | 7.0 | 6.7 | 6.6 | 6.1 | 6.5 | 6.7 |
| 5° C. | 9 | 8.7 | 6.4 | 7.4 | 7.6 | 6.7 | 6.6 | 8.2 |
| 5° C. | 12 | 7.1 | 7.5 | 6.7 | 7.2 | 5.9 | 6.7 | 7.3 |
| 5° C. | 18 | 8.9 | 7.4 | 6.1 | 6.9 | 6.1 | 7.4 | 7.1 |
| 5° C. | 24 | 8.1 | 6.2 | 7.6 | 8.0 | 7.2 | 6.7 | 7.1 |
| 5° C. | 36 | 9.2 | 8.5 | 9.1 | 7.0 | 7.5 | 6.7 | 7.2 |
| 25° C. | 1 | 7.4 | 6.5 | 6.4 | 6.8 | 6.6 | 6.1 | 6.2 |
| 25° C. | 3 | 8.1 | 7.0 | 7.4 | 7.7 | 7.0 | 7.7 | 7.7 |
| 25° C. | 6 | 8.1 | 7.3 | 9.4 | 8.9 | 9.9 | 9.6 | 10.8 |
| 25° C. | 9 | 7.2 | 11.9 | 10.2 | 9.8 | 11.2 | 11.3 | 12.6 |
| 25° C. | 12 | 8.4 | 11.1 | 12.0 | 13.6 | 12.8 | 15.1 | 16.1 |
| 40° C. | 1 | 7.7 | 8.1 | 9.2 | 11.2 | 8.7 | 9.5 | 8.6 |

TABLE 69
Average gliding force in N. The formulations
comprised varying amounts of PS20.

| | Storage | | | | | | | |
|---|---|---|---|---|---|---|---|---|
| Storage | time, | | | | | | | |
| condition | months | F1 | F2 | F3 | F4 | F5 | F6 | F7 |
| initial | 0 | 6.5 | 6.5 | 5.8 | 6.1 | 5.9 | 5.9 | 6.4 |
| 5° C. | 3 | 7.1 | 6.7 | 7.3 | 6.3 | 6.2 | 6.0 | 6.0 |
| 5° C. | 6 | 6.8 | 6.5 | 6.3 | 6.2 | 5.7 | 6.1 | 6.2 |
| 5° C. | 9 | 7.5 | 6.1 | 6.7 | 6.9 | 6.3 | 6.1 | 7.2 |
| 5° C. | 12 | 6.4 | 6.8 | 6.4 | 6.8 | 5.7 | 6.2 | 6.7 |
| 5° C. | 18 | 7.9 | 7.0 | 5.8 | 6.4 | 5.8 | 6.8 | 6.6 |
| 5° C. | 24 | 7.2 | 5.9 | 7.0 | 7.3 | 6.7 | 6.4 | 6.6 |
| 5° C. | 36 | 7.8 | 7.7 | 8.2 | 6.6 | 6.8 | 6.4 | 6.6 |
| 25° C. | 1 | 6.7 | 6.0 | 6.0 | 6.4 | 6.2 | 5.8 | 5.8 |
| 25° C. | 3 | 7.4 | 6.5 | 6.9 | 7.2 | 6.3 | 7.0 | 7.2 |
| 25° C. | 6 | 7.3 | 6.9 | 8.4 | 7.8 | 8.0 | 8.3 | 9.2 |
| 25° C. | 9 | 6.4 | 10.0 | 8.5 | 8.6 | 9.1 | 9.5 | 9.9 |
| 25° C. | 12 | 7.7 | 9.6 | 10.0 | 11.2 | 10.5 | 12.1 | 12.4 |
| 40° C. | 1 | 6.9 | 7.0 | 7.9 | 9.2 | 7.0 | 8.0 | 7.5 |

TABLE 70
Break loose force in N. The formulations
comprised varying amounts of PS20.

| | Storage | | | | | | | |
|---|---|---|---|---|---|---|---|---|
| Storage | time, | | | | | | | |
| condition | months | F1 | F2 | F3 | F4 | F5 | F6 | F7 |
| initial | 0 | 4.1 | 4.3 | 4.2 | 4.1 | 4.2 | 4.3 | 4.1 |
| 5° C. | 3 | 4.7 | 4.6 | 4.8 | 4.8 | 4.6 | 4.7 | 4.7 |
| 5° C. | 6 | 4.6 | 4.3 | 4.5 | 4.5 | 4.5 | 4.5 | 4.7 |
| 5° C. | 9 | 3.8 | 4.1 | 4.0 | 4.1 | 3.9 | 4.2 | 4.2 |
| 5° C. | 12 | 4.5 | 4.5 | 4.5 | 4.7 | 4.7 | 4.3 | 4.5 |
| 5° C. | 18 | 4.6 | 4.9 | 4.8 | 4.6 | 4.7 | 4.6 | 4.7 |
| 5° C. | 24 | 5.0 | 5.1 | 4.8 | 4.7 | 4.8 | 4.6 | 4.8 |
| 5° C. | 36 | 4.2 | 4.3 | 4.3 | 4.0 | 4.0 | 4.5 | 4.6 |
| 25° C. | 1 | 4.8 | 4.8 | 4.6 | 4.7 | 4.7 | 4.4 | 4.7 |
| 25° C. | 3 | 5.1 | 5.4 | 5.3 | 5.5 | 5.4 | 5.2 | 5.4 |
| 25° C. | 6 | 5.1 | 5.1 | 5.1 | 5.3 | 4.9 | 5.1 | 5.3 |
| 25° C. | 9 | 4.4 | 4.9 | 4.4 | 5.0 | 5.0 | 4.9 | 5.0 |
| 25° C. | 12 | 5.4 | 5.4 | 5.4 | 5.7 | 5.3 | 5.6 | 5.3 |
| 40° C. | 1 | 5.0 | 5.0 | 5.0 | 5.2 | 5.1 | 4.9 | 5.0 |
| 40° C. | 3 | 5.9 | 5.8 | 6.1 | 6.3 | 6.0 | 6.0 | 6.4 |

### Results and Discussion

The measurements of the gliding force revealed relatively high gliding forces for high contents of PS20 of 0.5 g/L in comparison to the lower contents. While a content of 0 g/L showed lowest increase in gliding force, an intermediate concentration of 0.2 g/L showed a good compromise between high and low gliding force. Noteworthy, essentially no differences between the formulations in respect to the break loose force were observed.

### 2.3. Further Analytics and Results

In addition, further analytics were performed for the seven formulations tested with the following results. Storage times and temperatures were as described above.
The IEC main peak, APG and BPG content remained constant over 24 and 36 months at 5° C. No differences between the formulations were observed regarding the main peak, APG and BGP.
The HIC main peak content remained constant over 24 months at 5° C. in a range of 96.5-97.3%, as well as 1.4-1.7% pre peak and 1.4-1.9% post peak. Over 36 months at 5° C. HIC main peak contents in a range of 95.9-97.3% were obtained, as well as 1.4-1.7% pre peak and 1.4-2.4% post peak. At 25° C. for up to 12 months storage time main peak contents between 93.9-96.8% were obtained, as well as 1.4-3.0% pre peak and 1.7-2.7% post peak. At 40° C. for up to 3 months storage time main peak contents between 90.3-95.2% were obtained, as well as 2.5-6.0% pre peak and 2.3-3.7% post peak. No differences between the formulations were observed regarding the main peak, pre peak and post peak.
The specific binding activity remained essentially constant up to a storage time of 24 and 36 months at the different storage temperatures tested. The specific binding activity was in a range of 97-101%.
The protein concentration remained essentially constant up to a storage time of 24 and 36 months at the different storage temperatures tested. Small deviations of protein concentration are due to analytical variations, leading to ranges of 147-155 mg/mL (24 months) and 147-159 mg/mL (36 months).
The pH value remained essentially constant up to a storage time of 24 and 36 months at the different storage temperatures tested. The pH was in a range of 5.7-5.9.
The osmolality remained essentially constant up to a storage time of 24 and 36 months at the different storage temperatures tested. The tested values ranged from 305-322 mOsm/kg.
The dynamic viscosity remained essentially constant up to a storage time of 24 and 36 months at the different storage temperatures tested. The dynamic viscosity was in a range of 9.2-11.0 mPas.
The protein related particles and foreign particles remained essentially constantly low over the storage time at the different storage temperatures tested.

### 2.4. Summary of Results

In summary, the tested formulations were stable over long term storage up to 24 and 36 months at temperatures ranging from 5° C. up to 40° C. In particular, formulations comprising a surfactant such as PS20 were found to be stable, whereas formulation lacking PS20 showed some formation of sub-visible particles and an increase in opalescence. Also the LMW content was slightly increased for formulations lacking PS20. A particularly suitable content of a surfactant such as the non-ionic surfactant PS20 appeared to be 0.2 g/L under the tested conditions.

### 3. Variation of the Trehalose Content

In this example the trehalose concentration was varied from 145, 165, 185, 205 to 225 mM and analyzed over different time points stored at three different temperatures (5° C., 25° C. and 40° C.). The prepared formulations are shown in Table 71.

TABLE 71
Composition of formulations.

| | Trehalose/ | PS20/ | | |
|---|---|---|---|---|
| Formulation | mM | mg/mL | Acetate/mM | pH |
| F1 | 145 | 0.2 | 10 | 5.7 |
| F2 | 165 | | | |
| F3 | 185 | | | |
| F4 | 205 | | | |
| F5 | 225 | | | |

### 3.1. Analytics

Measurements of the samples were performed at 1, 3, 6, 9, 12, 18, 24 and 36 months storage, as well as initially before storage. Further details on the utilized analysis methods are described below.

### 3.2. Results

### 3.2.1. Measurement of the Monomer Content

The stability of the formulations comprising varying trehalose amounts was assessed by measuring the monomer content using an UP-SEC analysis, revealing the results shown below.

TABLE 72
UP-SEC-Monomer-measurements in % of formulations
comprising different amounts of trehalose.

| | Storage | | | | | |
|---|---|---|---|---|---|---|
| Storage | time, | | | | | |
| condition | months | F1 | F2 | F3 | F4 | F5 |
| 5° C. | 0 | 98.1 | 98.0 | 98.0 | 98.1 | 98.0 |
| 5° C. | 3 | 97.7 | 97.7 | 97.7 | 97.7 | 97.6 |
| 5° C. | 6 | 97.6 | 97.5 | 97.5 | 97.5 | 97.5 |
| 5° C. | 9 | 97.2 | — | 97.2 | — | 97.1 |
| 5° C. | 12 | 97.2 | 97.3 | 97.2 | 97.3 | 97.2 |
| 5° C. | 18 | 97.5 | 97.5 | 97.4 | 97.4 | 97.3 |
| 5° C. | 24 | 97.2 | 97.1 | 97.2 | 97.2 | 97.1 |
| 5° C. | 36 | 97.0 | 97.0 | 97.0 | 97.0 | 96.9 |
| 25° C. | 0 | 98.1 | 98.0 | 98.0 | 98.1 | 98.0 |
| 25° C. | 1 | 97.2 | — | 97.3 | — | 97.2 |
| 25° C. | 3 | 96.5 | 96.5 | 96.4 | 96.4 | 96.3 |
| 25° C. | 6 | 95.7 | 95.6 | 95.6 | 95.6 | 95.6 |
| 25° C. | 9 | 94.6 | — | 94.6 | — | 94.6 |
| 25° C. | 12 | 94.1 | 94.1 | 94.1 | 94.1 | 94.1 |
| 40° C. | 0 | 98.1 | 98.0 | 98.0 | 98.1 | 98.0 |
| 40° C. | 1 | 95.0 | — | 95.0 | — | 95.0 |
| 40° C. | 3 | 90.7 | 90.8 | 90.8 | 90.7 | 90.8 |

### Results and Discussion

The monomer measurements show that the formulation is stable over a range of trehalose contents, indicating stability over a range of trehalose contents.

### 3.2.2. Measurement of the HMW Content

The HMW contents of the formulations were measured using UP-SEC. The results of the analysis are shown below.

TABLE 73
UP-SEC HMW measurements in % of formulations
comprising different amounts of trehalose.

| | Storage | | | | | |
|---|---|---|---|---|---|---|
| Storage | time, | | | | | |
| condition | months | F1 | F2 | F3 | F4 | F5 |
| 5° C. | 0 | 1.1 | 1.1 | 1.1 | 1.1 | 1.2 |
| 5° C. | 3 | 1.4 | 1.4 | 1.5 | 1.4 | 1.5 |
| 5° C. | 6 | 1.5 | 1.6 | 1.6 | 1.6 | 1.6 |
| 5° C. | 9 | 1.7 | — | 1.7 | — | 1.8 |
| 5° C. | 12 | 1.8 | 1.8 | 1.8 | 1.8 | 1.9 |
| 5° C. | 18 | 1.7 | 1.7 | 1.7 | 1.7 | 1.8 |
| 5° C. | 24 | 2.0 | 2.0 | 2.0 | 2.0 | 2.1 |
| 5° C. | 36 | 2.1 | 2.1 | 2.2 | 2.2 | 2.2 |
| 25° C. | 0 | 1.1 | 1.1 | 1.1 | 1.1 | 1.2 |
| 25° C. | 1 | 1.7 | — | 1.7 | — | 1.7 |
| 25° C. | 3 | 2.2 | 2.2 | 2.2 | 2.2 | 2.3 |
| 25° C. | 6 | 2.4 | 2.4 | 2.5 | 2.4 | 2.5 |
| 25° C. | 9 | 2.7 | — | 2.7 | — | 2.8 |
| 25° C. | 12 | 2.9 | 3.0 | 3.0 | 3.0 | 3.0 |
| 40° C. | 0 | 1.1 | 1.1 | 1.1 | 1.1 | 1.2 |
| 40° C. | 1 | 2.8 | — | 2.7 | — | 2.8 |
| 40° C. | 3 | 4.4 | 4.3 | 4.3 | 4.3 | 4.4 |

### Results and Discussion

The HMW content measurements show that the formulation is stable over a range of trehalose contents.

### 3.2.3. Measurement of the LMWV Content

Also the LMWV content was measured via UP-SEC for the formulations comprising varying amounts of trehalose. The results are shown below.

TABLE 74
UP-SEC-LMW-measurements in % of formulations
comprising different amounts of trehalose.

| | Storage | | | | | |
|---|---|---|---|---|---|---|
| Storage | time, | | | | | |
| condition | months | F1 | F2 | F3 | F4 | F5 |
| 5° C. | 0 | 0.9 | 0.9 | 0.9 | 0.9 | 0.9 |
| 5° C. | 3 | 0.9 | 0.9 | 0.9 | 0.9 | 0.9 |
| 5° C. | 6 | 0.9 | 0.9 | 0.9 | 0.9 | 0.9 |
| 5° C. | 9 | 1.1 | — | 1.1 | — | 1.1 |
| 5° C. | 12 | 1.0 | 1.0 | 1.0 | 1.0 | 1.0 |
| 5° C. | 18 | 0.9 | 0.9 | 0.9 | 0.9 | 0.9 |
| 5° C. | 24 | 0.8 | 0.8 | 0.8 | 0.8 | 0.8 |
| 5° C. | 36 | 0.9 | 0.8 | 0.9 | 0.9 | 0.9 |
| 25° C. | 0 | 0.9 | 0.9 | 0.9 | 0.9 | 0.9 |
| 25° C. | 1 | 1.1 | — | 1.0 | — | 1.0 |
| 25° C. | 3 | 1.4 | 1.4 | 1.4 | 1.4 | 1.4 |
| 25° C. | 6 | 1.9 | 1.9 | 1.9 | 1.9 | 1.9 |
| 25° C. | 9 | 2.6 | — | 2.6 | — | 2.6 |
| 25° C. | 12 | 2.9 | 2.9 | 2.9 | 3.0 | 2.9 |
| 40° C. | 0 | 0.9 | 0.9 | 0.9 | 0.9 | 0.9 |
| 40° C. | 1 | 2.3 | — | 2.3 | — | 2.3 |
| 40° C. | 3 | 4.9 | 4.9 | 4.9 | 4.9 | 4.9 |

### Results and Discussion

The LMW measurements show that the formulation is stable over a range of trehalose contents.

### 3.2.4. Measurement of Binding Activity

The binding activity of the risankizumab comprised in the formulations according to the present disclosure was measured. The measurements of antigen binding show high binding activity to IL-23 for all tested formulations ranging from 92-122% binding activity and 96-100% specific binding activity. These results support the advantageous stability of the tested formulations and indicate that trehalose containing formulations at various concentrations are applicable according to the present disclosure.

### 3.2.5. Measurement of the Osmolality

The osmolality was measured in order to assure that the tested formulations have a suitable osmolality for injection. The results are shown below:

TABLE 75
Measured osmolality in mOsm/kg of the formulations
comprising varying amounts of trehalose.

| | Storage | | | | | |
|---|---|---|---|---|---|---|
| Storage | time, | | | | | |
| condition | months | F1 | F2 | F3 | F4 | F5 |
| 5° C. | 0 | 246 | 274 | 309 | 337 | 376 |
| 5° C. | 3 | 247 | 277 | 307 | 340 | 378 |
| 5° C. | 6 | 247 | 275 | 309 | 338 | 375 |
| 5° C. | 9 | 251 | — | 310 | — | 380 |
| 5° C. | 12 | 245 | 274 | 305 | 335 | 375 |
| 5° C. | 18 | 248 | 271 | 303 | 340 | 370 |
| 5° C. | 24 | 248 | 277 | 308 | 339 | 376 |
| 5° C. | 36 | 248 | 275 | 307 | 338 | 374 |
| 25° C. | 0 | 246 | 274 | 309 | 337 | 376 |
| 25° C. | 1 | 248 | — | 307 | — | 373 |
| 25° C. | 3 | 252 | 287 | 311 | 338 | 378 |
| 25° C. | 6 | 246 | 276 | 310 | 335 | 371 |
| 25° C. | 9 | 253 | — | 310 | — | 380 |
| 25° C. | 12 | 241 | 278 | 302 | 336 | 377 |
| 40° C. | 0 | 246 | 274 | 309 | 337 | 376 |
| 40° C. | 1 | 245 | | 310 | | 373 |
| 40° C. | 3 | 249 | 278 | 312 | 340 | 379 |

### Results and Discussion

The osmolality values range from around 245 to 380 mOsm/kg for trehalose concentrations from 145 to 225 mM. As an optimal osmolality is around 310 mOsm/kg, it can be advantageous to provide formulations having such osmolality. This can for instance be achieved using a trehalose concentration of 185 mM in combination with the formulation according to the present example.

### 3.3. Further Analytics and Results

In addition, further analytics were performed for the five formulations tested (storage times and temperatures were as described above).
The IEC main peak, APG and BPG content remained constant over 24 and 36 months at 5° C. No differences between the formulations were observed regarding the main peak, APG and BGP.
The HIC main peak content remained constant over 24 months at 5° C. in a range of 96.4-97.4%, as well as 1.4-1.8% pre peak and 1.2-2.0% post peak. Over 36 months at 5° C. HIC main peak contents in a range of 96.0-97.4% were obtained, as well as 1.4-1.8% pre peak and 1.2-2.3% post peak. At 25° C. for up to 12 months storage time main peak contents between 94.2-97.4% were obtained, as well as 1.4-3.0% pre peak and 1.2-2.8% post peak. At 40° C. for up to 3 months storage time main peak contents between 90.3-97.4% were obtained, as well as 1.4-5.9% pre peak and 1.2-3.7% post peak. No differences between the formulations were observed regarding the main peak, pre peak and post peak.
The protein concentration remained essentially constant up to a storage time of 24 and 36 months at the different storage temperatures tested. Small deviations of protein concentration are due to analytical variations, leading to ranges of 145-153 mg/mL (24 months) and 148-158 mg/mL (36 months).
The pH value remained essentially constant up to a storage time of 24 and 36 months at the different storage temperatures tested. The pH was in a range of 5.7-5.9.
The opalescence remained essentially constant up to a storage time of 24 and 36 months at the different storage temperatures tested. The opalescence was in a range of 5-9 FNU.
The dynamic viscosity remained essentially constant up to a storage time of 24 and 36 months at the different storage temperatures tested. The dynamic viscosity was in a range of 8.9-10.3 mPas.
The gliding forces remained constant over 24 months at 5° C. in a range of 6.5-7.7 N (maximum) and 5.8-7.4 N (average), as well 3.9-5.0 N for break loose force. Over 36 months at 5° C. gliding forces remained constant in a range of 6.1-8.5 N (maximum) and 5.8-7.7 N (average), as well 3.9-5.0 N for break loose force. At 25° C. for up to 12 months storage time gliding forces range between 6.7-15.7 N (maximum) and 6.2-12.4 N (average), as well as 3.9-5.6 N for the break loose force. At 40° C. for up to 3 months storage time gliding forces range between 8.7-23.1 N (maximum) and 7.3-16.4 N (average), as well as 5.1-6.6 N for the break loose force.
The protein related particles and foreign particles remained essentially constantly low over the storage time at the different storage temperatures tested.

### 3.4. Summary of the Results

In summary, all tested formulations were stable, substantiating that the trehalose concentration can be varied while maintaining a high stability. Therefore, the indicated trehalose concentrations can be flexibly applied in order to produce stable protein formulations of 150 mg/mL risankizumab.

### Example 10: Analysis of Further Parameters of a Specific Formulation

In view of the results of the prior examples, a particularly suitable formulation comprises the following compounds:
150 mg/mL risankizumab,
10 mM acetate buffer,
185 mM trehalose, and
0.2 mg/mL PS20;
   wherein the formulation has a pH of 5.7.

The appearance of this formulation was clear to slightly opalescent and essentially free of foreign particles. The osmolality was about 310 mOsm/kg. The formulation is particularly suitable for injection, especially for subcutaneous injection. Moreover, a viscosity of about 9.6 mPas was measured, making it suitable for injection using a syringe. The conductivity at 20° C. was about 1.53 mS/cm, the density at 20° C. was about 1.067 g/cm³ and the density at 4° C. was about 1.071 g/cm³.

This 150 mg/ml risankizumab formulation may be provided as follows:

| | | | | In an embodiment wherein this |
|---|---|---|---|---|
| | Concen- | | | formulation is provided in a syringe (V = |
| | tration | Concen- | | 1 ml) the Nominal Amount |
| Ingredient | [mmol/L] | tration [g/1] | Function | [mg/syringe] is as follows |
| Risankizumab | 1.00 | 150 | Drug | 150 |
| | | | substance | |
| Sodium acetate | 9.10 | 1.24 | Buffer | 1.24 |
| trihydrate | | | | |
| Acetic acid | 0.900 | 0.0540 | Buffer | 0.0540 |
| Trehalose | 185 | 70.0 | Adjustment | 70.0 |
| dihydrate | | | tonicity | |
| Polysorbate 20 | 0.163 | 0.200 | Surfactant | 0.200 |

### List of Abbreviations

| Abbreviation | Full form |
|---|---|
| APG | Acidic peak group |
| AUC | Area under curve |
| BPG | Basic peak group |
| CGE | Capillary gel electrophoresis |
| FNU | Formazine nephelometric units |
| F/T | Freeze/thaw |
| HIC | Hydrophobic interaction chromatography |
| HMW | High molecular weight |
| HP-SEC | High pressure size exclusion chromatography |
| IEC | Ion exchange chromatography |
| IL-23 | Interleukin-23 |
| LMW | Low molecular weight |
| MFI | Micro flow imaging |
| mOsm/kg | Milliosmole/kilogram |
| mPas | Millipascal second |
| mS/cm | Millisiemens per centimeter |
| PS20 | Polysorbate 20 |
| RALS | Right-angle light scattering |
| r.h. | Relative humidity |
| rhIL-23 | Recombinant human interleukin-23 |
| SEC | Size exclusion chromatography |
| SPR | Surface plasmon resonance |
| STP | Sampling time point |
| SVP | Sub-visible particles |
| UF/DF | Ultrafiltration/Diafiltration |
| U/min | Revolutions per minute |
| UP-SEC | Ultra-performance size exclusion chromatography |
| WCX | Weak cation exchange chromatography |

What is claimed is:

1. A method for treating psoriasis, said method comprising administering Antibody A to a patient previously treated with a first agent, wherein the PASI score or sPGA score of said patient improves after the administration of Antibody A.

2. The method of claim 1, wherein said patient reaches PASI 90 after the administration of Antibody A.

3. The method of claim 1, wherein said patient reaches PASI 100 after the administration of Antibody A.

4. The method of claim 1, wherein said patient reaches a sPGA score of clear (score of 0) or almost clear (score of 1) after the administration of Antibody A.

5. The method of claim 1, wherein said first agent is a biological drug, for example a monoclonal antibody.

6. The method of claim 1, wherein said first agent is ustekinumab or adalimumab.

7. A stable liquid aqueous pharmaceutical formulation, comprising:
a) 150 mg/mL risankizumab;
b) a surfactant; and
c) arginine;
wherein:
the pH of the formulation is from about 5.0 to about 7.5.

8. The formulation of claim 7, wherein the pH of the formulation is from about 5.5 to about 5.9.

9. A pre-filled syringe comprising the formulation of claim 7.

10. The pre-filled syringe of claim 9, wherein the pH of the formulation is from about 5.5 to about 5.9.

11. A method for treating psoriasis, said method comprising administering a stable liquid aqueous pharmaceutical formulation to a patient previously treated with a first agent, wherein the stable liquid aqueous pharmaceutical formulation comprises:
a) 150 mg/mL of Antibody A;
b) water;
c) a surfactant; and
c) arginine;
wherein the PASI score or sPGA score of said patient improves after the administration of the formulation.

12. The method of claim 11, wherein said patient reaches PASI 90 after the administration of the formulation.

13. The method of claim 11, wherein said patient reaches PASI 100 after the administration of the formulation.

14. The method of claim 11, wherein said patient reaches a sPGA score of clear (score of 0) or almost clear (score of 1) after the administration of the formulation.

15. The method of claim 11, wherein said first agent is a biological drug, for example a monoclonal antibody.

16. The method of claim 11, wherein said first agent is ustekinumab or adalimumab.

17. The method of claim 11, wherein the pH of the formulation is from about 5.0 to about 7.5.

18. The method of claim 11, wherein the pH of the formulation is from about 5.5 to about 5.9.

19. The method of claim 11, wherein the formulation is delivered to the patient subcutaneously using a pre-filled syringe.

20. The method of claim 17, wherein the formulation is delivered to the patient subcutaneously using a pre-filled syringe.
